(19) United States
(12) Patent Application Publication
(10) Pub. No.: US 2025/0099383 A1
(43) Pub. Date: Mar. 27, 2025

(54) COMPOSITIONS AND METHODS FOR DELIVERY OF NUCLEIC ACIDS

(71) Applicants: Poseida Therapeutics, Inc., San Diego, CA (US); University of San Diego, San Diego, CA (US)

(72) Inventors: Nicholas Roman CAM, San Diego, CA (US); Alex SCHUDEL, San Diego, CA (US); Neha Nitin PARAYATH, San Diego, CA (US); Peter M. IOVINE, San Diego, CA (US); Gopi Nath VEMURI, San Diego, CA (US); Khalid A. HAJJ, San Diego, CA (US); Oscar ALVAREZ, San Diego, CA (US)

(21) Appl. No.: 18/730,413

(22) PCT Filed: Jan. 20, 2023
(86) PCT No.: PCT/US2023/061005

§ 371 Date: Jul. 19, 2024

Related U.S. Application Data
(60) Provisional application No. 63/376,849, filed on Sep. 23, 2022.
(60) Provisional application No. 63/348,614, filed on Jun. 3, 2022.
(60) Provisional application No. 63/301,855, filed on Jan. 21, 2022.

(51) Int. Cl. A61K 9/1271; A61K 9/51; A61K 48/00; A61P 1/16; C12N 9/12; C12N 9/22; C12N 15/11; C12N 15/88

(52) U.S. Cl. CPC A61K 9/1271; A61K 9/5123; A61K 48/0041; A61P 1/16; C12N 9/1241; C12N 9/22; C12N 15/111; C12N 15/88; C12N 2310/20

(57) ABSTRACT

Compositions comprising terpene-containing lipidoid compounds, methods of preparing such compositions, and the use of these compositions in gene delivery applications are disclosed.

### RELATED APPLICATIONS

This application claims priority to, and the benefit of, U.S. Provisional Application No. 63/301,855, filed on Jan. 21, 2022, U.S. Provisional Application No. 63/348,614, filed on Jun. 3, 2022, and U.S. Provisional Application No. 63/376,849, filed on Sep. 23, 2022. The contents of each of the aforementioned patent applications are incorporated herein by reference in their entireties for all purposes.

### SEQUENCE LISTING

The Sequence Listing XML associated with this application is provided electronically in XML format and is hereby incorporated by reference into the specification. The name of the XML file containing the Sequence Listing XML is “POTH-072_001WO_SeqList.xml”. The XML file is 32,446 bytes, created on Jan. 19, 2023, and is being submitted electronically via USPTO Patent Center.

### FIELD OF THE INVENTION

The present invention relates generally to novel lipid nanoparticles (“LNPs”) comprising terpene-derived compounds, methods of preparing these LNPs, and the use of these LNPs for gene therapy and cell-based therapy applications. The compositions and methods of the present disclosure have wide applicability to a diverse number of fields, including gene therapy and the production of cell-based therapeutics.

### BACKGROUND OF THE INVENTION

Efficient and safe delivery is one of the most significant challenges in the field of biological therapeutics. In the delivery of mRNA, for example, efficient transfection involves not only target specificity but also cell entry, endosomal escape, and translation. Lipid nanoparticles (LNPs) have proven effective for the delivery of biological therapeutics but especially nucleic acid-based therapies. A typical LNP contains a structural lipid, a PEG-containing lipid, and a phospholipid (often unsaturated). In addition, a pH sensitive (ionizable) lipid is included and serves a functional role in the delivery of the cargo. Typically, pH-sensitive lipids contain one or more amine functionalities that can be protonated (cationic) under certain physiological conditions. It is widely reported that the protonation of the amine-containing lipid facilitates endosomal escape thereby releasing the therapeutic agent to the interior of the cell.

Although a large number of cationic lipids have been discovered through high throughput screens, there remains a significant need for novel structures with strong safety profiles, efficient delivery characteristics, and high target specificity. Studies that correlate cationic lipid structure with desirable delivery characteristics are lacking. Lipid tail branching and unsaturation are two structural parameters that impact delivery efficiency

Terpenes are an abundant class of naturally occurring unsaturated hydrocarbon. The use of terpenes, chemically modified terpenes, or terpene-containing structures in the field of gene delivery is extremely limited. Given their abundance and structural diversity, terpenes represent an attractive feedstock for the construction of gene delivery agents.

The compositions and methods of the present disclosure have wide applicability to a diverse number of fields, including gene therapy and the production of cell-based therapeutics.

### SUMMARY OF THE INVENTION

In some aspects, provided are novel lipid nanoparticles (“LNPs”) comprising terpene-derived compounds. In one aspect, the terpene derived compound is a compound of Formula (I), Formula (II), or Formula (III).

In some aspects, provided are pharmaceutical compositions, comprising a composition of the present disclosure and at least one pharmaceutically-acceptable excipient or diluent.

In some aspects, provided are methods of delivering at least one nucleic acid to at least one cell comprising contacting the at least one cell with at least one composition of the present disclosure.

In some aspects, provided are methods of genetically modifying at least one cell comprising contacting the at least one cell with at least one composition of the present disclosure.

In some aspects, provided are methods of treating at least one disease or disorder in a subject in need thereof comprising administering to the subject at least one therapeutically effective amount of at least one composition of the present disclosure.

In some aspects, provided are methods of delivering at least one nucleic acid to at least one cell comprising contacting the at least one cell with at least one composition of the present disclosure.

In some aspects, provided are cells modified according to methods of the present

### DISCLOSURE

Any of the aspects and/or embodiments described herein can be combined with any other aspect and/or embodiment described herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### DETAILED DESCRIPTION OF THE INVENTION

The present disclosure provides novel lipid nanoparticle compositions (LNPs) comprising terpene-derived lipidoid compounds, methods for preparing the LNPs, and methods for using same. In a non-limiting example, the compositions and methods of the present limiting disclosure can be used for gene delivery and cell-based therapeutics. In a non-limiting example, the compositions and methods of the present disclosure can be broadly used to deliver a nucleic acid to liver cells, in vivo, ex vivo or in vitro, for the treatment of certain diseases and disorders, including, but not limited to liver disorders. In a non-limiting example, the compositions and methods of the present disclosure can be broadly used to deliver a nucleic acid to T-cells, in vivo, ex vivo or in vitro, for the treatment of certain disorders, including, but not limited to cancer. In a non-limiting example, the compositions and methods of the present disclosure can be broadly used to deliver a nucleic acid to primary, unactivated T-cells, in vivo, ex vivo or in vitro, for the treatment of certain diseases and disorders, including, but not limited to cancer. In a non-limiting example, the compositions and methods of the present disclosure can be broadly used to deliver a nucleic acid for the purpose of vaccination. In a non-limiting example, the compositions and methods of the present disclosure can be broadly used to deliver a nucleic acid to induce the expression of a secreted therapeutic protein.

### Compositions of the Present Disclosure-Lipid Nanoparticles

The present disclosure provides a composition comprising at least one lipid nanoparticle comprising a terpene-derived compound of the present disclosure and at least one nucleic acid molecule. In some aspects, a lipid nanoparticle can further comprise at least one structural lipid. In some aspects, a lipid nanoparticle can further comprise at least one phospholipid. In some aspects, a lipid nanoparticle can further comprise at least one PEGylated lipid.

### Compounds

In one aspect, the terpene-derived lipidoid compounds are represented by Formula (I):

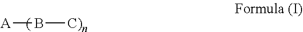

wherein:
A is A′ or A″,
A′ is:

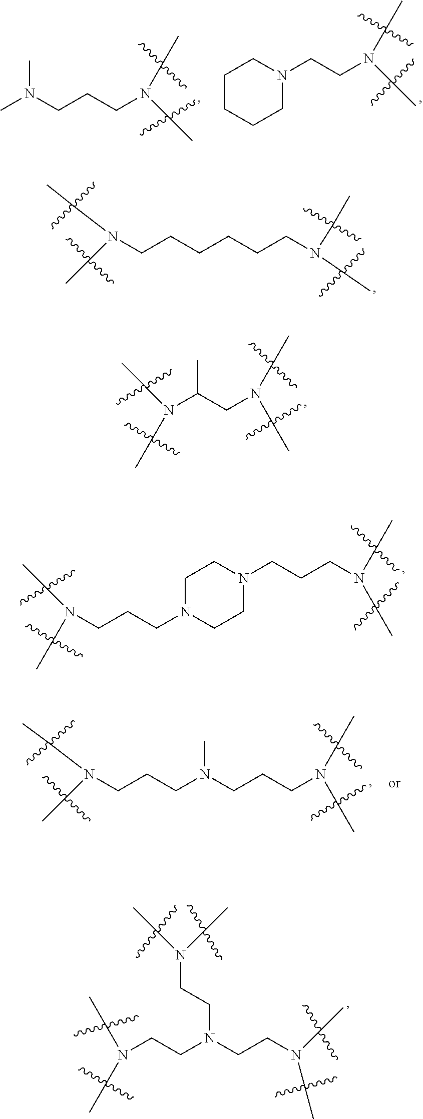

and

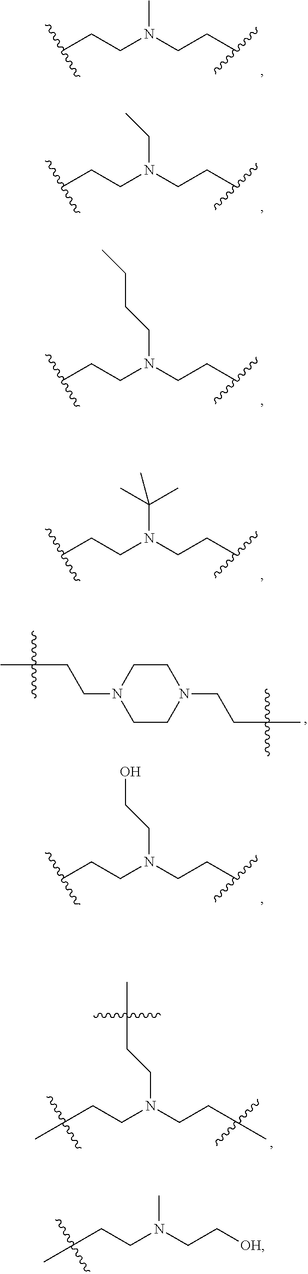

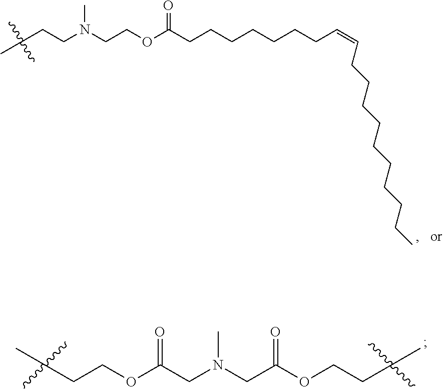 [SMILES: *CCN(C)CCOC(=O)CCCCCCC/C=C\CCCCCCCCCC ; *CCOC(=O)CN(C)CC(=O)OCC*]

A″ is:
B is B′ or B″,
B′ is:

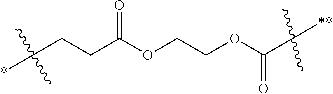

in which * indicates attachment to A, and ** indicates attachment to C:
B″ is:

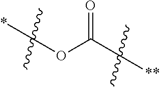

in which * indicates attachment to A, and ** indicates attachment to C;
wherein when A is A′, then Bis B′, and wherein when A is A″, then B is B″;
C is:

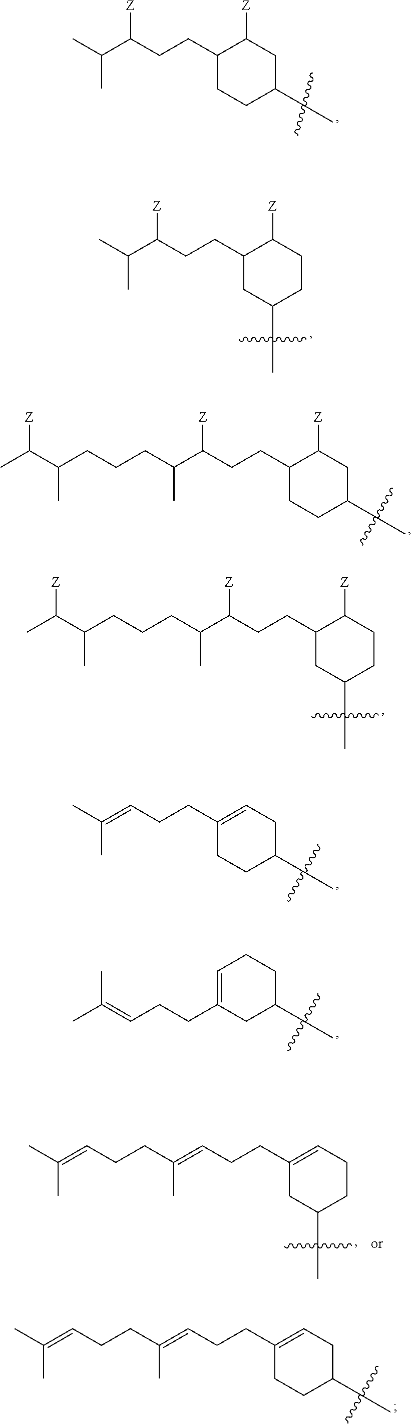

n is an integer between 1 to 6;
each Z is independently H or —S—R₁;
each R₁ is independently alkyl,

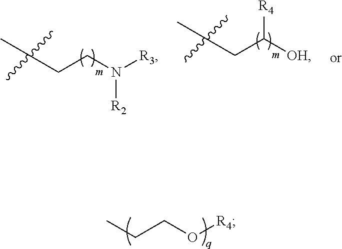

each R₂ is independently hydrogen, C₁-C₆ alkyl, C₁-C₆ alkenyl, aralkyl, or hydroxyalkyl;
each R₃ is independently hydrogen, C₁-C₆ alkyl, C₁-C₆ alkenyl, aralkyl, or hydroxyalkyl;
each R₄ is independently hydrogen or C₁-C₃ alkyl;
each m is an integer independently selected from 1-10; and
each q is an integer independently selected from 1-200.

In some aspects, n is 1.

In some aspects, n is 2.

In some aspects, n is 3.

In some aspects, n is 4.

In some aspects, n is 5.

In some aspects, n is 6.

In some aspects, A is A′.

In some aspects, A is A″.

In some aspects, A′ is

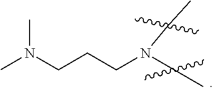 [SMILES: *N(*)CCCN(C)C]

In some aspects, A′ is

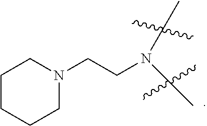 [SMILES: *N(*)CCN1CCCCC1]

In some aspects, A′ is

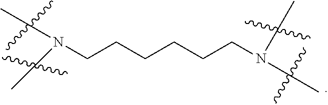 [SMILES: *N(*)CCCCCCN(*)*]

In some aspects, A′ is

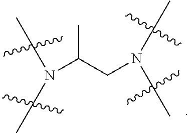 [SMILES: *N(*)CC(C)N(*)*]

In some aspects, A′ is

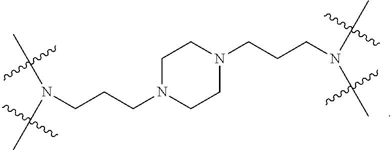 [SMILES: *N(*)CCCN1CCN(CCCN(*)*)CC1]

In some aspects, A′ is

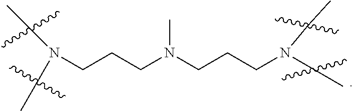 [SMILES: *N(*)CCCN(C)CCCN(*)*]

In some aspects, A′ is

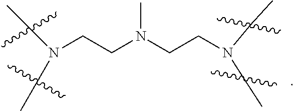 [SMILES: *N(*)CCN(C)CCN(*)*]

In some aspects, A″ is

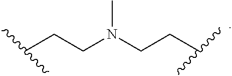 [SMILES: *CCN(C)CC*]

In some aspects, A″ is

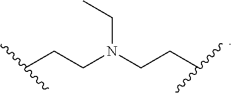 [SMILES: *CCN(CC)CC*]

In some aspects, A″ is

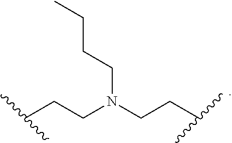 [SMILES: *CCN(CC*)CCCC]

In some aspects, A″ is

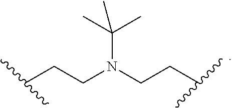 [SMILES: *CCN(CC*)C(C)(C)C]

In some aspects, A″ is

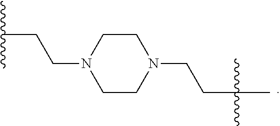 [SMILES: *CCN1CCN(CC*)CC1]

In some aspects, A″ is

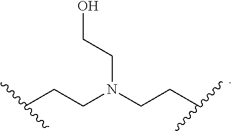 [SMILES: *CCN(CC*)CCO]

In some aspects, A″ is

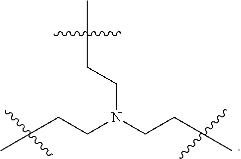 [SMILES: *CCN(CC*)CC*]

In some aspects, A″ is

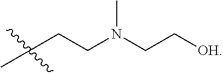 [SMILES: *CCN(C)CCO]

In some aspects, A″ is

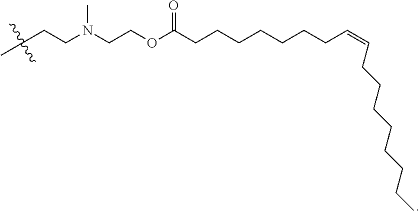 [SMILES: *CCN(C)CCOC(=O)CCCCCCC/C=C\CCCCCCCC]

In some aspects, A″ is

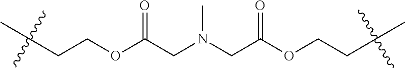 [SMILES: *CCOC(=O)CN(C)CC(=O)OCC*]

In some aspects, B is B′.

In some aspects, B is B″.

In some aspects, B′ is

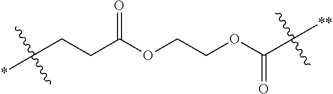

in which * indicates attachment to A, and ** indicates attachment to C.

In some aspects, B″ is

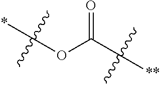

in which * indicates attachment to A, and ** indicates attachment to C.

In some aspects, the carbonyl group of B′ or B″ is attached to “C” part of the molecule and the other end of B′ or B″ is attached to “A” part of the molecule.

In some aspects, each C segment is:

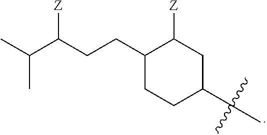

In some aspects, each C segment is:

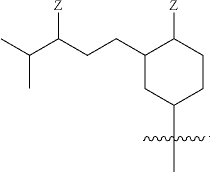

In some aspects, each C segment is:

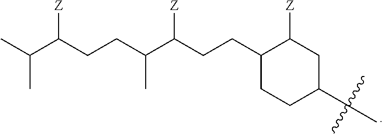

In some aspects, each C segment is:

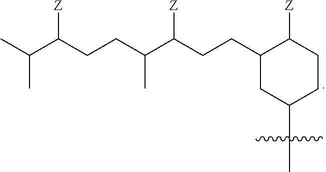

In some aspects, each C segment is:

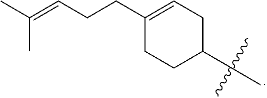 [SMILES: *C1CC=C(CCC=C(C)C)CC1]

In some aspects, each C segment is:

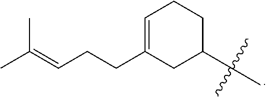 [SMILES: *C1CCC=C(CCC=C(C)C)C1]

In some aspects, each C segment is:

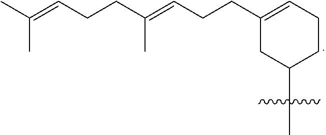 [SMILES: *C1CCC=C(CC/C=C(\C)CCC=C(C)C)C1]

In some aspects, each C segment is:

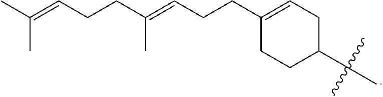 [SMILES: *C1CC=C(CC/C=C(\C)CCC=C(C)C)CC1]

In some aspects, at least one, at least two, or at least three, or at least four or more Z groups of the C segment is —S—R₁, wherein R₁ is alkyl.

In some aspects, R₁ is C₁₀H₂₁.

In some aspects, R₁ is C₂H₄(CH)(CH₃)₂.

In some aspects, at least one, at least two, or at least three, or at least four or more Z groups of the C segment is —S—R₁, wherein R₁ is

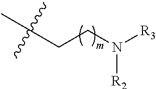

In one such embodiment, m is one and R₂, and R₃ are each independently C₁-C₆ alkyl. In one embodiment, each of the C₁-C₆ alkyl groups is methyl. In this embodiment, 2-(dimethylamino) ethanethiol or 2-(dimethylamino) ethanethiol hydrochloride can be used as the derivatizing agent.

In one such embodiment, m is one and R₂, and R₃ are each independently C₁-C₆ alkyl. In one embodiment, each of the C₁-C₆ alkyl groups is methyl or ethyl. In this embodiment, 2-(dimethylamino) ethanethiol or 2-(dimethylamino) ethanethiol hydrochloride can be used as the derivatizing agent. In the case where each of the C₁-C₆ alkyl groups are ethyl, 2-diethylaminoethane thiol or 2-diethylaminoethane thiol hydrochloride can be used as the derivatizing agent.

In some aspects, each Z is —S—R₁, wherein at least one, at least two, or at least three, or at least four or more R₁ is:

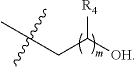

In one embodiment, m is one and R₄ is hydrogen. In this embodiment, ethane thiol can be used as the derivatizing agent.

In some aspects, each Z is —S—R₁, wherein at least one, at least two, or at least three, or at least four or more R₁ is:

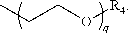

In one embodiment, q is one and R₄ is methyl.

In some aspects, A is A′, A′ is

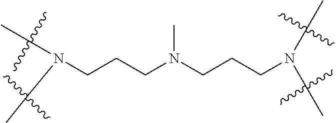 [SMILES: *N(*)CCCN(C)CCCN(*)*]

### and B is B′.

In some embodiments, C is

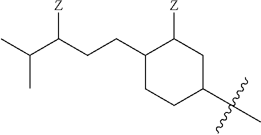

and each Z is independently H.

In some embodiments, n is 4.

In some aspects, the terpene-derived cationic lipidoid compound of Formula (1) is a compound selected from the following:

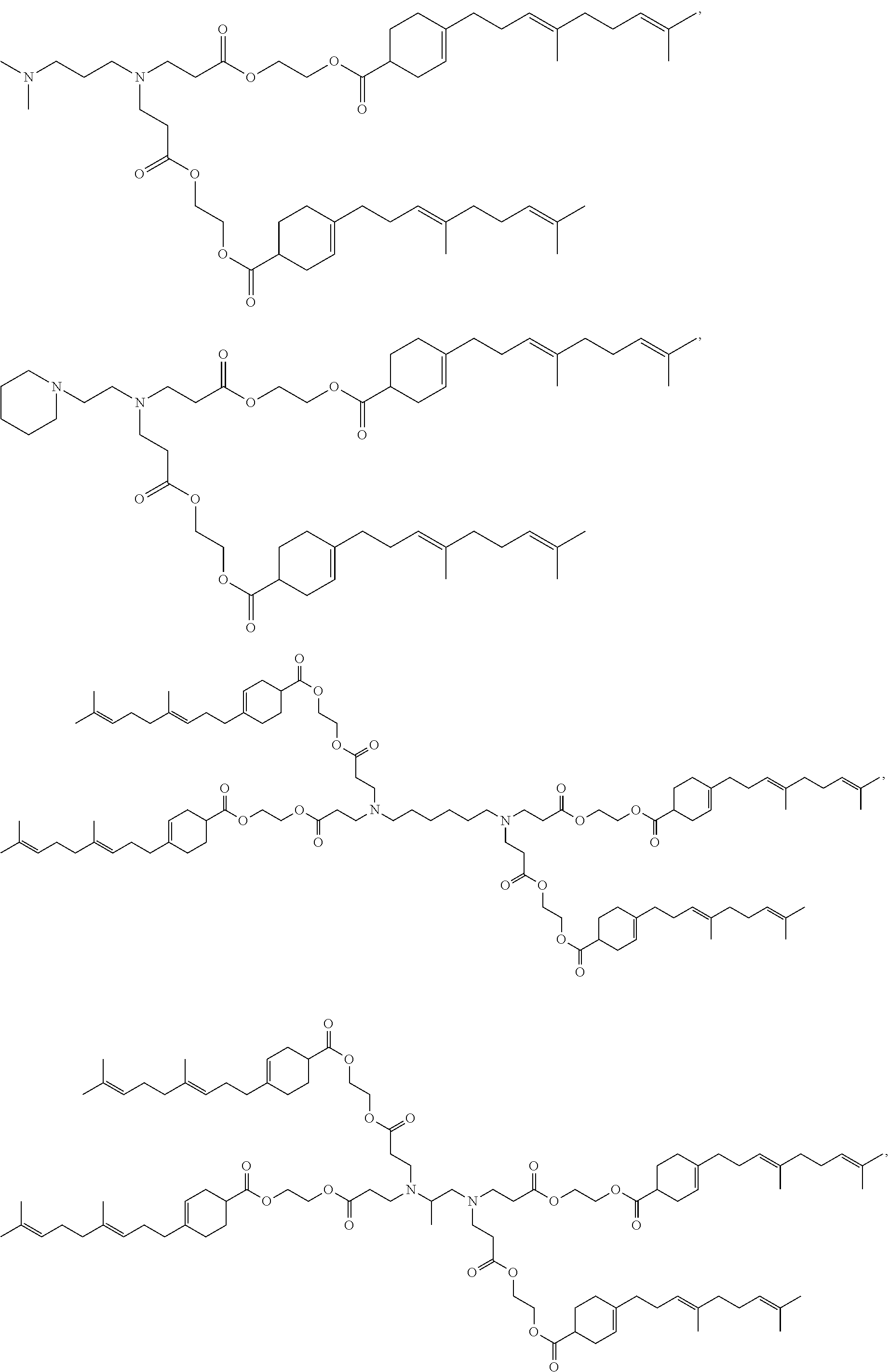

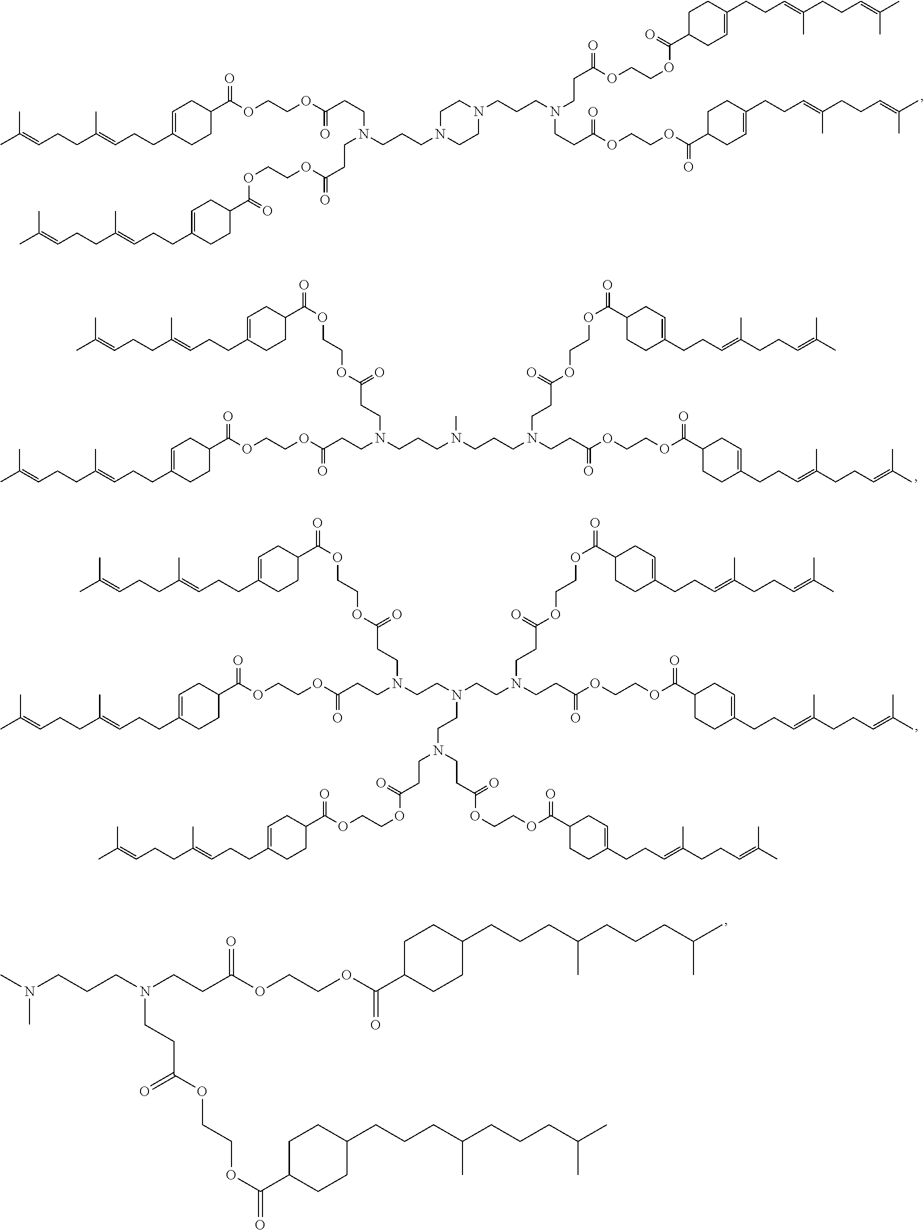

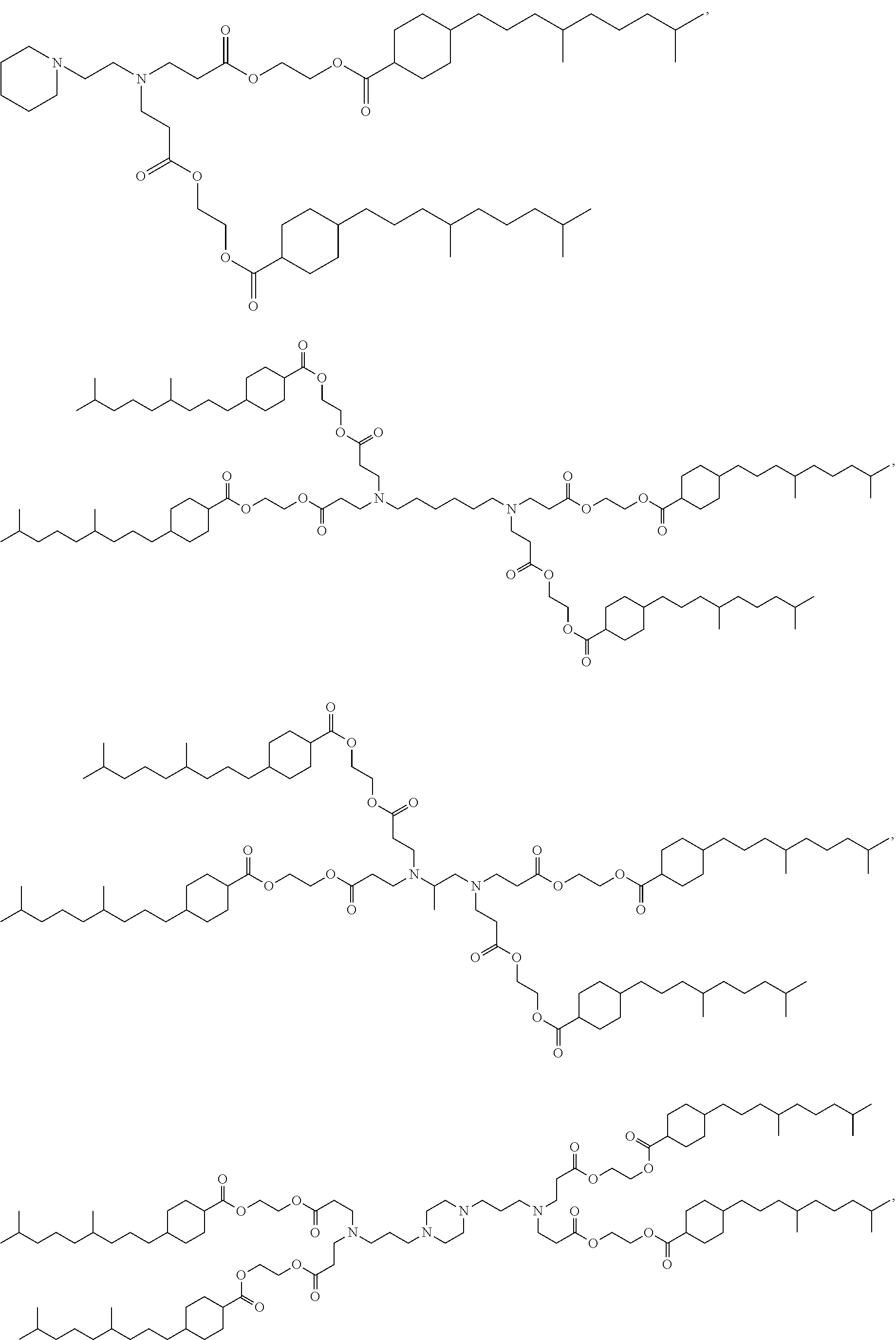

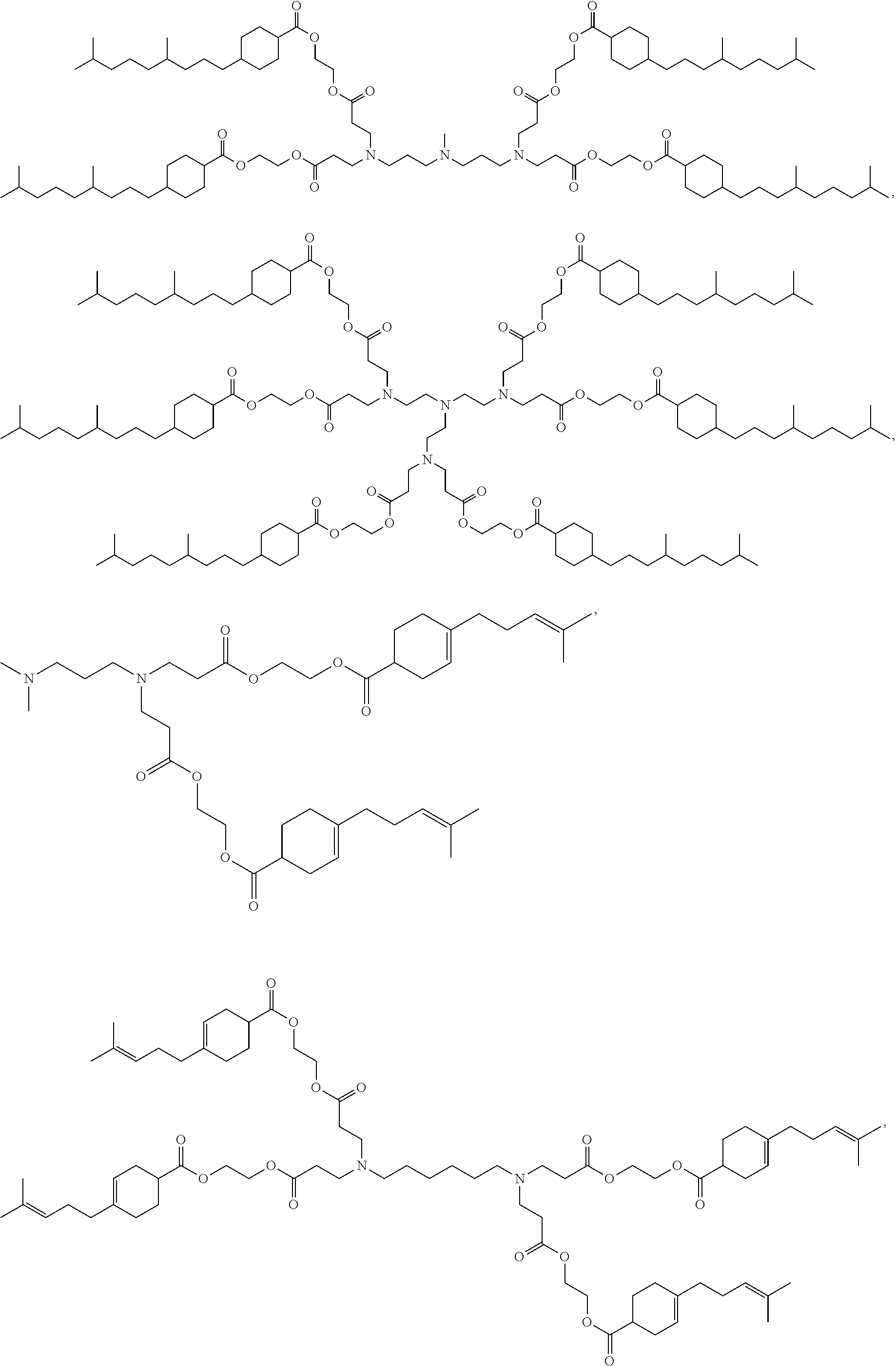

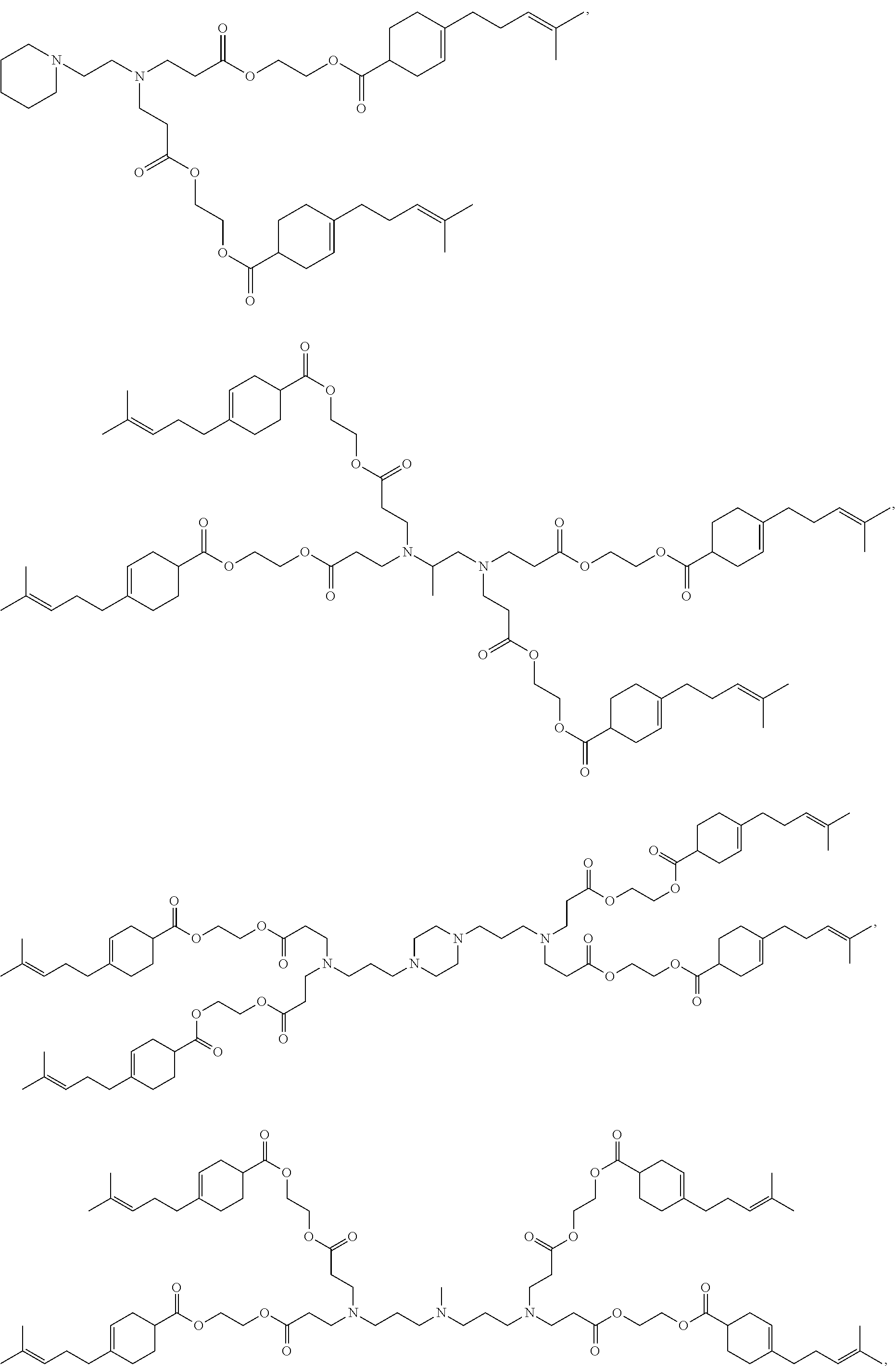

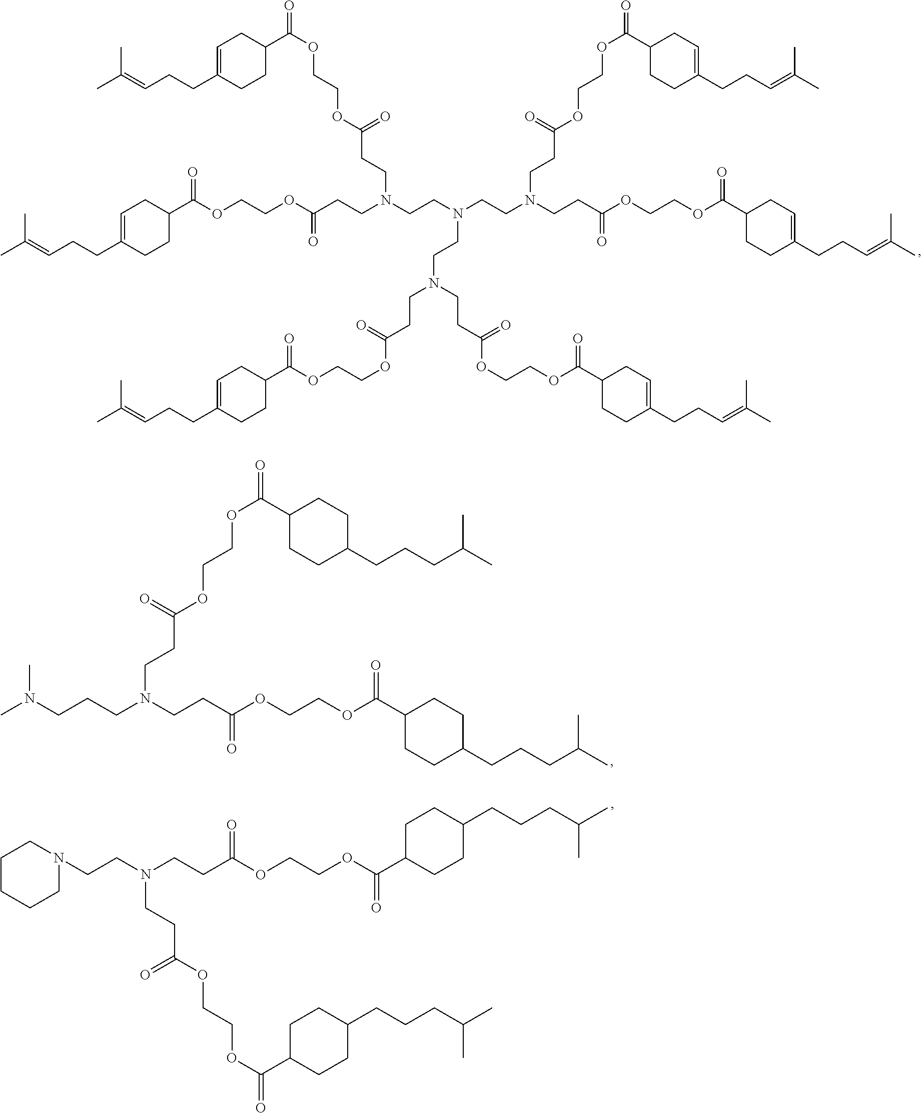

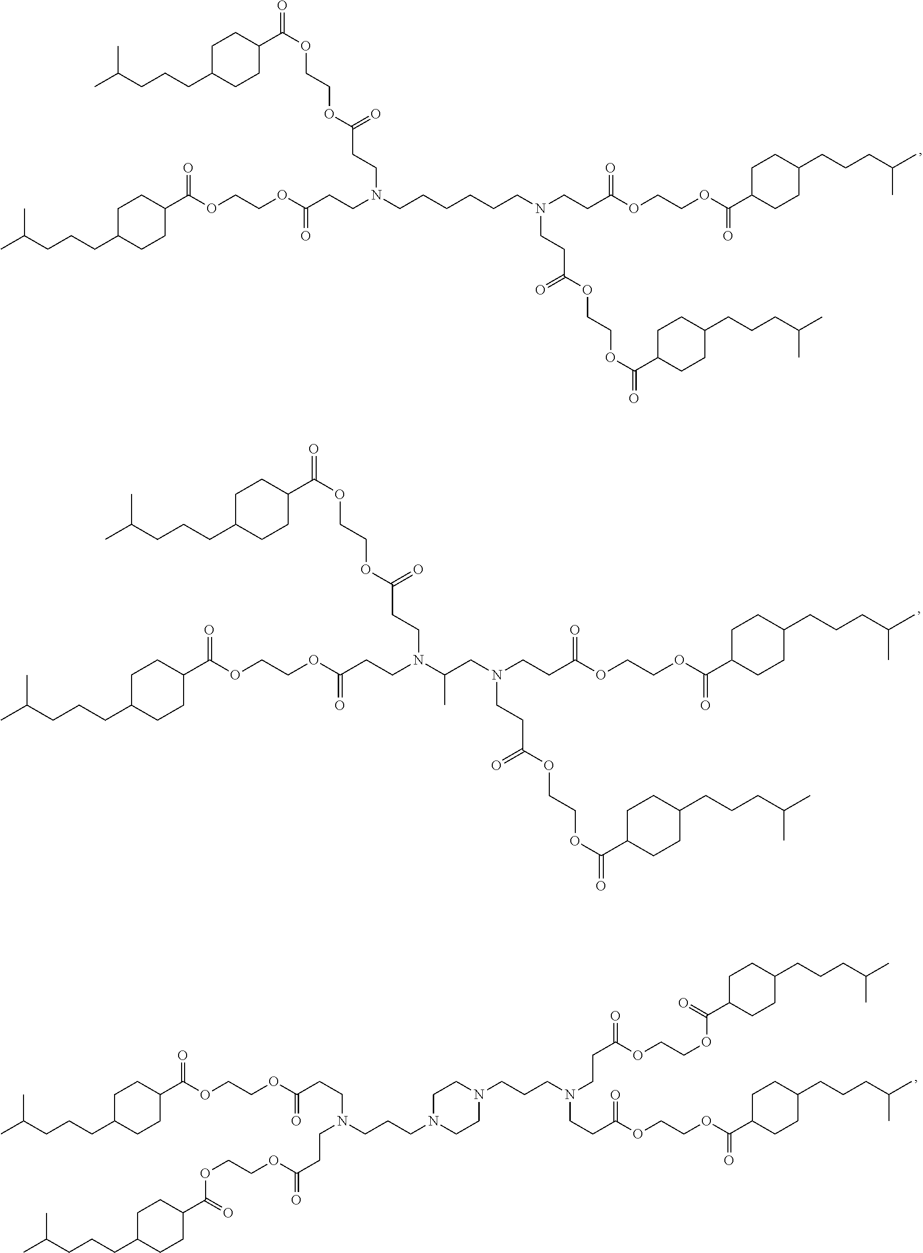

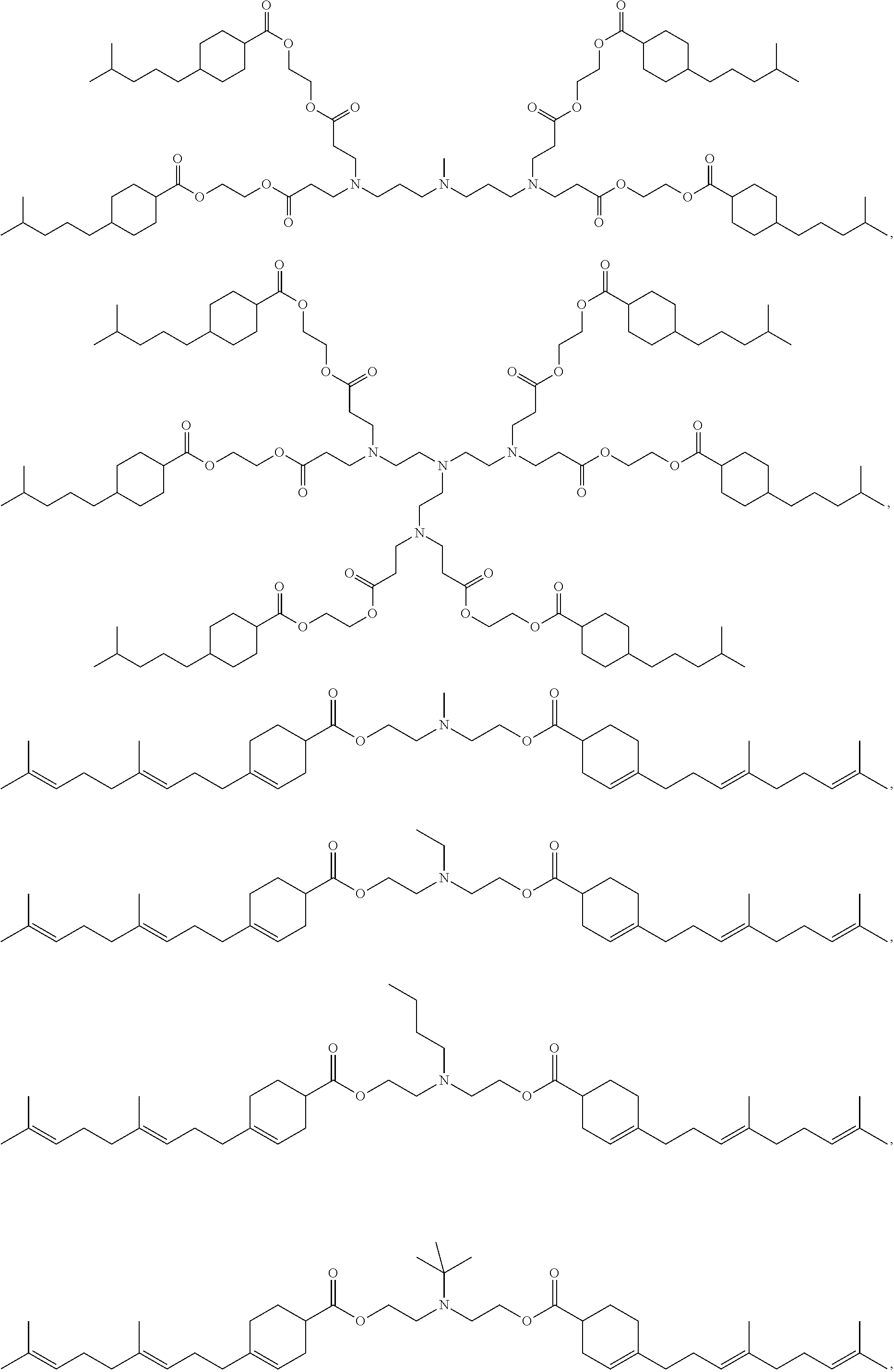

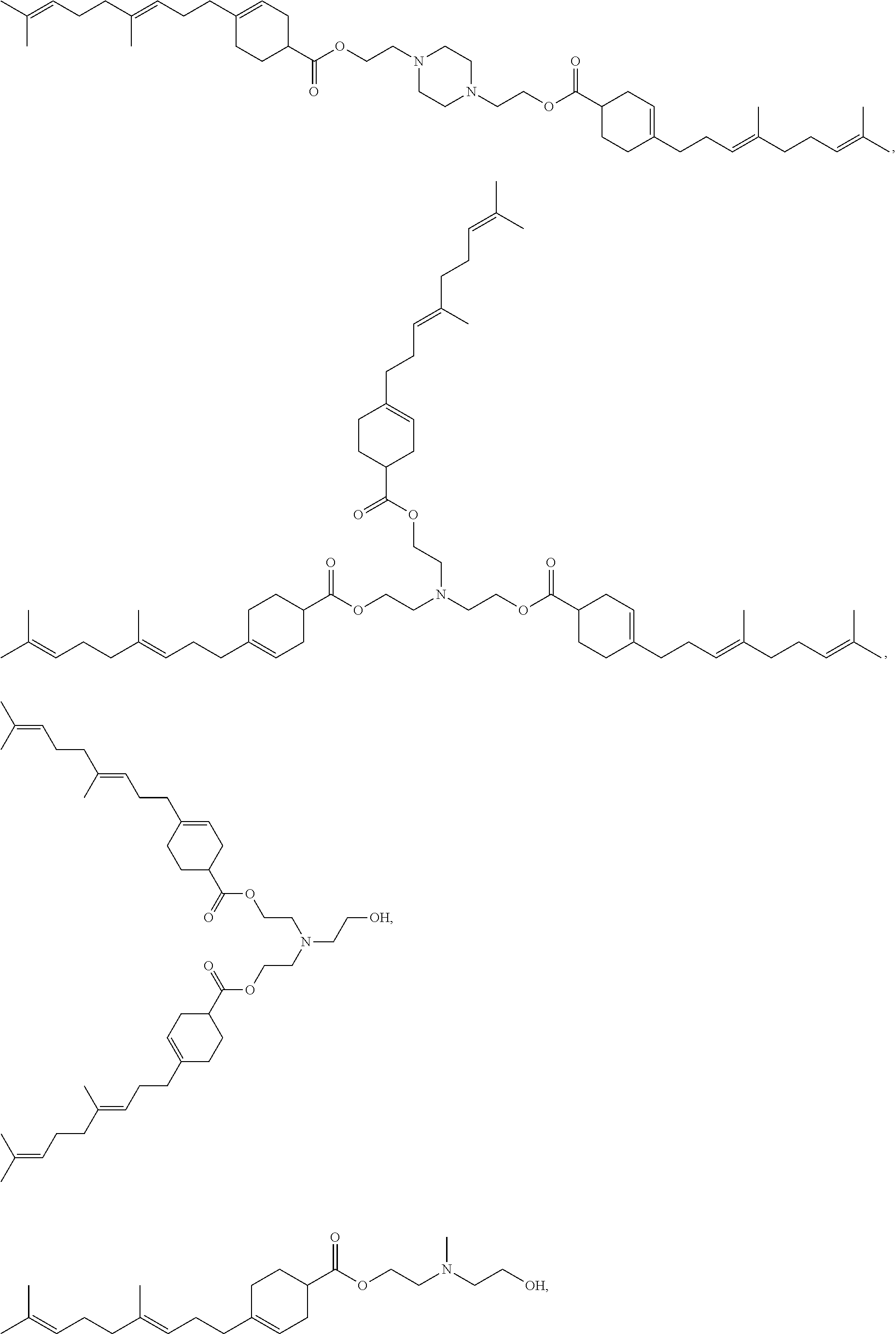

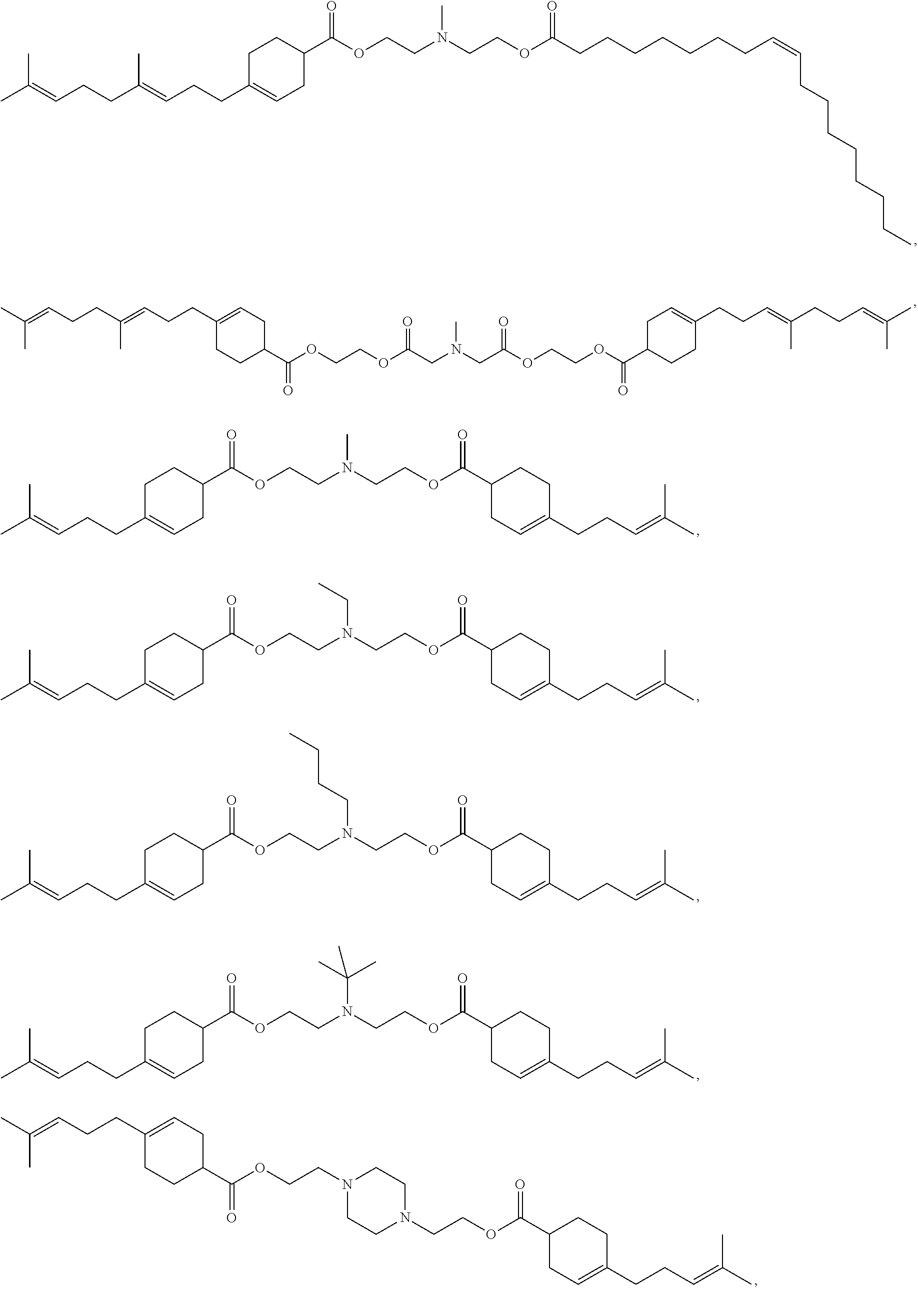

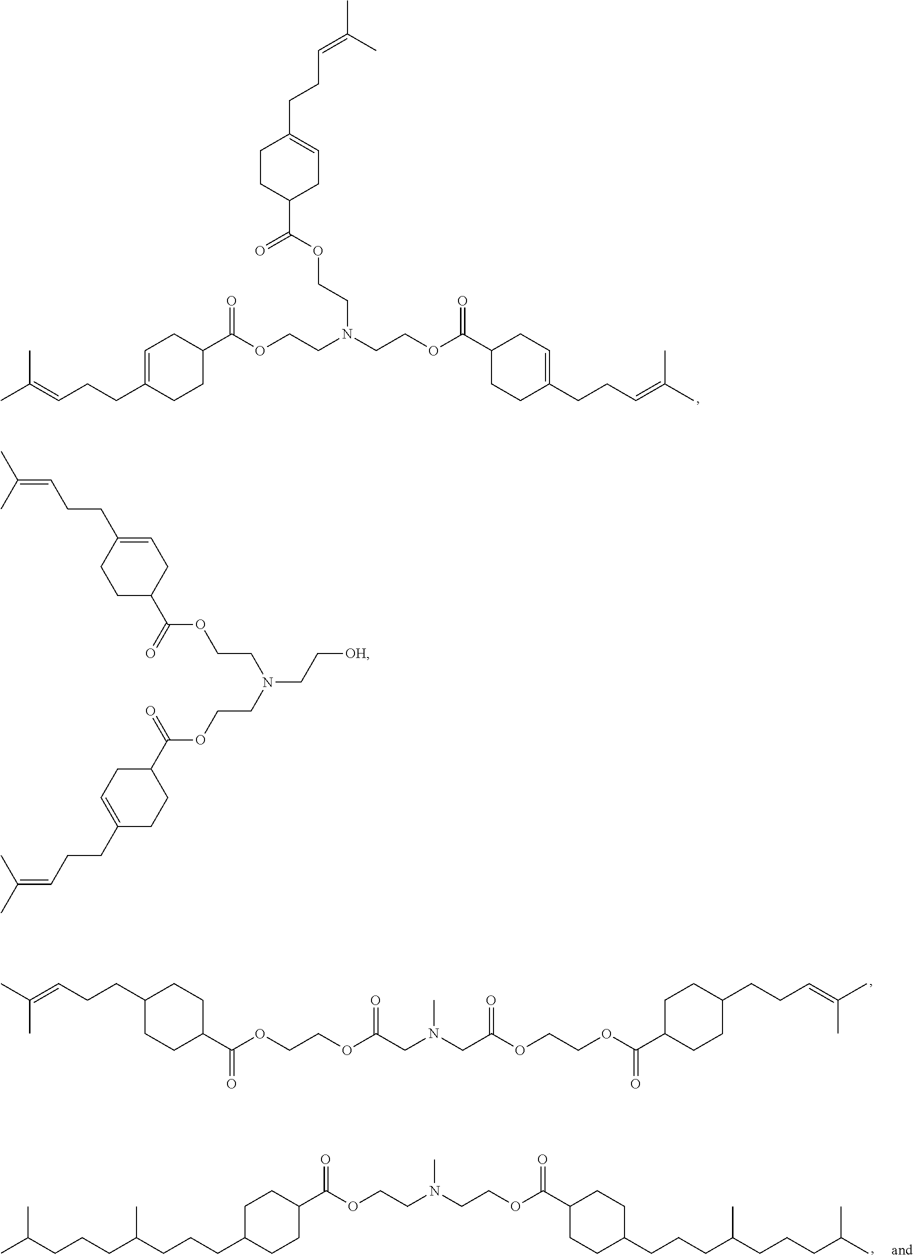

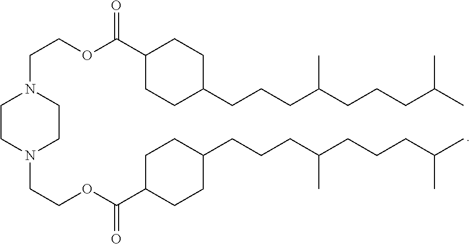 [SMILES: CC(C)CCCC(C)CCCC1CCC(C(=O)OCCN2CCN(CCOC(=O)C3CCC(CCCC(C)CCCC(C)C)CC3)CC2)CC1]

In one aspect, the terpene-derived lipidoid compounds are represented by Formula (II):

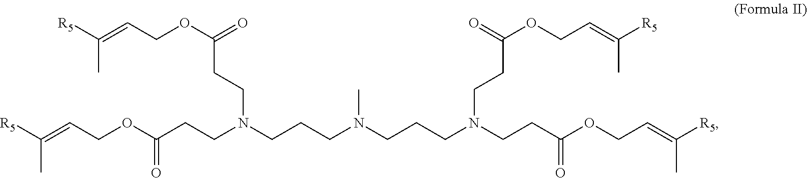

wherein each R₅ is independently

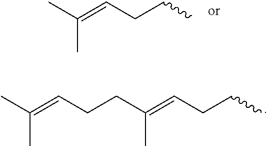

In some embodiments, each R₅ is

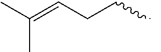

In some embodiments, each R₅ is

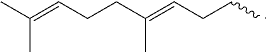

In some aspects, the terpene-derived cationic lipidoid compound of Formula (II) is a compound selected from the following:

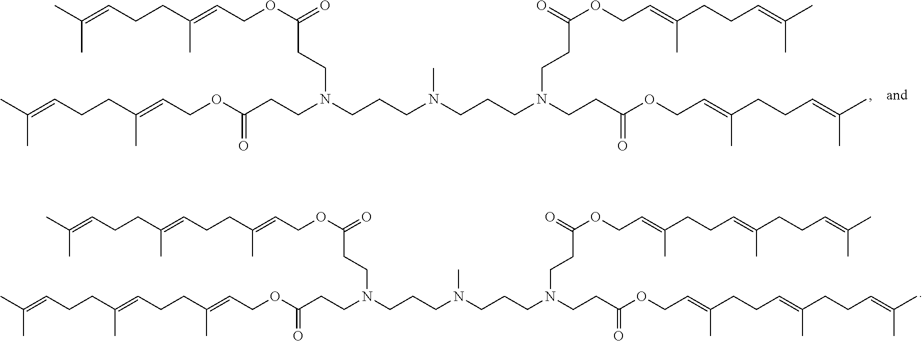

In one aspect, the terpene-derived lipidoid compounds are represented by Formula (III):

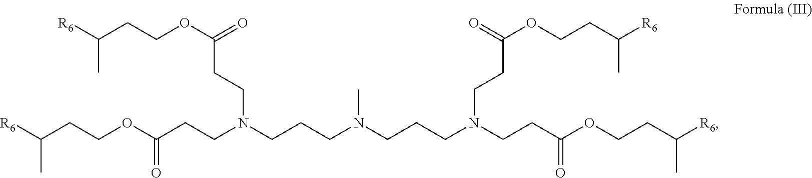

wherein each R₆ is

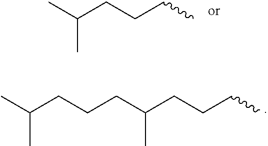

In some embodiments, each R₆ is

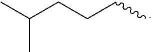

In some embodiments, each R₆ is

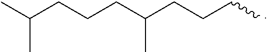

In some aspects, the terpene-derived cationic lipidoid compound of Formula (III) is a compound selected from the following:

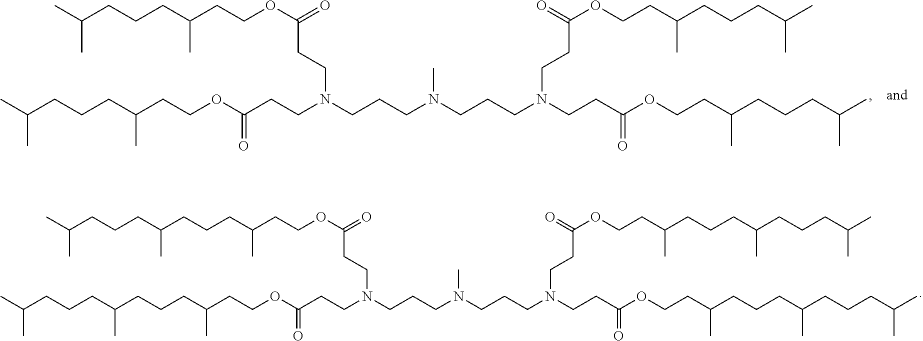

In addition to the structural formulas shown above, the exemplified compounds include compounds which are 1,3 isomers of the exemplified compounds (which are 1,4 isomers). In other words, all exemplified compounds include versions of the same compounds where the C unit is a 1,3 isomer. For example, when the invention describes compound:

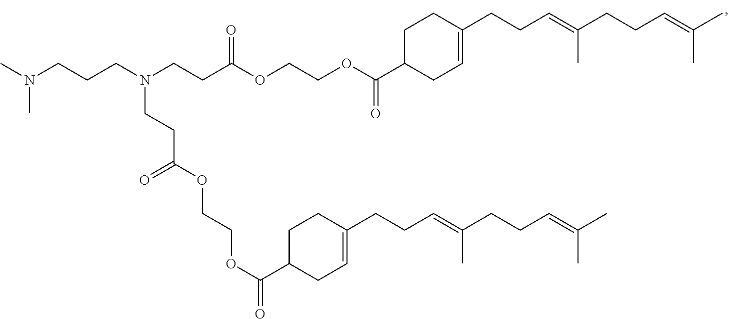 [SMILES: CC(C)=CCC/C(C)=C/CCC1=CCC(C(=O)OCCOC(=O)CCN(CCCN(C)C)CCC(=O)OCCOC(=O)C2CC=C(CC/C=C(\C)CCC=C(C)C)CC2)CC1]

it is understood that it also exemplifies compound with the following structure:

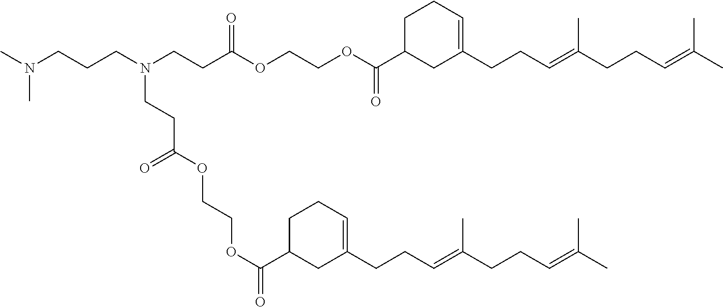 [SMILES: CC(C)=CCC/C(C)=C/CCC1=CCCC(C(=O)OCCOC(=O)CCN(CCCN(C)C)CCC(=O)OCCOC(=O)C2CCC=C(CC/C=C(\C)CCC=C(C)C)C2)C1]

In fact, usually the exemplified compounds exist as mixtures of 1,4 isomers and 1,3 isomers.

It will be understood that the compounds of any one of the Formulas disclosed herein and any pharmaceutically acceptable salts thereof, comprise stereoisomers, mixtures of stereoisomers, polymorphs of all isomeric forms of said compounds.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

It will be understood that in any of the formulae described herein, when a “-” is used to indicate linkage between two variables (e.g., A-B), the linkage could be one or more covalent bonds.

### General Methods for the Preparation of Compounds of Formula (I), Formula (II), or Formula (III) of the Present Disclosure

Compounds of Formula (I), Formula (II), or Formula (III) can be prepared using the reagents, intermediates, precursors, methods and schemes disclosed herein or using other commercially available reagents and methods known to those skilled in the art.

Some of the compounds of the invention, so called first generation lipidoids, can be prepared by the general schemes described below.

### Preparation of First Generation Lipidoids

In general, the first step in preparation of first generation terpene-derived cationic lipidoid compounds of Formula (I) of the present disclosure is the selection of an appropriate terpene, e.g., trans-beta-farnesene, beta-myrcene or other suitable biorenewable terpene, to prepare the multivalent “A” segment precursor. Mixtures of both 1,3-& 1,4-regioisomers are formed and the mixtures continued further without separation. For example:

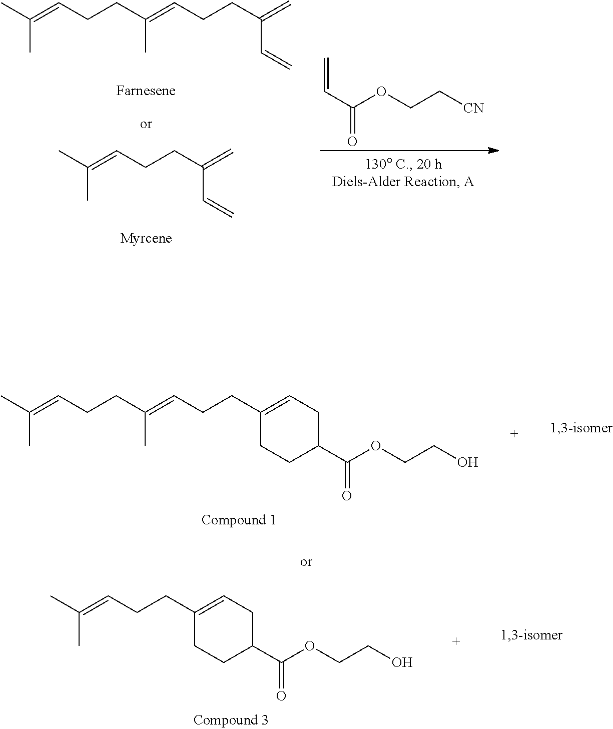

Suitable Diels-Alder products for use in the methods herein include:

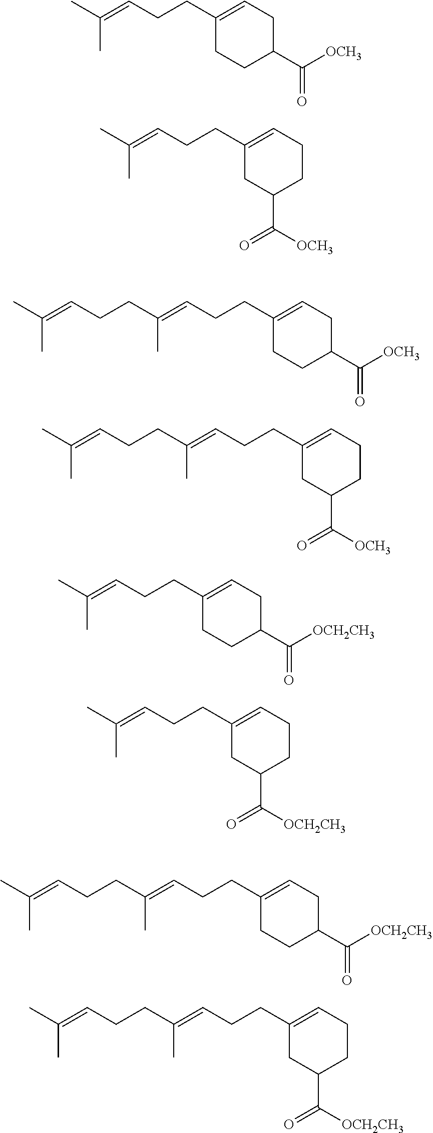

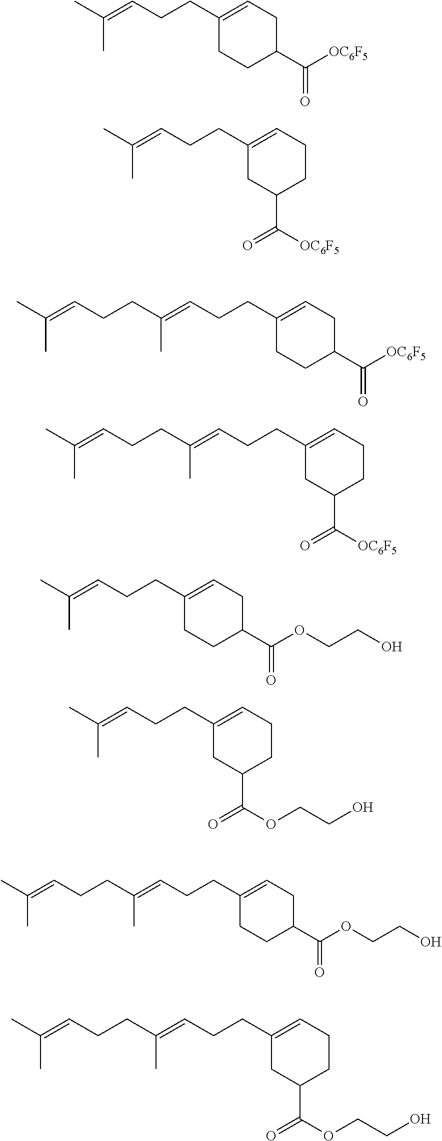

### General Procedure for Diels-Alder Reaction (A)

In a glass tube, trans-β-farnesene or myrcene (1.0 eq) and 2-hydroxyethyl acrylate (1.2 eq) were taken and the tube was sealed. The reaction was then stirred for 20 h at 130° C. The cooled reaction mixture was purified by silica gel flash chromatography (eluent: 20-30% EtOAc/hexane) to give compounds 1 or 3 as colorless oils.

Suitable temperatures are generally between 30° C. and 150° C. Higher temperatures are generally associated with higher yields.

Following the Diels-Alder Reaction, one optional step is hydrogenation which can be carried out as follows:

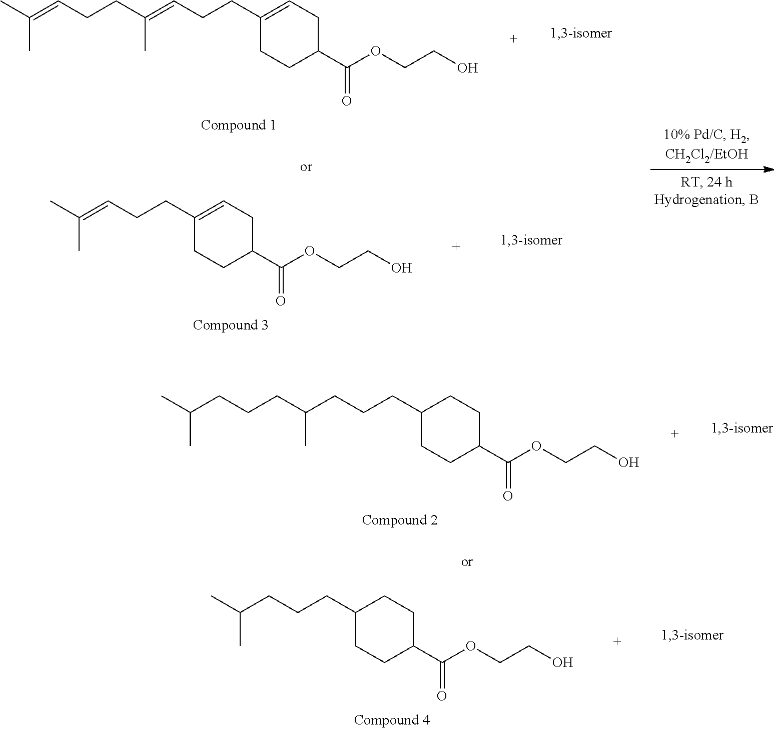 [SMILES: CC(C)=CCC/C(C)=C/CCC1=CCC(C(=O)OCCO)CC1 ; CC(C)=CCCC1=CCC(C(=O)OCCO)CC1 ; CC(C)CCCC(C)CCCC1CCC(C(=O)OCCO)CC1 ; CC(C)CCCC1CCC(C(=O)OCCO)CC1]

### General Procedure for Hydrogenation (B)

In a round-bottomed flask, compound 1 or 3 was dissolved in mixed solvents of ethanol and CH₂Cl₂ (4:1). To this solution, 10% Pd/C (10% w/w) was added in one portion and the resulting dark suspension was stirred under a hydrogen atmosphere for 24 h at room temperature. The reaction mixture was filtered through celite and rinsed with CH₂Cl₂ (3×). The filtrate was evaporated to give the hydrogenated compounds. The resulting residues proceeded to the next step without any purification.

Compounds which were prepared through either Diels-Alder reaction (A) or through hydrogenation (B) can then undergo acrylate installation (C) to provide myrcene acrylates (MA), farnesene acrylates (FA) (using products of Diels-Alder reaction) or hydrogenated myrcene acrylates (HMA) or hydrogenated farnesene acrylates (HFA) (using products of hydrogenation reaction):

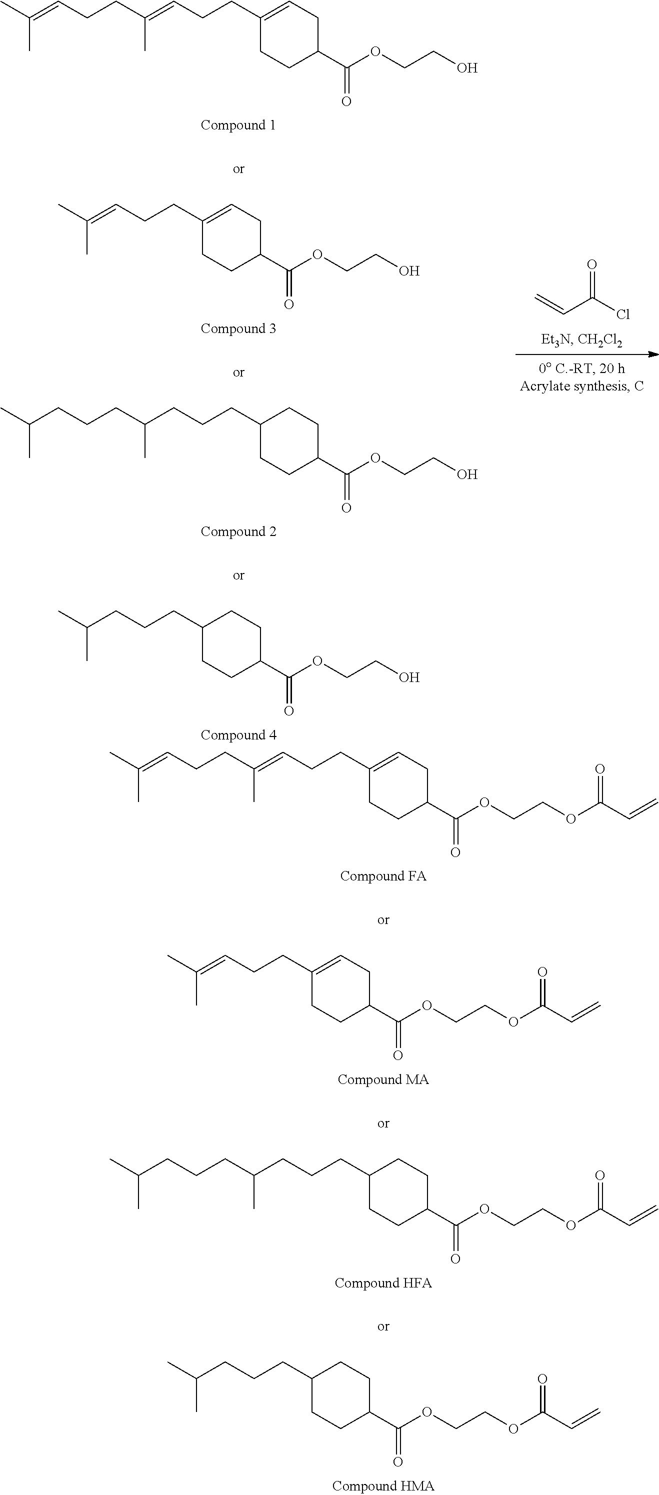

While the compounds in the scheme above are shown as 1,4 isomers, the compounds also include 1,3 isomers.

### General Procedure for Acrylate Synthesis (C)

In a round-bottomed flask, compound 1 or 2 or 3 or 4 (1.0 eq) was mixed with triethylamine (2.0 eq) in anhydrous CH₂Cl₂. The solution was cooled to 0° C. with an ice/water bath and acryloyl chloride (2.0 eq) was added dropwise over a period of 10 minutes. The resulting mixture was stirred for 20 h allowing the temperature to rise to room temperature. The reaction was quenched by adding a saturated aqueous solution of sodium bicarbonate (20 mL) followed by CH₂Cl₂ (20 mL). Both layers were separated and the aqueous layer was extracted with CH₂Cl₂ (3 x). Combined organic extracts were washed with brine solution (20 mL); dried over sodium sulfate; and evaporated. The reaction crude was then purified using silica gel flash chromatography (eluent: 10-15% EtOAc/hexane).

Finally, first generation lipidoids can be prepared from these acrylate compounds through Aza-Michael Addition (D) as follows:

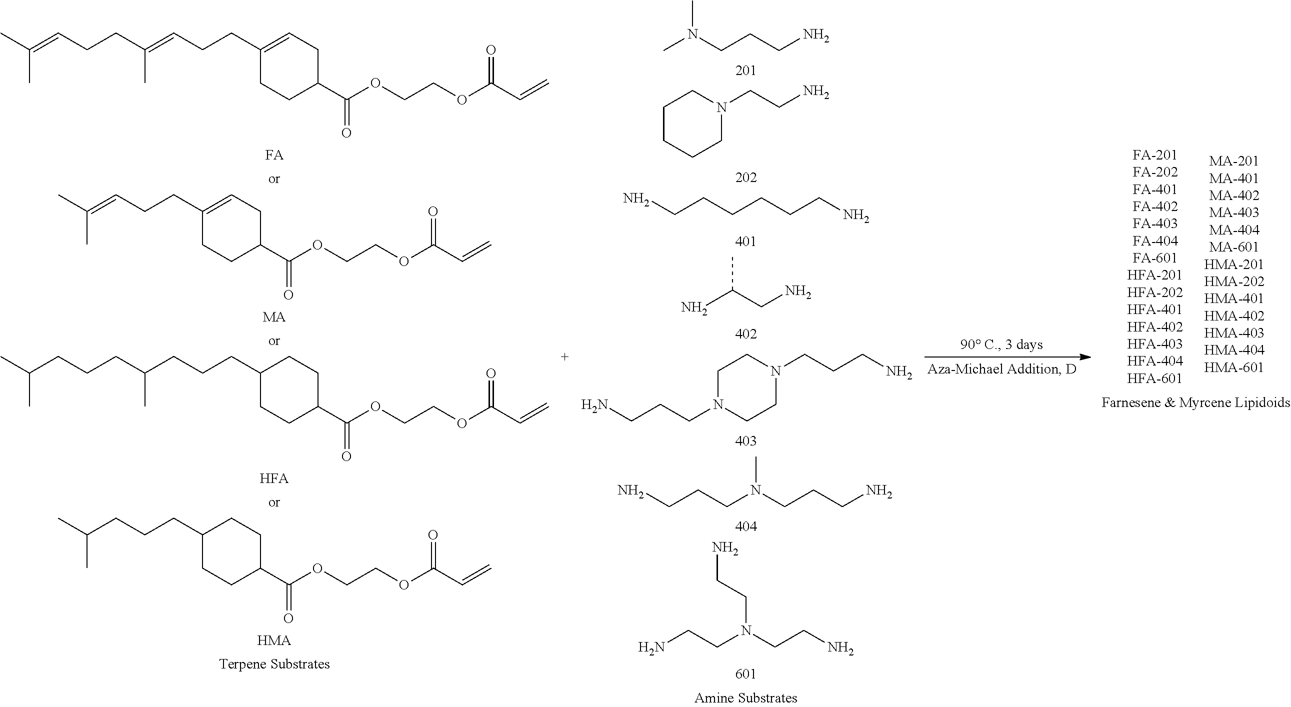

While the compounds in the scheme above are shown as 1,4 isomers, the compounds also include 1,3 isomers.

### General Procedure for Aza-Michael Addition (D)

In a 4-dram glass vial, acrylate (1.0 eq), and amine ([2n+1.0] eq; where n is the number of tails in the amine) were mixed and the vial was capped. The resulting reaction mixture was stirred for 3 days at 90° C. The reaction was cooled and purified by silica gel flash chromatography (eluent: 30-50% EtOAc/hexane or 0-10% MeOH/CH₂Cl₂).

Suitable temperatures are generally between 30° C. and 150° C.

### Further Derivatization of First Generation Lipidoids

Furthermore, the compounds of the invention can be functionalized further with alkyl chain thiols (for example, dodecane thiol and 3-methyl butyl thiol). Alkyl chains may be added to the unsaturated compounds through thiol-ene reaction as follows.

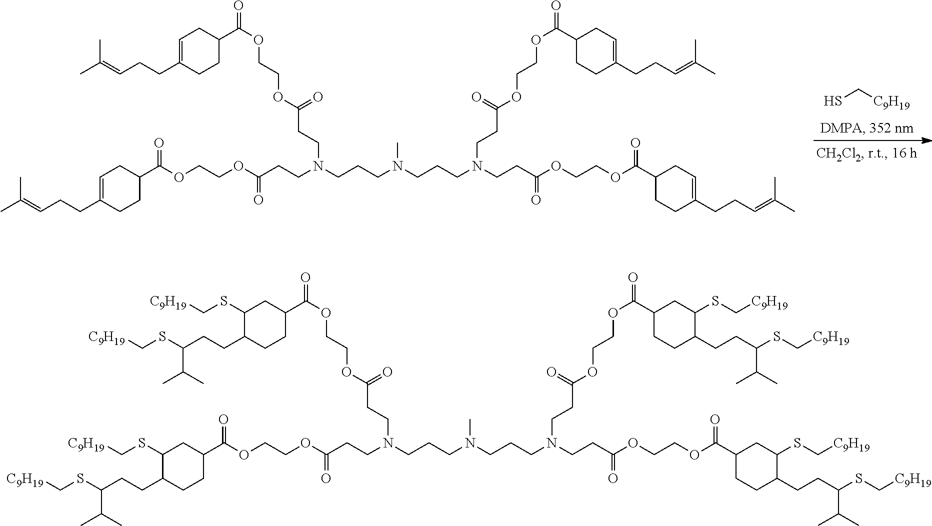

While the compounds in the scheme above are shown as 1,4 isomers, the compounds also include 1,3 isomers.

In a 4 mL quartz tube, MA-404 (50 mg, 1.0 eq), 1-dodecanethiol (63 mg, 10.0 eq), and DMPA (19 mg, 2.0 eq) were dissolved in anhydrous DCM (2.0 mL). The resulting solution was degassed with nitrogen for 5 minutes before irradiating with a UV light of wavelength 352 nm for 16 h at room temperature in a UV chamber. The tube was taken out of the UV chamber, reaction mixture was purified by silica gel column chromatography with 5% MeOH/DCM to afford 54 mg (53%) of MA-404-DT as a viscous oil. 1H NMR (400 MHZ, Chloroform-d) 8 4.36-4.13 (m, 20H), 3.51-2.29 (m, 61H), 2.27-1.46 (m, 59H), 1.42-0.54 (m, 160H).

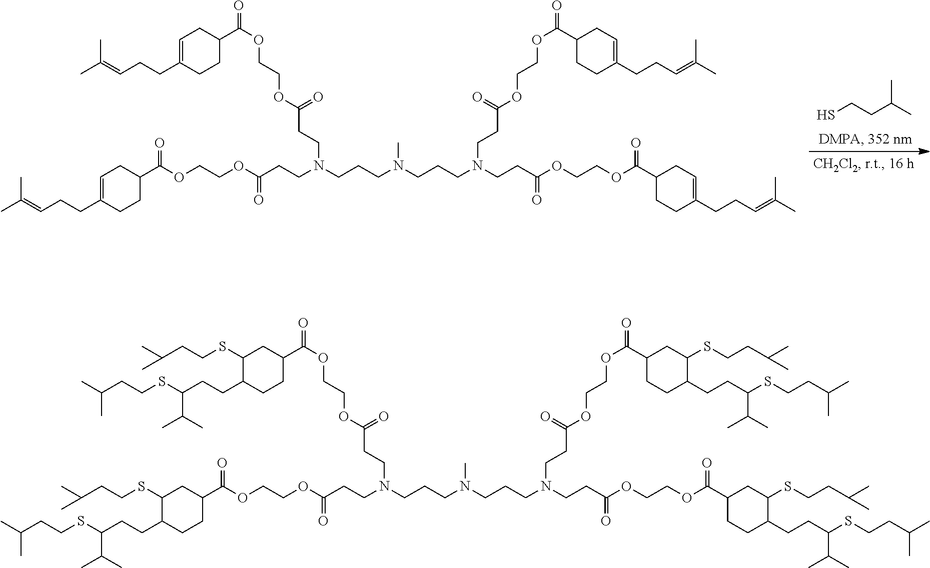

While the compounds in the scheme above are shown as 1,4 isomers, the compounds also include 1,3 isomers.

In a 4 mL quartz tube, MA-404 (50 mg, 1.0 eq), 3-methylbutyl thiol (38 mg, 10.0 eq), and DMPA (19 mg, 2.0 eq) were dissolved in anhydrous DCM (2.0 mL). The resulting solution was degassed with nitrogen for 5 minutes before irradiating with a UV light of wavelength 352 nm for 16 h at room temperature in a UV chamber. The tube was taken out of the UV chamber, reaction mixture was purified by silica gel column chromatography with 5% MeOH/DCM to afford 27 mg (34%) of MA-404-MBT as a viscous oil. 1H NMR (400 MHZ, Chloroform-d) 8 4.35-4.15 (m, 16H), 3.87-1.80 (m, 83H), 1.78-1.06 (m, 58H), 1.04-0.48 (m, 62H).

### Preparation of Second Generation Lipidoids

Second generation lipidoids of the invention (which also fall within the generic Formula I) can be prepared as follows.

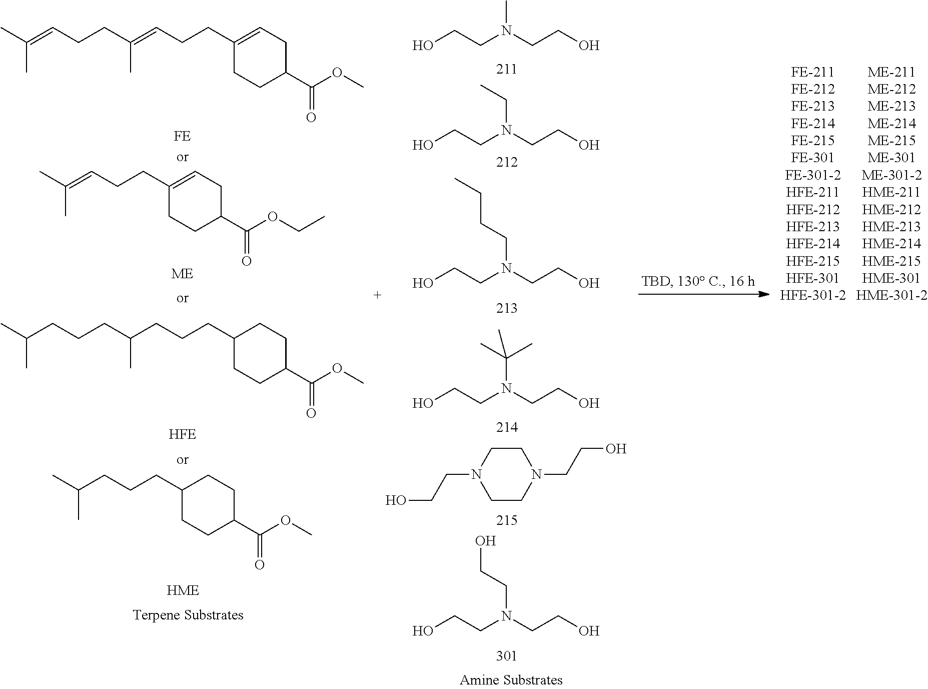

While the compounds in the scheme above are shown as 1,4 isomers, the compounds also include 1,3 isomers.

Structures of farnesene lipidoids that can be obtained by such scheme are as follows:

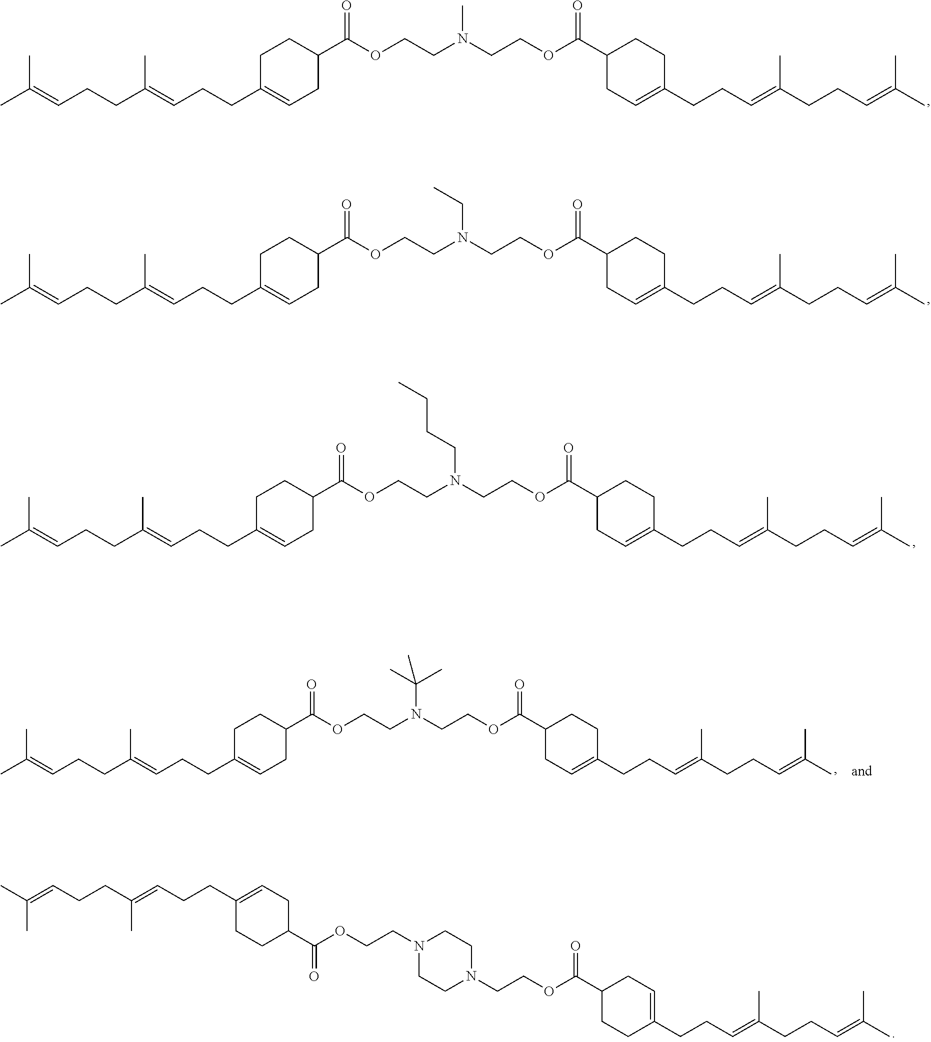

Structures of hybrid farnesene lipidoids that can be obtained by such scheme are as follows:

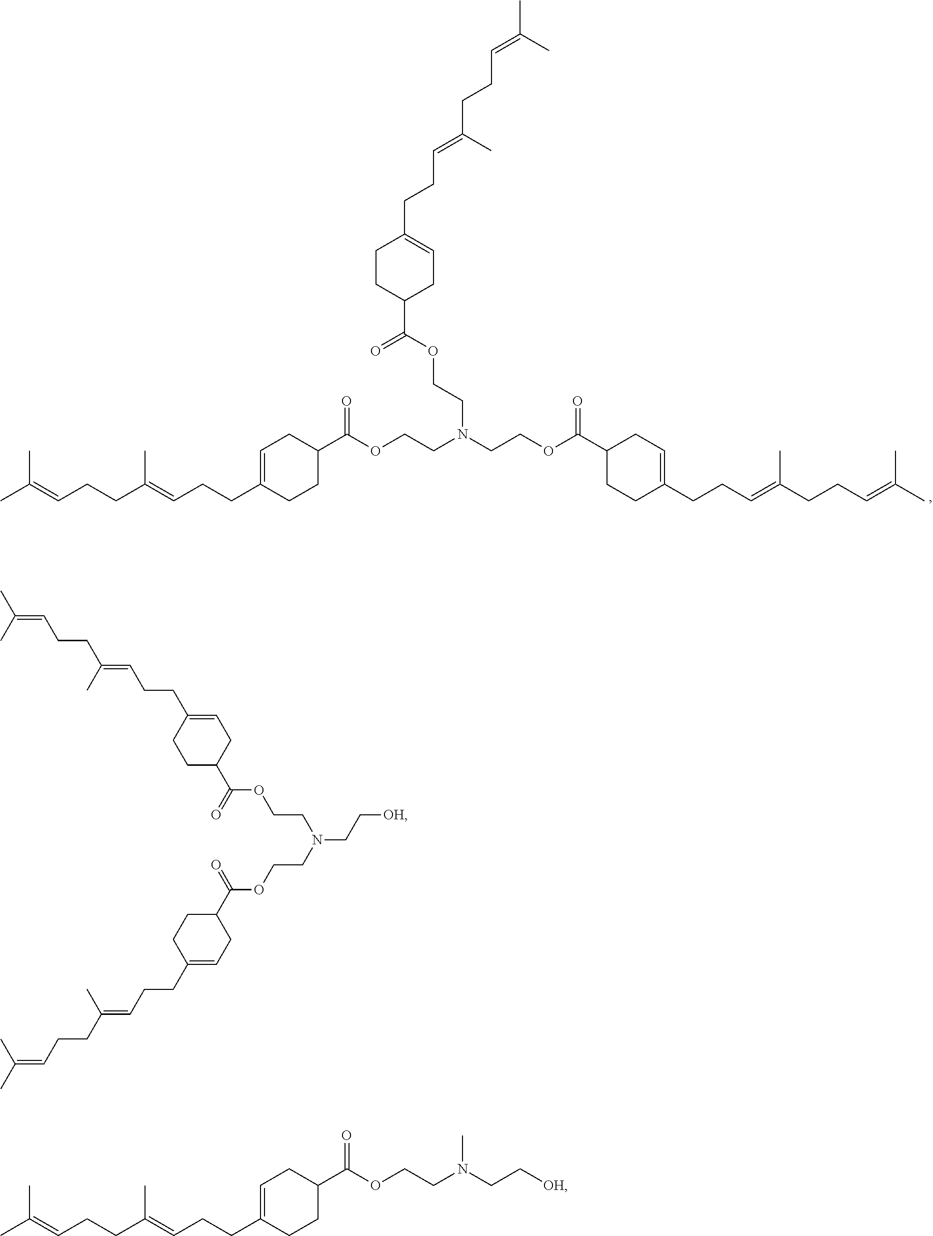

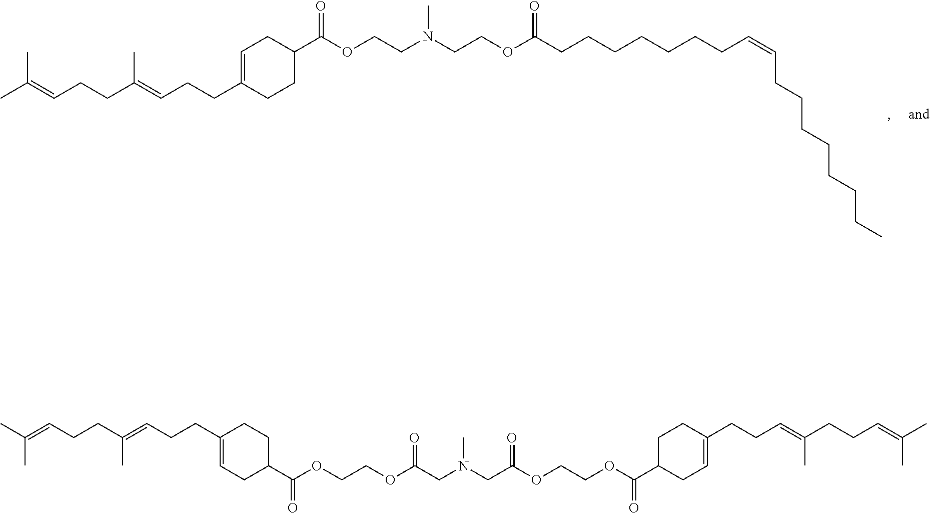

Structures of myrcene lipidoids that can be obtained by such scheme are as follows:

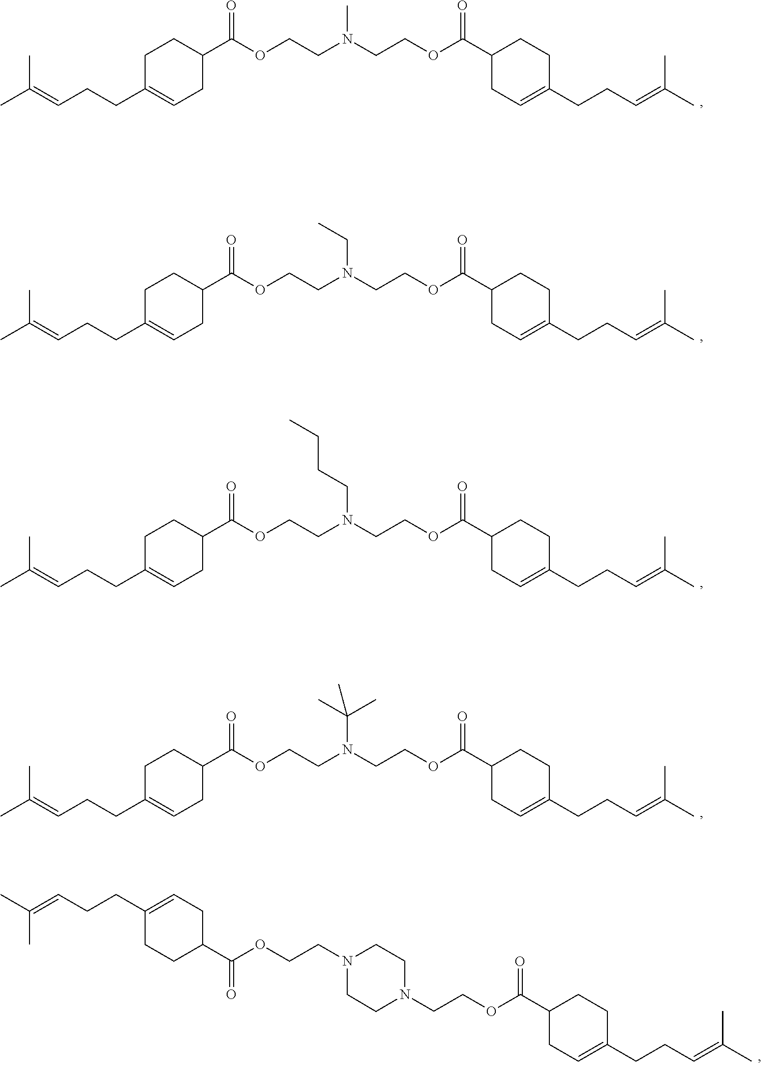

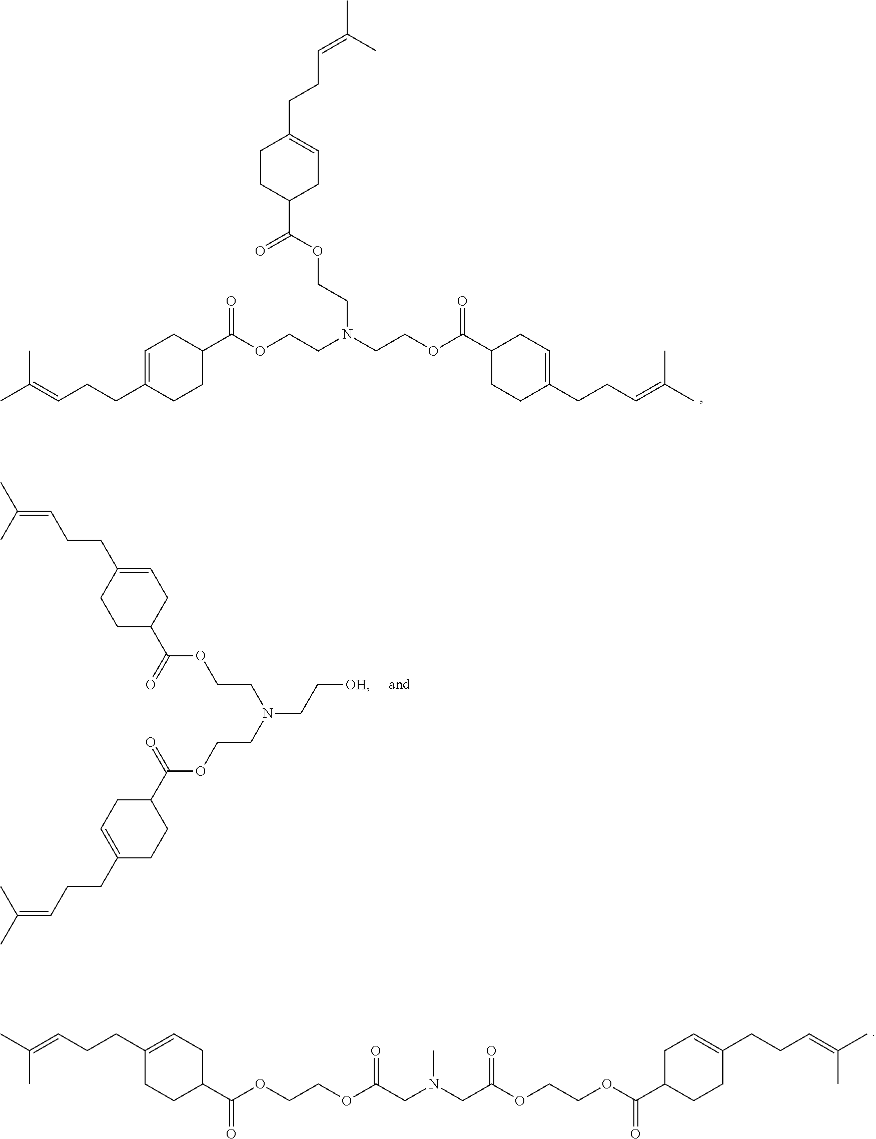

Reaction conditions for the synthesis of second generation lipidoids are generally as follows.

### General Procedure for Diels-Alder Reaction (E)

In a 4 mL sealed tube, farnesene or myrcene (1.0 eq) is combined with methyl acrylate (1.1 eq). The tube was sealed and the reaction was stirred for 20 h at 130° C. The cooled reaction mixture was purified by silica gel flash column chromatography with 3-5% EtOAc/hexane eluants. This reaction produces an unequal mixture of 1,3- and 1,4-regioisomers that cannot be separated using conventional column chromatographic methods. This mixture of regioisomers was used in subsequent reactions without isomer separation.

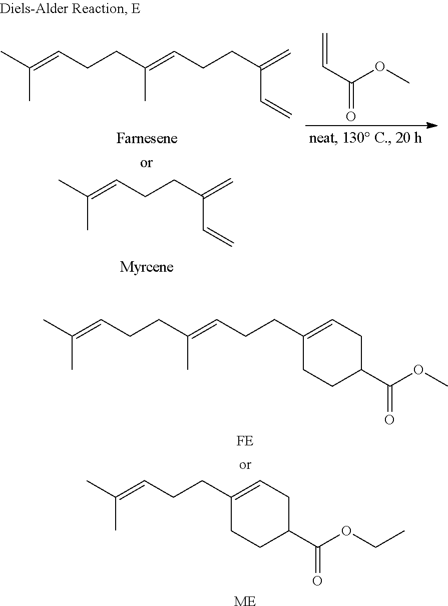

Suitable temperatures are generally between 30° C. and 150° C. Higher temperatures are generally associated with higher yields.

While the compounds in the scheme above are shown as 1,4 isomers, the compounds also include 1,3 isomers.

### General Procedure for Hydrogenation Reaction (F)

In a round-bottomed flask, compounds FE or ME was dissolved in mixed solvents of ethanol and CH₂Cl₂ (4:1). To this solution, 10% Pd/C (10% w/w) was added in one portion and the resulting dark suspension was stirred under a hydrogen atmosphere for 20 h at room temperature. The reaction mixture was filtered through celite and rinsed with CH₂Cl₂ (3 x). The filtrate was evaporated to give the hydrogenated compounds. The resulting residues proceeded to the next step without any purification.

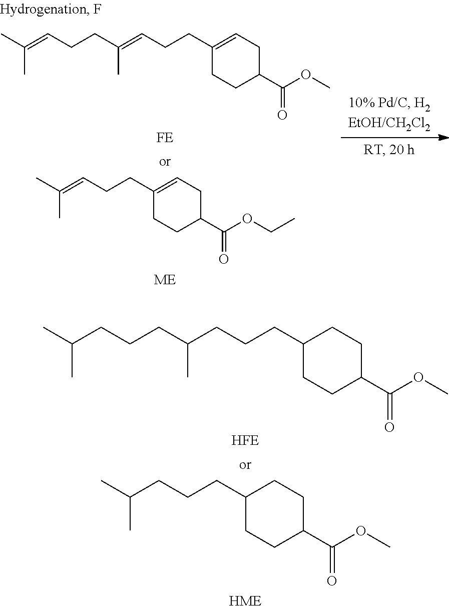

While the compounds in the scheme above are shown as 1,4 isomers, the compounds also include 1,3 isomers.

### General Procedure for Transesterification Reaction (G)

In a 4 mL glass tube, FE or ME (2.5 eq) is combined with an appropriate amine (1.0 eq) and triazabicyclodecene (TBD) (0.5 eq). The tube was sealed and the reaction mixture was stirred for 16 h at 130° C. The cooled reaction mixture was purified by silica gel flash column chromatography with 10-50% EtOAc/hexane or 5-10% MeOH/DCM eluants.

### General Procedure for Synthesis of FE-216 and ME-216 Lipidoids (H)

In a 20 mL scintillation glass vial, methyliminodiacetic acid (216) (1.0 eq), 1-ethyl-3-(3-dimethylamino propyl) carbodiimide (EDC-HCl) (2.2 eq), and dimethyl aminopyridine (DMAP) (2.2 eq) were dissolved in dichloromethane. The mixture was stirred for 15 minutes at room temperature before adding Compound 1 or Compound 3 (2.2 eq) dissolved in dichloromethane. The reaction was then stirred for 20 h at room temperature. The reaction mixture was transferred to a separatory funnel, added 20 mL of distilled water, and extracted with dichloromethane (3x). Combined organic extracts were dried over Na₂SO₄, filtered to a round bottom flask and the solvent was evaporated under reduced pressure. The crude residue was purified by silica gel flash column chromatography with 20% EtOAc/hexane eluant.

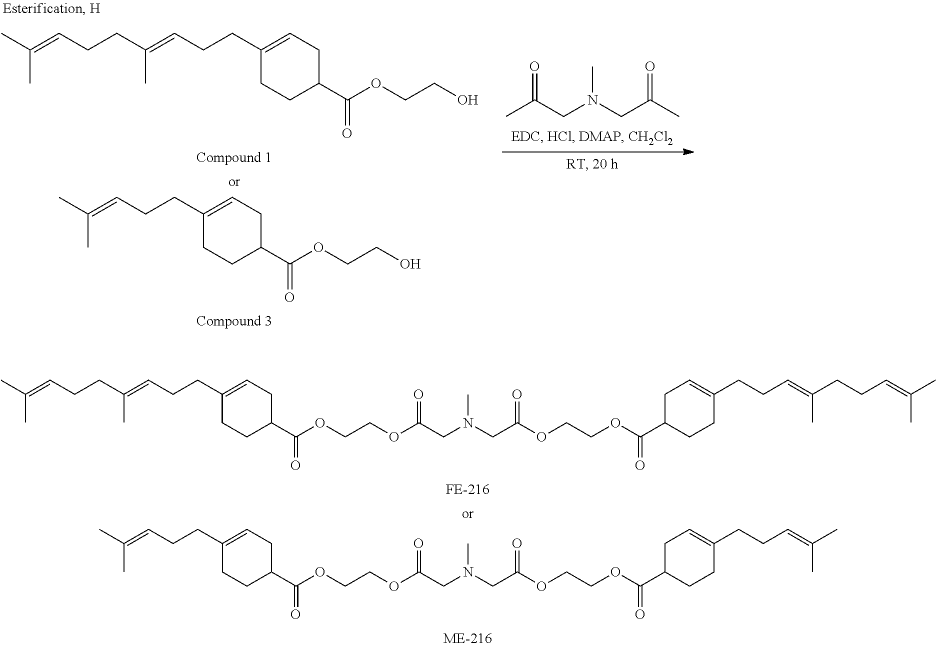

### General Procedure for Mono-transesterification Reaction (I)

In a 4 mL glass tube, FE or ME (1.1 eq) is combined with an appropriate amine (1.0 eq). The tube was sealed and the reaction mixture was stirred for 20 h at 130° C. The cooled reaction mixture was purified by silica gel flash column chromatography with 5-10% MeOH/DCM eluants.

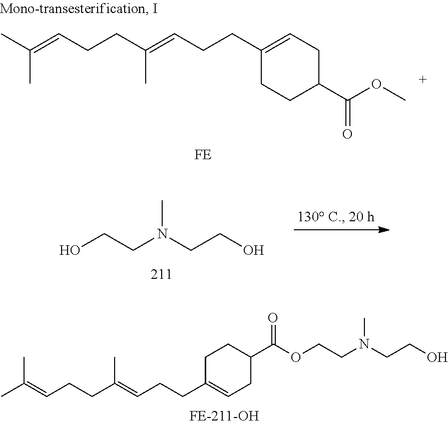

### General Procedure for Synthesis of Unsymmetrical Lipidoids (J)

In a 20 mL scintillation glass vial, appropriate carboxylic acid (1.0 eq), 1-ethyl-3-(3-dimethyl aminopropyl) carbodiimide (EDC-HCl) (1.1 eq), and dimethyl aminopyridine (DMAP) (1.1 eq) were dissolved in dichloromethane. The mixture was stirred for 15 minutes at room temperature before adding monoester (1.1 eq) dissolved in dichloromethane. The reaction was then stirred for 20 h at room temperature. The reaction mixture was transferred to a separatory funnel, added 20 mL of distilled water, and extracted with dichloromethane (3x). Combined organic extracts were dried over Na₂SO₄, filtered to a round bottom flask and the solvent was evaporated under reduced pressure.

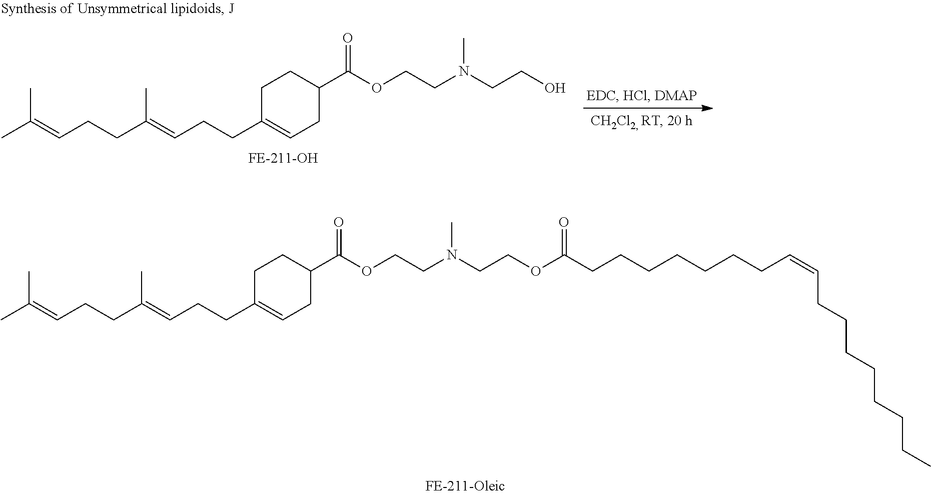 [SMILES: CC(C)=CCC/C(C)=C/CCC1=CCC(C(=O)OCCN(C)CCO)CC1 ; CCCCCCCC/C=C\CCCCCCCC(=O)OCCN(C)CCOC(=O)C1CC=C(CC/C=C(\C)CCC=C(C)C)CC1]

### Alternative General Procedure for Synthesis of Lipidoids (K)

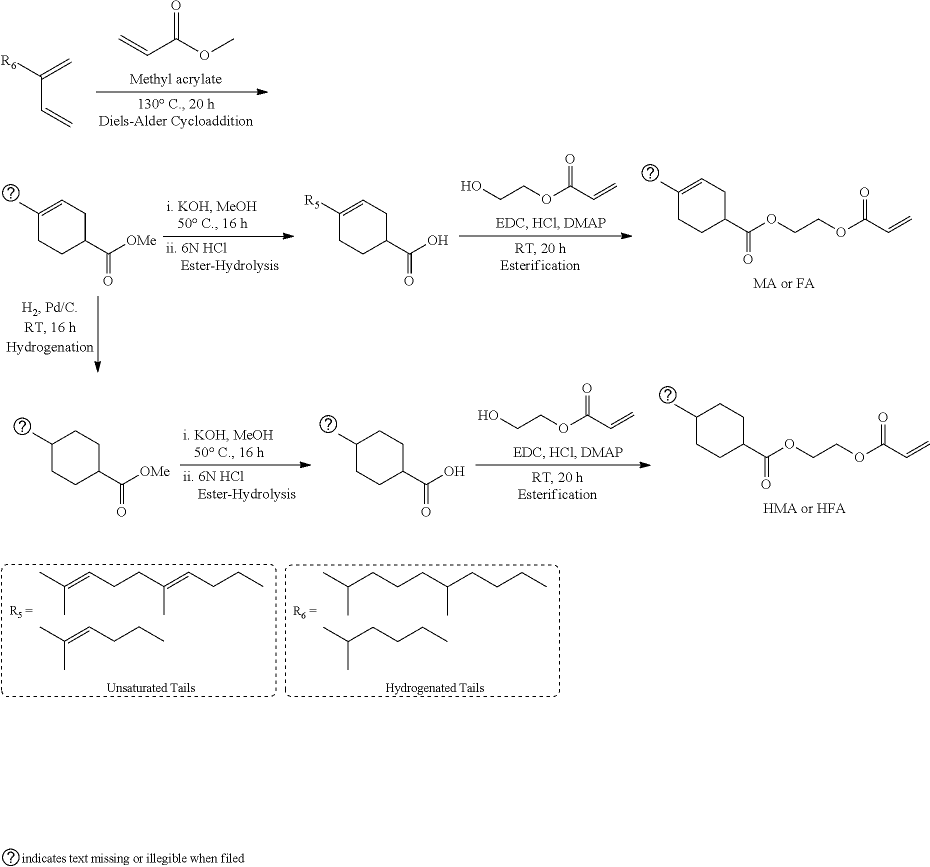

### Synthesis of MA

Step-1-Diels-Alder cycloaddition:

Myrcene (7.2 g, 52.8 mmol, 1.0 eq) and methyl acrylate (5.0 g, 58.1 mmol, 1.1 eq) were taken in a sealed tube. The tube was then sealed and stirred for 16 h at 130° C. The cooled reaction mixture was transferred to a round-bottom flask with CH₂Cl₂ and evaporated. The crude residue was purified by silica gel flash column chromatography with 3% EtOAc/hexane as eluants. TLC: 5% EtOAc/hexane, Iodine stain. 9.1 g (77%), colorless oil. 1H NMR (400 MHz, Chloroform-d) δ 5.42-5.35 (m, 1H), 5.12-5.04 (m, 1H), 3.69-3.66 (m, 3H), 2.60-2.43 (m, 1H), 2.30-1.86 (m, 10H), 1.67 (s, 3H), 1.59 (s, 3H).

Suitable temperatures are generally between 30° C. and 150° C. Higher temperatures are generally associated with higher yields.

Step-2-Ester hydrolysis:

In a 20 mL scintillation vial, the myrcene Diels-Alder adduct (634 mg, 1.0 eq) was dissolved in methanol (10 mL) and was added potassium hydroxide (239 mg, 1.5 eq). The reaction mixture was stirred for 16 h at 50° C. Water (50 mL) was added to the cooled reaction and extracted with diethyl ether (3×50 mL). The aqueous layer was acidified with 6N HCl until pH 2 and extracted with ethyl acetate (4×50 mL). The combined ethyl acetate extracts were washed with brine solution (50 mL); dried over Na₂SO₄; filtered; and evaporated. The resulting residue proceeded to the subsequent reaction without any purification. 561 mg (94%), colorless oil. 1H NMR (500 MHz, Chloroform-d) δ 5.45-5.37 (m, 1H), 5.14-5.05 (m, 1H), 2.66-2.50 (m, 1H), 2.36-1.93 (m, 10H), 1.69 (s, 3H), 1.61 (s, 3H).

This reaction can be achieved at lower temperatures with longer times for better yields.

### Step-3-Esterification:

In a 20 mL scintillation vial, the myrcene-based carboxylic acid (530 mg, 1.0 eq) from the above reaction was dissolved in CH₂Cl₂ (5 mL). To the solution, N-(3-dimethylaminopropyl)-N′-ethylcarbodiimide hydrochloride (585 mg, 1.2 eq) and 4-dimethylaminopyridine (124 mg, 0.4 eq) were added. The reaction mixture was then stirred for 15 mins at room temperature. To this solution was added 2-hydroxyethyl acrylate (0.35 mL, 1.2 eq) dropwise and the resulting solution was stirred for 20 h at room temperature. Water (50 mL) was added and extracted with CH₂Cl₂ (4×50 mL). The combined organic extracts were washed with brine (50 mL); dried over Na₂SO₄; filtered; and evaporated. The crude product was purified by silica gel flash column chromatography with 5-6% EtOAc/hexane as eluants to give the title product MA as a colorless oil. 498 mg (64%), colorless oil. 1H NMR (400 MHZ, Chloroform-d) 8 6.42 (dt, J=17.3, 1.2 Hz, 1H), 6.12 (ddd, J=17.4, 10.4, 1.0 Hz, 1H), 5.85 (dd, J=10.4, 1.5 Hz, 1H), 5.44-5.31 (m, 1H), 5.14-4.99 (m, 1H), 4.42-4.27 (m, 4H), 2.66-2.42 (m, 1H), 2.28-1.87 (m, 10H), 1.66 (s, 3H), 1.58 (s, 3H).

### Synthesis of HMA

### Step-2-Hydrogenation:

In a 100 mL round bottom flask, the myrcene Diels-Alder adduct (3.86 g) was dissolved in mixed solvents of CH₂Cl₂ and ethanol (30 mL, 1:5 v/v). To this solution, 10% Pd/C (512 mg, 15% w/w to Diels-Alder adduct) was added in one portion and the resulting dark suspension was stirred for 16 h at room temperature under a hydrogen atmosphere. The hydrogen balloon was taken off and the reaction mixture was filtered through celite and rinsed with CH₂Cl₂ (5×50 mL). The filtrate was evaporated to dryness. The compound obtained was proceeded to the next reactions without any purification. TLC: 5% EtOAc/hexane, Iodine stain (disappearance of starting material). 3.76 g (quant.), colorless oil. 1H NMR (400 MHZ, Chloroform-d) 8 3.67-3.64 (m, 3H), 2.55-2.15 (m, 1H), 2.03-1.03 (m, 15H), 0.95-0.81 (m, 7H).

Step-3-Ester hydrolysis:

In a 100 mL round bottom flask, the myrcene-based hydrogenated acid (6.5 g, 1.0 eq) was dissolved in methanol (40 mL) and was added potassium hydroxide (2.4 g, 1.5 eq). The reaction mixture was stirred for 16 h at 50° C. Water (100 mL) was added to the cooled reaction and extracted with diethyl ether (3×50 mL). The aqueous layer was acidified with 6N HCl until pH 2 and extracted with ethyl acetate (4×100 mL). The combined ethyl acetate extracts were washed with brine solution (50 mL); dried over Na₂SO₄; filtered; and evaporated. The resulting residue proceeded to the subsequent reaction without any purification. 5.2 g, colorless oil. 1H NMR (500 MHZ, Chloroform-d) 8 2.61-2.19 (m, 1H), 2.10-1.63 (m, 4H), 1.62-1.00 (m, 12H), 0.98-0.81 (m, 7H).

This reaction can be achieved at lower temperatures with longer times for better yields.

### Step-4-Esterification:

In a 100 mL round bottom flask, the myrcene-based carboxylic acid (5.16 g, 1.0 eq) from the above reaction was dissolved in CH₂Cl₂ (50 mL). To the solution, N-(3-dimethylaminopropyl)-N′-ethylcarbodiimide hydrochloride (5.6 g, 1.2 eq) and 4-dimethylaminopyridine (1.2 g, 0.4 eq) were added. The reaction mixture was then stirred for 15 mins at room temperature. To this solution was added 2-hydroxyethyl acrylate (3.35 mL, 1.2 eq) dropwise and the resulting solution was stirred for 20 h at room temperature. Water (50 mL) was added and extracted with CH₂Cl₂ (4×100 mL). The combined organic extracts were washed with brine (50 mL); dried over Na₂SO₄; filtered; and evaporated. The crude product was purified by silica gel flash column chromatography with 5-6% EtOAc/hexane as eluants to give the title product HMA as a colorless oil. 5 7 g (76%), colorless oil. 1H NMR (500 MHZ, Chloroform-d) § 6.51-6.33 (m, 1H), 6.22-6.05 (m, 1H), 5.92-5.80 (m, 1H), 4.43-4.24 (m, 4H), 2.75-2.16 (m, 1H), 2.02-1.64 (m, 4H), 1.59-0.98 (m, 12H), 0.89-0.84 (m, 6H).

### Alternative General Procedure for Synthesis of Lipidoids (L)

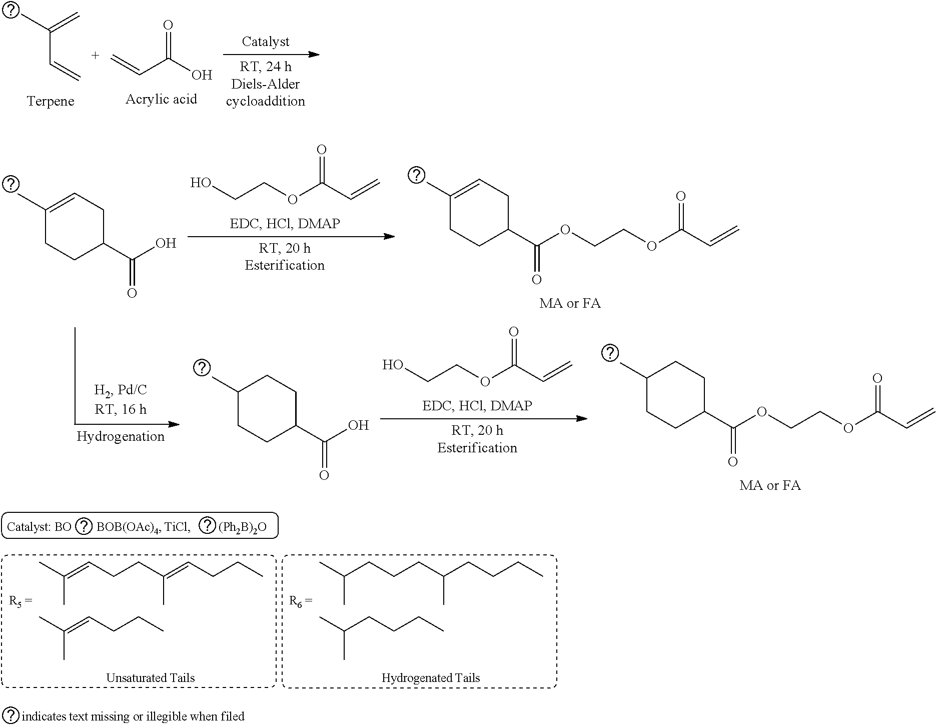

### Synthesis of HMA

Diels-Alder cycloaddition:

In a 4 mL sealed tube, acrylic acid (0.5 g, 1.0 eq), catalyst tetraacetyl diborate (BOB (OAc) 4) (40 mg, 2 mol %), and myrcene (1.04 g, 1.1 eq) were taken. The tube was then sealed and stirred for 3 days at room temperature. The reaction was then transferred to a separatory funnel with CH₂Cl₂ (50 mL) and added water (20 mL) followed by brine (20 mL). The separatory funnel was shaken and the organic layer was separated. The aqueous layer was extracted with CH₂Cl₂ (3×50 mL). Combined organic layers were dried over Na₂SO₄, filtered, and evaporated. The crude residue was adsorbed onto silica gel and purified through silica gel flash column chromatography with 10% EtOAc/hexane. 583 mg (40%), colorless semi-solid. ¹H NMR (500 MHz, Chloroform-d) δ 5.45-5.37 (m, 1H), 5.14-5.05 (m, 1H), 2.66-2.50 (m, 1H), 2.36-1.93 (m, 10H), 1.69 (s, 3H), 1.61 (s, 3H).

Suitable temperatures are generally between 30° C. and 150° C. Higher temperatures are generally associated with higher yields.

### Hydrogenation:

In a 100 mL round bottom flask, the myrcene-based Diels-Alder adduct (440 mg) was dissolved in mixed solvents of CH₂Cl₂ and ethanol (30 mL, 1:5 v/v). To this solution, 10% Pd/C (98 mg, 15% w/w to Diels-Alder adduct) was added in one portion and the resulting dark suspension was stirred for 16 h at room temperature under a hydrogen atmosphere. The hydrogen balloon was taken off and the reaction mixture was filtered through celite and rinsed with CH₂Cl₂ (5×50 mL). The filtrate was evaporated to dryness. The compound obtained was proceeded to the next reactions without any purification. TLC: 5% EtOAc/hexane, Iodine stain (disappearance of starting material). 425 mg (quant.), colorless oil. 1H NMR (500 MHZ, Chloroform-d) 8 2.61-2.19 (m, 1H), 2.10-1.63 (m, 4H), 1.62-1.00 (m, 12H), 0.98-0.81 (m, 7H).

### Esterification:

In a 20 mL scintillation vial, the hydrogenated myrcene-based carboxylic acid (425 mg, 1.0 eq) from the above reaction was dissolved in CH₂Cl₂ (5 mL). To the solution, N-(3-dimethylaminopropyl)-N′-ethylcarbodiimide hydrochloride (461 mg, 1.2 eq) and 4-dimethylaminopyridine (98 mg, 0.4 eq) were added. The reaction mixture was then stirred for 15 mins at room temperature. To this solution was added 2-hydroxyethyl acrylate (0.28 mL, 1.2 eq) dropwise and the resulting solution was stirred for 20 h at room temperature. Water (50 mL) was added and extracted with CH₂Cl₂ (4×50 mL). The combined organic extracts were washed with brine (50 mL); dried over Na₂SO₄; filtered; and evaporated. The crude product was purified by silica gel flash column chromatography with 5-6% EtOAc/hexane as eluants to give the title product HMA as a colorless oil. 538 mg (86%), colorless oil. 1H NMR (500 MHZ, Chloroform-d) 8 6.51-6.33 (m, 1H), 6.22-6.05 (m, 1H), 5.92-5.80 (m, 1H), 4.43-4.24 (m, 4H), 2.75-2.16 (m, 1H), 2.02-1.64 (m, 4H), 1.59-0.98 (m, 12H), 0.89-0.84 (m, 6H).

### Alternative General Procedure for Synthesis of Lipidoids (M)

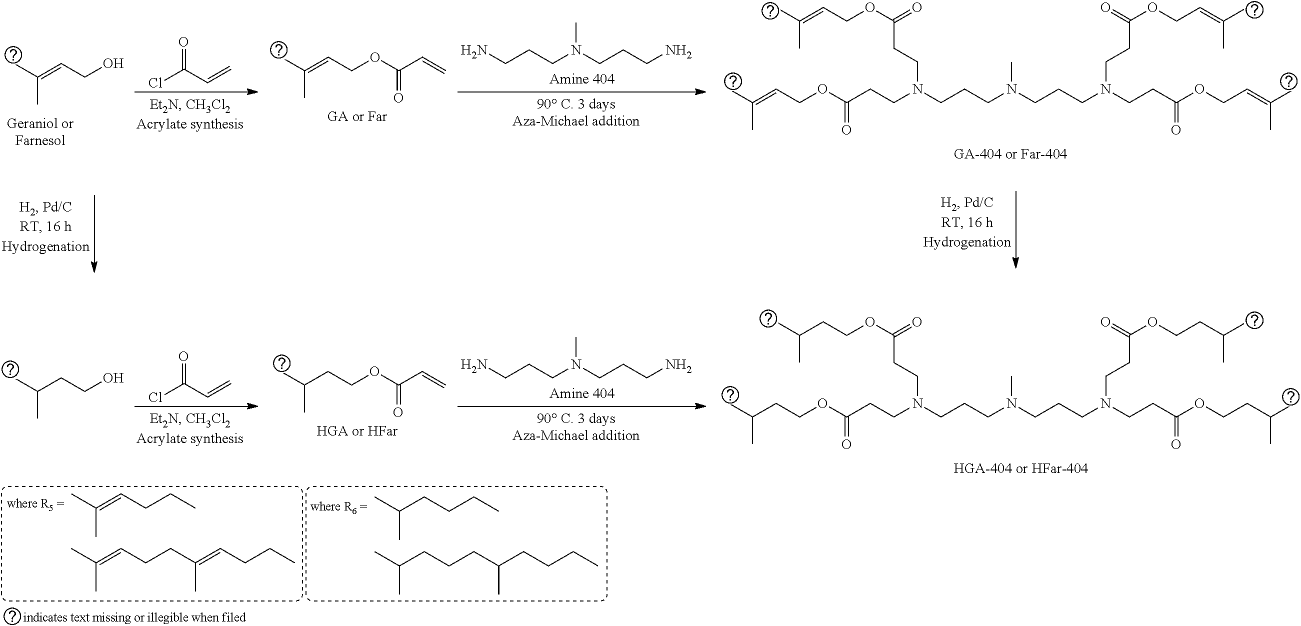

### Synthesis of GA-404

Acrylate synthesis:

In a 100 mL round bottom flask, geraniol (1.0 eq) was dissolved in anhydrous CH₂C₁₂ ( ) and was added triethylamine (1.5 eq). The reaction solution was cooled to 0° C. with ice/water bath. Acryloyl chloride (1.2 eq) was added to the reaction dropwise for 15 mins and the resulting pale-yellow solution was stirred for 16 h allowing the temperature to raise to room temperature (25° C.). The solvent in the reaction was evaporated and purified by silica gel flash column chromatography with 4-5% EtOAc/hexane as eluants. Colorless oil. ¹H NMR (400 MHz, Chloroform-d) § 6.40 (dd, J=17.3, 1.5 Hz, 1H), 6.13 (dd, J=17.3, 10.4 Hz, 1H), 5.81 (dd, J=10.4, 1.5 Hz, 1H), 5.37 (tq, J=7.1, 1.3 Hz, 1H), 5.12-5.04 (m, 1H), 4.68 (dd, J=7.2, 0.9 Hz, 2H), 2.15-2.01 (m, 4H), 1.72 (s, 3H), 1.69-1.66 (m, 3H), 1.60 (s, 3H).

Aza-Michael addition:

In a 4 mL scintillation vial, the amine-404 (1.0 eq) and acrylate (4.5 eq) from the above reaction was taken. The capped vial was then stirred for 3 days at 90° C. The cooled reaction mixture was purified by silica gel flash column chromatography with 4-5% MeOH/CH₂Cl₂ as eluants. Pale-yellow oil. 1H NMR (400 MHZ, Chloroform-d) 8 5.31 (tq, J=7.0, 1.4 Hz, 4H), 5.06 (tp, J=6.8, 1.5 Hz, 4H), 4.56 (d, J=7.1 Hz, 8H), 2.73 (t, J=7.1 Hz, 8H), 2.50-2.36 (m, 15H), 2.19-1.90 (m, 18H), 1.87-1.70 (m, 6H), 1.70-1.67 (m, 12H), 1.66 (s, 12H), 1.58 (s, 12H).

Suitable temperatures are generally between 30° C. and 150° C.

### Alternative General Procedure for Synthesis of Lipidoids (N)

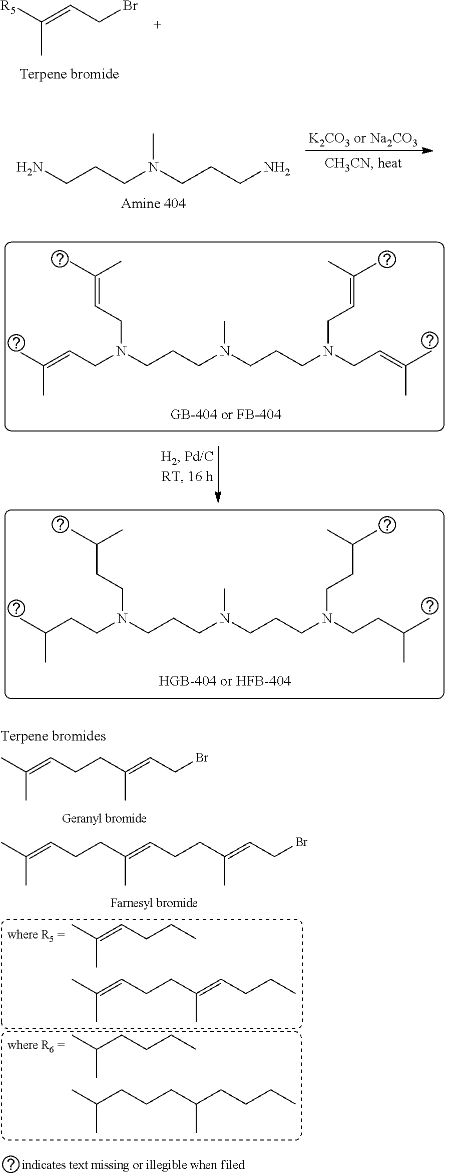

For all of the schemes listed above, similar solvents can be used instead of the solvents depicted in the schemes.

Further, all hydrogenated lipids (HMA-series, HFA-series, HGA-series, and HFar-series) can be prepared from their unsaturated analogs (MA-series, FA-series, GA-series, and Far-series) by direct hydrogenation with higher catalyst loading and longer times. HMA-404, for example, can be synthesized directly from MA-404 using any catalytic hydrogenation method (including the hydrogenation protocol given in the application with reaction conditions modified).

### Lnp Components

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one terpene-derived compound of the present disclosure by moles. In some aspects, the at least one terpene-derived compound is at least one compound of Formula (I), Formula (II), or Formula (III), as described herein. In some aspects, the at least one compound of the present disclosure is a mixture of two or more compounds of Formula (I), Formula (II), or Formula (III).

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one terpene-derived compound of the present disclosure by moles. In some aspects, the at least one terpene-derived compound is at least one compound of Formula (I), Formula (II), or Formula (III), as described herein. In some aspects, the at least one compound of the present disclosure is a mixture of two or more compounds of Formula (I), Formula (II), or Formula (III).

In some aspects, an LNP can further comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one structural lipid by moles.

In some aspects, an LNP can further comprise at least about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one structural lipid by moles.

In some aspects, a LNP can further comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one phospholipid by moles.

In some aspects, an LNP can further comprise at least about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one phospholipid by moles.

In some aspects, a LNP can further comprise at least about 0.25%, or at least about 0.5%, or at least about 0.75%, or at least about 1.0%, or at least about 2.5%, or at least about 5%, or at least about 7.5%, or at least about 10% PEGylated lipid by moles.

### A. Structural Lipid

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

### B. Phospholipid

As used herein, the term “phospholipid” is used in its broadest sent to refer to any amphiphilic molecule that comprises a polar (hydrophilic) headgroup comprising phosphate and two hydrophobic fatty acid chains. In some aspects, a phospholipid can comprise dioleoylphosphatidylethanolamine (DOPE). In some aspects, a phospholipid can comprise 1,2-Distearoyl-sn-glycero-3-phosphocholine (DSPC). In some aspects, a phospholipid can comprise 1,2-Dioleoyl-sn-glycero-3-phosphocholine (DOPC). In some aspects, a phospholipid can comprise DDPC (1,2-Didecanoyl-sn-glycero-3-phosphocholine), DEPA-NA (1,2-Dierucoyl-sn-glycero-3-phosphate (Sodium Salt)), DEPC (1,2-Dierucoyl-sn-glycero-3-phosphocholine), DEPE (1,2-Dierucoyl-sn-glycero-3-phosphoethanolamine), DEPG-NA (1,2-Dierucoyl-sn-glycero-3 [Phospho-rac-(1-glycerol) (Sodium Salt)), DLOPC (1,2-Dilinoleoyl-sn-glycero-3-phosphocholine), DLPA-NA (1,2-Dilauroyl-sn-glycero-3-phosphate (Sodium Salt)), DLPC (1,2-Dilauroyl-sn-glycero-3-phosphocholine), DLPE (1,2-Dilauroyl-sn-glycero-3-phosphoethanolamine), DLPG-NA (1,2-Dilauroyl-sn-glycero-3 [Phospho-rac-(1-glycerol) (Sodium Salt)), DLPG-NH4 (1,2-Dilauroyl-sn-glycero-3 [Phospho-rac-(1-glycerol) (Ammonium Salt)), DLPS-NA (1,2-Dilauroyl-sn-glycero-3-phosphoserine (Sodium Salt)), DMPA-NA (1,2-Dimyristoyl-sn-glycero-3-phosphate (Sodium Salt)), DMIPC (1,2-Dimyristoyl-sn-glycero-3-phosphocholine), DMPE (1,2-Dimyristoyl-sn-glycero-3-phosphoethanolamine), DMPG-NA (1,2-Dimyristoyl-sn-glycero-3 [Phospho-rac-(1-glycerol) (Sodium Salt)), DMPG-NH4 (1,2-Dimyristoyl-sn-glycero-3 [Phospho-rac-(1-glycerol) (Ammonium Salt)), DMPG-NH4/NA (1,2-Dimyristoyl-sn-glycero-3 [Phospho-rac-(1-glycerol) (Sodium/Ammonium Salt)), DMPS-NA (1,2-Dimyristoyl-sn-glycero-3-phosphoserine (Sodium Salt)), DOPA-NA (1,2-Dioleoyl-sn-glycero-3-phosphate (Sodium Salt)), DOPC (1,2-Dioleoyl-sn-glycero-3-phosphocholine), DOPE (1,2-Dioleoyl-sn-glycero-3-phosphoethanolamine), DOPG-NA (1,2-Dioleoyl-sn-glycero-3 [Phospho-rac-(1-glycerol) (Sodium Salt)), DOPS-NA (1,2-Dioleoyl-sn-glycero-3-phosphoserine (Sodium Salt)), DPPA-NA (1,2-Dipalmitoyl-sn-glycero-3-phosphate (Sodium Salt)), DPPC (1,2-Dipalmitoyl-sn-glycero-3-phosphocholine), DPPE (1,2-Dipalmitoyl-sn-glycero-3-phosphoethanolamine), DPPG-NA (1,2-Dipalmitoyl-sn-glycero-3 [Phospho-rac-(1-glycerol) (Sodium Salt)), DPPG-NH4 (1,2-Dipalmitoyl-sn-glycero-3 [Phospho-rac-(1-glycerol) (Ammonium Salt)), DPPS-NA (1,2-Dipalmitoyl-sn-glycero-3-phosphoserine (Sodium Salt)), DSPA-NA (1,2-Distearoyl-sn-glycero-3-phosphate (Sodium Salt)), DSPC (1,2-Distearoyl-sn-glycero-3-phosphocholine), DSPE (1,2-Distearoyl-sn-glycero-3-phosphoethanolamine), DSPG-NA (1,2-Distearoyl-sn-glycero-3 [Phospho-rac-(1-glycerol) (Sodium Salt)), DSPG-NH4 (1,2-Distearoyl-sn-glycero-3 [Phospho-rac-(1-glycerol) (Ammonium Salt)), DSPS-NA (1,2-Distearoyl-sn-glycero-3-phosphoserine (Sodium Salt)), EPC (Egg-PC), HEPC

(Hydrogenated Egg PC), HSPC (Hydrogenated Soy PC), LYSOPC MYRISTIC (1-Myristoyl-sn-glycero-3-phosphocholine), LYSOPC PALMITIC (1-Palmitoyl-sn-glycero-3-phosphocholine), LYSOPC STEARIC (1-Stearoyl-sn-glycero-3-phosphocholine), Milk Sphingomyelin (MPPC; 1-Myristoyl-2-palmitoyl-sn-glycero 3-phosphocholine), MSPC (1-Myristoyl-2-stearoyl-sn-glycero-3-phosphocholine), PMPC (1-Palmitoyl-2-myristoyl-sn-glycero-3-phosphocholine), POPC (1-Palmitoyl-2-oleoyl-sn-glycero-3-phosphocholine), POPE (1-Palmitoyl-2-oleoyl-sn-glycero-3-phosphoethanolamine), POPG-NA (1-Palmitoyl-2-oleoyl-sn-glycero-3 [Phospho-rac-(1-glycerol)] (Sodium Salt)), PSPC (1-Palmitoyl-2-stearoyl-sn-glycero-3-phosphocholine), SMPC (1-Stearoyl-2-myristoyl-sn-glycero-3-phosphocholine), SOPC (1-Stearoyl-2-oleoyl-sn-glycero-3-phosphocholine), SPPC (1-Stearoyl-2-palmitoyl-sn-glycero-3-phosphocholine), or any combination thereof.

### C. PEGylated Lipid

As used herein, the term “PEGylated lipid” is used to refer to any lipid that is modified (e.g. covalently linked to) at least one polyethylene glycol molecule. In some aspects, a PEGylated lipid can comprise 1,2-dimyristoyl-rac-glycero-3-methoxypolyethylene glycol-2000, hereafter referred to as DMG-PEG2000.

### LNP Compositions

In some aspects, a lipid nanoparticle can comprise at least one nucleic acid molecule, at least one compound of the present disclosure, and at least one structural lipid.

In some aspects, a lipid nanoparticle can comprise at least one nucleic acid molecule, at least one compound of the present disclosure, and at least one PEGylated lipid.

In some aspects, the at least one structural lipid is a mixture of two structural lipids.

In some aspects, the at least one PEGylated lipid is a mixture of two PEGylated lipids.

In some aspects, a lipid nanoparticle can comprise at least one nucleic acid molecule, at least one compound of the present disclosure, at least one structural lipid, at least one PEGylated lipid or any combination thereof.

In some aspects, a lipid nanoparticle can comprise at least one nucleic acid, at least one compound of the present disclosure, at least one structural lipid, and at least one PEGylated lipid.

In some aspects, a lipid nanoparticle can comprise at least one nucleic acid molecule, at least one compound of the present disclosure, at least one structural lipid, at least one phospholipid, at least one PEGylated lipid or any combination thereof.

In some aspects, a lipid nanoparticle can comprise at least one nucleic acid, at least one compound of the present disclosure, at least one structural lipid, at least one phospholipid and at least one PEGylated lipid.

In some aspects, the at least one compound of the present disclosure is a compound of Formula (I), Formula (II), or Formula (III).

In some aspects, the at least one compound of the present disclosure is a mixture of two or more compounds of Formula (I), Formula (II), or Formula (III).

In some aspects, a lipid nanoparticle comprising at least one nucleic acid can comprise about 41.4% of at least one compound of Formula (I), Formula (II), or Formula (III) by moles, about 10% of at least one structural lipid by moles, about 45.9% of at least one phospholipid by moles, and about 2.7% of at least one PEGylated lipid by moles. In some aspects, a lipid nanoparticle comprising at least one nucleic acid can comprise about 31.4% to about 51.4% of at least one compound of Formula (I), Formula (II), or Formula (III) by moles, about 1% to about 20% of at least one structural lipid by moles, about 35.9% to about 55.9% of at least one phospholipid by moles, and about 0.1% to about 12.7% of at least one PEGylated lipid by moles. In some aspects, a lipid nanoparticle comprising at least one nucleic acid can comprise about 36.4% to about 46.4% of at least one compound of Formula (I), Formula (II), or Formula (III) by moles, about 5% to about 15% of at least one structural lipid by moles, about 40.9% to about 50.9% of at least one phospholipid by moles, and about 1% to about 7.7% of at least one PEGylated lipid by moles.

In some aspects, a lipid nanoparticle comprising at least one nucleic acid can comprise about 33.5% of at least one compound of Formula (I), Formula (II), or Formula (III) by moles, about 33.5% of at least one structural lipid by moles, about 32% of at least one phospholipid by moles, and about 1% of at least one PEGylated lipid by moles. In some aspects, a lipid nanoparticle comprising at least one nucleic acid can comprise about 23.5% to about 43.5% of at least one compound of Formula (I), Formula (II), or Formula (III) by moles, about 23.5% to about 43.5% of at least one structural lipid by moles, about 22% to about 42% of at least one phospholipid by moles, and about 0.1% to about 11% of at least one PEGylated lipid by moles. In some aspects, a lipid nanoparticle comprising at least one nucleic acid can comprise about 28.5% to about 38.5% of at least one compound of Formula (I), Formula (II), or Formula (III) by moles, about 28.5% to about 38.5% of at least one structural lipid by moles, about 27% to about 37% of at least one phospholipid by moles, and about 0.5% to about 6% of at least one PEGylated lipid by moles.

In some aspects, a lipid nanoparticle comprising at least one nucleic acid can comprise about 38% of at least one compound of Formula (I), Formula (II), or Formula (III) by moles, about 10% of at least one structural lipid by moles, about 50% of at least one phospholipid by moles, and about 2% of at least one PEGylated lipid by moles. In some aspects, a lipid nanoparticle comprising at least one nucleic acid can comprise about 28% to about 48% of at least one compound of Formula (I), Formula (II), or Formula (III) by moles, about 1% to about 20% of at least one structural lipid by moles, about 40% to about 60% of at least one phospholipid by moles, and about 0.1% to about 12% of at least one PEGylated lipid by moles. In some aspects, a lipid nanoparticle comprising at least one nucleic acid can comprise about 33% to about 43% of at least one compound of Formula (I), Formula (II), or Formula (III) by moles, about 5% to about 15% of at least one structural lipid by moles, about 45% to about 55% of at least one phospholipid by moles, and about 1% to about 6% of at least one PEGylated lipid by moles.

In some aspects, a lipid nanoparticle comprising at least one nucleic acid can comprise about 35% of at least one compound of Formula (I), Formula (II), or Formula (III) by moles, about 41.8% of at least one structural lipid by moles, about 20% of at least one phospholipid by moles, and about 3.2% of at least one PEGylated lipid by moles. In some aspects, a lipid nanoparticle comprising at least one nucleic acid can comprise about 25% to about 45% of at least one compound of Formula (I), Formula (II), or Formula (III) by moles, about 31.8% to about 51.8% of at least one structural lipid by moles, about 10% to about 30% of at least one phospholipid by moles, and about 0.1% to about 13.2% of at least one PEGylated lipid by moles. In some aspects, a lipid nanoparticle comprising at least one nucleic acid can comprise about 30% to about 40% of at least one compound of Formula (I), Formula (II), or Formula (III) by moles, about 36.8% to about 46.8% of at least one structural lipid by moles, about 15% to about 25% of at least one phospholipid by moles, and about 1% to about 8.2% of at least one PEGylated lipid by moles.

In some aspects of the LNPs described herein, the compound of Formula (I) comprised in the LNP composition is one of COMPOUNDS 5-49.

In some aspects of the LNPs described herein, the compound of Formula (I) comprised in the LNP composition is COMPOUND 9.

In some aspects of the LNPs described herein, the compound of Formula (I) comprised in the LNP composition is COMPOUND 16.

In some aspects of the LNPs described herein, the compound of Formula (I) comprised in the LNP composition is COMPOUND 17.

In some aspects of the LNPs described herein, the compound of Formula (I) comprised in the LNP composition is COMPOUND 18.

In some aspects of the LNPs described herein, the compound of Formula (I) comprised in the LNP composition is COMPOUND 22.

In some aspects of the LNPs described herein, the compound of Formula (I) comprised in the LNP composition is COMPOUND 23.

In some aspects of the LNPs described herein, the compound of Formula (I) comprised in the LNP composition is COMPOUND 29.

In some aspects of the LNPs described herein, the compound of Formula (I) comprised in the LNP composition is COMPOUND 30.

In some aspects of the LNPs described herein, the compound of Formula (I) comprised in the LNP composition is COMPOUND 42.

In some aspects of the LNPs described herein, the compound of Formula (I) comprised in the LNP composition is COMPOUND 55.

In some aspects of the LNPs described herein, the structural lipid can be cholesterol. In some aspects of the LNPs described herein, the phospholipid can be DOPE. In some aspects of the preceding LNPs, the phospholipid can be DSPC. In some aspects of the preceding LNPs, the phospholipid can be DOPC. In some aspects of the LNPs described herein, the PEGylated lipid can be DMG-PEG2000.

In some aspects of the LNPs described herein, the structural lipid can be cholesterol, the phospholipid can be DOPE and the PEGylated lipid can be DMG-PEG2000

In some aspects of the LNPs described herein, the structural lipid can be cholesterol, the phospholipid can be DOPC and the PEGylated lipid can be DMG-PEG2000.

In some aspects of the LNPs described herein, the at least one nucleic acid molecule is a DNA molecule. In one aspect, the at least one DNA molecule is a DoggyBone DNA molecule. In some aspects, the at least one DNA molecule is a DNA nanoplasmid.

In some aspects, the DNA molecule can comprise, consist of, or consist essentially of a nucleic acid sequence at least 65%, 70%, 75%, 80%, 85%, 90%, 95%, 96%, 97%, 98%, 99% or 100% (or any percentage in between) identical to SEQ ID NO: 6.

In some aspects, a lipid nanoparticle comprising at least one nucleic acid can comprise at least one nucleic acid molecule. In some aspects, a lipid nanoparticle can comprise a plurality of nucleic acid molecules. In some aspects, the at least one nucleic acid molecule or the plurality of nucleic acid molecules can be formulated in a lipid nanoparticle.

In some aspects, an at least one nucleic acid molecule can comprise at least one RNA molecule and at least one DNA molecule. That is, the LNPs of the present disclosure can comprise both RNA molecules and DNA molecules.

In some aspects, the LNPs of the present disclosure can comprise both RNA molecules and DNA molecules, wherein the RNA molecules comprise at least one nucleic acid sequence that encodes a transposase and wherein the DNA molecules comprise at least one nucleic acid sequence that comprises a transposon. In some aspects, the transposase can be any of the transposases described herein. In some aspects, the transposon can be a transposon comprising at least one nucleic acid sequence encoding a FVIII polypeptide. In some aspects, the DNA molecule can comprise, consist of, or consist essentially of a nucleic acid sequence at least 65%, 70%, 75%, 80%, 85%, 90%, 95%, 96%, 97%, 98%, 99% or 100% (or any percentage in between) identical to SEQ ID NO: 6.

In some aspects, the LNPs of the present disclosure can comprise both mRNA molecules and gRNA molecules, wherein the mRNA molecules comprise at least one nucleic acid sequence that encodes a fusion protein, wherein the fusion protein comprises: (i) an inactivated Cas9 (dCas9) protein or an inactivated nuclease domain thereof; and (ii) a Clo051 protein or a nuclease domain thereof, and wherein the gRNA molecules encode guide RNA sequence targeting one or more specific genomic loci. In some aspects, the fusion protein can be a Cas-CLOVER protein. In some aspects, the gRNA molecules can target the psk9 gene.

In some aspects wherein the LNPs of the present disclosure comprise both RNA (e.g. mRNA) and DNA, the ratio of RNA to DNA (RNA: DNA) in the LNPs can be about 1:1, or about 2:1, or about 3:1, or about 4:1, or about 5:1, or about 6:1, or about 7:1, or about 8:1, or about 9:1 or about 10:1.

In some aspects, wherein the LNPs of the present disclosure comprise mRNA, gRNA and DNA, the ratio of mRNA to gRNA to DNA (mRNA: gRNA: DNA) can be about 1:1:1, about 2:1:1, about 3:1:1, about 4:1:1 or about 5:1:1.

In some aspects, wherein the LNPs of the present disclosure comprise mRNA, gRNA and DNA, the ratio of gRNA to mRNA to DNA (gRNA: mRNA: DNA) can be about 1:1:1, about 2:1:1, about 3:1:1, about 4:1:1 or about 5:1:1.

In some aspects, wherein the LNPs of the present disclosure comprise mRNA, gRNA and DNA, the ratio of DNA to gRNA to mRNA (DNA: gRNA: mRNA) can be about 1:1:1, about 2:1:1, about 3:1:1, about 4:1:1 or about 5:1:1.

In some aspects, a lipid nanoparticle can comprise lipid and nucleic acid at a specified ratio (weight/weight).

In some aspects, a lipid nanoparticle comprising at least one nucleic acid can comprise lipid and nucleic acid at a ratio of about 5:1 to about 15:1, or about 10:1 to about 20:1, or about 15:1 to about 25:1, or about 20:1 to about 30:1, or about 25:1 to about 35:1 or about 30:1 to about 40:1, or about 35:1 to about 45:1, or about 40:1 to about 50:1, or about 45:1 to about 55:1, or about 50:1 to about 60:1, or about 55:1 to about 65:1, or about 60:1 to about 70:1, or about 65:1 to about 75:1, or about 70:1 to about 80:1, or about 75:1 to about 85:1, or about 80:1 to about 90:1, or about 85:1 to about 95:1, or about 90:1 to about 100:1, or about 95:1 to about 105:1, or about 100:1 to about 110:1, or about 105:1 to about 115:1, or about 110:1 to about 120:1, or about 115:1 to about 125:1, or about 120:1 to about 130:1, or about 125:1 to about 135:1, or about 130:1 to about 140:1, or about 135:1 to about 145:1, or about 140:1 to about 150:1, lipid: nucleic acid, weight/weight.

In some aspects, a lipid nanoparticle comprising at least one nucleic acid can comprise lipid and nucleic acid at a ratio of about 5:1, or about 10:1, or about 15:1, or about 20:1, or about 25:1, or about 30:1, or about 35:1, or about 40:1, or about 45:1, or about 50:1, or about 55:1, or about 60:1, or about 65:1, or about 70:1, or about 75:1, or about 80:1, or about 85:1, or about 90:1, or about 95:1, or about 100:1, or about 105:1, or about 110:1, or about 115:1, or about 120:1, or about 125:1, or about 130:1, or about 135:1, or about 140:1, or about 145:1, or about 150:1, lipid: nucleic acid, weight/weight.

In some aspects, a lipid nanoparticle comprising at least one nucleic acid can comprise lipid and nucleic acid at a ratio of about 10:1, or about 25:1, or about 40:1, lipid: nucleic acid, weight/weight.

In some aspects, a lipid nanoparticle comprising at least one nucleic acid can comprise lipid and nucleic acid at a ratio of about 20:1, or about 40:1, or about 60:1, or about 80:1, or about 120:1 lipid: nucleic acid, weight/weight.

In some aspects of the preceding LNPs, the at least one nucleic acid molecule is an RNA molecule. In some aspects, the RNA molecule is an mRNA molecule. In some aspects, the RNA molecule is a guide RNA (gRNA) molecule. In some aspects, an LNP of the present disclosure can comprise a combination of mRNA molecules and gRNA molecules. In some aspects, the mRNA molecule further comprises a 5′-CAP.

In some aspects, a lipid nanoparticle is provided comprising about 41.4% of at least one compound of Formula (I), Formula (II), or Formula (III) by moles, about 10% of at least one structural lipid by moles, about 45.9% of at least one phospholipid by moles, and about 2.7% of at least one PEGylated lipid by moles, wherein the lipid nanoparticle further comprises at least one RNA molecule (e.g. mRNA molecule). In some aspects, the present disclosure provides a lipid nanoparticle comprising about 31.4% to about 51.4% of at least one compound of Formula (I), Formula (II), or Formula (III) by moles, about 1% to about 20% of at least one structural lipid by moles, about 35.9% to about 55.9% of at least one phospholipid by moles, and about 0.1% to about 12.7% of at least one PEGylated lipid by moles, wherein the lipid nanoparticle further comprises a at least one RNA molecule (e.g. mRNA molecule). In some aspects, the present disclosure provides a lipid nanoparticle comprising about 36.4% to about 46.4% of at least one compound of Formula (I), Formula (II), or Formula (III) by moles, about 5% to about 15% of at least one structural lipid by moles, about 40.9% to about 50 9% of at least one phospholipid by moles, and about 1% to about 7.7% of at least one PEGylated lipid by moles, wherein the lipid nanoparticle further comprises at least one RNA molecule (e.g. mRNA molecule). In some aspects, the mRNA molecule further comprises a 5′-CAP. In some aspects, the ratio of lipid to nucleic acid in the nanoparticle can be about 30:1 to about 50:1 (w/w), or about 35:1 to about 45:1 (w/w). In some aspects, the ratio of lipid to nucleic acid in the nanoparticle can be about 40:1 (w/w).

In some aspects, a lipid nanoparticle is provided comprising about 33.5% of at least one compound of Formula (I), Formula (II), or Formula (III) by moles, about 33.5% of at least one structural lipid by moles, about 32% of at least one phospholipid by moles, and about 1% of at least one PEGylated lipid by moles, wherein the lipid nanoparticle further comprises at least one RNA molecule (e.g. mRNA molecule). In some aspects, the present disclosure provides a lipid nanoparticle comprising about 23.5% to about 43.5% of at least one compound of Formula (I), Formula (II), or Formula (III) by moles, about 23.5% to about 43.5% of at least one structural lipid by moles, about 22% to about 42% of at least one phospholipid by moles, and about 0.1% to about 11% of at least one PEGylated lipid by moles, wherein the lipid nanoparticle further comprises a at least one RNA molecule (e.g. mRNA molecule). In some aspects, the present disclosure provides a lipid nanoparticle comprising about 28.5% to about 38.5% of at least one compound of Formula (I), Formula (II), or Formula (III) by moles, about 28.5% to about 38.5% of at least one structural lipid by moles, about 27% to about 37% of at least one phospholipid by moles, and about 0.5% to about 6% of at least one PEGylated lipid by moles, wherein the lipid nanoparticle further comprises at least one RNA molecule (e.g. mRNA molecule). In some aspects, the mRNA molecule further comprises a 5′-CAP. In some aspects, the ratio of lipid to nucleic acid in the nanoparticle can be about 30:1 to about 50:1 (w/w), or about 35:1 to about 45:1 (w/w). In some aspects, the ratio of lipid to nucleic acid in the nanoparticle can be about 40:1 (w/w).

In some aspects, a lipid nanoparticle is provided comprising about 60% of at least one compound of Formula (I), Formula (II), or Formula (III) by moles, about 29% of at least one structural lipid by moles, about 10% of at least one phospholipid by moles, and about 1% of at least one PEGylated lipid by moles, wherein the at least one nucleic acid comprises at least one RNA molecule (e.g. mRNA molecule). In some aspects, the present disclosure provides a lipid nanoparticle comprising about 50% to about 70% of at least one compound of Formula (I), Formula (II), or Formula (III) by moles, about 19% to about 39% of at least one structural lipid by moles, about 0.1% to about 20% of at least one phospholipid by moles, and about 0.1% to about 11% of at least one PEGylated lipid by moles, wherein the at least one nucleic acid comprises at least one RNA molecule (e.g. mRNA molecule). In some aspects, the present disclosure provides a lipid nanoparticle comprising about 55% to about 65% of at least one compound of Formula (I), Formula (II), or Formula (III) by moles, about 24% to about 34% of at least one structural lipid by moles, about 1% to about 15% of at least one phospholipid by moles, and about 0.5% to about 6% of at least one PEGylated lipid by moles, wherein the at least one nucleic acid comprises at least one RNA molecule (e.g. mRNA molecule). In some aspects, the mRNA molecule further comprises a 5′-CAP. In some aspects, the at least one nucleic acid further comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 40% to about 45.1% of at least one compound of Formula (I), Formula (II), or Formula (III) by moles, about 40.6% to about 51.8% of at least one structural lipid by moles, about 5% to about 13.5% of at least one phospholipid by moles, and about 1.7% to about 2.6% of at least one PEGylated lipid by moles, wherein the at least one nucleic acid comprises at least one RNA molecule (e.g. mRNA molecule). In some aspects, the present disclosure provides a lipid nanoparticle comprising about 30% to about 55.1% of at least one compound of Formula (I), Formula (II), or Formula (III) by moles, about 30.6% to about 61.8% of at least one structural lipid by moles, about 0.1% to about 23.5% of at least one phospholipid by moles, and about 0.1% to about 12.6% of at least one PEGylated lipid by moles, wherein the at least one nucleic acid comprises at least one RNA molecule (e.g. mRNA molecule). In some aspects, the present disclosure provides a lipid nanoparticle comprising about 35% to about 50.1% of at least one compound of Formula (I), Formula (II), or Formula (III) by moles, about 35.6% to about 56.8% of at least one structural lipid by moles, about 1% to about 18.5% of at least one phospholipid by moles, and about 1% to about 7.6% of at least one PEGylated lipid by moles, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 90:1 to about 130:1 (w/w), or about 100:1 to about 120:1 (w/w). In some aspects, the ratio of lipid to nucleic acid in the nanoparticle can be about 100:1 (w/w). In some aspects, the ratio of lipid to nucleic acid in the nanoparticle can be about 120:1 (w/w).

In some aspects, a lipid nanoparticle is provided comprising about 34% to about 37.8% of at least one compound of Formula (I), Formula (II), or Formula (III) by moles, about 46.7% to about 55.1% of at least one structural lipid by moles, about 5% to about 14.3% of at least one phospholipid by moles, and about 1.7% to about 2.7% of at least one PEGylated lipid by moles, wherein the at least one nucleic acid comprises at least one RNA molecule (e.g. mRNA molecule). In some aspects, the present disclosure provides a lipid nanoparticle comprising about 24% to about 47.8% of at least one compound of Formula (I), Formula (II), or Formula (III) by moles, about 36.7% to about 65.1% of at least one structural lipid by moles, about 0.1% to about 24.3% of at least one phospholipid by moles, and about 0.1% to about 12.7% of at least one PEGylated lipid by moles, wherein the at least one nucleic acid comprises at least one RNA molecule (e.g. mRNA molecule). In some aspects, the present disclosure provides a lipid nanoparticle comprising about 29% to about 42.8% of at least one compound of Formula (I), Formula (II), or Formula (III) by moles, about 41.7% to about 60.1% of at least one structural lipid by moles, about 1% to about 19.3% of at least one phospholipid by moles, and about 1% to about 7.7% of at least one PEGylated lipid by moles, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 37.78% of at least one compound of Formula (I), Formula (II), or Formula (III) by moles, about 55.07% of at least one structural lipid by moles, about 5% of at least one phospholipid by moles, and about 2.15% of at least one PEGylated lipid by moles, wherein the at least one nucleic acid comprises at least one RNA molecule (e.g. mRNA molecule). In some aspects, the present disclosure provides a lipid nanoparticle comprising about 27.78% to about 47.78% of at least one compound of Formula (I), Formula (II), or Formula (III) by moles, about 45.07% to about 65.07% of at least one structural lipid by moles, about 0.1% to about 15% of at least one phospholipid by moles, and about 0.1% to about 12.15% of at least one PEGylated lipid by moles, wherein the at least one nucleic acid comprises at least one RNA molecule (e.g. mRNA molecule). In some aspects, the present disclosure provides a lipid nanoparticle comprising about 32.78% to about 42.78% of at least one compound of Formula (I), Formula (II), or Formula (III) by moles, about 50.07% to about 60.07% of at least one structural lipid by moles, about 1% to about 10% of at least one phospholipid by moles, and about 1% to about 7.15% of at least one PEGylated lipid by moles, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 44.38% of at least one compound of Formula (I), Formula (II), or Formula (III) by moles, about 48.46% of at least one structural lipid by moles, about 5.43% of at least one phospholipid by moles, and about 1.73% of at least one PEGylated lipid by moles, wherein the at least one nucleic acid comprises at least one RNA molecule (e.g. mRNA molecule). In some aspects, the present disclosure provides a lipid nanoparticle comprising about 34.38% to about 54.38% of at least one compound of Formula (I), Formula (II), or Formula (III) by moles, about 38.46% to about 58.46% of at least one structural lipid by moles, about 0.1% to about 15.43% of at least one phospholipid by moles, and about 0.1% to about 11.73% of at least one PEGylated lipid by moles, wherein the at least one nucleic acid comprises at least one RNA molecule (e.g. mRNA molecule). In some aspects, the present disclosure provides a lipid nanoparticle comprising about 39.38% to about 49.38% of at least one compound of Formula (I), Formula (II), or Formula (III) by moles, about 43.46% to about 53.46% of at least one structural lipid by moles, about 1% to about 10.43% of at least one phospholipid by moles, and about 1% to about 6.73% of at least one PEGylated lipid by moles, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 36.97% of at least one compound of Formula (I), Formula (II), or Formula (III) by moles, about 46.69% of at least one structural lipid by moles, about 14.34% of at least one phospholipid by moles, and about 2% of at least one PEGylated lipid by moles, wherein the at least one nucleic acid comprises at least one RNA molecule (e.g. mRNA molecule). In some aspects, the present disclosure provides a lipid nanoparticle comprising about 26.97% to about 46.97% of at least one compound of Formula (I), Formula (II), or Formula (III) by moles, about 36.69% to about 56.69% of at least one structural lipid by moles, about 4.34% to about 24.34% of at least one phospholipid by moles, and about 0.1% to about 12% of at least one PEGylated lipid by moles, wherein the at least one nucleic acid comprises at least one RNA molecule (e.g. mRNA molecule). In some aspects, the present disclosure provides a lipid nanoparticle comprising about 31.97% to about 41.97% of at least one compound of Formula (I), Formula (II), or Formula (III) by moles, about 41.69% to about 51.69% of at least one structural lipid by moles, about 9.34% to about 19.34% of at least one phospholipid by moles, and about 1% to about 7% of at least one PEGylated lipid by moles, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 43.11% of at least one compound of Formula (I), Formula (II), or Formula (III) by moles, about 45.09% of at least one structural lipid by moles, about 9.59% of at least one phospholipid by moles, and about 2.21% of at least one PEGylated lipid by moles, wherein the at least one nucleic acid comprises at least one RNA molecule (e.g. mRNA molecule). In some aspects, the present disclosure provides a lipid nanoparticle comprising about 33.11% to about 53.11% of at least one compound of Formula (I), Formula (II), or Formula (III) by moles, about 35.09% to about 55.09% of at least one structural lipid by moles, about 0.1% to about 19.59% of at least one phospholipid by moles, and about 0.1% to about 12.21% of at least one PEGylated lipid by moles, wherein the at least one nucleic acid comprises at least one RNA molecule (e.g. mRNA molecule). In some aspects, the present disclosure provides a lipid nanoparticle comprising about 38.11% to about 48.11% of at least one compound of Formula (I), Formula (II), or Formula (III) by moles, about 40.09% to about 50.09% of at least one structural lipid by moles, about 1% to about 14.59% of at least one phospholipid by moles, and about 1% to about 7.21% of at least one PEGylated lipid by moles, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 40.75% of at least one compound of Formula (I), Formula (II), or Formula (III) by moles, about 51.75% of at least one structural lipid by moles, about 5% of at least one phospholipid by moles, and about 2.5% of at least one PEGylated lipid by moles, wherein the at least one nucleic acid comprises at least one RNA molecule (e.g. mRNA molecule). In some aspects, the present disclosure provides a lipid nanoparticle comprising about 30.75% to about 50.75% of at least one compound of Formula (I), Formula (II), or Formula (III) by moles, about 41.75% to about 61.75% of at least one structural lipid by moles, about 0.1% to about 15% of at least one phospholipid by moles, and about 0.1% to about 12.5% of at least one PEGylated lipid by moles, wherein the at least one nucleic acid comprises at least one RNA molecule (e.g. mRNA molecule). In some aspects, the present disclosure provides a lipid nanoparticle comprising about 35.75% to about 45.75% of at least one compound of Formula (I), Formula (II), or Formula (III) by moles, about 46.75% to about 56.75% of at least one structural lipid by moles, about 1% to about 10% of at least one phospholipid by moles, and about 1% to about 7.5% of at least one PEGylated lipid by moles, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w). In some aspects, the ratio of lipid to nucleic acid in the nanoparticle can be about 20:1, 40:1, 60:1, 80:1, or 100:1.

In some aspects, the nucleic acid molecule is a DNA molecule. Thus, the present disclosure provides a lipid nanoparticle comprising about 38% of at least one compound of Formula (I), Formula (II), or Formula (III) by moles, about 10% of at least one structural lipid by moles, about 50% of at least one phospholipid by moles, and about 2% of at least one PEGylated lipid by moles, wherein the at least one nucleic acid comprises at least one DNA molecule. In some aspects, the present disclosure provides a lipid nanoparticle comprising about 28% to about 48% of at least one compound of Formula (I), Formula (II), or Formula (III) by moles, about 1% to about 20% of at least one structural lipid by moles, about 40% to about 60% of at least one phospholipid by moles, and about 0.1% to about 12% of at least one PEGylated lipid by moles, wherein the at least one nucleic acid comprises at least one DNA molecule. In some aspects, the present disclosure provides a lipid nanoparticle comprising about 33% to about 43% of at least one compound of Formula (I), Formula (II), or Formula (III) by moles, about 5% to about 15% of at least one structural lipid by moles, about 45% to about 55% of at least one phospholipid by moles, and about 1% to about 6% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 35:1 to about 55:1 (w/w), or about 40:1 to about 50:1 (w/w). In some aspects, the ratio of lipid to nucleic acid in the nanoparticle can be about 45:1 (w/w). In some aspects, the ratio of lipid to nucleic acid in the nanoparticle can be about 40:1 (w/w).

In some aspects, a lipid nanoparticle is provided comprising about 35% of at least one compound of Formula (I), Formula (II), or Formula (III) by moles, about 41.8% of at least one structural lipid by moles, about 20% of at least one phospholipid by moles, and about 3.2% of at least one PEGylated lipid by moles, wherein the at least one nucleic acid comprises at least one DNA molecule. In some aspects, the present disclosure provides a lipid nanoparticle comprising about 25% to about 45% of at least one compound of Formula (I), Formula (II), or Formula (III) by moles, about 31.8% to about 51.8% of at least one structural lipid by moles, about 10% to about 30% of at least one phospholipid by moles, and about 0.1% to about 13.2% of at least one PEGylated lipid by moles, wherein the at least one nucleic acid comprises at least one DNA molecule. In some aspects, the present disclosure provides a lipid nanoparticle comprising about 30% to about 40% of at least one compound of Formula (I), Formula (II), or Formula (III) by moles, about 36.8% to about 46.8% of at least one structural lipid by moles, about 15% to about 25% of at least one phospholipid by moles, and about 1% to about 8.2% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 70:1 to about 90:1 (w/w), or about 75:1 to about 85:1 (w/w). In some aspects, the ratio of lipid to nucleic acid in the nanoparticle can be about 80:1 (w/w).

In some aspects, a lipid nanoparticle is provided comprising about 40% to about 45.1% of at least one compound of Formula (I), Formula (II), or Formula (III) by moles, about 40.6% to about 51.8% of at least one structural lipid by moles, about 5% to about 13.5% of at least one phospholipid by moles, and about 1.7% to about 2.6% of at least one PEGylated lipid by moles, wherein the at least one nucleic acid comprises at least one DNA molecule. In some aspects, the present disclosure provides a lipid nanoparticle comprising about 30% to about 55.1% of at least one compound of Formula (I), Formula (II), or Formula (III) by moles, about 30.6% to about 61.8% of at least one structural lipid by moles, about 0.1% to about 23.5% of at least one phospholipid by moles, and about 0.1% to about 12.6% of at least one PEGylated lipid by moles, wherein the at least one nucleic acid comprises at least one DNA molecule. In some aspects, the present disclosure provides a lipid nanoparticle comprising about 35% to about 50.1% of at least one compound of Formula (I), Formula (II), or Formula (III) by moles, about 35.6% to about 56.8% of at least one structural lipid by moles, about 1% to about 18.5% of at least one phospholipid by moles, and about 1% to about 7.6% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 130:1 (w/w), or about 100:1 to about 120:1 (w/w). In some aspects, the ratio of lipid to nucleic acid in the nanoparticle can be about 100:1 (w/w). In some aspects, the ratio of lipid to nucleic acid in the nanoparticle can be about 120:1 (w/w).

In some aspects, a lipid nanoparticle is provided comprising about 34% to about 37.8% of at least one compound of Formula (I), Formula (II), or Formula (III) by moles, about 46.7% to about 55% of at least one structural lipid by moles, about 5% to about 14.3% of at least one phospholipid by moles, and about 1.7% to about 2.7% of at least one PEGylated lipid by moles, wherein the at least one nucleic acid comprises at least one DNA molecule. In some aspects, the present disclosure provides a lipid nanoparticle comprising about 24% to about 47.8% of at least one compound of Formula (I), Formula (II), or Formula (III) by moles, about 36.7% to about 65% of at least one structural lipid by moles, about 0.1% to about 24.3% of at least one phospholipid by moles, and about 0.1% to about 12.7% of at least one PEGylated lipid by moles, wherein the at least one nucleic acid comprises at least one DNA molecule. In some aspects, the present disclosure provides a lipid nanoparticle comprising about 29% to about 42.8% of at least one compound of Formula (I), Formula (II), or Formula (III) by moles, about 41.7% to about 60% of at least one structural lipid by moles, about 1% to about 19.3% of at least one phospholipid by moles, and about 1% to about 7.7% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 35.28% of at least one compound of Formula (I), Formula (II), or Formula (III) by moles, about 53.68% of at least one structural lipid by moles, about 8.35% of at least one phospholipid by moles, and about 2.69% of at least one PEGylated lipid by moles, wherein the at least one nucleic acid comprises at least one DNA molecule. In some aspects, the present disclosure provides a lipid nanoparticle comprising about 25.28% to about 45.28% of at least one compound of Formula (I), Formula (II), or Formula (III) by moles, about 43.68% to about 63.68% of at least one structural lipid by moles, about 0.1% to about 18.35% of at least one phospholipid by moles, and about 0.1% to about 12.69% of at least one PEGylated lipid by moles, wherein the at least one nucleic acid comprises at least one DNA molecule. In some aspects, the present disclosure provides a lipid nanoparticle comprising about 30.28% to about 40.28% of at least one compound of Formula (I), Formula (II), or Formula (III) by moles, about 48.68% to about 58.68% of at least one structural lipid by moles, about 1% to about 13.35% of at least one phospholipid by moles, and about 1% to about 7.69% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 37.78% of at least one compound of Formula (I), Formula (II), or Formula (III) by moles, about 55.07% of at least one structural lipid by moles, about 5% of at least one phospholipid by moles, and about 2.15% of at least one PEGylated lipid by moles, wherein the at least one nucleic acid comprises at least one DNA molecule. In some aspects, the present disclosure provides a lipid nanoparticle comprising about 27.78% to about 47.78% of at least one compound of Formula (I), Formula (II), or Formula (III) by moles, about 45.07% to about 65.07% of at least one structural lipid by moles, about 0.1% to about 15% of at least one phospholipid by moles, and about 0.1% to about 12.15% of at least one PEGylated lipid by moles, wherein the at least one nucleic acid comprises at least one DNA molecule. In some aspects, the present disclosure provides a lipid nanoparticle comprising about 32.78% to about 42.78% of at least one compound of Formula (I), Formula (II), or Formula (III) by moles, about 50.07% to about 60.07% of at least one structural lipid by moles, about 1% to about 10% of at least one phospholipid by moles, and about 1% to about 7.15% of at least one PEGylated lipid by moles, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 44.38% of at least one compound of Formula (I), Formula (II), or Formula (III) by moles, about 48.46% of at least one structural lipid by moles, about 5.43% of at least one phospholipid by moles, and about 1.73% of at least one PEGylated lipid by moles, wherein the at least one nucleic acid comprises at least one DNA molecule. In some aspects, the present disclosure provides a lipid nanoparticle comprising about 34.38% to about 54.38% of at least one compound of Formula (I), Formula (II), or Formula (III) by moles, about 38.46% to about 58.46% of at least one structural lipid by moles, about 0.1% to about 15.43% of at least one phospholipid by moles, and about 0.1% to about 11.73% of at least one PEGylated lipid by moles, wherein the at least one nucleic acid comprises at least one DNA molecule. In some aspects, the present disclosure provides a lipid nanoparticle comprising about 39.38% to about 49.38% of at least one compound of Formula (I), Formula (II), or Formula (III) by moles, about 43.46% to about 53.46% of at least one structural lipid by moles, about 1% to about 10.43% of at least one phospholipid by moles, and about 1% to about 6.73% of at least one PEGylated lipid by moles, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 39.98% of at least one compound of Formula (I), Formula (II), or Formula (III) by moles, about 50.47% of at least one structural lipid by moles, about 7.17% of at least one phospholipid by moles, and about 2.39% of at least one PEGylated lipid by moles, wherein the at least one nucleic acid comprises at least one DNA molecule. In some aspects, the present disclosure provides a lipid nanoparticle comprising about 29.98% to about 49.98% of at least one compound of Formula (I), Formula (II), or Formula (III) by moles, about 40.47% to about 60.47% of at least one structural lipid by moles, about 0.1% to about 17.17% of at least one phospholipid by moles, and about 0.1% to about 12.39% of at least one PEGylated lipid by moles, wherein the at least one nucleic acid comprises at least one DNA molecule. In some aspects, the present disclosure provides a lipid nanoparticle comprising about 34.98% to about 44.98% of at least one compound of Formula (I), Formula (II), or Formula (III) by moles, about 45.47% to about 55.47% of at least one structural lipid by moles, about 1% to about 12.17% of at least one phospholipid by moles, and about 1% to about 7.39% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 36.97% of at least one compound of Formula (I), Formula (II), or Formula (III) by moles, about 46.69% of at least one structural lipid by moles, about 14.34% of at least one phospholipid by moles, and about 2% of at least one PEGylated lipid by moles, wherein the at least one nucleic acid comprises at least one DNA molecule. In some aspects, the present disclosure provides a lipid nanoparticle comprising about 26.97% to about 46.97% of at least one compound of Formula (I), Formula (II), or Formula (III) by moles, about 36.69% to about 56.69% of at least one structural lipid by moles, about 4.34% to about 24.34% of at least one phospholipid by moles, and about 0.1% to about 12% of at least one PEGylated lipid by moles, wherein the at least one nucleic acid comprises at least one DNA molecule. In some aspects, the present disclosure provides a lipid nanoparticle comprising about 31.97% to about 41.97% of at least one compound of Formula (I), Formula (II), or Formula (III) by moles, about 41.69% to about 51.69% of at least one structural lipid by moles, about 9.34% to about 19.34% of at least one phospholipid by moles, and about 1% to about 7% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 33.96% of at least one compound of Formula (I), Formula (II), or Formula (III) by moles, about 51.44% of at least one structural lipid by moles, about 12.89% of at least one phospholipid by moles, and about 1.7% of at least one PEGylated lipid by moles, wherein the at least one nucleic acid comprises at least one DNA molecule. In some aspects, the present disclosure provides a lipid nanoparticle comprising about 23.96% to about 43.96% of at least one compound of Formula (I), Formula (II), or Formula (III) by moles, about 41.44% to about 61.44% of at least one structural lipid by moles, about 2.89% to about 22.89% of at least one phospholipid by moles, and about 0.1% to about 11.7% of at least one PEGylated lipid by moles, wherein the at least one nucleic acid comprises at least one DNA molecule. In some aspects, the present disclosure provides a lipid nanoparticle comprising about 28.96% to about 38.96% of at least one compound of Formula (I), Formula (II), or Formula (III) by moles, about 46.44% to about 56.44% of at least one structural lipid by moles, about 7.89% to about 17.89% of at least one phospholipid by moles, and about 1% to about 6.7% of at least one PEGylated lipid by moles, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 44.95% of at least one compound of Formula (I), Formula (II), or Formula (III) by moles, about 40.77% of at least one structural lipid by moles, about 12.4% of at least one phospholipid by moles, and about 1.88% of at least one PEGylated lipid by moles, wherein the at least one nucleic acid comprises at least one DNA molecule. In some aspects, the present disclosure provides a lipid nanoparticle comprising about 34.95% to about 54.95% of at least one compound of Formula (I), Formula (II), or Formula (III) by moles, about 30.77% to about 50.77% of at least one structural lipid by moles, about 2.4% to about 22.4% of at least one phospholipid by moles, and about 0.1% to about 11.88% of at least one PEGylated lipid by moles, wherein the at least one nucleic acid comprises at least one DNA molecule. In some aspects, the present disclosure provides a lipid nanoparticle comprising about 39.95% to about 49.95% of at least one compound of Formula (I), Formula (II), or Formula (III) by moles, about 35.77% to about 45.77% of at least one structural lipid by moles, about 7.4% to about 17.4% of at least one phospholipid by moles, and about 1% to about 6.88% of at least one PEGylated lipid by moles, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 43.11% of at least one compound of Formula (I), Formula (II), or Formula (III) by moles, about 45.09% of at least one structural lipid by moles, about 9.59% of at least one phospholipid by moles, and about 2.21% of at least one PEGylated lipid by moles, wherein the at least one nucleic acid comprises at least one DNA molecule. In some aspects, the present disclosure provides a lipid nanoparticle comprising about 33.11% to about 53.11% of at least one compound of Formula (I), Formula (II), or Formula (III) by moles, about 35.09% to about 55.09% of at least one structural lipid by moles, about 0.1% to about 19.59% of at least one phospholipid by moles, and about 0.1% to about 12.21% of at least one PEGylated lipid by moles, wherein the at least one nucleic acid comprises at least one DNA molecule. In some aspects, the present disclosure provides a lipid nanoparticle comprising about 38.11% to about 48.11% of at least one compound of Formula (I), Formula (II), or Formula (III) by moles, about 40.09% to about 50.09% of at least one structural lipid by moles, about 1% to about 14.59% of at least one phospholipid by moles, and about 1% to about 7.21% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 41.02% of at least one compound of Formula (I), Formula (II), or Formula (III) by moles, about 42.83% of at least one structural lipid by moles, about 13.55% of at least one phospholipid by moles, and about 2.61% of at least one PEGylated lipid by moles, wherein the at least one nucleic acid comprises at least one DNA molecule. In some aspects, the present disclosure provides a lipid nanoparticle comprising about 31.02% to about 51.02% of at least one compound of Formula (I), Formula (II), or Formula (III) by moles, about 32.83% to about 52.83% of at least one structural lipid by moles, about 3.55% to about 23.55% of at least one phospholipid by moles, and about 0.1% to about 12.61% of at least one PEGylated lipid by moles, wherein the at least one nucleic acid comprises at least one DNA molecule. In some aspects, the present disclosure provides a lipid nanoparticle comprising about 36.02% to about 46.02% of at least one compound of Formula (I), Formula (II), or Formula (III) by moles, about 37.83% to about 47.83% of at least one structural lipid by moles, about 8.55% to about 18.55% of at least one phospholipid by moles, and about 1% to about 7.61% of at least one PEGylated lipid by moles, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 36.71% of at least one compound of Formula (I), Formula (II), or Formula (III) by moles, about 48.44% of at least one structural lipid by moles, about 12.46% of at least one phospholipid by moles, and about 2.39% of at least one PEGylated lipid by moles, wherein the at least one nucleic acid comprises at least one DNA molecule. In some aspects, the present disclosure provides a lipid nanoparticle comprising about 26.71% to about 46.71% of at least one compound of Formula (I), Formula (II), or Formula (III) by moles, about 38.44% to about 58.44% of at least one structural lipid by moles, about 2.46% to about 22.46% of at least one phospholipid by moles, and about 0.1% to about 12.39% of at least one PEGylated lipid by moles, wherein the at least one nucleic acid comprises at least one DNA molecule. In some aspects, the present disclosure provides a lipid nanoparticle comprising about 31.71% to about 41.71% of at least one compound of Formula (I), Formula (II), or Formula (III) by moles, about 43.44% to about 53.44% of at least one structural lipid by moles, about 7.46% to about 17.46% of at least one phospholipid by moles, and about 1% to about 7.39% of at least one PEGylated lipid by moles, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle is provided comprising about 45.12% of at least one compound of Formula (I), Formula (II), or Formula (III) by moles, about 40.58% of at least one structural lipid by moles, about 12.55% of at least one phospholipid by moles, and about 1.75% of at least one PEGylated lipid by moles, wherein the at least one nucleic acid comprises at least one DNA molecule. In some aspects, the present disclosure provides a lipid nanoparticle comprising about 35.12% to about 55.12% of at least one compound of Formula (I), Formula (II), or Formula (III) by moles, about 30.58% to about 50.58% of at least one structural lipid by moles, about 2.55% to about 22.55% of at least one phospholipid by moles, and about 0.1% to about 11.75% of at least one PEGylated lipid by moles, wherein the at least one nucleic acid comprises at least one DNA molecule. In some aspects, the present disclosure provides a lipid nanoparticle comprising about 40.12% to about 50.12% of at least one compound of Formula (I), Formula (II), or Formula (III) by moles, about 35.58% to about 45.58% of at least one structural lipid by moles, about 7.55% to about 17.55% of at least one phospholipid by moles, and about 1% to about 6.75% of at least one PEGylated lipid by moles, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 40.75% of at least one compound of Formula (I), Formula (II), or Formula (III) by moles, about 51.75% of at least one structural lipid by moles, about 5% of at least one phospholipid by moles, and about 2.5% of at least one PEGylated lipid by moles, wherein the at least one nucleic acid comprises at least one DNA molecule. In some aspects, the present disclosure provides a lipid nanoparticle comprising about 30.75% to about 50.75% of at least one compound of Formula (I), Formula (II), or Formula (III) by moles, about 41.75% to about 61.75% of at least one structural lipid by moles, about 0.1% to about 15% of at least one phospholipid by moles, and about 0.1% to about 12.5% of at least one PEGylated lipid by moles, wherein the at least one nucleic acid comprises at least one DNA molecule. In some aspects, the present disclosure provides a lipid nanoparticle comprising about 35.75% to about 45.75% of at least one compound of Formula (I), Formula (II), or Formula (III) by moles, about 46.75% to about 56.75% of at least one structural lipid by moles, about 1% to about 10% of at least one phospholipid by moles, and about 1% to about 7.5% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the at least one nucleic acid further comprises at least one RNA molecule. In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w). In some aspects, the ratio of lipid to nucleic acid in the nanoparticle can be about 20:1, 40:1, 60:1, 80:1, or 100:1.

In some aspects, a lipid nanoparticle is provided comprising about 40.68% of at least one compound of Formula (I), Formula (II), or Formula (III) by moles, about 47.12% of at least one structural lipid by moles, about 10% of at least one phospholipid by moles, and about 2.2% of at least one PEGylated lipid by moles, wherein the at least one nucleic acid comprises at least one DNA molecule. In some aspects, the present disclosure provides a lipid nanoparticle comprising about 30.68% to about 50.68% of at least one compound of Formula (I), Formula (II), or Formula (III) by moles, about 37.12% to about 57.12% of at least one structural lipid by moles, about 0.1% to about 20% of at least one phospholipid by moles, and about 0.1% to about 12.2% of at least one PEGylated lipid by moles, wherein the at least one nucleic acid comprises at least one DNA molecule. In some aspects, the present disclosure provides a lipid nanoparticle comprising about 35.68% to about 45.68% of at least one compound of Formula (I), Formula (II), or Formula (III) by moles, about 42.12% to about 52.12% of at least one structural lipid by moles, about 1% to about 15% of at least one phospholipid by moles, and about 1% to about 7.2% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the at least one nucleic acid further comprises at least one RNA molecule. In some aspects, the mRNA molecule further comprises a 5′-CAP. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 32.5% of at least one compound of Formula (I), Formula (II), or Formula (III) by moles, about 60% of at least one structural lipid by moles, about 5% of at least one phospholipid by moles, and about 2.5% of at least one PEGylated lipid by moles, wherein the at least one nucleic acid comprises at least one DNA molecule. In some aspects, the present disclosure provides a lipid nanoparticle comprising about 22.5% to about 42.5% of at least one compound of Formula (I), Formula (II), or Formula (III) by moles, about 50% to about 70% of at least one structural lipid by moles, about 0.1% to about 15% of at least one phospholipid by moles, and about 0.1% to about 12.5% of at least one PEGylated lipid by moles, wherein the at least one nucleic acid comprises at least one DNA molecule. In some aspects, the present disclosure provides a lipid nanoparticle comprising about 27.5% to about 37.5% of at least one compound of Formula (I), Formula (II), or Formula (III) by moles, about 55% to about 65% of at least one structural lipid by moles, about 1% to about 10% of at least one phospholipid by moles, and about 1% to about 7.5% of at least one PEGylated lipid by moles, wherein the at least one nucleic acid comprises at least one DNA molecule. In one aspect, the at least one DNA molecule is a DoggyBone DNA molecule. In some aspects, the at least one DNA molecule is a DNA nanoplasmid. In some aspects, the at least one nucleic acid further comprises at least one RNA molecule. In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects, a lipid nanoparticle comprising at least one nucleic acid can comprise at least one nucleic acid molecule, wherein the at least one nucleic acid molecule is at least one RNA molecule or at least one DNA molecule.

In some aspects, a lipid nanoparticle is provided comprising about 40.68% of at least one compound of Formula (I), Formula (II), or Formula (III) by moles, about 47.12% of at least one structural lipid by moles, about 10% of at least one phospholipid by moles, and about 2.2% of at least one PEGylated lipid by moles, wherein the at least one nucleic acid comprises at least one RNA molecule or at least one DNA molecule. In some aspects, the present disclosure provides a lipid nanoparticle comprising about 30.68% to about 50.68% of at least one compound of Formula (I), Formula (II), or Formula (III) by moles, about 37.12% to about 57.12% of at least one structural lipid by moles, about 0.1% to about 20% of at least one phospholipid by moles, and about 0.1% to about 12.2% of at least one PEGylated lipid by moles, wherein the at least one nucleic acid comprises at least one RNA molecule or at least one DNA molecule. In some aspects, the present disclosure provides a lipid nanoparticle comprising about 35.68% to about 45.68% of at least one compound of Formula (I), Formula (II), or Formula (III) by moles, about 42.12% to about 52.12% of at least one structural lipid by moles, about 1% to about 15% of at least one phospholipid by moles, and about 1% to about 7.2% of at least one PEGylated lipid by moles, wherein the at least one nucleic acid comprises at least one RNA molecule or at least one DNA molecule. In some aspects, the mRNA molecule further comprises a 5′-CAP. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 60% of at least one compound of Formula (I), Formula (II), or Formula (III) by moles, about 29% of at least one structural lipid by moles, about 10% of at least one phospholipid by moles, and about 1% of at least one PEGylated lipid by moles, wherein the at least one nucleic acid comprises at least one RNA molecule or at least one DNA molecule. In some aspects, the present disclosure provides a lipid nanoparticle comprising about 50% to about 70% of at least one compound of Formula (I), Formula (II), or Formula (III) by moles, about 19% to about 39% of at least one structural lipid by moles, about 0.1% to about 20% of at least one phospholipid by moles, and about 0.1% to about 11% of at least one PEGylated lipid by moles, wherein the at least one nucleic acid comprises at least one RNA molecule or at least one DNA molecule. In some aspects, the present disclosure provides a lipid nanoparticle comprising about 55% to about 65% of at least one compound of Formula (I), Formula (II), or Formula (III) by moles, about 24% to about 34% of at least one structural lipid by moles, about 1% to about 15% of at least one phospholipid by moles, and about 0.5% to about 6% of at least one PEGylated lipid by moles, wherein the at least one nucleic acid comprises at least one RNA molecule or at least one DNA molecule. In some aspects, the mRNA molecule further comprises a 5′-CAP. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 24.3% of at least one compound of Formula (I), Formula (II), or Formula (III) by moles, about 46.4% of at least one structural lipid by moles, about 28.3% of at least one phospholipid by moles, and about 1% of at least one PEGylated lipid by moles, wherein the at least one nucleic acid comprises at least one RNA molecule or at least one DNA molecule. In some aspects, the present disclosure provides a lipid nanoparticle comprising about 14.3% to about 34.3% of at least one compound of Formula (I), Formula (II), or Formula (III) by moles, about 36.4% to about 56.4% of at least one structural lipid by moles, about 18.3% to about 38.3% of at least one phospholipid by moles, and about 0.1% to about 11% of at least one PEGylated lipid by moles, wherein the at least one nucleic acid comprises at least one RNA molecule or at least one DNA molecule. In some aspects, the present disclosure provides a lipid nanoparticle comprising about 19.3% to about 29.3% of at least one compound of Formula (I), Formula (II), or Formula (III) by moles, about 41.4% to about 51.4% of at least one structural lipid by moles, about 23.3% to about 33.3% of at least one phospholipid by moles, and about 0.5% to about 6% of at least one PEGylated lipid by moles, wherein the at least one nucleic acid comprises at least one RNA molecule or at least one DNA molecule. In some aspects, the mRNA molecule further comprises a 5′-CAP. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 40% to about 60% of at least one compound of Formula (I), Formula (II), or Formula (III) by moles, about 29% to about 47.1% of at least one structural lipid by moles, about 10% of at least one phospholipid by moles, and about 1% to about 2.2% of at least one PEGylated lipid by moles, wherein the at least one nucleic acid comprises at least one RNA molecule or at least one DNA molecule. In some aspects, the present disclosure provides a lipid nanoparticle comprising about 30% to about 70% of at least one compound of Formula (I), Formula (II), or Formula (III) by moles, about 19% to about 57.1% of at least one structural lipid by moles, about 0.1% to about 20% of at least one phospholipid by moles, and about 0.1% to about 12.2% of at least one PEGylated lipid by moles, wherein the at least one nucleic acid comprises at least one RNA molecule or at least one DNA molecule. In some aspects, the present disclosure provides a lipid nanoparticle comprising about 35% to about 65% of at least one compound of Formula (I), Formula (II), or Formula (III) by moles, about 24% to about 52.1% of at least one structural lipid by moles, about 1% to about 15% of at least one phospholipid by moles, and about 0.5% to about 7.2% of at least one PEGylated lipid by moles, wherein the at least one nucleic acid comprises at least one RNA molecule or at least one DNA molecule. In some aspects, the mRNA molecule further comprises a 5′-CAP. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle comprising at least one nucleic acid can comprise at least one nucleic acid molecule, wherein the at least one nucleic acid molecule is at least one RNA molecule and at least one DNA molecule.

In some aspects, a lipid nanoparticle is provided comprising about 40.68% of at least one compound of Formula (I), Formula (II), or Formula (III) by moles, about 47.12% of at least one structural lipid by moles, about 10% of at least one phospholipid by moles, and about 2.2% of at least one PEGylated lipid by moles, wherein the at least one nucleic acid comprises at least one RNA molecule and at least one DNA molecule. In some aspects, the present disclosure provides a lipid nanoparticle comprising about 30.68% to about 50.68% of at least one compound of Formula (I), Formula (II), or Formula (III) by moles, about 37.12% to about 57.12% of at least one structural lipid by moles, about 0.1% to about 20% of at least one phospholipid by moles, and about 0.1% to about 12.2% of at least one PEGylated lipid by moles, wherein the at least one nucleic acid comprises at least one RNA molecule and at least one DNA molecule. In some aspects, the present disclosure provides a lipid nanoparticle comprising about 35.68% to about 45.68% of at least one compound of Formula (I), Formula (II), or Formula (III) by moles, about 42.12% to about 52.12% of at least one structural lipid by moles, about 1% to about 15% of at least one phospholipid by moles, and about 1% to about 7.2% of at least one PEGylated lipid by moles, wherein the at least one nucleic acid comprises at least one RNA molecule and at least one DNA molecule. In some aspects, the mRNA molecule further comprises a 5′-CAP. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 60% of at least one compound of Formula (I), Formula (II), or Formula (III) by moles, about 29% of at least one structural lipid by moles, about 10% of at least one phospholipid by moles, and about 1% of at least one PEGylated lipid by moles, wherein the at least one nucleic acid comprises at least one RNA molecule and at least one DNA molecule. In some aspects, the present disclosure provides a lipid nanoparticle comprising about 50% to about 70% of at least one compound of Formula (I), Formula (II), or Formula (III) by moles, about 19% to about 39% of at least one structural lipid by moles, about 0.1% to about 20% of at least one phospholipid by moles, and about 0.1% to about 11% of at least one PEGylated lipid by moles, wherein the at least one nucleic acid comprises at least one RNA molecule and at least one DNA molecule. In some aspects, the present disclosure provides a lipid nanoparticle comprising about 55% to about 65% of at least one compound of Formula (I), Formula (II), or Formula (III) by moles, about 24% to about 34% of at least one structural lipid by moles, about 1% to about 15% of at least one phospholipid by moles, and about 0.5% to about 6% of at least one PEGylated lipid by moles, wherein the at least one nucleic acid comprises at least one RNA molecule and at least one DNA molecule. In some aspects, the mRNA molecule further comprises a 5′-CAP. In one aspect, the at least one DNA molecule is a DoggyBone DNA molecule. In some aspects, the at least one DNA molecule is a DNA nanoplasmid. In some aspects, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, a lipid nanoparticle is provided comprising about 40.75% of at least one compound of Formula (I), Formula (II), or Formula (III) by moles, about 51.75% of at least one structural lipid by moles, about 5% of at least one phospholipid by moles, and about 2.5% of at least one PEGylated lipid by moles, wherein the at least one nucleic acid comprises at least one RNA molecule and at least one DNA molecule. In some aspects, the present disclosure provides a lipid nanoparticle comprising about 30.75% to about 50.75% of at least one compound of Formula (I), Formula (II), or Formula (III) by moles, about 41.75% to about 61.75% of at least one structural lipid by moles, about 0.1% to about 15% of at least one phospholipid by moles, and about 0.1% to about 12.5% of at least one PEGylated lipid by moles, wherein the at least one nucleic acid comprises at least one RNA molecule and at least one DNA molecule. In some aspects, the present disclosure provides a lipid nanoparticle comprising about 35.75% to about 45.75% of at least one compound of Formula (I), Formula (II), or Formula (III) by moles, about 46.75% to about 56.75% of at least one structural lipid by moles, about 1% to about 10% of at least one phospholipid by moles, and about 1% to about 7.5% of at least one PEGylated lipid by moles, wherein the at least one nucleic acid comprises at least one RNA molecule and at least one DNA molecule. In one aspect, the at least one DNA molecule is a Doggy Bone DNA molecule. In some aspects, the at least one DNA molecule is a DNA nanoplasmid. In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w). In some aspects, the ratio of lipid to nucleic acid in the nanoparticle can be about 20:1, 40:1, 60:1, 80:1, or 100:1. In some aspects, the at least one nucleic acid comprises at least one RNA molecule and at least one DNA molecule in a 1:1, 1:2, 1:3, 1:4, 2:1 or 4:1 ratio.

In some aspects, a lipid nanoparticle is provided comprising about 32.5% of at least one compound of Formula (I), Formula (II), or Formula (III) by moles, about 60% of at least one structural lipid by moles, about 5% of at least one phospholipid by moles, and about 2.5% of at least one PEGylated lipid by moles, wherein the at least one nucleic acid comprises at least one at least one RNA molecule and at least one DNA molecule. In some aspects, the present disclosure provides a lipid nanoparticle comprising about 22.5% to about 42.5% of at least one compound of Formula (I), Formula (II), or Formula (III) by moles, about 50% to about 70% of at least one structural lipid by moles, about 0.1% to about 15% of at least one phospholipid by moles, and about 0.1% to about 12.5% of at least one PEGylated lipid by moles, wherein the at least one nucleic acid comprises at least one RNA molecule and at least one DNA molecule. In some aspects, the present disclosure provides a lipid nanoparticle comprising about 27.5% to about 37.5% of at least one compound of Formula (I), Formula (II), or Formula (III) by moles, about 55% to about 65% of at least one structural lipid by moles, about 1% to about 10% of at least one phospholipid by moles, and about 1% to about 7.5% of at least one PEGylated lipid by moles, wherein the at least one nucleic acid comprises at least one RNA molecule and at least one DNA molecule. In one aspect, the at least one DNA molecule is a DoggyBone DNA molecule. In some aspects, the at least one DNA molecule is a DNA nanoplasmid. In some aspects, the mRNA molecule further comprises a 5′-CAP. In some aspects, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

Table A shows further exemplary LNP compositions of the present disclosure.

| | At least one compound | | | |
|---|---|---|---|---|
| | of Formula (I), | | | |
| | Formula (II) or | | | |
| | Formula (III) | Structural lipid | Phospholipid | PEGylated lipid |
| LNP # | (% by moles) | (% by moles) | (% by moles) | (% by moles) |
| 1 | 41.4 | 10 | 45.9 | 2.7 |
| 2 | about 31.4 to about 51.4 | about 0 to about 20 | about 35.9 to about | about 0.1 to about 12.7 |
| | | | 55.9 | |
| 3 | about 36.4 to about 46.4 | about 5 to about 15 | about 40.9 to about | about 0.2 to about 7.7 |
| | | | 50.9 | |
| 4 | about 38.9 to about 43.9 | about 7.5 to about 12.5 | about 43.4 to about | about 1 to about 5.2 |
| | | | 48.4 | |
| 5 | 41.44 | 10 | 45.85 | 2.7 |
| 6 | about 31.44 to about | about 0 to about 20 | about 35.85 to about | about 0.1 to about 12.7 |
| | 51.44 | | 55.85 | |
| 7 | about 36.44 to about | about 5 to about 15 | about 40.85 to about | about 0.2 to about 7.7 |
| | 46.44 | | 50.85 | |
| 8 | about 38.94 to about | about 7.5 to about 12.5 | about 43.35 to about | about 1 to about 5.2 |
| | 43.94 | | 48.35 | |
| 9 | 33.5 | 33.5 | 32 | 1 |
| 10 | about 23.5 to about 43.5 | about 23.5 to about 43.5 | about 22 to about 42 | about 0.1 to about 11 |
| 11 | about 28.5 to about 38.5 | about 28.5 to about 38.5 | about 27 to about 37 | about 0.2 to about 6 |
| 12 | about 31 to about 36 | about 31 to about 36 | about 29.5 to about | about 0.5 to about 3.5 |
| | | | 34.5 | |
| 13 | 40.7 | 47.1 | 10 | 2.2 |
| 14 | about 30.7 to about 50.7 | about 37.1 to about 57.1 | about 0.1 to about 20 | about 0.1 to about 12.2 |
| 15 | about 35.7 to about 45.7 | about 42.1 to about 52.1 | about 5 to about 15 | about 0.2 to about 7.2 |
| 16 | about 38.2 to about 43.2 | about 44.6 to about 49.6 | about 7.5 to about 12.5 | about 1 to about 4.7 |
| 17 | 40.68 | 47.12 | 10 | 2.2 |
| 18 | about 30.68 to about | about 37.12 to about | about 0.1 to about 20 | about 0.1 to about 12.2 |
| | 50.68 | 57.12 | | |
| 19 | about 35.68 to about | about 42.12 to about | about 5 to about 15 | about 0.2 to about 7.2 |
| | 45.68 | 52.12 | | |
| 20 | about 38.18 to about | about 44.62 to about | about 7.5 to about 12.5 | about 1 to about 4.7 |
| | 43.18 | 49.62 | | |
| 21 | 60 | 29 | 10 | 1 |
| 22 | about 50 to about 70 | about 19 to about 39 | about 0.1 to about 20 | about 0.1 to about 11 |
| 23 | about 55 to about 65 | about 24 to about 34 | about 5 to about 15 | about 0.2 to about 6 |
| 24 | about 57.5 to about 62.5 | about 26.5 to about 31.5 | about 7.5 to about 12.5 | about 0.5 to about 3.5 |
| 25 | 24.3 | 46.4 | 28.3 | 1 |
| 26 | about 14.3 to about 34.3 | about 36.4 to about 56.4 | about 18.3 to about | about 0.1 to about 11 |
| | | | 38.3 | |
| 27 | about 19.3 to about 29.3 | about 41.4 to about 51.4 | about 23.3 to about | about 0.2 to about 6 |
| | | | 33.3 | |
| 28 | about 21.8 to about 26.8 | about 43.9 to about 48.9 | about 25.8 to about | about 0.5 to about 3.5 |
| | | | 30.8 | |
| 29 | 24.29 | 46.41 | 28.3 | 1 |
| 30 | about 14.29 to about | about 36.41 to about | about 18.3 to about | about 0.1 to about 11 |
| | 34.29 | 56.41 | 38.3 | |
| 31 | about 19.29 to about | about 41.41 to about | about 23.3 to about | about 0.2 to about 6 |
| | 29.29 | 51.41 | 33.3 | |
| 32 | about 21.79 to about | about 43.91 to about | about 25.8 to about | about 0.5 to about 3.5 |
| | 26.79 | 48.91 | 30.8 | |
| 33 | 35 | 41.8 | 20 | 3.2 |
| 34 | about 25 to about 45 | about 31.8 to about 51.8 | about 10 to about 30 | about 0.1 to about 13.2 |
| 35 | about 30 to about 40 | about 36.8 to about 46.8 | about 15 to about 25 | about 0.2 to about 8.2 |
| 36 | about 32.5 to about 37.5 | about 39.3 to about 44.3 | about 17.5 to about | about 1 to about 5.7 |
| | | | 22.5 | |
| 37 | 35 | 41.8 | 20 | 3.16 |
| 38 | about 25 to about 45 | about 31.8 to about 51.8 | about 10 to about 30 | about 0.1 to about 13.16 |
| 39 | about 30 to about 40 | about 36.8 to about 46.8 | about 15 to about 25 | about 0.2 to about 8.16 |
| 40 | about 32.5 to about 37.5 | about 39.3 to about 44.3 | about 17.5 to about | about 1 to about 5.66 |
| | | | 22.5 | |
| 41 | 38 | 10 | 50 | 2 |
| 42 | about 28 to about 48 | about 0 to about 20 | about 40 to about 60 | about 0.1 to about 12 |
| 43 | about 33 to about 43 | about 5 to about 15 | about 45 to about 55 | about 0.2 to about 7 |
| 44 | about 35.5 to about 40.5 | about 7.5 to about 12.5 | about 47.5 to about | about 1 to about 4.5 |
| | | | 52.5 | |
| 45 | 35.3 | 53.7 | 8.3 | 2.7 |
| 46 | about 25.3 to about 45.3 | about 43.7 to about 63.7 | about 0.1 to about 18.3 | about 0.1 to about 12.7 |
| 47 | about 30.3 to about 40.3 | about 48.7 to about 58.7 | about 3.3 to about 13.3 | about 0.2 to about 7.7 |
| 48 | about 32.8 to about 37.8 | about 51.2 to about 56.2 | about 5.8 to about 10.8 | about 1 to about 5.2 |
| 49 | 35.28 | 53.68 | 8.35 | 2.69 |
| 50 | about 25.28 to about | about 43.68 to about | about 0.1 to about | about 0.1 to about 12.69 |
| | 45.28 | 63.68 | 18.35 | |
| 51 | about 30.28 to about | about 48.68 to about | about 3.35 to about | about 0.2 to about 7.69 |
| | 40.28 | 58.68 | 13.35 | |
| 52 | about 32.78 to about | about 51.18 to about | about 5.85 to about | about 1 to about 5.19 |
| | 37.78 | 56.18 | 10.85 | |
| 53 | 37.8 | 55.1 | 5 | 2.2 |
| 54 | about 27.8 to about 47.8 | about 45.1 to about 65.1 | about 0.1 to about 15 | about 0.1 to about 12.2 |
| 55 | about 32.8 to about 42.8 | about 50.1 to about 60.1 | about 1 to about 10 | about 0.2 to about 7.2 |
| 56 | about 35.3 to about 40.3 | about 52.6 to about 57.6 | about 2.5 to about 7.5 | about 1 to about 4.7 |
| 57 | 37.77 | 55.07 | 5 | 2.15 |
| 58 | about 27.77 to about | about 45.07 to about | about 0.1 to about 15 | about 0.1 to about 12.15 |
| | 47.77 | 65.07 | | |
| 59 | about 32.77 to about | about 50.07 to about | about 1 to about 10 | about 0.2 to about 7.15 |
| | 42.77 | 60.07 | | |
| 60 | about 35.27 to about | about 52.57 to about | about 2.5 to about 7.5 | about 1 to about 4.65 |
| | 40.27 | 57.57 | | |
| 61 | 44.4 | 48.5 | 5.4 | 1.7 |
| 62 | about 34.4 to about 54.4 | about 38.5 to about 58.5 | about 0.1 to about 15.4 | about 0.1 to about 11.7 |
| 63 | about 39.4 to about 49.4 | about 43.5 to about 53.5 | about 0.4 to about 10.4 | about 0.2 to about 6.7 |
| 64 | about 41.9 to about 46.9 | about 46 to about 51 | about 2.9 to about 7.9 | about 1 to about 4.2 |
| 65 | 44.38 | 48.46 | 5.43 | 1.73 |
| 66 | about 34.38 to about | about 38.46 to about | about 0.1 to about | about 0.1 to about 11.73 |
| | 54.38 | 58.46 | 15.43 | |
| 67 | about 39.38 to about | about 43.46 to about | about 0.43 to about | about 0.2 to about 6.73 |
| | 49.38 | 53.46 | 10.43 | |
| 68 | about 41.88 to about | about 45.96 to about | about 2.93 to about | about 1 to about 4.23 |
| | 46.88 | 50.96 | 7.93 | |
| 69 | 40 | 50.5 | 7.2 | 2.4 |
| 70 | about 30 to about 50 | about 40.5 to about 60.5 | about 0.1 to about 17.2 | about .1 to about 12.4 |
| 71 | about 35 to about 45 | about 45.5 to about 55.5 | about 2.2 to about 12.2 | about 0.2 to about 7.4 |
| 72 | about 37.5 to about 42.5 | about 48 to about 53 | about 4.7 to about 9.7 | about 1 to about 4.9 |
| 73 | 39.98 | 50.47 | 7.17 | 2.39 |
| 74 | about 29.98 to about | about 40.47 to about | about 0.1 to about | about 0.1 to about 12.39 |
| | 49.98 | 60.47 | 17.17 | |
| 75 | about 34.98 to about | about 45.47 to about | about 2.17 to about | about 0.2 to about 7.39 |
| | 44.98 | 55.47 | 12.17 | |
| 76 | about 37.48 to about | about 47.97 to about | about 4.67 to about | about 1 to about 4.89 |
| | 42.48 | 52.97 | 9.67 | |
| 77 | 37 | 46.7 | 14.3 | 2 |
| 78 | about 27 to about 47 | about 36.7 to about 56.7 | about 4.3 to about 24.3 | about 0.1 to about 12 |
| 79 | about 32 to about 42 | about 41.7 to about 51.7 | about 9.3 to about 19.3 | about 0.2 to about 7 |
| 80 | about 34.5 to about 39.5 | about 44.2 to about 49.2 | about 11.8 to about | about 1 to about 4.5 |
| | | | 16.8 | |
| 81 | 36.97 | 46.69 | 14.34 | 2 |
| 82 | about 26.97 to about | about 36.69 to about | about 4.34 to about | about 0.1 to about 12 |
| | 46.97 | 56.69 | 24.34 | |
| 83 | about 31.97 to about | about 41.69 to about | about 9.34 to about | about 0.2 to about 7 |
| | 41.97 | 51.69 | 19.34 | |
| 84 | about 34.47 to about | about 44.19 to about | about 11.84 to about | about 1 to about 4.5 |
| | 39.47 | 49.19 | 16.84 | |
| 85 | 34 | 51.4 | 12.9 | 1.7 |
| 86 | about 24 to about 44 | about 41.4 to about 61.4 | about 2.9 to about 22.9 | about 0.1 to about 11.7 |
| 87 | about 29 to about 39 | about 46.4 to about 56.4 | about 7.9 to about 17.9 | about 0.2 to about 6.7 |
| 88 | about 31.5 to about 36.5 | about 48.9 to about 53.9 | about 10.4 to about | about 1 to about 4.2 |
| | | | 15.4 | |
| 89 | 33.96 | 51.44 | 12.89 | 1.7 |
| 90 | about 23.96 to about | about 41.44 to about | about 2.89 to about | about 0.1 to about 11.7 |
| | 43.96 | 61.44 | 22.89 | |
| 91 | about 28.96 to about | about 46.44 to about | about 7.89 to about | about 0.2 to about 6.7 |
| | 38.96 | 56.44 | 17.89 | |
| 92 | about 31.46 to about | about 48.94 to about | about 10.39 to about | about 1 to about 4.2 |
| | 36.46 | 53.94 | 15.39 | |
| 93 | 45 | 40.8 | 12.4 | 1.9 |
| 94 | about 35 to about 55 | about 30.8 to about 50.8 | about 2.4 to about 22.4 | about 0.1 to about 11.9 |
| 95 | about 40 to about 50 | about 35.8 to about 45.8 | about 7.4 to about 17.4 | about 0.2 to about 6.9 |
| 96 | about 42.5 to about 47.5 | about 38.3 to about 43.3 | about 9.9 to about 14.9 | about 1 to about 4.4 |
| 97 | 44.95 | 40.77 | 12.4 | 1.88 |
| 98 | about 34.95 to about | about 30.77 to about | about 2.4 to about 22.4 | about 0.1 to about 11.88 |
| | 54.95 | 50.77 | | |
| 99 | about 39.95 to about | about 35.77 to about | about 7.4 to about 17.4 | about 0.2 to about 6.88 |
| | 49.95 | 45.77 | | |
| 100 | about 42.45 to about | about 38.27 to about | about 9.9 to about 14.9 | about 1 to about 4.38 |
| | 47.45 | 43.27 | | |
| 101 | 43.1 | 45.1 | 9.6 | 2.2 |
| 102 | about 33.1 to about 53.1 | about 35.1 to about 55.1 | about 0.1 to about 19.6 | about 0.1 to about 12.2 |
| 103 | about 38.1 to about 48.1 | about 40.1 to about 50.1 | about 4.6 to about 14.6 | about 0.2 to about 7.2 |
| 104 | about 40.6 to about 45.6 | about 42.6 to about 47.6 | about 7.1 to about 12.1 | about 1 to about 4.7 |
| 105 | 43.11 | 45.09 | 9.59 | 2.21 |
| 106 | about 33.11 to about | about 35.09 to about | about 0.1 to about | about 0.1 to about 12.21 |
| | 53.11 | 55.09 | 19.59 | |
| 107 | about 38.11 to about | about 40.09 to about | about 4.59 to about | about 0.2 to about 7.21 |
| | 48.11 | 50.09 | 14.59 | |
| 108 | about 40.61 to about | about 42.59 to about | about 7.09 to about | about 1 to about 4.71 |
| | 45.61 | 47.59 | 12.09 | |
| 109 | 41 | 42.8 | 13.5 | 2.6 |
| 110 | about 31 to about 51 | about 32.8 to about 52.8 | about 3.5 to about 23.5 | about 0.1 to about 12.6 |
| 111 | about 36 to about 46 | about 37.8 to about 47.8 | about 8.5 to about 18.5 | about 0.2 to about 7.6 |
| 112 | about 38.5 to about 43.5 | about 40.3 to about 45.3 | about 11 to about 16 | about 1 to about 5.1 |
| 113 | 41.02 | 42.83 | 13.55 | 2.61 |
| 114 | about 31.02 to about | about 32.83 to about | about 3.55 to about | about 0.1 to about 12.61 |
| | 51.02 | 52.83 | 23.55 | |
| 115 | about 36.02 to about | about 37.83 to about | about 8.55 to about | about 0.2 to about 7.61 |
| | 46.02 | 47.83 | 18.55 | |
| 116 | about 38.52 to about | about 40.33 to about | about 11.05 to about | about 1 to about 5.11 |
| | 43.52 | 45.33 | 16.05 | |
| 117 | 36.7 | 48.4 | 12.5 | 2.4 |
| 118 | about 26.7 to about 46.7 | about 38.4 to about 58.4 | about 2.5 to about 22.5 | about 0.1 to about 12.4 |
| 119 | about 31.7 to about 41.7 | about 43.4 to about 53.4 | about 7.5 to about 17.5 | about 0.2 to about 7.4 |
| 120 | about 34.2 to about 39.2 | about 45.9 to about 50.9 | about 10 to about 15 | about 1 to about 4.9 |
| 121 | 36.71 | 48.44 | 12.46 | 2.39 |
| 122 | about 26.71 to about | about 38.44 to about | about 2.46 to about | about 0.1 to about 12.39 |
| | 46.71 | 58.44 | 22.46 | |
| 123 | about 31.71 to about | about 43.44 to about | about 7.46 to about | about 0.2 to about 7.39 |
| | 41.71 | 53.44 | 17.46 | |
| 124 | about 34.21 to about | about 45.94 to about | about 9.96 to about | about 1 to about 4.89 |
| | 39.21 | 50.94 | 14.96 | |
| 125 | 45.1 | 40.6 | 12.6 | 1.8 |
| 126 | about 35.1 to about 55.1 | about 30.6 to about 50.6 | about 2.6 to about 22.6 | about 0.1 to about 11.8 |
| 127 | about 40.1 to about 50.1 | about 35.6 to about 45.6 | about 7.6 to about 17.6 | about 0.2 to about 6.8 |
| 128 | about 42.6 to about 47.6 | about 38.1 to about 43.1 | about 10.1 to about | about 1 to about 4.3 |
| | | | 15.1 | |
| 129 | 45.12 | 40.58 | 12.55 | 1.75 |
| 130 | about 35.12 to about | about 30.58 to about | about 2.55 to about | about 0.1 to about 11.75 |
| | 55.12 | 50.58 | 22.55 | |
| 131 | about 40.12 to about | about 35.58 to about | about 7.55 to about | about 0.2 to about 6.75 |
| | 50.12 | 45.58 | 17.55 | |
| 132 | about 42.62 to about | about 38.08 to about | about 10.05 to about | about 1 to about 4.25 |
| | 47.62 | 43.08 | 15.05 | |
| 133 | 40.8 | 51.8 | 5 | 2.5 |
| 134 | about 30.8 to about 50.8 | about 41.8 to about 61.8 | about 0.1 to about 15 | about 0.1 to about 12.5 |
| 135 | about 35.8 to about 45.8 | about 46.8 to about 56.8 | about 1 to about 10 | about 0.2 to about 7.5 |
| 136 | about 38.3 to about 43.3 | about 49.3 to about 54.3 | about 2.5 to about 7.5 | about 1 to about 5 |
| 137 | 40.75 | 51.75 | 5 | 2.5 |
| 138 | about 30.75 to about | about 41.75 to about | about 0.1 to about 15 | about 0.1 to about 12.5 |
| | 50.75 | 61.75 | | |
| 139 | about 35.75 to about | about 46.75 to about | about 1 to about 10 | about 0.2 to about 7.5 |
| | 45.75 | 56.75 | | |
| 140 | about 38.25 to about | about 49.25 to about | about 2.5 to about 7.5 | about 1 to about 5 |
| | 43.25 | 54.25 | | |
| 141 | 32.5 | 60 | 5 | 2.5 |
| 142 | about 22.5 to about 42.5 | about 50 to about 70 | about 0.1 to about 15 | about 0.1 to about 12.5 |
| 143 | about 27.5 to about 37.5 | about 55 to about 65 | about 1 to about 10 | about 0.5 to about 7.5 |
| 144 | about 30 to about 35 | about 57.5 to about 62.5 | about 2.5 to about 7.5 | about 1 to about 5 |
| 145 | 34.4 | 56.18 | 7.86 | 1.54 |
| 146 | about 24.4 to about 44.4 | about 46.18 to about | about 0.1 to about | about 0.1 to about 11.54 |
| | | 66.18 | 17.86 | |
| 147 | about 29.4 to about 39.4 | about 51.18 to about | about 2.86 to about | about 0.2 to about 6.54 |
| | | 61.18 | 12.86 | |
| 148 | about 31.9 to about 36.9 | about 53.68 to about | about 5.36 to about | about 1 to about 4.04 |
| | | 58.68 | 10.36 | |
| 149 | 47.03 | 46.28 | 3.76 | 2.93 |
| 150 | about 37.03 to about | about 36.28 to about | about 0.1 to about | about 0.1 to about 12.93 |
| | 57.03 | 56.28 | 13.76 | |
| 151 | about 42.03 to about | about 41.28 to about | about 0.5 to about 8.76 | about 0.2 to about 7.93 |
| | 52.03 | 51.28 | | |
| 152 | about 44.53 to about | about 43.78 to about | about 1.26 to about | about 0.43 to about 5.43 |
| | 49.53 | 48.78 | 6.26 | |
| 153 | 44.48 | 48.46 | 4.68 | 2.34 |
| 154 | about 34.48 to about | about 38.46 to about | about 0.1 to about | about 0.1 to about 12.34 |
| | 54.48 | 58.46 | 14.68 | |
| 155 | about 39.48 to about | about 43.46 to about | about 0.5 to about 9.68 | about 0.2 to about 7.34 |
| | 49.48 | 53.46 | | |
| 156 | about 41.98 to about | about 45.96 to about | about 2.18 to about | about 1 to about 4.84 |
| | 46.98 | 50.96 | 7.18 | |
| 157 | 30.3 | 50.45 | 17.27 | 1.98 |
| 158 | about 20.3 to about 40.3 | about 40.45 to about | about 7.27 to about | about 0.1 to about 11.98 |
| | | 60.45 | 27.27 | |
| 159 | about 25.3 to about 35.3 | about 45.45 to about | about 12.27 to about | about 0.2 to about 6.98 |
| | | 55,45 | 22.27 | |
| 160 | about 27.8 to about 32.8 | about 47.95 to about | about 14.77 to about | about 1 to about 4.48 |
| | | 52.95 | 19.77 | |
| 161 | 66.98 | 30.96 | 0.35 | 1.71 |
| 162 | about 56.98 to about | about 20.96 to about | about 0.1 to about | about 0.1 to about 11.71 |
| | 76.98 | 40.96 | 10.35 | |
| 163 | about 61.98 to about | about 25.96 to about | about 0.2 to about 5.35 | about 0.2 to about 6.71 |
| | 71.98 | 35.96 | | |
| 164 | about 64.48 to about | about 28.46 to about | about 0.3 to about 2.85 | about 1 to about 4.21 |
| | 69.48 | 33.46 | | |
| 165 | 30 | 55 | 12 | 3 |
| 166 | about 20 to about 40 | about 45 to about 65 | about 2 to about 22 | about 0.1 to about 13 |
| 167 | about 25 to about 35 | about 50 to about 60 | about 7 to about 17 | about 0.2 to about 8 |
| 168 | about 27.5 to about 32.5 | about 52.5 to about 57.5 | about 9.5 to about 14.5 | about 0.5 to about 5.5 |
| 169 | 51.71 | 40.46 | 5.99 | 1.85 |
| 170 | about 41.71 to about | about 30.46 to about | about 0.1 to about | about 0.1 to about 11.85 |
| | 61.71 | 50.46 | 15.99 | |
| 171 | about 46.71 to about | about 35.46 to about | about 0.99 to about | about 0.2 to about 6.85 |
| | 56.71 | 45.46 | 10.99 | |
| 172 | about 49.21 to about | about 37.96 to about | about 3.49 to about | about 1 to about 4.35 |
| | 54.21 | 42.96 | 8.49 | |
| 173 | 52.5 | 30 | 16 | 1.5 |
| 174 | about 42.5 to about 62.5 | about 20 to about 40 | about 6 to about 26 | about 0.1 to about 11.5 |
| 175 | about 47.5 to about 57.5 | about 25 to about 35 | about 11 to about 21 | about 0.2 to about 6.5 |
| 176 | about 50 to about 55 | about 27.5 to about 32.5 | about 13.5 to about | about 1 to about 4 |
| | | | 18.5 | |
| 177 | 47 | 30 | 20 | 3 |
| 178 | about 37 to about 57 | about 20 to about 40 | about 10 to about 30 | about 0.1 to about 13 |
| 179 | about 42 to about 52 | about 25 to about 35 | about 15 to about 25 | about 0.2 to about 8 |
| 180 | about 44.5 to about 49.5 | about 27.5 to about 32.5 | about 17.5 to about | about 0.5 to about 5.5 |
| | | | 22.5 | |
| 181 | 54.11 | 33.4 | 9.49 | 3 |
| 182 | about 44.11 to about | about 23.4 to about 43.4 | about 0.1 to about | about 0.1 to about 13 |
| | 64.11 | | 19.49 | |
| 183 | about 49.11 to about | about 28.4 to about 38.4 | about 4.49 to about | about 0.2 to about 8 |
| | 59.11 | | 14.49 | |
| 184 | about 51.61 to about | about 30.9 to about 35.9 | about 6.99 to about | about 1 to about 5.5 |
| | 56.61 | | 11.99 | |
| 185 | 29 | 60 | 10 | 1 |
| 186 | about 19 to about 39 | about 50 to about 70 | about 0 to about 20 | about 0.1 to about 11 |
| 187 | about 24 to about 34 | about 55 to about 65 | about 5 to about 15 | about 0.2 to about 6 |
| 188 | about 26.5 to about 31.5 | about 57.5 to about 62.5 | about 7.5 to about 12.5 | about 0.5 to about 3.5 |
| 189 | 60 | 10 | 27.8 | 2.2 |
| 190 | about 50 to about 70 | about 0 to about 20 | about 17.8 to about | about 0.1 to about 12.2 |
| | | | 37.8 | |
| 191 | about 55 to about 65 | about 5 to about 15 | about 22.8 to about | about 0.2 to about 7.2 |
| | | | 32.8 | |
| 192 | about 57.5 to about 62.5 | about 7.5 to about 12.5 | about 25.3 to about | about 1 to about 4.7 |
| | | | 30.3 | |
| 193 | 44.5 | 10 | 44.5 | 1 |
| 194 | about 34.5 to about 54.5 | about 0 to about 20 | about 34.5 to about | about 0.1 to about 11 |
| | | | 54.5 | |
| 195 | about 39.5 to about 49.5 | about 5 to about 15 | about 39.5 to about | about 0.2 to about 6 |
| | | | 49.5 | |
| 196 | about 42 to about 47 | about 7.5 to about 12.5 | about 42 to about 47 | about 0.5 to about 3.5 |
| 197 | 46.32 | 39.48 | 10 | 4.2 |
| 198 | about 36.32 to about | about 29.48 to about | about 0.1 to about 20 | about 0.1 to about 14.2 |
| | 56.32 | 49.48 | | |
| 199 | about 41.32 to about | about 34.48 to about | about 5 to about 15 | about 0.2 to about 9.2 |
| | 51.32 | 44.48 | | |
| 200 | about 43.82 to about | about 36.98 to about | about 7.5 to about 12.5 | about 1.7 to about 6.7 |
| | 48.82 | 41.98 | | |

In some aspects of the preceding LNPs, the structural lipid can be cholesterol. In some aspects of the preceding LNPs, the phospholipid can be DOPE. In some aspects of the preceding LNPs, the phospholipid can be DSPC. In some aspects of the preceding LNPs, the phospholipid can be DOPC. In some aspects of the preceding LNPs, the PEGylated lipid can be DMG-PEG2000.

In some aspects of the preceding LNPs, the structural lipid can be cholesterol, the phospholipid can be DOPE and the PEGylated lipid can be DMG-PEG2000.

In some aspects of the preceding LNPs, the structural lipid can be cholesterol, the phospholipid can be DOPC and the PEGylated lipid can be DMG-PEG2000.

### Pharmaceutical Compositions of the Present Disclosure

In some aspects, the present disclosure provides a pharmaceutical composition comprising at least one lipid nanoparticle of the present disclosure. In some aspects, the present disclosure provides a pharmaceutical composition comprising at least one first nanoparticle of the present disclosure and at least one second nanoparticle of the present disclosure, wherein the at least one first nanoparticle comprises at least one nucleic acid molecule encoding at least one transposase, wherein the at least one second nanoparticle comprises at least one nucleic acid molecule encoding at least one transposon. In some aspects, the at least one nucleic acid molecule encoding at least one transposase can be an RNA molecule (e.g. mRNA molecule) and the at least one nucleic acid molecule encoding at least one transposon can be a DNA molecule (e.g. a Doggy Bone DNA molecule or a DNA nanoplasmid).

In some aspects, the present disclosure provides a composition comprising at least one cell that has been contacted by at least one nanoparticle of the present disclosure. In some aspects, the present disclosure provides a composition comprising at least one cell that has been genetically modified using at least one nanoparticle of the present disclosure. In some aspects, the present disclosure provides a composition comprising at least one cell that has been genetically modified using any method of the present disclosure.

In some aspects, the present disclosure provides a pharmaceutical composition comprising at least one cell that has been contacted by at least one nanoparticle of the present disclosure. In some aspects, the present disclosure provides a pharmaceutical composition comprising at least one cell that has been genetically modified using at least one nanoparticle of the present disclosure. In some aspects, the present disclosure provides a pharmaceutical composition comprising at least one cell that has been genetically modified using any method of the present disclosure.

### Methods of the Present Disclosure

The present disclosure provides a method of delivering at least one nucleic acid to at least one cell comprising contacting the at least one cell with at least one composition of the present disclosure. The present disclosure provides a method of delivering at least one nucleic acid to at least one cell comprising contacting the at least one cell with at least one nanoparticle of the present disclosure.

In all methods, compositions and kits of the present disclosure, at least one cell can be a liver cell. A liver cell can include, but is not limited to, a hepatocyte, a hepatic stellate cell, Kupffer cell or a liver sinusoidal endothelial cell. In all methods, compositions and kits of the present disclosure, at least one cell can be a T-cell. A T-cell can be a resting T-cell, an activated T-cell, stem memory T cells (T_{SCM} cells), central memory T cells (T_{CM}), or stem cell-like T cells.

In some aspects of any methods of the present disclosure, a cell can be in vivo, ex vivo or in vitro. In some aspects, any of the methods of the present disclosure can be applied in vivo, ex vivo or in vitro.

The present disclosure provides a method of genetically modifying at least one cell comprising contacting the at least one cell with at least one composition of the present disclosure. The present disclosure provides a method of genetically modifying at least one cell comprising contacting the at least one cell with at least one nanoparticle of the present disclosure.

In some aspects, genetically modifying a cell can comprise delivering at least one exogenous nucleic acid to the cell such that the cell expresses at least one protein that the cell otherwise would not normally express, or such that the at least one cell expresses at least one protein at a level that is higher than the level that the cell would otherwise normally express the at least one protein, or such that the cell expresses at least one protein at a level that is lower than the level that the cell would otherwise normally express. In some aspects, genetically modifying a cell can comprise delivering at least one exogenous nucleic to the cell such that at least one exogenous nucleic acid is integrated into the genome of the at least one cell.

In all methods of the present disclosure, T-cells can be activated prior to, concurrently with, or after contacting the T-cells with at least one composition or at least one nanoparticle of the present disclosure. In some aspects, T-cells can be activated using standard techniques known in the art, including, but not limited to, contacting the T-cells with CD3/CD28/CD2 activator solution, anti-CD3 antibody beads, anti-CD28 antibody bead, anti-CD2 antibody beads, anti-CD3 and anti-CD28 antibody beads, tetrameric antibody complexes that bind CD3, CD28 and CD2 cell surface ligands, or any combination thereof.

In some aspects, T-cells can be activated at least 1 hour, or at least 2 hours, or at least 3 hours, or at least 4 hours, or at least 5 hours, or at least 6 hours, or at least 7 hours, or at least 8 hours, or at least 9 hours, or at least 10 hours, or at least 11 hours, or at least 12 hours, or at least 13 hours, or at least 14 hours, or at least 15 hours, or at least 16 hours, or at least 17 hours, or at least 18 hours, or at least 19 hours, or at least 20 hours, or at least 21 hours, or at least 22 hours, or at least 23 hours, or at least 24 hours, or at least 36 hours, or at least 48 hours, or at least 60 hours, or at least 72 hours prior to be contacted with at least one composition or nanoparticle of the present disclosure.

In some aspects, T-cells can be activated at least 1 hour, or at least 2 hours, or at least 3 hours, or at least 4 hours, or at least 5 hours, or at least 6 hours, or at least 7 hours, or at least 8 hours, or at least 9 hours, or at least 10 hours, or at least 11 hours, or at least 12 hours, or at least 13 hours, or at least 14 hours, or at least 15 hours, or at least 16 hours, or at least 17 hours, or at least 18 hours, or at least 19 hours, or at least 20 hours, or at least 21 hours, or at least 22 hours, or at least 23 hours, or at least 24 hours, or at least 36 hours, or at least 48 hours, or at least 60 hours, or at least 72 hours after being contacted with at least one composition or nanoparticle of the present disclosure.

In some aspects of the preceding methods, step c) can be performed at least 1 hour, or at least 2 hours, or at least 3 hours, or at least 4 hours, or at least 5 hours, or at least 6 hours, or at least 7 hours, or at least 8 hours, or at least 9 hours, or at least 10 hours, or at least 11 hours, or at least 12 hours, or at least 13 hours, or at least 14 hours, or at least 15 hours, or at least 16 hours, or at least 17 hours, or at least 18 hours, or at least 19 hours, or at least 20 hours, or at least 21 hours, or at least 22 hours, or at least 23 hours, or at least 24 hours, or at least 36 hours, or at least 48 hours, or at least 60 hours, or at least 72 hours after step b).

In some aspects of the preceding methods, step a) can be performed at least 1 hour, or at least 2 hours, or at least 3 hours, or at least 4 hours, or at least 5 hours, or at least 6 hours, or at least 7 hours, or at least 8 hours, or at least 9 hours, or at least 10 hours, or at least 11 hours, or at least 12 hours, or at least 13 hours, or at least 14 hours, or at least 15 hours, or at least 16 hours, or at least 17 hours, or at least 18 hours, or at least 19 hours, or at least 20 hours, or at least 21 hours, or at least 22 hours, or at least 23 hours, or at least 24 hours, or at least 36 hours, or at least 48 hours, or at least 60 hours, or at least 72 hours prior to step b).

In some aspects, the methods of the present disclosure can yield a plurality of cells, wherein at least about 1%, or at least about 2%, or at least about 3%, or at least about 4%, or at least about 5%, or at least 10%, or at least 15%, or at least 20%, or at least 25%, or at least 30%, or at least 35%, or at least 40%, or at least 45%, or at least 50%, or at least 55%, or at least 60%, or at least about 65%, or at least about 70%, or at least about 75%, or at least about 80%, or at least about 85%, or at least about 90%, or at least about 95%, or at least about 99% of the cell in the plurality express at least one protein that was encoded in at least one nucleic acid that was delivered to the plurality of cells via a nanoparticle of the present disclosure.

In some aspects, the methods of the present disclosure can yield a plurality of cells, wherein at least about 1%, or at least about 2%, or at least about 3%, or at least about 4%, or at least about 5%, or at least 10%, or at least 15%, or at least 20%, or at least 25%, or at least 30%, or at least 35%, or at least 40%, or at least 45%, or at least 50%, or at least 55%, or at least 60%, or at least about 65%, or at least about 70%, or at least about 75%, or at least about 80%, or at least about 85%, or at least about 90%, or at least about 95%, or at least about 99% of the cells in the plurality are stem memory T cells.

In some aspects, the methods of the present disclosure can yield a plurality of cells, wherein at least about 1%, or at least about 2%, or at least about 3%, or at least about 4%, or at least about 5%, or at least 10%, or at least 15%, or at least 20%, or at least 25%, or at least 30%, or at least 35%, or at least 40%, or at least 45%, or at least 50%, or at least 55%, or at least 60%, or at least about 65%, or at least about 70%, or at least about 75%, or at least about 80%, or at least about 85%, or at least about 90%, or at least about 95%, or at least about 99% of the cells are express one or more cell-surface marker(s) of a stem memory T cell (T_{SCM}) or a T_{SCM}-like cell and wherein the one or more cell-surface marker(s) comprises CD62L and CD45RA.

The present disclosure provides a method of treating at least one disease in a subject, the method comprising administering to the subject at least one therapeutically effective amount of at least one composition of the present disclosure comprising at least one nucleic acid encoding a therapeutic protein.

The present disclosure provides a method of treating at least one disease in a subject, the method comprising administering at least one therapeutically effective amount of at least one nanoparticle of the present disclosure comprising at least one nucleic acid encoding a therapeutic protein.

The present disclosure provides a method of treating at least one disease in a subject, the method comprising administering at least one therapeutically effective amount of cells, wherein the cells have been contacted by at least one nanoparticle of the present disclosure comprising at least one nucleic acid encoding a therapeutic protein. The present disclosure provides a method of treating at least one disease in a subject, the method comprising administering at least one therapeutically effective amount of cells, wherein the cells have been genetically modified using the compositions and/or methods of the present disclosure.

In some aspects, the at least one disease can be a malignant disease, including, but not limited to, cancer. In some aspects, the at least one disease can be a metabolic liver disorder (MLD). In some aspects, the at least one disease can be a urea cycle disorder (UCD). An MLD and/or UCD can include, but is not limited to, N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), methylmalonic acidemia (MMA) or any combination thereof. In some aspects, the at least one disease can be hemophilia A.

In some aspects, the at least one disease can be a disease and/or disorder characterized by increased LDL-cholesterol. Accordingly, the present disclosure provides methods of decreasing LDL-cholesterol in a subject in need thereof, the methods comprising administering to the at least one composition disclosed herein.

Accordingly, the present disclosure provides a method of treating hemophilia A in a subject in need thereof comprising administering to the subject at least one composition comprising at least one lipid nanoparticle of the present disclosure, wherein the lipid nanoparticle comprises a nucleic acid encoding a FVIII polypeptide.

According, the present disclosure provides a method of treating Ornithine Transcarbamylase (OTC) Deficiency in a subject in need thereof comprising administering to the subject at least one composition comprising at least one lipid nanoparticle of the present disclosure, wherein the lipid nanoparticle comprises a nucleic acid encoding an ornithine transcarbamylase (OTC) polypeptide.

Accordingly, the present disclosure provides a method of treating methylmalonic acidemia (MMA) in a subject in need thereof comprising administering to the subject at least one composition comprising at least one lipid nanoparticle of the present disclosure, wherein the lipid nanoparticle comprises a nucleic acid encoding a methylmalonyl-CoA mutase (MUT1) polypeptide.

The present disclosure provides methods of treating a disease and/or disorder characterized by increased LDL-cholesterol comprising administering to the subject at least one LNP of the present disclosure comprising a genomic editing composition, wherein the genomic editing composition comprises a nucleic acid molecule comprising a nucleic acid sequence encoding a fusion protein, wherein the fusion protein comprises: (i) an inactivated Cas9 (dCas9) protein or an inactivated nuclease domain thereof; and (ii) a Clo051 protein or a nuclease domain thereof. In some aspects, the fusion protein can be a Cas-CLOVER protein. In some aspects, the genomic editing composition can further comprise at least one species of guide RNA (gRNA) molecule targeting the pesk9 gene. In some aspects, the genomic editing composition can further comprise at least two gRNA molecules targeting the pesk9 gene. gRNA molecules targeting the pesk9 gene can comprise, consist of, or consist essentially of a nucleic acid sequence at least 65%, 70%, 75%, 80%, 85%, 90%, 95%, 96%, 97%, 98%, 99% or 100% (or any percentage in between) identical to the nucleotide sequences put forth in SEQ ID NOs: 1-2.

The present disclosure provides methods of decreasing LDL-cholesterol in a subject in need thereof, the method comprising administering to the subject at least one LNP of the present disclosure comprising a genomic editing composition, wherein the genomic editing composition comprises a nucleic acid molecule comprising a nucleic acid sequence encoding a fusion protein, wherein the fusion protein comprises: (i) an inactivated Cas9 (dCas9) protein or an inactivated nuclease domain thereof; and (ii) a Clo051 protein or a nuclease domain thereof. In some aspects, the fusion protein can be a Cas-CLOVER protein. In some aspects, the genomic editing composition can further comprise at least one species of guide RNA (gRNA) molecule targeting the pesk9 gene. In some aspects, the genomic editing composition can further comprise at least two gRNA molecules targeting the pesk9 gene. gRNA molecules targeting the pesk9 gene can comprise, consist of, or consist essentially of a nucleic acid sequence at least 65%, 70%, 75%, 80%, 85%, 90%, 95%, 96%, 97%, 98%, 99% or 100% (or any percentage in between) identical to the nucleotide sequences put forth in SEQ ID NOs: 1-2.

In some aspects, an inactivated Cas9 (dCas9) protein or an inactivated nuclease domain thereof can comprise, consist of, or consist essentially of an amino acid sequence at least 65%, 70%, 75%, 80%, 85%, 90%, 95%, 96%, 97%, 98%, 99% or 100% (or any percentage in between) identical to SEQ ID NO: 3.

In some aspects, a Clo051 protein or a nuclease domain thereof can comprise, consist of, or consist essentially of an amino acid sequence at least 65%, 70%, 75%, 80%, 85%, 90%, 95%, 96%, 97%, 98%, 99% or 100% (or any percentage in between) identical to SEQ ID NO: 4.

In some aspects, a Cas-CLOVER protein can comprises, consist of, or consist essentially of an amino acid sequence at least 65%, 70%, 75%, 80%, 85%, 90%, 95%, 96%, 97%, 98%, 99% or 100% (or any percentage in between) identical to SEQ ID NO: 5.

### Nucleic Acid Molecules

In some aspects, a nucleic acid molecule can be an RNA molecule. Thus, in some aspects, a lipid nanoparticle can comprise at least one RNA molecule. The at least one RNA molecule can be encapsulated within the lipid nanoparticle. In some aspects, an RNA molecule can be an mRNA molecule. In some aspects, a lipid nanoparticle can comprise at least one mRNA molecule. The mRNA molecule can be encapsulated within the lipid nanoparticle.

In some aspects, a nucleic acid molecule can be a synthetic nucleic acid molecule. In some aspects, a nucleic acid molecule can be a non-naturally occurring nucleic acid molecule. In some aspects, a non-naturally occurring nucleic acid molecule can comprise at least one non-naturally occurring nucleotide. The at least one non-naturally occurring nucleotide can be any non-naturally occurring nucleotide known in the art. In some aspects, a nucleic acid molecule can be a modified nucleic acid molecule. In some aspects, a modified nucleic acid molecule can comprise at least one modified nucleotide. The at least one modified nucleotide can be any modified nucleic acid known in the art.

In some aspects, an mRNA molecule can be capped using any method and/or capping moiety known in the art. An mRNA molecule can be capped with m7G (5′) ppp (5′) G moiety. A m7G (5′) ppp (5′) G moiety is also referred to herein as a “Cap0”. An mRNA molecule can be capped with a CleanCap® moiety. A CleanCap® moiety can comprise a m7G (5′) ppp (5′) (2′OMeA) (CleanCap® AG) moiety. A CleanCap® moiety can comprise a m7G (5′) ppp (5′) (2′OMeG) (CleanCap® GG) moiety. An mRNA molecule can be capped with an anti-reverse cap analog (ARCA®) moiety. An ARCA® moiety can comprise a m7 (3′-O-methyl) G (5′) ppp (5′) G moiety. An mRNA molecule can be capped with a CleanCap® 3′OMe moiety (CleanCap®+ARCA®).

In some aspects, an mRNA molecule can comprise at least one modified nucleic acid.

The at least one modified nucleic acid can comprise 5-methoxyuridine (5moU). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the uridine bases in an mRNA molecule are 5-methoxyuridine bases. In some aspects, all of the uridine bases in an mRNA molecule are 5-methoxyuridine bases. Without wishing to be bound by theory, 5-methoxyuridine can improve protein expression and reduce immunogenicity (see Li et al., Bioconjugate Chem. 2016, 27, 3, 849-853 and Vaidyanathan et al. Molecular Therapy Nucleic Acids, 2018, 12, 530-542).

In some aspects, an mRNA molecule can comprise at least one modified nucleic acid.

The at least one modified nucleic acid can comprise N1-methylpseudouridine (mely′). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the uridine bases in an mRNA Ni-methylpseudouridine bases. In some aspects, all of the uridine bases in an mRNA molecule are Ni-methylpseudouridine bases. Without wishing to be bound by theory, N1-methylpseudouridine can improve protein expression (see Li et al., Bioconjugate Chem. 2016, 27, 3, 849-853).

In some aspects, an mRNA molecule can comprise at least one modified nucleic acid.

The at least one modified nucleic acid can comprise pseudouridine (Y). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the uridine bases in an mRNA pseudouridine bases. In some aspects, all of the uridine bases in an mRNA molecule are pseudouridine bases. Without wishing to be bound by theory, pseudouridine can improve protein expression and reduce immunogenicity (see Li et al., Bioconjugate Chem. 2016, 27, 3, 849-853 and Vaidyanathan et al. Molecular Therapy Nucleic Acids, 2018, 12, 530-542).

In some aspects, an mRNA molecule can comprise at least one modified nucleic acid.

The at least one modified nucleic acid can comprise 5-methylcytidine (5-MeC). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the cytidine bases in an mRNA 5-MeC bases. In some aspects, all of the cytidine bases in an mRNA molecule are 5-MeC bases.

In some aspects, a nucleic acid molecule can comprise a DNA molecule. Thus, in some aspects, a lipid nanoparticle can comprise a DNA molecule. In some aspects, the DNA molecule can be a circular DNA molecule, such as, but not limited to, a DNA plasmid or DNA nanoplasmid. Thus, in some aspects, a lipid nanoparticle can comprise a circular DNA molecule. In some aspects, a lipid nanoparticle can comprise a Doggybone DNA molecule. In some aspects, a lipid nanoparticle can comprise a DNA plasmid. In some aspects, a lipid nanoparticle can comprise a DNA nanoplasmid. In some aspects, a DNA molecule can be a linearized DNA molecule, such as, but not limited to, a linearized DNA plasmid or a linearized DNA nanoplasmid.

A DNA plasmid or DNA nanoplasmid can comprise can be at least about 0.25 kb, or at least about 0.5 kb, or at least about 0.75 kb, or at least about 1.0 kb, or at least about 1.25 kb, or at least about 1.5 kb, or at least about 1.75 kb, or at least about 2.0 kb, or at least about 2.25 kb, or at least about 2.5 kb, or at least about 2.75 kb, or at least about 3.0 kb, or at least about 3.25 kb, or at least about 3.5 kb, or at least about 3.75 kb, or at least about 4.0 kb, or at least about 4.25 kb, or at least about 4.5 kb, or at least about 4.75 kb, or at least about 5.0 kb, or at least about 5.25 kb, or at least about 5.5 kb, or at least about 5.75 kb, or at least about 6.0 kb, or at least about 6.25 kb, or at least about 6.5 kb, or at least about 6.75 kb, or at least about 7.0 kb, or at least about 7.25 kb, or at least about 7.5 kb, or at least about 7.75 kb, or at least about 8.0 kb, or at least about 8.25 kb, or at least about 8.5 kb, or at least about 8.75 kb, or at least about 9.0 kb, or at least about 9.25 kb, or at least about 9.5 kb, or at least about 9.75 kb, or at least about 10.0 kb, or at least about 10.25 kb, or at least about 10.5 kb, or at least about 10.75 kb, or at least about 11.0 kb, or at least about 11.25 kb, or at least about 11.5 kb, or at least about 11.75 kb, or at least about 12 kb, or at least about 12.25 kb, or at least about 12.5 kb, or at least about 12.75 kb, or at least about 13.0 kb, or at least about 13.25 kb, or at least about 13.5 kb, or at least about 13.75 kb, or at least about 14.0 kb, or at least about 14.25 kb, or at least about 14.5 kb, or at least about 14.75 kb or at least about 15.0 kb in length.

In some aspects, a nucleic acid molecule formulated in a lipid nanoparticle of the present disclosure can comprise at least one transgene sequence. In some aspects, a transgene sequence can comprise a nucleotide sequence encoding at least one therapeutic protein. In some aspects, a transgene sequence can comprise a nucleotide sequence encoding at least one transposase. In some aspects, a transgene sequence can comprise a nucleotide sequence encoding at least one transposon. In some aspects, a transposon can comprise a nucleotide sequence encoding at least one therapeutic protein. In some aspects, a transposon can comprise a nucleotide sequence encoding at least one therapeutic protein and at least one protomer sequence, wherein the at least one therapeutic protein is operatively linked to the at least one promoter sequence.

In some aspects, a therapeutic protein can be an ornithine transcarbamylase (OTC) polypeptide, a methylmalonyl-CoA mutase (MUT1) polypeptide, a chimeric antigen receptor, or a Factor VIII (FVIII) polypeptide.

In some aspects, the lipid nanoparticles of the present disclosure can be produced using a microfluidic-mixing platform. In some aspects, the microfluidic-mixing platform can be a non-turbulent microfluidic mixing platform.

In some aspects, a microfluidic-mixing platform can produce the lipid nanoparticles of the present invention by combining a miscible solvent phase comprising the lipid components of the nanoparticle and an aqueous phase comprising the lipid nanoparticle cargo (e.g. nucleic acid, DNA, mRNA, etc.) using a microfluidic device. In some aspects, the miscible solvent phase and the aqueous phase are mixed in the microfluidic device under laminar flow conditions that do not allow for immediate mixing of the two phases. As the two phases move under laminar flow in a microfluidic channel, microscopic features in the channel can allow for controlled, homogenous mixing to produce the lipid nanoparticles of the present disclosure.

In some aspects, the microfluidic-mixing platform can include, but are not limited to the NanoAssemblr® Spark (Precision NanoSystems), the NanoAssemblr® Ignite™ (Precision NanoSystems), the NanoAssemblr® Benchtop (Precision NanoSystems), the NanoAssemblr® Blaze (Precision NanoSystems) or the NanoAssemblr® GMP System (Precision NanoSystems).

In some aspects, the lipid nanoparticles of the present disclosure can be produced using a microfluidic-mixing platform, wherein the microfluidic mixing platform mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a microfluidic-mixing platform, wherein the microfluidic mixing platform mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent: aqueous, v: v.

piggyBac ITR Sequences

In some aspects, a nucleic acid can comprise a piggBac ITR sequence. In some aspects, a nucleic acid can comprise a first piggyBac ITR sequence and a second piggBac ITR sequence.

In some aspects, a piggyBac ITR sequence can comprise any piggyBac ITR sequence known in the art.

In some aspects of the methods of the present disclosure, a piggyBac ITR sequence, such as a first piggyBac ITR sequence and/or a second piggyBac ITR sequence in an AAV piggy Bac transposon can comprise, consist essentially of, or consist of a Sleeping Beauty transposon ITR, a Helraiser transposon ITR, a Tol2 transposon ITR, a TcBuster transposon ITR or any combination thereof.

### Promoter Sequences

In some aspects, a nucleic acid can comprise a promoter sequence. In some aspects, a promoter sequence can comprise any promoter sequence known in the art. In some aspects, a promoter sequence can comprise any liver-specific promoter sequence known in the art.

In some aspects, a promoter sequence can comprise a hybrid liver promoter (HLP). In some aspects, a promoter sequence can comprise an LP1 promoter. In some aspects, a promoter sequence can comprise a leukocyte-specific expression of the pp52 (LSP1) long promoter. In some aspects, a promoter sequence can comprise a thyroxine binding globulin (TBG) promoter. In some aspects, a promoter sequence can comprise a wTBG promoter. In some aspects, a promoter sequence can comprise a hepatic combinatorial bundle (HCB) promoter. In some aspects, a promoter sequence can comprise a 2xApoE-hAAT promoter. In some aspects, a promoter sequence can comprise a leukocyte-specific expression of the pp52 (LSP1) plus chimeric intron promoter. In some aspects, a promoter sequence can comprise a cytomegalovirus (CMV) promoter.

### Transgene Sequences

In some aspects, a transgene sequence can comprise a nucleic acid sequence that encodes for a methylmalonyl-CoA mutase (MUT1) polypeptide. The MUT1 polypeptide can be any MUTI polypeptide known in the art.

In some aspects, a transgene sequence can comprise a nucleic acid sequence that encodes for an ornithine transcarbamylase (OTC) polypeptide. The OTC polypeptide can be any OTC polypeptide known in the art.

In some aspects, a transgene sequence can comprise a nucleic acid sequence that encodes for a Factor VIII (FVIII) polypeptide. The FVIII polypeptide can be any FVIII polypeptide known in the art.

In some aspects, a transgene sequence can comprise a nucleic acid sequence that encodes for an iCAS9 polypeptide.

In some aspects, a transgene sequence can be codon optimized according to methods known in the art.

In some aspects, an at least one transgene sequence can be operatively linked to at least one promoter sequence present in the same polynucleotide.

polyA Sequences

In some aspects, a nucleic acid can comprise a polyA sequence. In some aspects, a polyA sequence can comprise any polyA sequence known in the art.

### Self-Cleaving Peptide Sequence

In some aspects, a nucleic acid can comprise a self-cleaving peptide sequence. In some aspects, a self-cleaving peptide sequence can comprise any self-cleaving peptide sequence known in the art. In some aspects, a self-cleaving peptide sequence can comprise an 2A self-cleaving peptide sequence known in the art. Non-limiting examples of self-cleaving peptides include a T2A peptide, GSG-T2A peptide, an E2A peptide, a GSG-E2A peptide, an F2A peptide, a GSG-F2A peptide, a P2A peptide, or a GSG-P2A peptide.

In some aspects, a self-cleaving peptide sequence can comprise a nucleic acid sequence that encodes for a T2A peptide.

In some aspects, a self-cleaving peptide sequence can comprise a nucleic acid sequence that encodes for a GSG-T2A peptide.

In some aspects, a self-cleaving peptide sequence can comprise a nucleic acid sequence that encodes for an E2A peptide.

In some aspects, a self-cleaving peptide sequence can comprise a nucleic acid sequence that encodes for a GSG-E2A peptide.

In some aspects, a self-cleaving peptide sequence can comprise a nucleic acid sequence that encodes for a F2A peptide.

In some aspects, a self-cleaving peptide sequence can comprise a nucleic acid sequence that encodes for a GSG-F2A peptide.

In some aspects, a self-cleaving peptide sequence can comprise a nucleic acid sequence that encodes for a P2A peptide.

In some aspects, a self-cleaving peptide sequence can comprise a nucleic acid sequence that encodes for a GSG-P2A peptide.

### Chimeric Antigen Receptor (CAR)

A transgene sequence can comprise a nucleic acid sequence encoding a CAR, wherein the CAR comprises an ectodomain comprising at least one antigen recognition region; a transmembrane domain, and an endodomain comprising at least one costimulatory domain. The CAR can further comprise a hinge region between the antigen recognition domain and the transmembrane domain.

The antigen recognition region can comprise at least one single chain variable fragment (scFv), Centyrin, single domain antibody, or a combination thereof. In an aspect, the at least one single domain antibody is a VHH. In an aspect, the at least one single domain antibody is a VH. scFv

In some aspects, the antigen recognition region of the CAR can comprise one or more scFv compositions to recognize and bind to a specific target protein/antigen. The antigen recognition region can comprise at least two scFvs. The antigen recognition region can comprise at least three scFvs. In an aspect, a CAR of the disclosure is a bi-specific CAR comprising at least two scFvs that specifically bind two distinct antigens.

The scFv compositions can comprise a heavy chain variable region and a light chain variable region of an antibody. An scFv is a fusion protein of the variable regions of the heavy (VH) and light (VL) chains of immunoglobulins, and the VH and VL domains are connected with a short peptide linker. An scFv can retain the specificity of the original immunoglobulin, despite removal of the constant regions and the introduction of the linker.

### Centyrin

In some aspects, the antigen recognition region of the CAR can comprise one or more Centyrin compositions to recognize and bind to a specific target protein/antigen. Centyrins that specifically bind an antigen may be used to direct the specificity of a cell, (e.g., a cytotoxic immune cell) towards the specific antigen. A CAR comprising a Centyrin is referred to herein as a CARTyrin.

Centyrins of the disclosure may comprise a protein scaffold, wherein the scaffold is capable of specifically binding an antigen. Centyrins of the disclosure may comprise a protein scaffold comprising a consensus sequence of at least one fibronectin type III (FN3) domain, wherein the scaffold is capable of specifically binding an antigen. The at least one fibronectin type III (FN3) domain may be derived from a human protein. The human protein may be Tenascin-C.

The consensus sequence can be modified at one or more positions within (a) a A-B loop at positions 13-16 of the consensus sequence: (b) a B-C loop at positions 22-28 of the consensus sequence; (c) a C-D at positions 38-43 of the consensus sequence; (d) a D-E loop at positions 51-54 of the consensus sequence; (e) a E-F loop at positions 60-64 of the consensus sequence; (f) a F-G loop at positions 75-81 of the consensus sequence; or (g) any combination of (a)-(f). Centyrins of the disclosure may comprise a consensus sequence of at least 5 fibronectin type III (FN3) domains, at least 10 fibronectin type III (FN3) domains or at least 15 fibronectin type III (FN3) domains.

The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g., resistance to enzymatic degradation), and lower production costs. Exemplary antibody mimetics include, but are not limited to, an affibody, an afflilin, an affimer, an affitin, an alphabody, an anticalin, and avimer (also known as avidity multimer), a DARPin (Designed Ankyrin Repeat Protein), a Fynomer, a Kunitz domain peptide, and a monobody.

In some aspects, a transgene sequence can comprise a nucleic acid sequence encoding at least one affibody molecule. Affibody molecules of the disclosure comprise a protein scaffold comprising or consisting of one or more alpha helix without any disulfide bridges. Preferably, affibody molecules of the disclosure comprise or consist of three alpha helices. For example, an affibody molecule of the disclosure may comprise an immunoglobulin binding domain. An affibody molecule of the disclosure may comprise the Z domain of protein A.

In some aspects, a transgene sequence can comprise a nucleic acid sequence encoding at least one affilin molecule. In Affilin molecules of the disclosure comprise a protein scaffold produced by modification of exposed amino acids of, for example, either gamma-B crystallin or ubiquitin. Affilin molecules functionally mimic an antibody's affinity to antigen, but do not structurally mimic an antibody. In any protein scaffold used to make an affilin, those amino acids that are accessible to solvent or possible binding partners in a properly-folded protein molecule are considered exposed amino acids. Any one or more of these exposed amino acids may be modified to specifically bind to a target sequence or antigen.

In some aspects, a transgene sequence can comprise a nucleic acid sequence encoding at least one affimer molecule. Affimer molecules of the disclosure comprise a protein scaffold comprising a highly stable protein engineered to display peptide loops that provide a high affinity binding site for a specific target sequence. Exemplary affimer molecules of the disclosure comprise a protein scaffold based upon a cystatin protein or tertiary structure thereof. Exemplary affimer molecules of the disclosure may share a common tertiary structure of comprising an alpha-helix lying on top of an anti-parallel beta-sheet.

In some aspects, a transgene sequence can comprise a nucleic acid sequence encoding at least one affitin molecule. Affitin molecules of the disclosure comprise an artificial protein scaffold, the structure of which may be derived, for example, from a DNA binding protein (e.g., the DNA binding protein Sac7d). Affitins of the disclosure selectively bind a target sequence, which may be the entirety or part of an antigen. Exemplary affitins of the disclosure are manufactured by randomizing one or more amino acid sequences on the binding surface of a DNA binding protein and subjecting the resultant protein to ribosome display and selection.

Target sequences of affitins of the disclosure may be found, for example, in the genome or on the surface of a peptide, protein, virus, or bacteria. In some aspects, an affitin molecule may be used as a specific inhibitor of an enzyme. Affitin molecules of the disclosure may include heat-resistant proteins or derivatives thereof.

In some aspects, a transgene sequence can comprise a nucleic acid sequence encoding at least one Alphabody molecule. Alphabody molecules of the disclosure may also be referred to as Cell-Penetrating Alphabodies (CPAB). Alphabody molecules of the disclosure comprise small proteins (typically of less than 10 kDa) that bind to a variety of target sequences (including antigens). Alphabody molecules are capable of reaching and binding to intracellular target sequences. Structurally, alphabody molecules of the disclosure comprise an artificial sequence forming single chain alpha helix (similar to naturally occurring coiled-coil structures). Alphabody molecules of the disclosure may comprise a protein scaffold comprising one or more amino acids that are modified to specifically bind target proteins. Regardless of the binding specificity of the molecule, alphabody molecules of the disclosure maintain correct folding and thermostability.

In some aspects, a transgene sequence can comprise a nucleic acid sequence encoding at least one Anticalin molecule. Anticalin molecules of the disclosure comprise artificial proteins that bind to target sequences or sites in either proteins or small molecules. Anticalin molecules of the disclosure may comprise an artificial protein derived from a human lipocalin. Anticalin molecules of the disclosure may be used in place of, for example, monoclonal antibodies or fragments thereof. Anticalin molecules may demonstrate superior tissue penetration and thermostability than monoclonal antibodies or fragments thereof. Exemplary anticalin molecules of the disclosure may comprise about 180 amino acids, having a mass of approximately 20 kDa. Structurally, anticalin molecules of the disclosure comprise a barrel structure comprising antiparallel beta-strands pairwise connected by loops and an attached alpha helix. In some aspects, anticalin molecules of the disclosure comprise a barrel structure comprising eight antiparallel beta-strands pairwise connected by loops and an attached alpha helix.

In some aspects, a transgene sequence can comprise a nucleic acid sequence encoding at least one Avimer molecule. Avimer molecules of the disclosure comprise an artificial protein that specifically binds to a target sequence (which may also be an antigen). Avimers of the disclosure may recognize multiple binding sites within the same target or within distinct targets. When an avimer of the disclosure recognize more than one target, the avimer mimics function of a bi-specific antibody. The artificial protein avimer may comprise two or more peptide sequences of approximately 30-35 amino acids each. These peptides may be connected via one or more linker peptides. Amino acid sequences of one or more of the peptides of the avimer may be derived from an A domain of a membrane receptor. Avimers have a rigid structure that may optionally comprise disulfide bonds and/or calcium. Avimers of the disclosure may demonstrate greater heat stability compared to an antibody.

In some aspects, a transgene sequence can comprise a nucleic acid sequence encoding at least one DARPin. DARPins (Designed Ankyrin Repeat Proteins) of the disclosure comprise genetically-engineered, recombinant, or chimeric proteins having high specificity and high affinity for a target sequence. In some aspects, DARPins of the disclosure are derived from ankyrin proteins and, optionally, comprise at least three repeat motifs (also referred to as repetitive structural units) of the ankyrin protein. Ankyrin proteins mediate high-affinity protein-protein interactions. DARPins of the disclosure comprise a large target interaction surface.

In some aspects, a transgene sequence can comprise a nucleic acid sequence encoding at least one Fynomer. Fynomers of the disclosure comprise small binding proteins (about 7 kDa) derived from the human Fyn SH3 domain and engineered to bind to target sequences and molecules with equal affinity and equal specificity as an antibody.

In some aspects, a transgene sequence can comprise a nucleic acid sequence encoding at least one Kunitz domain peptide. Kunitz domain peptides of the disclosure comprise a protein scaffold comprising a Kunitz domain. Kunitz domains comprise an active site for inhibiting protease activity. Structurally, Kunitz domains of the disclosure comprise a disulfide-rich alpha+beta fold. This structure is exemplified by the bovine pancreatic trypsin inhibitor. Kunitz domain peptides recognize specific protein structures and serve as competitive protease inhibitors. Kunitz domains of the disclosure may comprise Ecallantide (derived from a human lipoprotein-associated coagulation inhibitor (LACI)).

In some aspects, a transgene sequence can comprise a nucleic acid sequence encoding at least one monobody. Monobodies of the disclosure are small proteins (comprising about 94 amino acids and having a mass of about 10 kDa) comparable in size to a single chain antibody. These genetically engineered proteins specifically bind target sequences including antigens. Monobodies of the disclosure may specifically target one or more distinct proteins or target sequences. In some aspects, monobodies of the disclosure comprise a protein scaffold mimicking the structure of human fibronectin, and more preferably, mimicking the structure of the tenth extracellular type III domain of fibronectin. The tenth extracellular type III domain of fibronectin, as well as a monobody mimetic thereof, contains seven beta sheets forming a barrel and three exposed loops on each side corresponding to the three complementarity determining regions (CDRs) of an antibody. In contrast to the structure of the variable domain of an antibody, a monobody lacks any binding site for metal ions as well as a central disulfide bond. Multispecific monobodies may be optimized by modifying the loops BC and FG. Monobodies of the disclosure may comprise an adnectin.

### VHH

In some aspects, the antigen recognition region of the CAR can comprise at least one single domain antibodies (SdAb) to recognize and bind to a specific target protein/antigen. In an aspect, the single domain antibody is a VHH. A VHH is a heavy chain antibody found in camelids. A VHH that specifically binds an antigen may be used to direct the specificity of a cell, (e.g., a cytotoxic immune cell) towards the specific antigen. The antigen recognition region can comprise at least two VHHs. The antigen recognition region can comprise at least three VHHs. In an aspect, a CAR of the disclosure is a bi-specific CAR comprising at least two VHHs that specifically bind two distinct antigens. A CAR comprising a VHH is referred to herein as a VCAR.

At least one VHH protein or VCAR of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from a VHH protein can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, VHH proteins can be engineered with retention of high affinity for the antigen and other favorable biological properties. To achieve this goal, the VHH proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate VHH protein to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used. Screening VHH for specific binding to similar proteins or fragments can be conveniently achieved using nucleotide (DNA or RNA display) or peptide display libraries, for example, in vitro display. Competitive assays can be performed with the VHH or VCAR of the disclosure in order to determine what proteins, antibodies, and other antagonists compete for binding to a target protein with the VHH or VCAR of the present disclosure and/or share the epitope region. These assays as readily known to those of ordinary skill in the art evaluate competition between antagonists or ligands for a limited number of binding sites on a protein

### VH

In some aspects, the antigen recognition region of the CAR can comprise at least one single domain antibodies (SdAb) to recognize and bind to a specific target protein/antigen. In an aspect, the single domain antibody is a VH. A VH is a single domain binder derived from common IgG. A VH that specifically binds an antigen may be used to direct the specificity of a cell, (e.g., a cytotoxic immune cell) towards the specific antigen. The antigen recognition region can comprise at least two VHs. The antigen recognition region can comprise at least three VHs. In an aspect, a CAR of the disclosure is a bi-specific CAR comprising at least two VHs that specifically bind two distinct antigens.

The VH can be isolated or derived from a human sequence. The VH can comprise a human CDR sequence and/or a human framework sequence and a non-human or humanized sequence (e.g., a rat Fc domain). In some aspects, the VH is a fully humanized VH. In some aspects, the VH is neither a naturally occurring antibody nor a fragment of a naturally occurring antibody. In some aspects, the VH is not a fragment of a monoclonal antibody. In some aspects, the VH is a UniDab antibody (TeneoBio). In some aspects, the VH is be modified to remove an Fc domain or a portion thereof. In some aspects, a framework sequence of the VH is modified to, for example, improve expression, decrease immunogenicity or to improve function.

The VH can be fully engineered using the UniRat (TeneoBio) system and “NGS-based Discovery” to produce the VH. Using this method, the specific VH are not naturally-occurring and are generated using fully engineered systems. The VH are not derived from naturally-occurring monoclonal antibodies (mAbs) that were either isolated directly from the host (for example, a mouse, rat or human) or directly from a single clone of cells or cell line (hybridoma). These VHIs were not subsequently cloned from said cell lines. Instead, VH sequences are fully-engineered using the UniRat system as transgenes that comprise human variable regions (VH domains) with a rat Fc domain, and are thus human/rat chimeras without a light chain and are unlike the standard mAb format. The native rat genes are knocked out and the only antibodies expressed in the rat are from transgenes with VH domains linked to a Rat Fc (UniAbs). These are the exclusive Abs expressed in the UniRat. Next generation sequencing (NGS) and bioinformatics are used to identify the full antigen-specific repertoire of the heavy-chain antibodies generated by UniRat after immunization. Then, a unique gene assembly method is used to convert the antibody repertoire sequence information into large collections of fully-human heavy-chain antibodies that can be screened in vitro for a variety of functions. In some aspects, fully humanized VH are generated by fusing the human VH domains with human Fcs in vitro (to generate a non-naturally occurring recombinant VH antibody). In some aspects, the VH are fully humanized, but they are expressed in vivo as human/rat chimera (human VH, rat Fc) without a light chain. Fully humanized VHs are expressed in vivo as human/rat chimera (human VH, rat Fc) without a light chain are about 80 kDa (vs 150 kDa).

A CAR of the present disclosure may bind human antigen with at least one affinity selected from a K_{D} of less than or equal to 10-9M, less than or equal to 10-10M, less than or equal to 10-11M, less than or equal to 10-12M, less than or equal to 10-13M, less than or equal to 10-14M, and less than or equal to 10-15M. The K_{D} may be determined by any means, including, but not limited to, surface plasmon resonance.

In an aspect, the antigen recognition region of the disclosed CAR comprises at least one anti-BCMA Centyrin. A CAR comprising the anti-BCMA Centyrin is referred to as a BCMA CARTyrin herein.

In some aspects, a nanoparticle of the present disclosure can comprise a nucleic acid sequence encoding a BCMA CARTyrin.

In an aspect, the antigen recognition region of the disclosed CAR comprises at least one anti-PSMA Centyrin. A CAR comprising the anti-PSMA Centyrin is referred to as a PSMA CARTyrin herein.

In some aspects, a nanoparticle of the present disclosure can comprise a nucleic acid sequence encoding a PSMA CARTyrin.

In an aspect, the antigen recognition region of the disclosed CAR comprises at least one anti-BCMA VH. A CAR comprising the anti-BCMA VH is referred to as a BCMA VCAR herein.

In some aspects, a nanoparticle of the present disclosure can comprise a nucleic acid sequence encoding a BCMA VCAR.

The ectodomain can comprise a signal peptide. The signal peptide can comprise a sequence encoding a human CD2, CD38, CD38, CD3γ, CD35, CD4, CD8a, CD19, CD28, 4-1BB or GM-CSFR signal peptide. In a preferred aspect, the signal peptide comprises, consists essentially of, or consists of: a human CD8 alpha (CD8a) signal peptide (SP) or a portion thereof.

The hinge domain or hinge region can comprise a human CD8a, IgG4, CD4 sequence, or a combination thereof. In a preferred aspect, the hinge can comprise, consist essentially of, or consist of a human CD8 alpha (CD8a) hinge or a portion thereof.

The transmembrane domain can comprise, consist essentially of, or consist of a sequence encoding a human CD2, CD38, CD3E, CD3Y, CD3ζ, CD4, CD8a, CD19, CD28, 4-1BB or GM-CSFR transmembrane domain. Preferably, the transmembrane domain can comprise, consist essentially of, or consist of a human CD8 alpha (CD8a) transmembrane domain, or a portion thereof.

The at least one costimulatory domain can comprise, consist essentially of, or consist of a human 4-1BB, CD28, CD3 zeta (CD3%), CD40, ICOS, MyD88, OX-40 intracellular domain, or any combination thereof. Preferably, the at least one costimulatory domain comprises a CD35, a 4-1BB costimulatory domain, or a combination thereof.

### Transposition Systems

In some aspects, a nucleic acid can comprise a transposon or a nanotransposon comprising: a first nucleic acid sequence comprising: (a) a first inverted terminal repeat (ITR) or a sequence encoding a first ITR, (b) a second ITR or a sequence encoding a second ITR, and (c) an intra-ITR sequence or a sequence encoding an intra-ITR, wherein the intra-ITR sequence comprises a transposon sequence or a sequence encoding a transposon.

In some aspects, a nucleic acid can comprise a transposon or a nanotransposon comprising: a first nucleic acid sequence comprising: (a) a first inverted terminal repeat (ITR) or a sequence encoding a first ITR, (b) a second ITR or a sequence encoding a second ITR, and (c) an intra-ITR sequence or a sequence encoding an intra-ITR, wherein the intra-ITR sequence comprises a transposon sequence or a sequence encoding a transposon, and a second nucleic acid sequence comprising an inter-ITR sequence or a sequence encoding an inter-ITR, wherein the length of the inter-ITR sequence is equal to or less than 700 nucleotides.

The transposon or nanotransposon of the disclosure comprises a protein scaffold (e.g., a CAR comprising at least one scFv, single domain antibody or Centyrin). The transposon or nanotransposon can be a plasmid DNA transposon comprising a sequence encoding a protein scaffold (e.g., a CAR comprising at least one scFv, single domain antibody or Centyrin) flanked by two cis-regulatory insulator elements. The transposon or nanotransposon can further comprises a plasmid comprising a sequence encoding a transposase. The sequence encoding the transposase may be a DNA sequence or an RNA sequence. Preferably, the sequence encoding the transposase is an mRNA sequence.

The transposon or nanotransposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB) transposase. Preferably, the sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequences for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 7.

The PB or PBL transposase can comprise or consist of an amino acid sequence having an amino acid substitution at two or more, at three or more or at each of positions 30, 165, 282, and/or 538 of the sequence of SEQ ID NO: 7. The transposase can be a SPB transposase that comprises or consists of the amino acid sequence of the sequence of SEQ ID NO: 7 wherein the amino acid substitution at position 30 can be a substitution of a valine (V) for an isoleucine (I), the amino acid substitution at position 165 can be a substitution of a serine(S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 8.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 7 or SEQ ID NO: 8 are described in more detail in PCT Publication No. WO 2019/173636 and PCT/US2019/049816.

In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 9.

The PB or PBL transposase can comprise or consist of an amino acid sequence having an amino acid substitution at two or more, at three or more or at each of positions 29, 164, 281, and/or 537 of the sequence of SEQ ID NO: 9. The transposase can be a SPB transposase that comprises or consists of the amino acid sequence of the sequence of SEQ ID NO: 9 wherein the amino acid substitution at position 29 can be a substitution of a valine (V) for an isoleucine (I), the amino acid substitution at position 164 can be a substitution of a serine(S) for a glycine (G), the amino acid substitution at position 281 can be a substitution of a valine (V) for a methionine (M), and the amino acid substitution at position 537 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 10.

In certain aspects wherein the transposase comprises the above-described mutations at positions 29, 164, 281, and/or 537, the PB, PBL and SPB transposases can further comprise an amino acid substitution at one or more of positions 2, 45, 81, 102, 118, 124, 176, 179, 184, 186, 199, 206, 208, 225, 234, 239, 240, 242, 257, 295, 297, 310, 314, 318, 326, 327, 339, 420, 435, 455, 469, 485, 502, 551, 569 and 590 of the sequence of SEQ ID NO: 9 or SEQ ID NO: 10 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

A transposon or nanotransposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100X) transposase.

A transposon or nanotransposon of the present disclosure can be a Helraiser transposon. An exemplary Helraiser transposon includes Helibat1. In some aspects, when the transposon is a Helraiser transposon, the transposase is a Helitron transposase (for example, as disclosed in WO 2019/173636).

A transposon or nanotransposon of the present disclosure can be a Tol2 transposon. In some aspects, when the transposon is a Tol2 transposon, the transposase is a Tol2 transposase (for example, as disclosed in WO 2019/173636).

A transposon or nanotransposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. The polynucleotide encoding a TcBuster transposase can comprise or consist of a naturally occurring nucleic acid sequence or a non-naturally occurring nucleic acid sequence.

In some aspects, a mutant TcBuster transposase comprises one or more sequence variations when compared to a wild type TcBuster transposase as described in more detail in PCT Publication No. WO 2019/173636 and PCT/US2019/049816.

The cell delivery compositions (e.g., transposons) disclosed herein can comprise a nucleic acid molecule encoding a therapeutic protein or therapeutic agent. Examples of therapeutic proteins include those disclosed in PCT Publication No. WO 2019/173636 and PCT/US2019/049816.

### Cells and Modified Cells of the Disclosure

Cells and modified cells of the disclosure can be mammalian cells. Preferably, the cells and modified cells are human cells. Cells and modified cells of the disclosure can be immune cells. The immune cells of the disclosure can comprise lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts.

The immune precursor cells can comprise any cells which can differentiate into one or more types of immune cells. The immune precursor cells can comprise multipotent stem cells that can self-renew and develop into immune cells. The immune precursor cells can comprise hematopoietic stem cells (HSCs) or descendants thereof. The immune precursor cells can comprise precursor cells that can develop into immune cells. The immune precursor cells can comprise hematopoietic progenitor cells (HPCs).

Hematopoietic stem cells (HSCs) are multipotent, self-renewing cells. All differentiated blood cells from the lymphoid and myeloid lineages arise from HSCs. HSCs can be found in adult bone marrow, peripheral blood, mobilized peripheral blood, peritoneal dialysis effluent and umbilical cord blood.

HSCs can be isolated or derived from a primary or cultured stem cell. HSCs can be isolated or derived from an embryonic stem cell, a multipotent stem cell, a pluripotent stem cell, an adult stem cell, or an induced pluripotent stem cell (iPSC).

Immune precursor cells can comprise an HSC or an HSC descendent cell. Non-limiting examples of HSC descendent cells include multipotent stem cells, lymphoid progenitor cells, natural killer (NK) cells, T lymphocyte cells (T-cells), B lymphocyte cells (B-cells), myeloid progenitor cells, neutrophils, basophils, eosinophils, monocytes and macrophages.

HSCs produced by the disclosed methods can retain features of “primitive” stem cells that, while isolated or derived from an adult stem cell and while committed to a single lineage, share characteristics of embryonic stem cells. For example, the “primitive” HSCs produced by the disclosed methods retain their “stemness” following division and do not differentiate. Consequently, as an adoptive cell therapy, the “primitive” HSCs produced by the disclosed methods not only replenish their numbers, but expand in vivo. “Primitive” HSCs produced by disclosed the methods can be therapeutically-effective when administered as a single dose.

Primitive HSCs can be CD34+. Primitive HSCs can be CD34+ and CD38−. Primitive HSCs can be CD34+, CD38− and CD90+. Primitive HSCs can be CD34+, CD38−, CD90− and CD45RA−. Primitive HSCs can be CD34+, CD38−, CD90+, CD45RA−, and CD49f+. Primitive HSCs can be CD34+, CD38−, CD90+, CD45RA−, and CD49f+.

Primitive HSCs, HSCs, and/or HSC descendent cells can be modified according to the disclosed methods to express an exogenous sequence (e.g., a chimeric antigen receptor or therapeutic protein). Modified primitive HSCs, modified HSCs, and/or modified HSC descendent cells can be forward differentiated to produce a modified immune cell including, but not limited to, a modified T cell, a modified natural killer cell and/or a modified B-cell.

The modified immune or immune precursor cells can be NK cells. The NK cells can be cytotoxic lymphocytes that differentiate from lymphoid progenitor cells. Modified NK cells can be derived from modified hematopoietic stem and progenitor cells (HSPCs) or modified HSCs. In some aspects, non-activated NK cells are derived from CD3-depleted leukapheresis (containing CD14/CD19/CD56+ cells).

The modified immune or immune precursor cells can be B cells. B cells are a type of lymphocyte that express B cell receptors on the cell surface. B cell receptors bind to specific antigens. Modified B cells can be derived from modified hematopoietic stem and progenitor cells (HSPCs) or modified HSCs.

Modified T cells of the disclosure may be derived from modified hematopoietic stem and progenitor cells (HSPCs) or modified HSCs. Unlike traditional biologics and chemotherapeutics, the disclosed modified-T cells the capacity to rapidly reproduce upon antigen recognition, thereby potentially obviating the need for repeat treatments. To achieve this, in some aspects, modified-T cells not only drive an initial response, but also persist in the patient as a stable population of viable memory T cells to prevent potential relapses. Alternatively, in some aspects, when it is not desired, the modified-T cells do not persist in the patient.

Intensive efforts have been focused on the development of antigen receptor molecules that do not cause T cell exhaustion through antigen-independent (tonic) signaling, as well as of a modified-T cell product containing early memory T cells, especially stem cell memory (T_{SCM}) or stem cell-like T cells. Stem cell-like modified-T cells of the disclosure exhibit the greatest capacity for self-renewal and multipotent capacity to derive central memory (T_{CM}) T cells or T_{CM} like cells, effector memory (T_{EM}) and effector T cells (T_{E}), thereby producing better tumor eradication and long-term modified-T cell engraftment. A linear pathway of differentiation may be responsible for generating these cells: Naïve T cells (T_{N})>T_{SCM}>T_{CM}>T_{EM}>T_{E}>T_{TE}, whereby T_{N} is the parent precursor cell that directly gives rise to T_{SCM}, which then, in turn, directly gives rise to T_{CM}, etc. Compositions of T cells of the disclosure can comprise one or more of each parental T cell subset with T_{SCM} cells being the most abundant (e.g., T_{SCM}>T_{CM}>T_{EM}>T_{E}>T_{TE}).

The immune cell precursor can be differentiated into or is capable of differentiating into an early memory T cell, a stem cell like T-cell, a Naïve T cells (T_{N}), a T_{SCM}, a T_{CM}, a T_{EM}, a T_{E}, or a T_{TE}. The immune cell precursor can be a primitive HSC, an HSC, or a HSC descendent cell of the disclosure. The immune cell can be an early memory T cell, a stem cell like T-cell, a Naïve T cells™, a T_{SCM}, a T_{CM}, a T_{EM}, a T_{E}, or a T_{TE}.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of a plurality of modified T cells in the population expresses one or more cell-surface marker(s) of an early memory T cell. The population of modified early memory T cells comprises a plurality of modified stem cell-like T cells. The population of modified early memory T cells comprises a plurality of modified T_{SCM} cells. The population of modified early memory T cells comprises a plurality of modified T_{CM} cells.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a stem cell-like T cell. The population of modified stem cell-like T cells comprises a plurality of modified T_{SCM} cells. The population of modified stem cell-like T cells comprises a plurality of modified T_{CM} cells.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2RB. The cell-surface markers can comprise one or more of CD45RA, CD95, IL-2Rβ, CCR7, and CD62L.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the plurality of modified T cells in the population expresses one or more cell-surface marker(s) of a central memory T cell (T_{CM}) or a T_{CM}-like cell; and wherein the one or more cell-surface marker(s) comprise CD45RO and CD62L. The cell-surface markers can comprise one or more of CD45RO, CD95, IL-2RB, CCR7, and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a naïve T cell (T_{N}). The cell-surface markers can comprise one or more of CD45RA, CCR7 and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of an effector T-cell (modified TEFF). The cell-surface markers can comprise one or more of CD45RA, CD95, and IL-2RB.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells of the population expresses one or more cell-surface marker(s) of a stem cell-like T cell, a stem memory T cell (T_{SCM}) or a central memory T cell (T_{CM}).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 or wherein at least about 70% to about 99%, about 75% to about 95% or about 85% to about 95% of the population of modified cells express one or more cell-surface marker(s) comprising CD34 (e.g., comprise the cell-surface marker phenotype CD34+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and do not express one or more cell-surface marker(s) comprising CD38, or wherein at least about 45% to about 90%, about 50% to about 80% or about 65% to about 75% of the population of modified cells express one or more cell-surface marker(s) comprising CD34 and do not express one or more cell-surface marker(s) comprising CD38 (e.g., comprise the cell-surface marker phenotype CD34+ and CD38−).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38, or wherein at least about 0.2% to about 40%, about 0.2% to about 30%, about 0.2% to about 2% or 0.5% to about 1.5% of the population of modified cells express one or more cell-surface marker(s) comprising CD34 and CD90 and do not express one or more cell-surface marker(s) comprising CD38 (e.g., comprise the cell-surface marker phenotype CD34+, CD38- and CD90+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38 and CD45RA, or wherein at least about 0.2% to about 40%, about 0.2% to about 30%, about 0.2% to about 2% or 0.5% to about 1.5% of the population of modified cells express one or more cell-surface marker(s) comprising CD34 and CD90 and do not express one or more cell-surface marker(s) comprising CD38 and CD45RA (e.g., comprise the cell-surface marker phenotype CD34+, CD38−, CD90+, CD45RA−).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34, CD90 and CD49f and do not express one or more cell-surface marker(s) comprising CD38 and CD45RA, or wherein at least about 0.02% to about 30%, about 0.02% to about 2%, about 0.04% to about 2% or about 0.04% to about 1% of the population of modified cells express one or more cell-surface marker(s) comprising CD34, CD90 and CD49f and do not express one or more cell-surface marker(s) comprising CD38 and CD45RA (e.g., comprise the cell-surface marker phenotype CD34+, CD38−, CD90+, CD45RA− and CD49f+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD45RA, or wherein at least about 0.2% to about 5%, about 0.2% to about 3% or about 0.4% to about 3% of the population of modified cells express one or more cell-surface marker(s) comprising CD34 and CD90 and do not express one or more cell-surface marker(s) comprising CD45RA (e.g., comprise the cell-surface marker phenotype CD34+, CD90+ and CD45RA−).

Compositions and methods of producing and/or expanding the immune cells or immune precursor cells (e.g., the disclosed modified T-cells) and buffers for maintaining or enhancing a level of cell viability and/or a stem-like phenotype of the immune cells or immune precursor cells (e.g., the disclosed modified T-cells) are disclosed elsewhere herein and are disclosed in more detail in U.S. Pat. No. 10,329,543 and PCT Publication No. WO 2019/173636.

Cells and modified cells of the disclosure can be somatic cells. Cells and modified cells of the disclosure can be differentiated cells. Cells and modified cells of the disclosure can be autologous cells or allogenic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

### Methods of Expressing a Chimeric Antigen Receptor

The disclosure provides methods of expressing a CAR on the surface of a cell. The method comprises (a) obtaining a cell population; (b) contacting the cell population to a composition of the present disclosure comprising a CAR or a sequence encoding the CAR, under conditions sufficient to transfer the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface

In some aspects, the cell population can comprise leukocytes and/or CD4+ and CD8+leukocytes. The cell population can comprise CD4- and CD8-leukocytes in an optimized ratio. The optimized ratio of CD4+to CD8+leukocytes does not naturally occur in vivo. The cell population can comprise a tumor cell.

In some aspects, the conditions sufficient to transfer the CAR or the sequence encoding the CAR, transposon, or vector across a cell membrane of at least one cell in the cell population comprises at least one of an application of one or more pulses of electricity at a specified voltage, a buffer, and one or more supplemental factor(s). In some aspects, the conditions suitable for integration of the sequence encoding the CAR comprise at least one of a buffer and one or more supplemental factor(s).

The buffer can comprise PBS, HBSS, OptiMEM, BTXpress, Amaxa Nucleofector, Human T cell nucleofection buffer or any combination thereof. The one or more supplemental factor(s) can comprise (a) a recombinant human cytokine, a chemokine, an interleukin or any combination thereof; (b) a salt, a mineral, a metabolite or any combination thereof; (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof can comprise IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L or any combination thereof. The salt, the mineral, the metabolite or any combination thereof can comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, antibiotics, pH adjusters, Earle's Salts, 2-Mercaptoethanol, Human transferrin, Recombinant human insulin, Human serum albumin, Nucleofector PLUS Supplement, KCL, MgCl2, NazHPO4, NAH2PO4, Sodium lactobionate, Mannitol, Sodium succinate, Sodium Chloride, CINa, Glucose, Ca (NO3) 2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5, or any combination thereof. The cell medium can comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof. The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspasel, Pro-IL1B, PI3K, Akt, Wnt3A, inhibitors of glycogen synthase kinase-3 (GSK-3 B) (e.g. TWS119), or any combination thereof. Examples of such inhibitors can include Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

The expansion and selection steps can occur concurrently or sequentially. The expansion can occur prior to selection. The expansion can occur following selection, and, optionally, a further (i.e. second) selection can occur following expansion. Concurrent expansion and selection can be simultaneous. The expansion and/or selection steps can proceed for a period of 10 to 14 days, inclusive of the endpoints.

The expansion can comprise contacting at least one cell of the modified cell population with an antigen to stimulate the at least one cell through the CAR, thereby generating an expanded cell population. The antigen can be presented on the surface of a substrate. The substrate can have any form, including, but not limited to a surface, a well, a bead or a plurality thereof, and a matrix. The substrate can further comprise a paramagetic or magnetic component. The antigen can be presented on the surface of a substrate, wherein the substrate is a magnetic bead, and wherein a magnet can be used to remove or separate the magnetic beads from the modified and expanded cell population. The antigen can be presented on the surface of a cell or an artificial antigen presenting cell. Artificial antigen presenting cells can include, but are not limited to, tumor cells and stem cells.

In some aspects wherein the transposon or vector comprises a selection gene, the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

The disclosure provides a composition comprising the modified, expanded and selected cell population of the methods described herein.

A more detailed description of methods for expressing a CAR on the surface of a cell is disclosed in PCT Publication No. WO 2019/049816 and PCT/US2019/049816.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The composition can modify gene expression by decreasing gene expression. The composition can modify gene expression by transiently modifying gene expression (e.g., for the duration of binding of the ligand to the exogenous receptor). The composition can modify gene expression acutely (e.g., the ligand reversibly binds to the exogenous receptor). The composition can modify gene expression chronically (e.g., the ligand irreversibly binds to the exogenous receptor).

In some aspects, a nucleic acid can comprise a transgene comprising a nucleic acid molecule encoding at least one exogenous receptor. The exogenous receptor can comprise an endogenous receptor with respect to the genomic sequence of the cell. Exemplary receptors include, but are not limited to, intracellular receptors, cell-surface receptors, transmembrane receptors, ligand-gated ion channels, and G-protein coupled receptors.

The exogenous receptor can comprise a non-naturally occurring receptor. The non-naturally occurring receptor can be a synthetic, modified, recombinant, mutant or chimeric receptor. The non-naturally occurring receptor can comprise one or more sequences isolated or derived from a T-cell receptor (TCR). The non-naturally occurring receptor can comprise one or more sequences isolated or derived from a scaffold protein. In some aspects, including those wherein the non-naturally occurring receptor does not comprise a transmembrane domain, the non-naturally occurring receptor interacts with a second transmembrane, membrane-bound and/or an intracellular receptor that, following contact with the non-naturally occurring receptor, transduces an intracellular signal. The non-naturally occurring receptor can comprise a transmembrane domain. The non-naturally occurring receptor can interact with an intracellular receptor that transduces an intracellular signal. The non-naturally occurring receptor can comprise an intracellular signaling domain. The non-naturally occurring receptor can be a chimeric ligand receptor (CLR). The CLR can be a chimeric antigen receptor (CAR).

The sequence encoding the inducible promoter of comprises a sequence encoding an NFKB promoter, a sequence encoding an interferon (IFN) promoter or a sequence encoding an interleukin-2 promoter. In some aspects, the IFN promoter is an IFNγ promoter. The inducible promoter can be isolated or derived from the promoter of a cytokine or a chemokine. The cytokine or chemokine can comprise IL2, IL3, IL4, IL5, IL6, IL10, IL12, IL13, IL 17A/F, IL21, IL22, IL23, transforming growth factor beta (TGFβ), colony stimulating factor 2 (GM-CSF), interferon gamma (IFNγ), Tumor necrosis factor alpha (TNFα), LTα, perforin, Granzyme C (Gzmc), Granzyme B (Gzmb), C-C motif chemokine ligand 5 (CCL5), C-C motif chemokine ligand 4 (Ccl4), C-C motif chemokine ligand 3 (Ccl3), X-C motif chemokine ligand 1 (Xcl1) or LIF interleukin 6 family cytokine (Lif).

The inducible promoter can be isolated or derived from the promoter of a gene comprising a surface protein involved in cell differentiation, activation, exhaustion and function. In some aspects, the gene comprises CD69, CD71, CTLA4, PD-1, TIGIT, LAG3, TIM-3, GITR, MHCII, COX-2, FASL or 4-1BB.

The inducible promoter can be isolated or derived from the promoter of a gene involved in CD metabolism and differentiation. The inducible promoter can be isolated or derived from the promoter of Nr4a1, Nr4a3, Tnfrsf9 (4-1BB), Sema7a, Zfp3612, Gadd45b, Dusp5, Dusp6 and Neto2.

In some aspects, the inducible transgene construct comprises or drives expression of a signaling component downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of which are disclosed in PCT Publication No. WO 2019/173636 and PCT Application No. PCT/US2019/049816.

### Armored Cells

The modified cells of disclosure (e.g., CAR T-cells) can be further modified to enhance their therapeutic potential. Alternatively, or in addition, the modified cells may be further modified to render them less sensitive to immunologic and/or metabolic checkpoints. Modifications of this type “armor” the cells, which, following the modification, may be referred to here as “armored” cells (e.g., armored T-cells) Armored cells may be produced by, for example, blocking and/or diluting specific checkpoint signals delivered to the cells (e.g., checkpoint inhibition) naturally, within the tumor immunosuppressive microenvironment.

An armored cell of the disclosure can be derived from any cell, for example, a T cell, a NK cell, a hematopoietic progenitor cell, a peripheral blood (PB) derived T cell (including a T cell isolated or derived from G-CSF-mobilized peripheral blood), or an umbilical cord blood (UCB) derived T cell. An armored cell (e.g., armored T-cell) can comprise one or more of a chimeric ligand receptor (CLR comprising a protein scaffold, an antibody, an ScFv, or an antibody mimetic)/chimeric antigen receptor (CAR comprising a protein scaffold, an antibody, an ScFv, or an antibody mimetic), a CARTyrin (a CAR comprising a Centyrin), and/or a VCAR (a CAR comprising a camelid VHH or a single domain VH). An armored cell (e.g., armored T-cell) can comprise an inducible proapoptotic polypeptide as disclosed herein. An armored cell (e.g., armored T-cell) can comprise an exogenous sequence. The exogenous sequence can comprise a sequence encoding a therapeutic protein. Exemplary therapeutic proteins may be nuclear, cytoplasmic, intracellular, transmembrane, cell-surface bound, or secreted proteins. Exemplary therapeutic proteins expressed by the armored cell (e.g., armored T-cell) may modify an activity of the armored cell or may modify an activity of a second cell. An armored cell (e.g., armored T-cell) can comprise a selection gene or a selection marker. An armored cell (e.g., armored T-cell) can comprise a synthetic gene expression cassette (also referred to herein as an inducible transgene construct).

The modified cells of disclosure (e.g., CAR T-cells) can be further modified to silence or reduce expression one or more gene(s) encoding receptor(s) of inhibitory checkpoint signals to produce an armored cell (e.g., armored CAR T-cell). Receptors of inhibitory checkpoint signals are expressed on the cell surface or within the cytoplasm of a cell. Silencing or reducing expressing of the gene encoding the receptor of the inhibitory checkpoint signal results a loss of protein expression of the inhibitory checkpoint receptors on the surface or within the cytoplasm of an armored cell. Thus, armored cells having silenced or reduced expression of one or more genes encoding an inhibitory checkpoint receptor is resistant, non-receptive or insensitive to checkpoint signals. The resistance or decreased sensitivity of the armored cell to inhibitory checkpoint signals enhances the therapeutic potential of the armored cell in the presence of these inhibitory checkpoint signals. Non-limiting examples of inhibitory checkpoint signals (and proteins that induce immunosuppression) are disclosed in PCT Publication No. WO 2019/173636. Preferred examples of inhibitory checkpoint signals that may be silenced include, but are not limited to, PD-1 and TGFβRII.

The modified cells of disclosure (e.g., CAR T-cells) can be further modified to silence or reduce expression of one or more gene(s) encoding intracellular proteins involved in checkpoint signaling to produce an armored cell (e.g., armored CAR T-cell). The activity of the modified cells may be enhanced by targeting any intracellular signaling protein involved in a checkpoint signaling pathway, thereby achieving checkpoint inhibition or interference to one or more checkpoint pathways. Non-limiting examples of intracellular signaling proteins involved in checkpoint signaling are disclosed in PCT Publication No. WO 2019/173636.

The modified cells of disclosure (e.g., CAR T-cells) can be further modified to silence or reduce expression of one or more gene(s) encoding a transcription factor that hinders the efficacy of a therapy to produce an armored cell (e.g., armored CAR T-cell). The activity of modified cells may be enhanced or modulated by silencing or reducing expression (or repressing a function) of a transcription factor that hinders the efficacy of a therapy. Non-limiting examples of transcription factors that may be modified to silence or reduce expression or to repress a function thereof include, but are not limited to, the exemplary transcription factors are disclosed in PCT Publication No. WO 2019/173636.

The modified cells of disclosure (e.g., CAR T-cells) can be further modified to silence or reduce expression of one or more gene(s) encoding a cell death or cell apoptosis receptor to produce an armored cell (e.g., armored CAR T-cell). Interaction of a death receptor and its endogenous ligand results in the initiation of apoptosis. Disruption of an expression, an activity, or an interaction of a cell death and/or cell apoptosis receptor and/or ligand render a modified cell less receptive to death signals, consequently, making the armored cell more efficacious in a tumor environment. Non-limiting examples of cell death and/or cell apoptosis receptors and ligands are disclosed in PCT Publication No. WO 2019/173636. A preferred example of cell death receptor which may be modified is Fas (CD95).

The modified cells of disclosure (e.g., CAR T-cells) can be further modified to silence or reduce expression of one or more gene(s) encoding a metabolic sensing protein to produce an armored cell (e.g., armored CAR T-cell). Disruption to the metabolic sensing of the immunosuppressive tumor microenvironment (characterized by low levels of oxygen, pH, glucose and other molecules) by a modified cell leads to extended retention of T-cell function and, consequently, more tumor cells killed per cell. Non-limiting examples of metabolic sensing genes and proteins are disclosed in PCT Publication No. WO 2019/173636. A preferred example, HIFla and VHL play a role in T-cell function while in a hypoxic environment. An armored T-cell may have silenced or reduced expression of one or more genes encoding HIFla or VHL.

The modified cells of disclosure (e.g., CAR T-cells) can be further modified to silence or reduce expression of one or more gene(s) encoding proteins that that confer sensitivity to a cancer therapy, including a monoclonal antibody, to produce an armored cell (e.g., armored CAR T-cell). Thus, an armored cell can function and may demonstrate superior function or efficacy whilst in the presence of a cancer therapy (e.g., a chemotherapy, a monoclonal antibody therapy, or another anti-tumor treatment). Non-limiting examples of proteins involved in conferring sensitivity to a cancer therapy are disclosed in PCT Publication No. WO 2019/173636.

The modified cells of disclosure (e.g., CAR T-cells) can be further modified to silence or reduce expression of one or more gene(s) encoding a growth advantage factor to produce an armored cell (e.g., armored CAR T-cell). Silencing or reducing expression of an oncogene can confer a growth advantage for the cell. For example, silencing or reducing expression (e.g., disrupting expression) of a TET2 gene during a CAR T-cell manufacturing process results in the generation of an armored CAR T-cell with a significant capacity for expansion and subsequent eradication of a tumor when compared to a non-armored CAR T-cell lacking this capacity for expansion. This strategy may be coupled to a safety switch (e.g., an iC9 safety switch described herein), which permits the targeted disruption of an armored CAR T-cell in the event of an adverse reaction from a subject or uncontrolled growth of the armored CAR T-cell. Non-limiting examples of growth advantage factors are disclosed in PCT Publication No. WO 2019/173636.

The modified cells of disclosure (e.g., CAR T-cells) can be further modified to express a modified/chimeric checkpoint receptor to produce an armored T-cell of the disclosure.

The modified/chimeric checkpoint receptor can comprise a null receptor, decoy receptor or dominant negative receptor. A null receptor, decoy receptor or dominant negative receptor can be modified/chimeric receptor/protein. A null receptor, decoy receptor or dominant negative receptor can be truncated for expression of the intracellular signaling domain. Alternatively, or in addition, a null receptor, decoy receptor or dominant negative receptor can be mutated within an intracellular signaling domain at one or more amino acid positions that are determinative or required for effective signaling. Truncation or mutation of null receptor, decoy receptor or dominant negative receptor can result in loss of the receptor's capacity to convey or transduce a checkpoint signal to the cell or within the cell.

For example, a dilution or a blockage of an immunosuppressive checkpoint signal from a PD-L1 receptor expressed on the surface of a tumor cell may be achieved by expressing a modified/chimeric PD-1 null receptor on the surface of an armored cell (e.g., armored CAR T-cell), which effectively competes with the endogenous (non-modified) PD-1 receptors also expressed on the surface of the armored cell to reduce or inhibit the transduction of the immunosuppressive checkpoint signal through endogenous PD-1 receptors of the armored cell. In this non-limiting example, competition between the two different receptors for binding to PD-L1 expressed on the tumor cell reduces or diminishes a level of effective checkpoint signaling, thereby enhancing a therapeutic potential of the armored cell expressing the PD-1 null receptor.

The modified/chimeric checkpoint receptor can comprise a null receptor, decoy receptor or dominant negative receptor that is a transmembrane receptor, a membrane-associated or membrane-linked receptor/protein or an intracellular receptor/protein. Exemplary null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636.

The modified/chimeric checkpoint receptor can comprise a switch receptor. Exemplary switch receptors comprise a modified/chimeric receptor/protein wherein a native or wild type intracellular signaling domain is switched or replaced with a different intracellular signaling domain that is either non-native to the protein and/or not a wild-type domain. For example, replacement of an inhibitory signaling domain with a stimulatory signaling domain would switch an immunosuppressive signal into an immunostimulatory signal. Alternatively, replacement of an inhibitory signaling domain with a different inhibitory domain can reduce or enhance the level of inhibitory signaling. Expression or overexpression, of a switch receptor can result in the dilution and/or blockage of a cognate checkpoint signal via competition with an endogenous wild-type checkpoint receptor (not a switch receptor) for binding to the cognate checkpoint receptor expressed within the immunosuppressive tumor microenvironment. Armored cells (e.g., armored CAR T-cells) can comprise a sequence encoding a switch receptor, leading to the expression of one or more switch receptors, and consequently, altering an activity of an armored cell. Armored cells (e.g., armored CAR T-cells) can express a switch receptor that targets an intracellularly expressed protein downstream of a checkpoint receptor, a transcription factor, a cytokine receptor, a death receptor, a metabolic sensing molecule, a cancer therapy, an oncogene, and/or a tumor suppressor protein or gene.

Exemplary switch receptors can comprise or can be derived from a protein including, but are not limited to, the signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene.

The modified cells of disclosure (e.g., CAR T-cells) can be further modified to express a CLR/CAR that mediates conditional gene expression to produce an armored T-cell. The combination of the CLR/CAR and the condition gene expression system in the nucleus of the armored T-cell constitutes a synthetic gene expression system that is conditionally activated upon binding of cognate ligand(s) with CLR or cognate antigen(s) with CAR. This system may help to ‘armor’ or enhance therapeutic potential of modified T-cells by reducing or limiting synthetic gene expression at the site of ligand or antigen binding, at or within the tumor environment for example.

The present disclosure provides a gene editing composition and/or a cell comprising the gene editing composition. The gene editing composition can comprise a nanoparticle comprising a nucleic acid, wherein the nucleic acid comprises a sequence encoding a DNA binding domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. In some aspects, the fusion protein can further comprise at least one nuclear localization signal (NLS). In some aspects, the fusion protein can further comprise at least two NLSs. The gene editing composition can further comprise a guide sequence. The guide sequence can comprise an RNA sequence.

A transgene can comprise a nucleic sequence encoding a small, Cas9 (Cas9) operatively-linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease.

A transgene can comprise a nucleic sequence encoding an inactivated, small, Cas9 (dSaCas9) operatively-linked to an effector. A transgene can comprise a nucleic sequence encoding a fusion protein comprising, consisting essentially of or consisting of a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease.

A transgene can comprise a nucleic sequence encoding an inactivated Cas9 (dCas9) operatively-linked to an effector. A transgene can comprise a nucleic sequence encoding a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptoccocus pyogenes. The dCas9 can comprise a dCas9 with substitutions at amino acid positions 10 and 840, which inactivate the catalytic site. In some aspects, these substitutions are D10A and H₈₄₀A.

A cell comprising the gene editing composition can express the gene editing composition stably or transiently. Preferably, the gene editing composition is expressed transiently. The guide RNA can comprise a sequence complementary to a target sequence within a genomic DNA sequence. The target sequence within a genomic DNA sequence can be a target sequence within a safe harbor site of a genomic DNA sequence.

Gene editing compositions, including Cas-CLOVER, and methods of using these compositions for gene editing are described in detail in U.S. Patent Publication Nos. 2017/0107541, 2017/0114149, 2018/0187185 and U.S. Pat. No. 10,415,024. In some aspects, a Cas-CLOVER protein can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 5.

Accordingly, the present disclosure provides any of the lipid nanoparticle compositions described herein, wherein the lipid nanoparticle comprises at least one genomic editing composition, wherein the at least one genomic editing composition comprises: a) a nucleic acid molecule comprising a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof; and b) at least one gRNA molecule. In some aspects, the fusion protein can further comprise at least one NLS. In some aspects, the at least one genomic editing composition can comprise at least two species of gRNA molecules.

### Chimeric Stimulator Receptors and Recombinant HLA-E Polypeptides

Adoptive cell compositions that are “universally” safe for administration to any patient requires a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated to prevent T-cell mediated GvH that could cause death to the subject. Thus, in a preferred aspect, the disclosure provides a pure TCR-negative allogeneic T-cell composition (e.g., each cell of the composition expresses at a level so low as to either be undetectable or non-existent).

Expression and/or function of MHC class I (MHC-I, specifically, HLA-A, HLA-B, and HLA-C) is reduced or eliminated to prevent HvG and, consequently, to improve engraftment of cells in a subject. Improved engraftment results in longer persistence of the cells, and, therefore, a larger therapeutic window for the subject. Specifically, expression and/or function of a structural element of MHC-I, Beta-2-Microglobulin (B2M), is reduced or eliminated.

The above strategies induce further challenges. T Cell Receptor (TCR) knockout (KO) in T cells results in loss of expression of CD3-zeta (CD3z or CD3° C.), which is part of the TCR complex. The loss of CD32 in TCR-KO T-cells dramatically reduces the ability of optimally activating and expanding these cells using standard stimulation/activation reagents, including, but not limited to, agonist anti-CD3 mAb. When the expression or function of any one component of the TCR complex is interrupted, all components of the complex are lost, including TCR-alpha (TCRα), TCR-beta (TCRβ), CD3-gamma (CD3γ), CD3-epsilon (CD3ε), CD3-delta (CD3δ), and CD3-zeta (CD3ξ). Both CD3δ and CD3ξ are required for T cell activation and expansion. Agonist anti-CD3 mAbs typically recognize CD3ε and possibly another protein within the complex which, in turn, signals to CD3ξ. CD3ξ provides the primary stimulus for T cell activation (along with a secondary co-stimulatory signal) for optimal activation and expansion. Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb In fact, T cell expansion is reduced to only 20-40% of the normal level of expansion when stimulated using standard activation/stimulation reagents, including agonist anti-CD3 mAb.

Thus, the present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical.

In some aspects, a transgene sequence can comprise a nucleic acid sequence encoding a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical.

The activation component can comprise a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds. The activation component can comprise a CD2 extracellular domain or a portion thereof to which an agonist binds.

The signal transduction domain can comprise one or more of a component of a human signal transduction domain, T-cell Receptor (TCR), a component of a TCR complex, a component of a TCR co-receptor, a component of a TCR co-stimulatory protein, a component of a TCR inhibitory protein, a cytokine receptor, and a chemokine receptor. The signal transduction domain can comprise a CD3 protein or a portion thereof. The CD3 protein can comprise a CD32 protein or a portion thereof.

The endodomain can further comprise a cytoplasmic domain. The cytoplasmic domain can be isolated or derived from a third protein. The first protein and the third protein can be identical. The ectodomain can further comprise a signal peptide. The signal peptide can be derived from a fourth protein. The first protein and the fourth protein can be identical. The transmembrane domain can be isolated or derived from a fifth protein. The first protein and the fifth protein can be identical.

In some aspects, the activation component does not bind a naturally-occurring molecule. In some aspects, the activation component binds a naturally-occurring molecule but the CSR does not transduce a signal upon binding of the activation component to a naturally-occurring molecule. In some aspects, the activation component binds to a non-naturally occurring molecule. In some aspects, the activation component does not bind a naturally-occurring molecule but binds a non-naturally occurring molecule. The CSR can selectively transduces a signal upon binding of the activation component to a non-naturally occurring molecule.

In a preferred aspect, the present disclosure provides a non-naturally occurring chimeric stimulatory receptor (CSR) comprising: (a) an ectodomain comprising a signal peptide and an activation component, wherein the signal peptide comprises a CD2 signal peptide or a portion thereof and wherein the activation component comprises a CD2 extracellular domain or a portion thereof to which an agonist binds; (b) a transmembrane domain, wherein the transmembrane domain comprises a CD2 transmembrane domain or a portion thereof; and (c) an endodomain comprising a cytoplasmic domain and at least one signal transduction domain, wherein the cytoplasmic domain comprises a CD2 cytoplasmic domain or a portion thereof and wherein the at least one signal transduction domain comprises a CD35 protein or a portion thereof.

The present disclosure also provides a non-naturally occurring chimeric stimulatory receptor (CSR) wherein the ectodomain comprises a modification. The modification can comprise a mutation or a truncation of the amino acid sequence of the activation component or the first protein when compared to a wild type sequence of the activation component or the first protein. The mutation or a truncation of the amino acid sequence of the activation component can comprise a mutation or truncation of a CD2 extracellular domain or a portion thereof to which an agonist binds. The mutation or truncation of the CD2 extracellular domain can reduce or eliminate binding with naturally occurring CD58.

In a preferred aspect, the present disclosure provides non-naturally occurring chimeric stimulatory receptor (CSR) comprising: (a) an ectodomain comprising a signal peptide and an activation component, wherein the signal peptide comprises a CD2 signal peptide or a portion thereof and wherein the activation component comprises a CD2 extracellular domain or a portion thereof to which an agonist binds and wherein the CD2 extracellular domain or a portion thereof to which an agonist binds comprises a mutation or truncation; (b) a transmembrane domain, wherein the transmembrane domain comprises a CD2 transmembrane domain or a portion thereof, and (c) an endodomain comprising a cytoplasmic domain and at least one signal transduction domain, wherein the cytoplasmic domain comprises a CD2 cytoplasmic domain or a portion thereof and wherein the at least one signal transduction domain comprises a CD33 protein or a portion thereof.

The present disclosure provides a nucleic acid sequence encoding any CSR disclosed herein. The present disclosure provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

A modified cell disclosed herein can be an allogeneic cell or an autologous cell. In some preferred aspects, the modified cell is an allogeneic cell. In some aspects, the modified cell is an autologous T-cell or a modified autologous CAR T-cell. In some preferred aspects, the modified cell is an allogeneic T-cell or a modified allogeneic CAR T-cell.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

The present disclosure provides a modified T lymphocyte (T-cell), comprising: (a) a modification of an endogenous sequence encoding a T-cell Receptor (TCR), wherein the modification reduces or eliminates a level of expression or activity of the TCR; and (b) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The modified T-cell can further comprise an inducible proapoptotic polypeptide. The modified T-cell can further comprise a modification of an endogenous sequence encoding Beta-2-Microglobulin (B2M), wherein the modification reduces or eliminates a level of expression or activity of a major histocompatibility complex (MHC) class I (MHC-I).

The modified T-cell can further comprise a non-naturally occurring polypeptide comprising an HLA class I histocompatibility antigen, alpha chain E (HLA-E) polypeptide. The non-naturally occurring polypeptide comprising a HLA-E polypeptide can further comprise a B2M signal peptide. The non-naturally occurring polypeptide comprising a HLA-E polypeptide can further comprise a B2M polypeptide. The non-naturally occurring polypeptide comprising an HLA-E polypeptide can further comprise a linker, wherein the linker is positioned between the B2M polypeptide and the HLA-E polypeptide. The non-naturally occurring polypeptide comprising an HLA-E polypeptide can further comprise a peptide and a B2M polypeptide. The non-naturally occurring polypeptide comprising an HLA-E can further comprise a first linker positioned between the B2M signal peptide and the peptide, and a second linker positioned between the B2M polypeptide and the peptide encoding the HLA-E.

The modified T-cell can further comprise a non-naturally occurring antigen receptor, a sequence encoding a therapeutic polypeptide, or a combination thereof. The non-naturally occurring antigen receptor can comprise a chimeric antigen receptor (CAR).

The CSR can be transiently expressed in the modified T-cell. The CSR can be stably expressed in the modified T-cell. The polypeptide comprising the HLA-E polypeptide can be transiently expressed in the modified T-cell. The polypeptide comprising the HLA-E polypeptide can be stably expressed in the modified T-cell. The inducible proapoptotic polypeptide can be transiently expressed in the modified T-cell. The inducible proapoptotic polypeptide can be stably expressed in the modified T-cell. The non-naturally occurring antigen receptor or a sequence encoding a therapeutic protein can be transiently expressed in the modified T-cell. The non-naturally occurring antigen receptor or a sequence encoding a therapeutic protein can be stably expressed in the modified T-cell.

Gene editing compositions, including but not limited to, RNA-guided fusion proteins comprising dCas9-Clo051, as described in detail herein, can be used to target and decrease or eliminate expression of an endogenous T-cell receptor. In preferred aspects, the gene editing compositions target and delete a gene, a portion of a gene, or a regulatory element of a gene (such as a promoter) encoding an endogenous T-cell receptor. Non-limiting examples of primers (including a T7 promoter, genome target sequence, and gRNA scaffold) for the generation of guide RNA (gRNA) templates for targeting and deleting TCR-alpha (TCR-α), targeting and deleting TCR-beta (TCR-β), and targeting and deleting beta-2-microglobulin (B2M) are disclosed in PCT Application No. PCT/US2019/049816.

Gene editing compositions, including but not limited to, RNA-guided fusion proteins comprising dCas9-Clo051, can be used to target and decrease or eliminate expression of an endogenous MHCI, MHCII, or MHC activator. In preferred aspects, the gene editing compositions target and delete a gene, a portion of a gene, or a regulatory element of a gene (such as a promoter) encoding one or more components of an endogenous MHCI, MHCII, or MHC activator. Non-limiting examples of guide RNAs (gRNAs) for targeting and deleting MHC activators are disclosed in PCT Application No. PCT/US2019/049816.

A detailed description of non-naturally occurring chimeric stimulatory receptors, genetic modifications of endogenous sequences encoding TCR-alpha (TCR-α), TCR-beta (TCR-β), and/or Beta-2-Microglobulin (β2M), and non-naturally occurring polypeptides comprising an HLA class I histocompatibility antigen, alpha chain E (HLA-E) polypeptide is disclosed in PCT Application No. PCT/US2019/049816.

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the composition as well known in the art or as described herein.

For example, the disclosed LNP compositions of the present invention can further comprise a diluent. In some compositions, the diluent can be phosphate buffered saline (“PBS”).

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate. In some aspects, the buffer can include sucrose.

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994,), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized.

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

In aspects where the compositions to be administered to a subject in need thereof are modified cells as disclosed herein, the cells can be administered between about 1×10³ and 1×10¹⁵ cells; 1×10³ and 1×10¹⁵ cells, about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In an aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/04981.

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In an aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Preferably, the malignant disease is cancer. Non-limiting examples of a malignant disease or disorder include leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In preferred aspects, the treatment of a malignant disease or disorder comprises adoptive cell therapy. For example, in an aspect, the disclosure provides modified cells that express at least one disclosed protein scaffold and/or CAR comprising a protein scaffold (e.g., scFv, single domain antibody, Centyrin, delivered to the cell with a composition of the disclosure) that have been selected and/or expanded for administration to a subject in need thereof. Modified cells can be formulated for storage at any temperature including room temperature and body temperature. Modified cells can be formulated for cryopreservation and subsequent thawing. Modified cells can be formulated in a pharmaceutically acceptable carrier for direct administration to a subject from sterile packaging. Modified cells can be formulated in a pharmaceutically acceptable carrier with an indicator of cell viability and/or CAR expression level to ensure a minimal level of cell function and CAR expression. Modified cells can be formulated in a pharmaceutically acceptable carrier at a prescribed density with one or more reagents to inhibit further expansion and/or prevent cell death.

Any can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In an aspect, the administration is systemic. Systemic administration can be any means known in the art and described in detail herein. Preferably, systemic administration is by an intravenous injection or an intravenous infusion. In an aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In one example, the disclosure provides a method of treating cancer in a subject in need thereof, comprising administering to the subject a composition comprising a protein scaffold or a CAR comprising a protein scaffold (e.g., e.g., scFv, single domain antibody, Centyrin) the antibody or CAR specifically binds to an antigen on a tumor cell. In aspects where the composition comprises a modified cell or cell population, the cell or cell population may be autologous or allogeneic.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

### Protein Scaffold Production, Screening and Purification

At least one protein scaffold (e.g., monoclonal antibody, a chimeric antibody, a single domain antibody, a VHH, a VH, a single chain variable fragment (scFv), a Centyrin, an antigen-binding fragment (Fab) or a Fab fragment) of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y (1989): Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from a protein scaffold can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, a protein scaffold can be engineered with retention of high affinity for the antigen and other favorable biological properties. To achieve this goal, the scaffold proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate protein scaffold to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

Screening of a protein scaffold for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed nucleotide or peptide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. WO 91/17271, WO 91/18980, WO 91/19818, and WO 93/08278.

Other systems for generating libraries of peptides have aspects of both in vitro chemical synthesis and recombinant methods. See, PCT Patent Publication Nos. WO 92/05258, WO 92/14843, and WO 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, 5,427,908, 5,580,717, assigned to Affymax; 5,885,793, assigned to Cambridge Antibody Technologies; 5,750,373, assigned to Genentech, 5,618,920, 5,595,898, 5,576, 195, 5,698,435, 5,693,493, 5,698,417, assigned to Xoma, Colligan, supra; Ausubel, supra; or Sambrook, supra.

A protein scaffold of the disclosure can bind human or other mammalian proteins with a wide range of affinities (K_{D}). In a preferred aspect, at least one protein scaffold of the present disclosure can optionally bind to a target protein with high affinity, for example, with a K_{D} equal to or less than about 10-7 M, such as but not limited to, 0.1-9.9 (or any range or value therein)×10-8, 10-9, 10-10, 10-11, 10-12, 10-13, 10-14, 10-15 or any range or value therein, as determined by surface plasmon resonance or the Kinexa method, as practiced by those of skill in the art.

The affinity or avidity of a protein scaffold for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein). The measured affinity of a particular protein scaffold-antigen interaction can vary if measured under different conditions (e.g., salt concentration, pH). Thus, measurements of affinity and other antigen-binding parameters (e.g., K_{D}, Kon, Koff) are preferably made with standardized solutions of protein scaffold and antigen, and a standardized buffer, such as the buffer described herein.

Competitive assays can be performed with a protein scaffold in order to determine what proteins, antibodies, and other antagonists compete for binding to a target protein with the protein scaffold and/or share the epitope region. These assays as readily known to those of ordinary skill in the art evaluate competition between antagonists or ligands for a limited number of binding sites on a protein. The protein and/or antibody is immobilized or insolubilized before or after the competition and the sample bound to the target protein is separated from the unbound sample, for example, by decanting (where the protein/antibody was pre-insolubilized) or by centrifuging (where the protein/antibody was precipitated after the competitive reaction). Also, the competitive binding may be determined by whether function is altered by the binding or lack of binding of the protein scaffold to the target protein, e.g., whether the protein scaffold inhibits or potentiates the enzymatic activity of, for example, a label. ELISA and other functional assays may be used, as well known in the art.

### Nucleic Acid Molecules

Nucleic acid molecules of the disclosure encoding a protein scaffold can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified portion of at least one protein scaffold; nucleic acid molecules comprising the coding sequence for a protein scaffold or loop region that binds to the target protein; and nucleic acid molecules which comprise a nucleotide sequence substantially different from those described above but which, due to the degeneracy of the genetic code, still encode the protein scaffold as described herein and/or as known in the art. Of course, the genetic code is well known in the art. Thus, it would be routine for one skilled in the art to generate such degenerate nucleic acid variants that code for a specific protein scaffold of the present disclosure. See, e.g., Ausubel, et al., supra, and such nucleic acid variants are included in the present disclosure.

As indicated herein, nucleic acid molecules of the disclosure which comprise a nucleic acid molecule encoding a protein scaffold can include, but are not limited to, those encoding the amino acid sequence of a protein scaffold fragment, by itself; the coding sequence for the entire protein scaffold or a portion thereof; the coding sequence for a protein scaffold, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a protein scaffold can be fused to a marker sequence, such as a sequence encoding a peptide that facilitates purification of the fused protein scaffold comprising a protein scaffold fragment or portion.

### Polynucleotides Selectively Hybridizing to a Polynucleotide as Described Herein

The disclosure provides isolated nucleic acids that hybridize under selective hybridization conditions to a polynucleotide disclosed herein. Thus, the polynucleotides can be used for isolating, detecting, and/or quantifying nucleic acids comprising such polynucleotides. For example, polynucleotides of the present disclosure can be used to identify, isolate, or amplify partial or full-length clones in a deposited library. The polynucleotides can be genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of a protein scaffold encoded by the polynucleotides described herein. The polynucleotides embrace nucleic acid sequences that can be employed for selective hybridization to a polynucleotide encoding a protein scaffold of the present disclosure. See, e.g., Ausubel, supra; Colligan, supra, each entirely incorporated herein by reference.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965, 188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid molecules of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygß gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124 (8): 1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid molecule encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Protein Scaffold Purification

A protein scaffold can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

A protein scaffold of the disclosure include purified products, products of chemical synthetic procedures, and products produced by recombinant techniques from a prokaryotic or eukaryotic host, including, for example, E. coli, yeast, higher plant, insect and mammalian cells. Depending upon the host employed in a recombinant production procedure, the protein scaffold of the disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A protein scaffold of the disclosure can include one or more amino acid substitutions, deletions or additions, from spontaneous or mutations and/or human manipulation, as specified herein. Amino acids in a protein scaffold of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for protein scaffold binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active protein scaffold of the disclosure. Biologically active protein scaffolds have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to protein scaffolds and fragments, as described herein, which are modified by the covalent attachment of an organic moiety. Such modification can produce a protein scaffold fragment with improved pharmacokinetic properties (e.g., increased in vivo serum half-life). The organic moiety can be a linear or branched hydrophilic polymeric group, fatty acid group, or fatty acid ester group. In particular aspect, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified protein scaffolds and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a protein scaffold modified by the covalent attachment of polylysine is encompassed by the disclosure. Hydrophilic polymers suitable for modifying protein scaffolds of the disclosure can be linear or branched and include, for example, polyalkane glycols (e.g.,

PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein scaffold of the disclosure has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying protein scaffolds of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-49-octadecanoate (C18, oleate), all cis-45,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified protein scaffolds and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C₁-C₁₂ group wherein one or more carbon atoms can be replaced by a heteroatom, such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified protein scaffolds of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified protein scaffolds and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al.,

Protein Sci. 6 (10): 2233-2241 (1997); Itoh et al., Bioorg. Chem., 24 (1): 59-68 (1996); Capellas et al., Biotechnol. Bioeng., 56 (4): 456-463 (1997)), and the methods described in Hermanson, G. T., Bioconjugate Techniques, Academic Press: San Diego, Calif. (1996).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “alkyl” refers to straight and, when applicable, branched chain aliphatic groups having from 1 to 18 carbon atoms, As such, “alkyl” encompasses C₁, C₂, C₃, C₄, C₅, C₆, C₇, C₈, C₉, C₁₀, C₁₁ and C₁₂ groups. For instance, a C₁-C₆ alkyl group includes alkyl groups having 1 to 6 carbons. Examples of alkyl groups include, without limitation, methyl, ethyl, propyl, isopropyl, butyl, isobutyl, sec-butyl, tert-butyl, pentyl, and hexyl.

As used herein, the term “alkylene” is an alkyl, as defined hereinabove, that is positioned between and serves to connect two other chemical groups. Examples of alkylene groups include, without limitation, methylene, ethylene, propylene, butylene, pentylene and hexylene.

As used herein, the term “alkenyl” refers to an unsaturated straight or, when applicable, branched chain aliphatic group with one or more carbon-carbon double bonds, having from 2 to 18 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, and 1-methyl-2-buten-1-yl.

As used herein, the term “alkynyl” refers to an unsaturated straight or, when applicable, branched chain aliphatic group with one or more carbon-carbon triple bonds, having from 2 to 18 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

As used herein, the term “aralkyl” group comprises an aryl group covalently linked to an alkyl group, either of which may independently be optionally substituted or unsubstituted. An example of an aralkyl group is —(C₁-C₆)alkyl(C₆-C₁₀) aryl, including, without limitation, benzyl, phenethyl, and naphthylmethyl.

As used herein, the term “aryl” group is a C₆-C₁₄ aromatic moiety comprising one to three aromatic rings, which is optionally substituted. As such, “aryl” includes C₆, C₇, C₈, C₉, C₁₀, C₁₁, C₁₂ C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl.

As used herein, the term “C₂-C₁₀₀ hydrocarbon chain” refers to straight or branched chain, saturated or unsaturated comprising 2 to 100 carbon atoms. Examples of C₂-C₁₀₀ hydrocarbon chains groups include methyl, ethyl, propyl, isopropyl, butyl, isobutyl, sec-butyl, tert-butyl, pentyl, hexyl, heptanyl, octanyl, nonanyl, decanyl, undecanyl, dodecanyl, tridecanyl, icosanyl, triacontanyl, and tetracontanyl, and unsaturated counterparts thereof, e.g., propenyl and propynyl.

As used herein, the term “cycloalkyl” as employed herein includes saturated and partially unsaturated cyclic hydrocarbon groups having 3 to 12 carbons. As such, “cycloalkyl” includes C₃, C₄, C₅, C₆, C₇, C₈, C₉, C₁₀, C₁₁ and C₁₂ cyclic hydrocarbon groups. Representative cycloalkyl groups include, without limitation, cyclopropyl, cyclobutyl, cyclopentyl, cyclopentenyl, cyclohexyl, cyclohexenyl, cycloheptyl, and cyclooctyl.

As used herein, the term “hydroxyalkyl” refers to -alkyl-OH or an alkyl chain substituted with at least one-OH.

As used herein, the term “halo” or “halogen” refers to fluoro, chloro, bromo and iodo.

It will be understood that the compounds of any one of the Formulae disclosed herein and any pharmaceutically acceptable salts thereof, comprise stereoisomers, mixtures of stereoisomers, polymorphs of all isomeric forms of said compounds.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to an aspect hereof, the term “antibody hereof” in its broadest sense also covers such analogs. Generally, in such analogs, one or more amino acid residues may have been replaced, deleted and/or added, compared to the antibodies hereof as defined herein.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH₂, CH₃, and CH₄, depending on antibody isotype) of the Fc region of the intact antibody. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). The term further includes single domain antibodies (“sdAB”) which generally refers to an antibody fragment having a single monomeric variable antibody domain, (for example, from camelids). Such antibody fragment types will be readily understood by a person having ordinary skill in the art.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In some aspects, a specific binding interaction will discriminate between desirable and undesirable antigens in a sample. In some aspects, more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of =2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 1.

TABLE 1
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 2.

TABLE 2
Conservative Substitutions II

| Side Chain Characteristic | | Amino Acid |
|---|---|---|
| Non-polar (hydrophobic) | Aliphatic: | A L I V P |
| | Aromatic: | F W Y |
| | Sulfur- | M |
| | containing: | |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 3.

TABLE 3
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (L) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### Exemplary Embodiments

Embodiment 1. A composition comprising at least one lipid nanoparticle comprising at least one compound of Formula (I), Formula (II), or Formula (III):

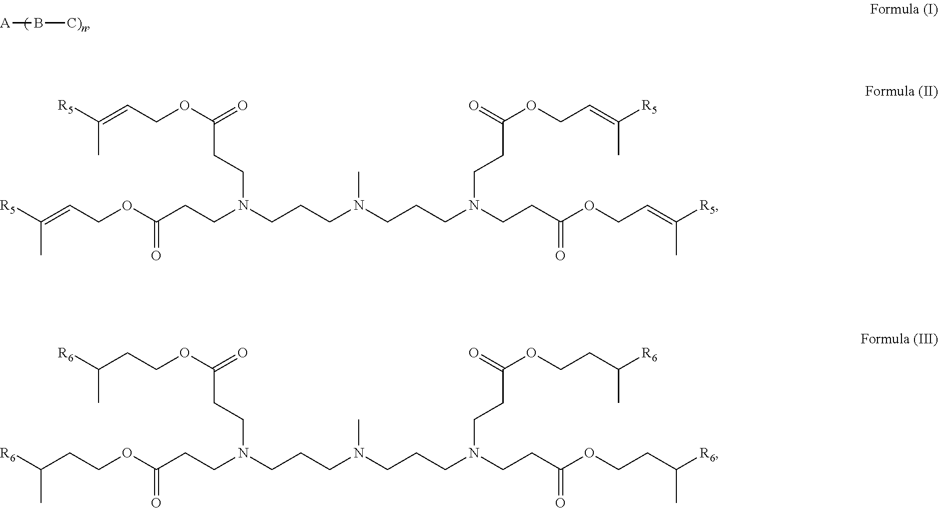

or a salt thereof,
wherein:
A is A′ or A″,
A′ is:

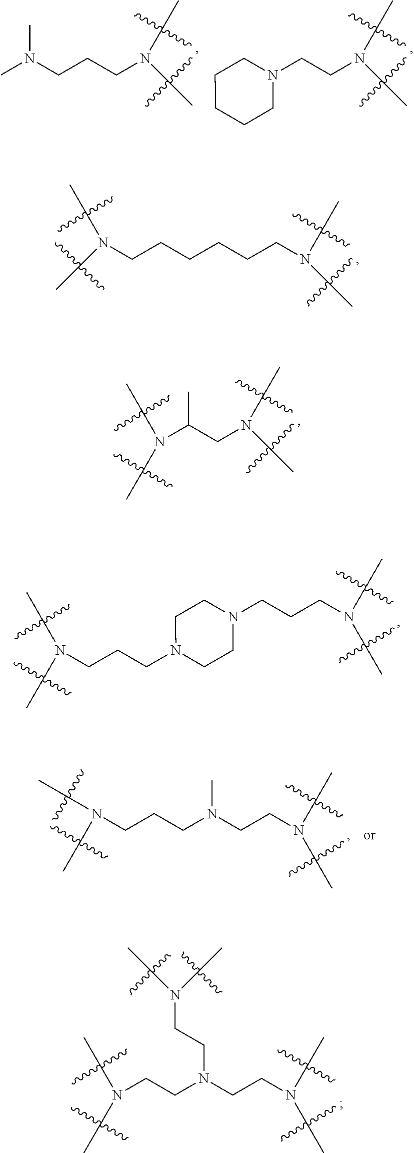

and
A″ is:

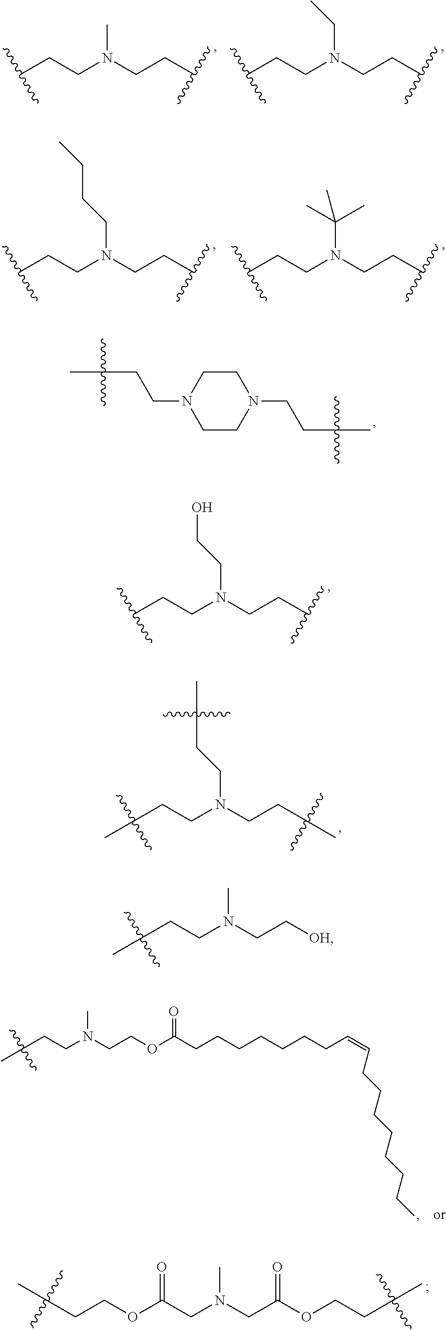

B is B′ or B″,
B′ is:

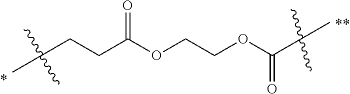 [SMILES: *CCC(=O)OCCOC(*)=O]

in which * indicates attachment to A, and ** indicates attachment to C:
B″ is:

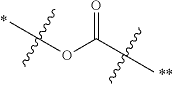 [SMILES: *OC(*)=O]

in which * indicates attachment to A, and ** indicates attachment to C; wherein when A is A′, then B is B′, and wherein when A is A″, then Bis B″:
C is:

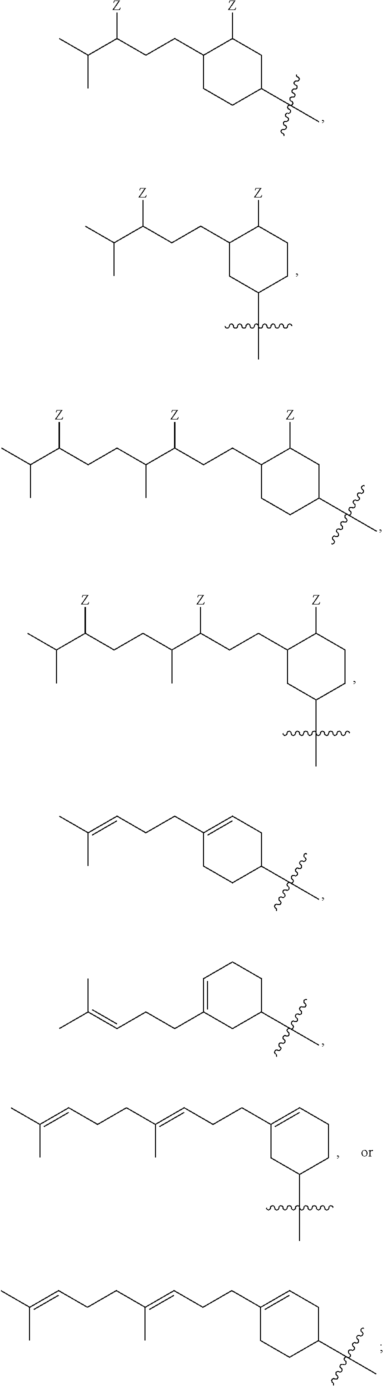

n is an integer between 1 to 6;
each Z is independently H or —S—R₁;
each R₁ is independently alkyl,

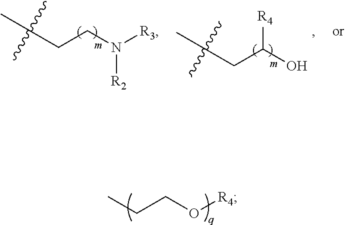

each R₂ is independently hydrogen, C₁-C₆ alkyl, C₁-C₆ alkenyl, aralkyl, or hydroxyalkyl;
each R₃ is independently hydrogen, C₁-C₆ alkyl, C₁-C₆ alkenyl, aralkyl, or hydroxyalkyl;
each R₄ is independently hydrogen or C₁-C₃ alkyl;
each R₅ is independently

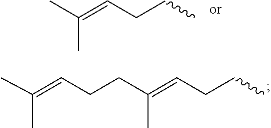

each R₆ is independently

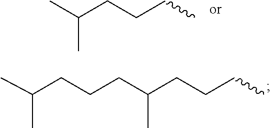

each m is an integer independently selected from 1-10; and
each q is an integer independently selected from 1-200,
wherein the at least one lipid nanoparticle further comprises at least one nucleic acid molecule.

Embodiment 2. The composition of embodiment 1, wherein the at least one lipid nanoparticle comprises about 41.4% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 10% of cholesterol by moles,
about 45.9% of DOPE by moles, and
about 2.7% of DMG-PEG2000 by moles; and
wherein the ratio of lipid to nucleic acid in the at least one nanoparticle is about 40:1 (w/w).

Embodiment 3. The composition of embodiment 1, wherein the at least one lipid nanoparticle comprises about 41.44% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 10% of cholesterol by moles,
about 45.85% of DOPE by moles, and
about 2.7% of DMG-PEG2000 by moles; and
wherein the ratio of lipid to nucleic acid in the at least one nanoparticle is about 40:1 (w/w).

Embodiment 4. The composition of embodiment 1, wherein the at least one lipid nanoparticle comprises about 33.5% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 33.5% of cholesterol by moles,
about 32% of DOPE by moles, and
about 1% of DMG-PEG2000 by moles; and
wherein the ratio of lipid to nucleic acid in the at least one nanoparticle is about 40:1 (w/w).

Embodiment 5. The composition of embodiment 1, wherein the at least one lipid nanoparticle comprises about 40.7% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 47.1% of cholesterol by moles,
about 10% of DOPE by moles, and
about 2.2% of DMG-PEG2000 by moles; and
wherein the ratio of lipid to nucleic acid in the at least one nanoparticle is about 100:1 (w/w).

Embodiment 6. The composition of embodiment 1, wherein the at least one lipid nanoparticle comprises about 40.68% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 47.12% of cholesterol by moles,
about 10% of DOPE by moles, and
about 2.2% of DMG-PEG2000 by moles; and
wherein the ratio of lipid to nucleic acid in the at least one nanoparticle is about 100:1 (w/w).

Embodiment 7. The composition of embodiment 1, wherein the at least one lipid nanoparticle comprises about 40.7% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 47.1% of cholesterol by moles,
about 10% of DOPC by moles, and
about 2.2% of DMG-PEG2000 by moles; and
wherein the ratio of lipid to nucleic acid in the at least one nanoparticle is about 100:1 (w/w).

Embodiment 8. The composition of embodiment 1, wherein the at least one lipid nanoparticle comprises about 40.68% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 47.12% of cholesterol by moles,
about 10% of DOPC by moles, and
about 2.2% of DMG-PEG2000 by moles; and
wherein the ratio of lipid to nucleic acid in the at least one nanoparticle is about 100:1 (w/w).

Embodiment 9. The composition of embodiment 1, wherein the at least one lipid nanoparticle comprises about 60% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 29% of cholesterol by moles,
about 10% of DOPE by moles, and
about 1% of DMG-PEG2000 by moles; and
wherein the ratio of lipid to nucleic acid in the at least one nanoparticle is about 100:1 (w/w).

Embodiment 10. The composition of embodiment 1, wherein the at least one lipid nanoparticle comprises about 60% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 29% of cholesterol by moles,
about 10% of DOPC by moles, and
about 1% of DMG-PEG2000 by moles; and
wherein the ratio of lipid to nucleic acid in the at least one nanoparticle is about 100:1 (w/w).

Embodiment 11. The composition of embodiment 1, wherein the at least one lipid nanoparticle comprises about 24.3% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 46.4% of cholesterol by moles,
about 28.3% of DOPE by moles, and
about 1% of DMG-PEG2000 by moles; and
wherein the ratio of lipid to nucleic acid in the at least one nanoparticle is about 100:1 (w/w).

Embodiment 12. The composition of embodiment 1, wherein the at least one lipid nanoparticle comprises about 24.29% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 46.41% of cholesterol by moles,
about 28.3% of DOPE by moles, and
about 1% of DMG-PEG2000 by moles; and
wherein the ratio of lipid to nucleic acid in the at least one nanoparticle is about 100:1 (w/w).

Embodiment 13. The composition of embodiment 1, wherein the at least one lipid nanoparticle comprises about 24.3% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 46.4% of cholesterol by moles,
about 28.3% of DOPC by moles, and
about 1% of DMG-PEG2000 by moles; and
wherein the ratio of lipid to nucleic acid in the at least one nanoparticle is about 100:1 (w/w).

Embodiment 14. The composition of embodiment 1, wherein the at least one lipid nanoparticle comprises about 24.29% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 46.41% of cholesterol by moles,
about 28.3% of DOPC by moles, and
about 1% of DMG-PEG2000 by moles; and
wherein the ratio of lipid to nucleic acid in the at least one nanoparticle is about 100:1 (w/w).

Embodiment 15. The composition of embodiment 1, wherein the at least one lipid nanoparticle comprises about 40% to about 60% of the least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 29% to about 47.1% of cholesterol by moles,
about 10% of DOPC or DOPE by moles, and
about 1% to about 2.2% of DMG-PEG2000 by moles,
and wherein the ratio of lipid to nucleic acid in the at least one nanoparticle is about 100:1 (w/w)

Embodiment 16. The composition of embodiment 1, wherein the at least one lipid nanoparticle comprises about 35% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 41.8% of cholesterol by moles,
about 20% of DOPE by moles, and
about 3.2% of DMG-PEG2000 by moles; and
wherein the ratio of lipid to nucleic acid in the at least one nanoparticle is about 80:1 (w/w).

Embodiment 17. The composition of embodiment 1, wherein the at least one lipid nanoparticle comprises about 35% of the at least one compound of Formula (I), Formula (II) or Formula (III) by moles,
wherein the at least one nucleic acid molecule comprises at least one DNA molecule, wherein the at least one lipid nanoparticle further comprises:
about 41.8% of cholesterol by moles,
about 20% of DOPE by moles, and
about 3.16% of DMG-PEG2000 by moles; and
wherein the ratio of lipid to nucleic acid in the at least one nanoparticle is about 80:1 (w/w).

Embodiment 18. The composition of embodiment 1, wherein the at least one lipid nanoparticle comprises about 38% of the at least one compound of Formula (I), Formula (II) or Formula (III) by moles,
wherein the at least one nucleic acid molecule comprises at least one DNA molecule, wherein the at least one lipid nanoparticle further comprises:
about 10% of cholesterol by moles,
about 50% of DOPE by moles, and
about 2% of DMG-PEG2000 by moles; and
wherein the ratio of lipid to nucleic acid in the at least one nanoparticle is about 45:1 (w/w) or about 40:1 (w/w).

Embodiment 19. The composition of embodiment 18, wherein the ratio of lipid to nucleic acid in the at least one nanoparticle is about 45:1 (w/w).

Embodiment 20. The composition of embodiment 18, wherein the ration of lipid to nucleic acid in the at least one nanoparticle is about 40:1 (w/w).

Embodiment 21. The composition of embodiment 1, wherein the at least one lipid nanoparticle comprises about 40% to about 45.1% of the least one compound of Formula (I), Formula (II) or Formula (III) by moles,
wherein the at least one nucleic acid molecule comprises at least one DNA molecule, wherein the at least one lipid nanoparticle further comprises:
about 40.6% to about 51.8% of cholesterol by moles,
about 5% to about 13.5% of DOPC by moles, and
about 1.7% to about 2.6% of DMG-PEG2000 by moles,
and wherein the ratio of lipid to nucleic acid in the at least one nanoparticle is about 100:1 (w/w) or about 120:1 (w/w).

Embodiment 22. The composition of embodiment 1, wherein the at least one lipid nanoparticle comprises about 34% to about 37.8% of the least one compound of Formula (I), Formula (II) or Formula (III) by moles,
wherein the at least one nucleic acid molecule comprises at least one DNA molecule, wherein the at least one lipid nanoparticle further comprises:
about 46.7% to about 55.1% of cholesterol by moles,
about 5% to about 14.3% of DOPC by moles, and
about 1.7% to about 2.7% of DMG-PEG2000 by moles,
and wherein the ratio of lipid to nucleic acid in the at least one nanoparticle is about 120:1 (w/w).

Embodiment 23. The composition of embodiment 1, wherein the at least one lipid nanoparticle comprises about 35.3% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 53.7% of cholesterol by moles,
about 8.3% of DOPC by moles, and
about 2.7% of DMG-PEG2000 by moles; and
wherein the ratio of lipid to nucleic acid in the at least one nanoparticle is about 120:1 (w/w).

Embodiment 24. The composition of embodiment 1, wherein the at least one lipid nanoparticle comprises about 35.28% of the at least one compound of Formula (I), Formula (II)
wherein the at least one nucleic acid molecule comprises at least one RNA molecule and/or at least one DNA molecule,
wherein the at least one lipid nanoparticle further comprises:
about 53.68% of cholesterol by moles,
about 8.35% of DOPC by moles, and
about 2.69% of DMG-PEG2000 by moles; and
wherein the ratio of lipid to nucleic acid in the at least one nanoparticle is about 120:1 (w/w).

Embodiment 25. The composition of embodiment 1, wherein the at least one lipid nanoparticle comprises about 37.8% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 55.1% of cholesterol by moles,
about 5% of DOPC by moles, and
about 2.2% of DMG-PEG2000 by moles; and
wherein the ratio of lipid to nucleic acid in the at least one nanoparticle is about 120:1 or about 100:1 (w/w).

Embodiment 26. The composition of embodiment 1, wherein the at least one lipid nanoparticle comprises about 37.77% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 55.07% of cholesterol by moles,
about 5% of DOPC by moles, and
about 2.15% of DMG-PEG2000 by moles; and
wherein the ratio of lipid to nucleic acid in the at least one nanoparticle is about 120:1 or about 100:1 (w/w).

Embodiment 27. The composition of embodiment 25 or embodiment 26, wherein the ratio of lipid to nucleic acid in the at least one nanoparticle is about 120:1 (w/w).

Embodiment 28. The composition of embodiment 25 or embodiment 26, wherein the ratio of lipid to nucleic acid in the at least one nanoparticle is about 100:1 (w/w).

Embodiment 29. The composition of embodiment 1, wherein the at least one lipid nanoparticle comprises about 44.4% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 48.5% of cholesterol by moles,
about 5.4% of DOPC by moles, and
about 1.7% of DMG-PEG2000 by moles; and
wherein the ratio of lipid to nucleic acid in the at least one nanoparticle is about 120:1 (w/w).

Embodiment 30. The composition of embodiment 1, wherein the at least one lipid nanoparticle comprises about 44.38% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 48.46% of cholesterol by moles,
about 5.43% of DOPC by moles, and
about 1.73% of DMG-PEG2000 by moles; and
wherein the ratio of lipid to nucleic acid in the at least one nanoparticle is about 120:1 (w/w).

Embodiment 31. The composition of embodiment 1, wherein the at least one lipid nanoparticle comprises about 40% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 50.5% of cholesterol by moles,
about 7.2% of DOPC by moles, and
about 2.4% of DMG-PEG2000 by moles; and
wherein the ratio of lipid to nucleic acid in the at least one nanoparticle is about 120:1 (w/w).

Embodiment 32. The composition of embodiment 1, wherein the at least one lipid nanoparticle comprises about 39.98% of the at least one compound of Formula (I), Formula (II)
wherein the at least one nucleic acid molecule comprises at least one RNA molecule and/or at least one DNA molecule,
wherein the at least one lipid nanoparticle further comprises:
about 50.47% of cholesterol by moles,
about 7.17% of DOPC by moles, and
about 2.39% of DMG-PEG2000 by moles; and
wherein the ratio of lipid to nucleic acid in the at least one nanoparticle is about 120:1 (w/w).

Embodiment 33. The composition of embodiment 1, wherein the at least one lipid nanoparticle comprises about 37% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 46.7% of cholesterol by moles,
about 14.3% of DOPC by moles, and
about 2% of DMG-PEG2000 by moles; and
wherein the ratio of lipid to nucleic acid in the at least one nanoparticle is about 120:1 (w/w).

Embodiment 34. The composition of embodiment 1, wherein the at least one lipid nanoparticle comprises about 36.97% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 46.69% of cholesterol by moles,
about 14.34% of DOPC by moles, and
about 2% of DMG-PEG2000 by moles; and
wherein the ratio of lipid to nucleic acid in the at least one nanoparticle is about 120:1 (w/w).

Embodiment 35. The composition of embodiment 1, wherein the at least one lipid nanoparticle comprises about 34% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 51.4% of cholesterol by moles,
about 12.9% of DOPC by moles, and
about 1.7% of DMG-PEG2000 by moles; and
wherein the ratio of lipid to nucleic acid in the at least one nanoparticle is about 120:1 (w/w).

Embodiment 36. The composition of embodiment 1, wherein the at least one lipid nanoparticle comprises about 33.96% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 51.44% of cholesterol by moles,
about 12.89% of DOPC by moles, and
about 1.7% of DMG-PEG2000 by moles; and
wherein the ratio of lipid to nucleic acid in the at least one nanoparticle is about 120:1 (w/w).

Embodiment 37. The composition of embodiment 1, wherein the at least one lipid nanoparticle comprises about 45% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 40.8% of cholesterol by moles,
about 12.4% of DOPC by moles, and
about 1.9% of DMG-PEG2000 by moles; and
wherein the ratio of lipid to nucleic acid in the at least one nanoparticle is about 120:1 (w/w).

Embodiment 38. The composition of embodiment 1, wherein the at least one lipid nanoparticle comprises about 44.95% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 40.77% of cholesterol by moles,
about 12.4% of DOPC by moles, and
about 1.88% of DMG-PEG2000 by moles; and
wherein the ratio of lipid to nucleic acid in the at least one nanoparticle is about 120:1 (w/w).

Embodiment 39. The composition of embodiment 1, wherein the at least one lipid nanoparticle comprises about 43.1% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 45.1% of cholesterol by moles,
about 9.6% of DOPC by moles, and
about 2.2% of DMG-PEG2000 by moles; and
wherein the ratio of lipid to nucleic acid in the at least one nanoparticle is about 120:1 (w/w).

Embodiment 40. The composition of embodiment 1, wherein the at least one lipid nanoparticle comprises about 43.11% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 45.09% of cholesterol by moles,
about 9.59% of DOPC by moles, and
about 2.21% of DMG-PEG2000 by moles; and
wherein the ratio of lipid to nucleic acid in the at least one nanoparticle is about 120:1 (w/w).

Embodiment 41. The composition of embodiment 1, wherein the at least one lipid nanoparticle comprises about 41% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 42.8% of cholesterol by moles,
about 13.5% of DOPC by moles, and
about 2.6% of DMG-PEG2000 by moles; and
wherein the ratio of lipid to nucleic acid in the at least one nanoparticle is about 120:1 (w/w).

Embodiment 42. The composition of embodiment 1, wherein the at least one lipid nanoparticle comprises about 41.02% of the at least one compound of Formula (I), Formula (II)
wherein the at least one nucleic acid molecule comprises at least one RNA molecule and/or at least one DNA molecule,
wherein the at least one lipid nanoparticle further comprises:
about 42.83% of cholesterol by moles,
about 13.55% of DOPC by moles, and
about 2.61% of DMG-PEG2000 by moles; and
wherein the ratio of lipid to nucleic acid in the at least one nanoparticle is about 120:1 (w/w).

Embodiment 43. The composition of embodiment 1, wherein the at least one lipid nanoparticle comprises about 36.7% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 48.4% of cholesterol by moles,
about 12.5% of DOPC by moles, and
about 2.4% of DMG-PEG2000 by moles; and
wherein the ratio of lipid to nucleic acid in the at least one nanoparticle is about 120:1 (w/w).

Embodiment 44. The composition of embodiment 1, wherein the at least one lipid nanoparticle comprises about 36.71% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 48.44% of cholesterol by moles,
about 12.46% of DOPC by moles, and
about 2.39% of DMG-PEG2000 by moles; and
wherein the ratio of lipid to nucleic acid in the at least one nanoparticle is about 120:1 (w/w).

Embodiment 45. The composition of embodiment 1, wherein the at least one lipid nanoparticle comprises about 45.1% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 40.6% of cholesterol by moles,
about 12.6% of DOPC by moles, and
about 1.8% of DMG-PEG2000 by moles; and
wherein the ratio of lipid to nucleic acid in the at least one nanoparticle is about 100:1 (w/w).

Embodiment 46. The composition of embodiment 1, wherein the at least one lipid nanoparticle comprises about 45.12% of the at least one compound of Formula (I), Formula (II) or Formula (III) by moles,
wherein the at least one nucleic acid molecule comprise at least one RNA molecule and/or s at least one DNA molecule,
wherein the at least one lipid nanoparticle further comprises:
about 40.58% of cholesterol by moles,
about 12.55% of DOPC by moles, and
about 1.75% of DMG-PEG2000 by moles; and
wherein the ratio of lipid to nucleic acid in the at least one nanoparticle is about 100:1 (w/w).

Embodiment 47. The composition of embodiment 1, wherein the at least one lipid nanoparticle comprises about 40.8% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 51.8% of cholesterol by moles,
about 5% of DOPC by moles, and
about 2.5% of DMG-PEG2000 by moles; and
wherein the ratio of lipid to nucleic acid in the at least one nanoparticle is about 120:1, 100:1, 80:1, 60:1 or 40:1 (w/w).

Embodiment 48. The composition of embodiment 1, wherein the at least one lipid nanoparticle comprises about 40.75% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 51.75% of cholesterol by moles,
about 5% of DOPC by moles, and
about 2.5% of DMG-PEG2000 by moles; and
wherein the ratio of lipid to nucleic acid in the at least one nanoparticle is about 120:1, 100:1, 80:1, 60:1 or 40:1 (w/w).

Embodiment 49. The composition of embodiment 47 or embodiment 48, wherein the ratio of lipid to nucleic acid in the at least one nanoparticle is about 120:1 (w/w).

Embodiment 50. The composition of embodiment 47 or embodiment 48, wherein the ratio of lipid to nucleic acid in the at least one nanoparticle is about 100:1 (w/w).

Embodiment 51. The composition of embodiment 47 or embodiment 48, wherein the ratio of lipid to nucleic acid in the at least one nanoparticle is about 80:1 (w/w).

Embodiment 52. The composition of embodiment 47 or embodiment 48, wherein the ratio of lipid to nucleic acid in the at least one nanoparticle is about 60:1 (w/w).

Embodiment 53. The composition of embodiment 47 or embodiment 48, wherein the ratio of lipid to nucleic acid in the at least one nanoparticle is about 40:1 (w/w).

Embodiment 54. The composition of embodiment 1, wherein the at least one lipid nanoparticle comprises about 32.5% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 60% of cholesterol by moles,
about 5% of DOPC by moles, and
about 2.5% of DMG-PEG2000 by moles; and
wherein the ratio of lipid to nucleic acid in the at least one nanoparticle is about 120:1 (w/w).

Embodiment 55. The composition of embodiment 1, wherein the at least one lipid nanoparticle comprises about 40% to about 45.1% of the least one compound of Formula (I), Formula (II) or Formula (III) by moles,
wherein the at least one nucleic acid molecule comprises at least one RNA molecule,
wherein the at least one lipid nanoparticle further comprises:
about 40.6% to about 51.8% of cholesterol by moles,
about 5% to about 13.5% of DOPC by moles, and
about 1.7% to about 2.6% of DMG-PEG2000 by moles,
and wherein the ratio of lipid to nucleic acid in the at least one nanoparticle is about 100:1 (w/w) or about 120:1 (w/w).

Embodiment 56. The composition of embodiment 1, wherein the at least one lipid nanoparticle comprises about 34% to about 37.8% of the least one compound of Formula (I), Formula (II) or Formula (III) by moles,
wherein the at least one nucleic acid molecule comprises at least one RNA molecule,
wherein the at least one lipid nanoparticle further comprises:
about 46.7% to about 55.1% of cholesterol by moles,
about 5% to about 14.3% of DOPC by moles, and
about 1.7% to about 2.7% of DMG-PEG2000 by moles,
and wherein the ratio of lipid to nucleic acid in the at least one nanoparticle is about 120:1 (w/w).

Embodiment 57. The composition of embodiment 1, wherein the at least one lipid nanoparticle comprises about 34.4% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 56.18% of cholesterol by moles,
about 7.86% of DOPC by moles, and
about 1.54% of DMG-PEG2000 by moles; and
wherein the ratio of lipid to nucleic acid in the at least one nanoparticle is about 100:1 (w/w).

Embodiment 58. The composition of embodiment 1, wherein the at least one lipid nanoparticle comprises about 47.03% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 46.28% of cholesterol by moles,
about 3.76% of DOPC by moles, and
about 2.93% of DMG-PEG2000 by moles; and
wherein the ratio of lipid to nucleic acid in the at least one nanoparticle is about 100:1 (w/w).

Embodiment 59. The composition of embodiment 1, wherein the at least one lipid nanoparticle comprises about 44.48% of the at least one compound of Formula (I), Formula (II)
wherein the at least one nucleic acid molecule comprises at least one RNA molecule and/or at least one DNA molecule,
wherein the at least one lipid nanoparticle further comprises:
about 48.46% of cholesterol by moles,
about 4.68% of DOPC by moles, and
about 2.34% of DMG-PEG2000 by moles; and
wherein the ratio of lipid to nucleic acid in the at least one nanoparticle is about 100:1 (w/w).

Embodiment 60. The composition of embodiment 1, wherein the at least one lipid nanoparticle comprises about 30.3% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 50.45% of cholesterol by moles,
about 17.27% of DOPC by moles, and
about 1.98% of DMG-PEG2000 by moles; and
wherein the ratio of lipid to nucleic acid in the at least one nanoparticle is about 100:1 (w/w).

Embodiment 61. The composition of embodiment 1, wherein the at least one lipid nanoparticle comprises about 66.98% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 30.96% of cholesterol by moles,
about 0.35% of DOPC by moles, and
about 1.71% of DMG-PEG2000 by moles; and
wherein the ratio of lipid to nucleic acid in the at least one nanoparticle is about 100:1 (w/w).

Embodiment 62. The composition of embodiment 1, wherein the at least one lipid nanoparticle comprises about 30% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 55% of cholesterol by moles,
about 12% of DOPC by moles, and
about 3% of DMG-PEG2000 by moles; and
wherein the ratio of lipid to nucleic acid in the at least one nanoparticle is about 100:1 (w/w).

Embodiment 63. The composition of embodiment 1, wherein the at least one lipid nanoparticle comprises about 51.71% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 40.46% of cholesterol by moles,
about 5.99% of DOPC by moles, and
about 1.85% of DMG-PEG2000 by moles; and
wherein the ratio of lipid to nucleic acid in the at least one nanoparticle is about 100:1 (w/w).

Embodiment 64. The composition of embodiment 1, wherein the at least one lipid nanoparticle comprises about 52.5% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 30% of cholesterol by moles,
about 16% of DOPC by moles, and
about 1.5% of DMG-PEG2000 by moles; and
wherein the ratio of lipid to nucleic acid in the at least one nanoparticle is about 100:1 (w/w).

Embodiment 65. The composition of embodiment 1, wherein the at least one lipid nanoparticle comprises about 47% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 30% of cholesterol by moles,
about 20% of DOPC by moles, and
about 3% of DMG-PEG2000 by moles; and
wherein the ratio of lipid to nucleic acid in the at least one nanoparticle is about 100:1 (w/w).

Embodiment 66. The composition of embodiment 1, wherein the at least one lipid nanoparticle comprises about 54.11% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 33.4% of cholesterol by moles,
about 9.49% of DOPC by moles, and
about 3% of DMG-PEG2000 by moles; and
wherein the ratio of lipid to nucleic acid in the at least one nanoparticle is about 100:1 (w/w).

Embodiment 67. The composition of embodiment 1, wherein the at least one lipid nanoparticle comprises about 29% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 60% of cholesterol by moles,
about 10% of DOPE by moles, and
about 1% of DMG-PEG2000 by moles; and
wherein the ratio of lipid to nucleic acid in the at least one nanoparticle is about 100:1 or about 40:1 (w/w).

Embodiment 68. The composition of embodiment 67, wherein the ratio of lipid to nucleic acid in the at least one nanoparticle is about 100:1 (w/w).

Embodiment 69. The composition of embodiment 67, wherein the ratio of lipid to nucleic acid in the at least one nanoparticle is about 40:1 (w/w).

Embodiment 70. The composition of embodiment 1, wherein the at least one lipid nanoparticle comprises about 60% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 10% of cholesterol by moles,
about 27.8% of DOPE by moles, and
about 2.2% of DMG-PEG2000 by moles; and
wherein the ratio of lipid to nucleic acid in the at least one nanoparticle is about 100:1 (w/w).

Embodiment 71. The composition of embodiment 1, wherein the at least one lipid nanoparticle comprises about 44.5% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 10% of cholesterol by moles,
about 44.5% of DOPE by moles, and
about 1% of DMG-PEG2000 by moles; and
wherein the ratio of lipid to nucleic acid in the at least one nanoparticle is about 100:1 (w/w).

Embodiment 72. The composition of embodiment 1, wherein the at least one lipid nanoparticle comprises about 46.32% of the at least one compound of Formula (I), Formula (II) or Formula (III) by moles,
wherein the at least one nucleic acid molecule comprises at least one RNA molecule and/or at least one DNA molecule,
wherein the at least one lipid nanoparticle further comprises:
about 39.48% of cholesterol by moles,
about 10% of DOPE by moles, and
about 4.2% of DMG-PEG2000 by moles; and
wherein the ratio of lipid to nucleic acid in the at least one nanoparticle is about 100:1 (w/w).

Embodiment 73. The composition of any of one of the preceding embodiments, wherein each C in the compound of Formula (I) is:

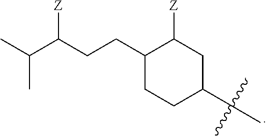

Embodiment 74. The composition of any of one of embodiments 1-72, wherein each C in the compound of Formula (I) is:

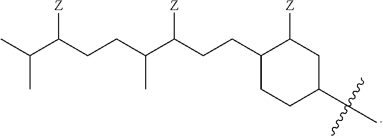

Embodiment 75. The composition of any of one of embodiments 1-72, wherein each C in the compound of Formula (I) is:

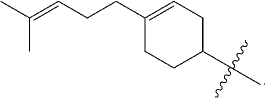 [SMILES: *C1CC=C(CCC=C(C)C)CC1]

Embodiment 76. The composition of any of one of embodiments 1-72, wherein each C in the compound of Formula (I) is:

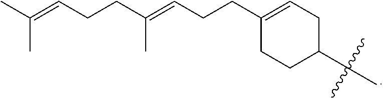 [SMILES: *C1CC=C(CC/C=C(\C)CCC=C(C)C)CC1]

Embodiment 77. The composition according to embodiments 73 or 74, wherein Z is H.

Embodiment 78. The composition according to embodiments 73 or 74, wherein Z is —S—R₁, and wherein at least one R₁ is alkyl.

Embodiment 79. The composition according to embodiment 78, wherein the at least one R₁ is C₁₀H₂₁.

Embodiment 80. The composition according to embodiment 78, wherein the least one R₁ is C₂H₄(CH)(CH₃)₂.

Embodiment 81. The composition according to any of embodiments 1-80, wherein the A in the compound of Formula (I) is A′ and the B in the compound of Formula (I) is B′.

Embodiment 82. The composition according to any of embodiments 1-80, wherein the A in the compound of Formula (I) is A′ and A′ is

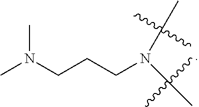 [SMILES: *N(*)CCCN(C)C]

Embodiment 83. The composition according to any of embodiments 1-80, wherein the A in the compound of Formula (I) is A′ and A′ is

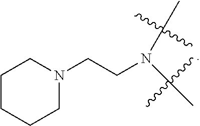 [SMILES: *N(*)CCN1CCCCC1]

Embodiment 84. The composition according to any of embodiments 1-80, wherein the A in the compound of Formula (I) is A′ and A′ is

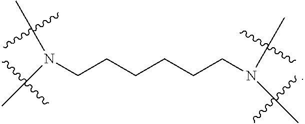 [SMILES: *N(*)CCCCCCN(*)*]

Embodiment 85. The composition according to any of embodiments 1-80, wherein the A in the compound of Formula (I) is A′ and A′ is

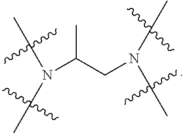 [SMILES: *N(*)CC(C)N(*)*]

Embodiment 86. The composition according to any of embodiments 1-80, wherein the A in the compound of Formula (I) is A′ and A′ is

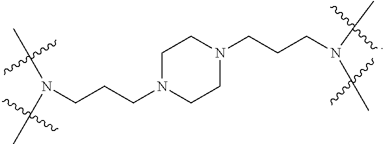

Embodiment 87. The composition according to any of embodiments 1-80, wherein the A in the compound of Formula (I) is A′ and A′ is

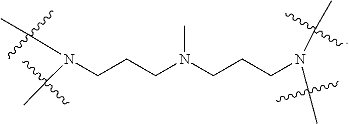 [SMILES: *N(*)CCCN(C)CCCN(*)*]

Embodiment 88. The composition according to any of embodiments 1-72, wherein the A in the compound of Formula (I) is A′, A′ is

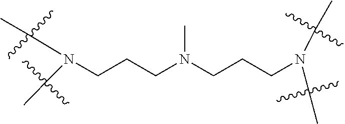 [SMILES: *N(*)CCCN(C)CCCN(*)*]

### and B is B′.

Embodiment 89. The composition of embodiment 88, wherein Cis

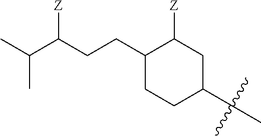

and each Z is independently H.

Embodiment 90. The composition of embodiment 89, wherein n is 4.

Embodiment 91. The composition according to any of embodiments 1-80, wherein the A in the compound of Formula (I) is A′ and A′ is

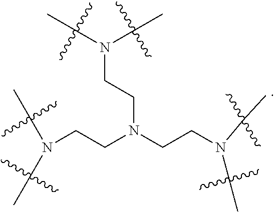 [SMILES: *N(*)CCN(CCN(*)*)CCN(*)*]

Embodiment 92. The composition according to any of embodiments 1-80, wherein the A in the compound of Formula (I) is A″ and B is B″.

Embodiment 93. The composition according to any of embodiments 1-80, wherein the A in the compound of Formula (I) is A″ and A″ is

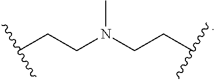 [SMILES: *CCN(C)CC*]

Embodiment 94. The composition according to any of embodiments 1-80, wherein the A in the compound of Formula (I) is A″ and A″ is

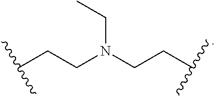 [SMILES: *CCN(CC)CC*]

Embodiment 95. The composition according to any of embodiments 1-80, wherein the A in the compound of Formula (I) is A″ and A″ is

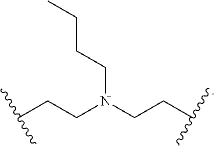 [SMILES: *CCN(CC*)CCCC]

Embodiment 96. The composition according to any of embodiments 1-80, wherein the A in the compound of Formula (I) is A″ and A″ is

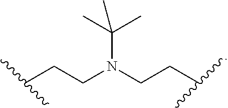 [SMILES: *CCN(CC*)C(C)(C)C]

Embodiment 97. The composition according to any of embodiments 1-80, wherein the A in the compound of Formula (I) is A″ and A″ is

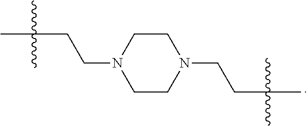 [SMILES: *CCN1CCN(CC*)CC1]

Embodiment 98. The composition according to any of embodiments 1-80, wherein the A in the compound of Formula (I) is A″ and A″ is

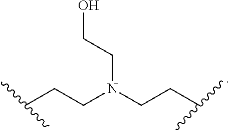 [SMILES: *CCN(CC*)CCO]

Embodiment 99. The composition according to any of embodiments 1-80, wherein the A in the compound of Formula (I) is A″ and A″ is

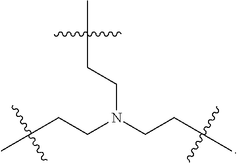 [SMILES: *CCN(CC*)CC*]

Embodiment 100. The composition according to any of embodiments 1-80, wherein the A in the compound of Formula (I) is A″ and A″ is

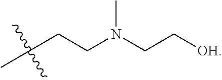 [SMILES: *CCN(C)CCO]

Embodiment 101. The composition according to any of embodiments 1-80, wherein the A in the compound of Formula (I) is A″ and A″ is

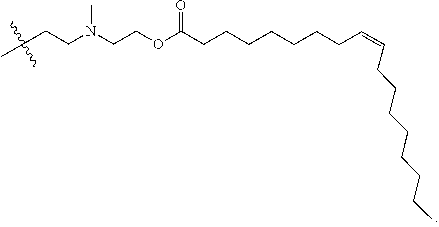 [SMILES: *CCN(C)CCOC(=O)CCCCCCC/C=C\CCCCCCCC]

Embodiment 102. The composition according to any of embodiments 1-80, wherein the A in the compound of Formula (I) is A″ and A″ is

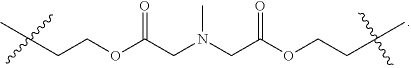 [SMILES: *CCOC(=O)CN(C)CC(=O)OCC*]

Embodiment 103. The composition of any of one of the preceding embodiments, wherein the compound of Formula (I) is:

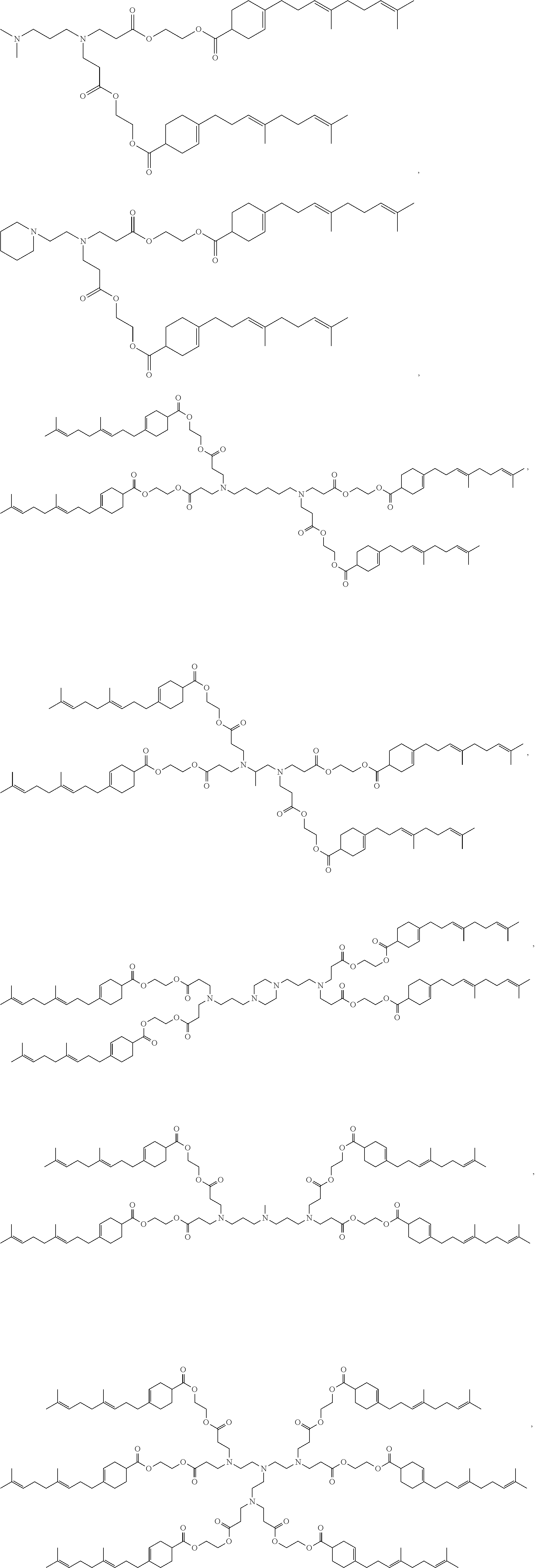

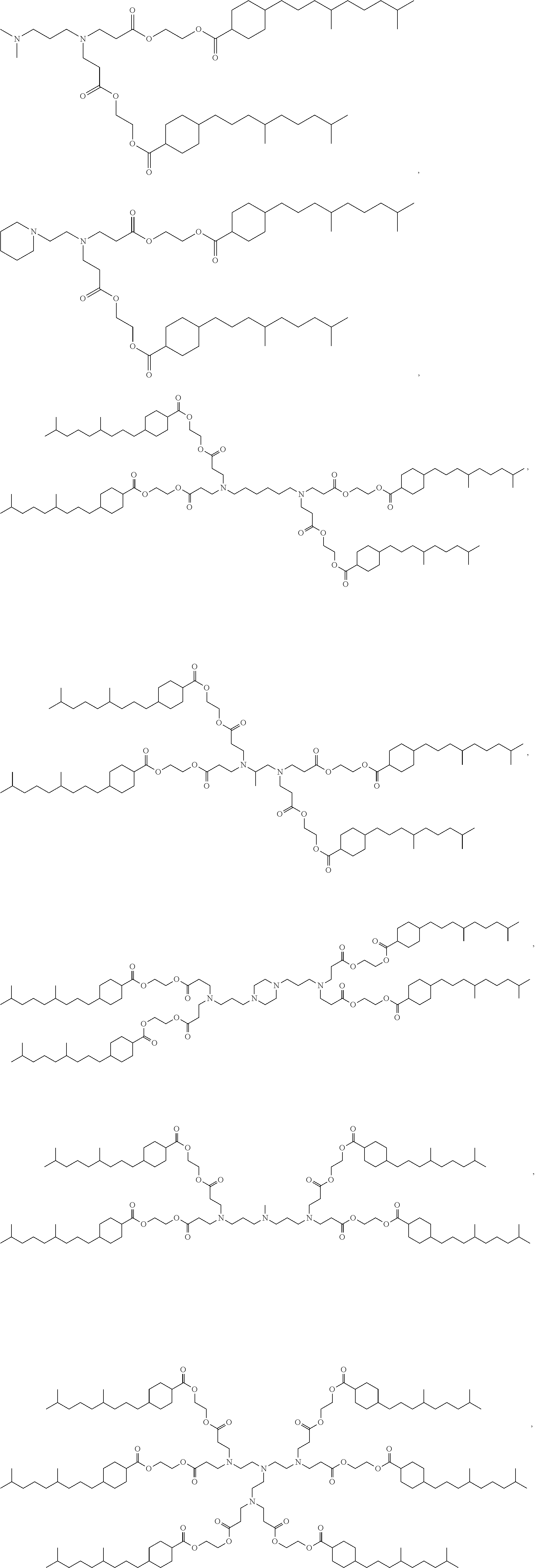

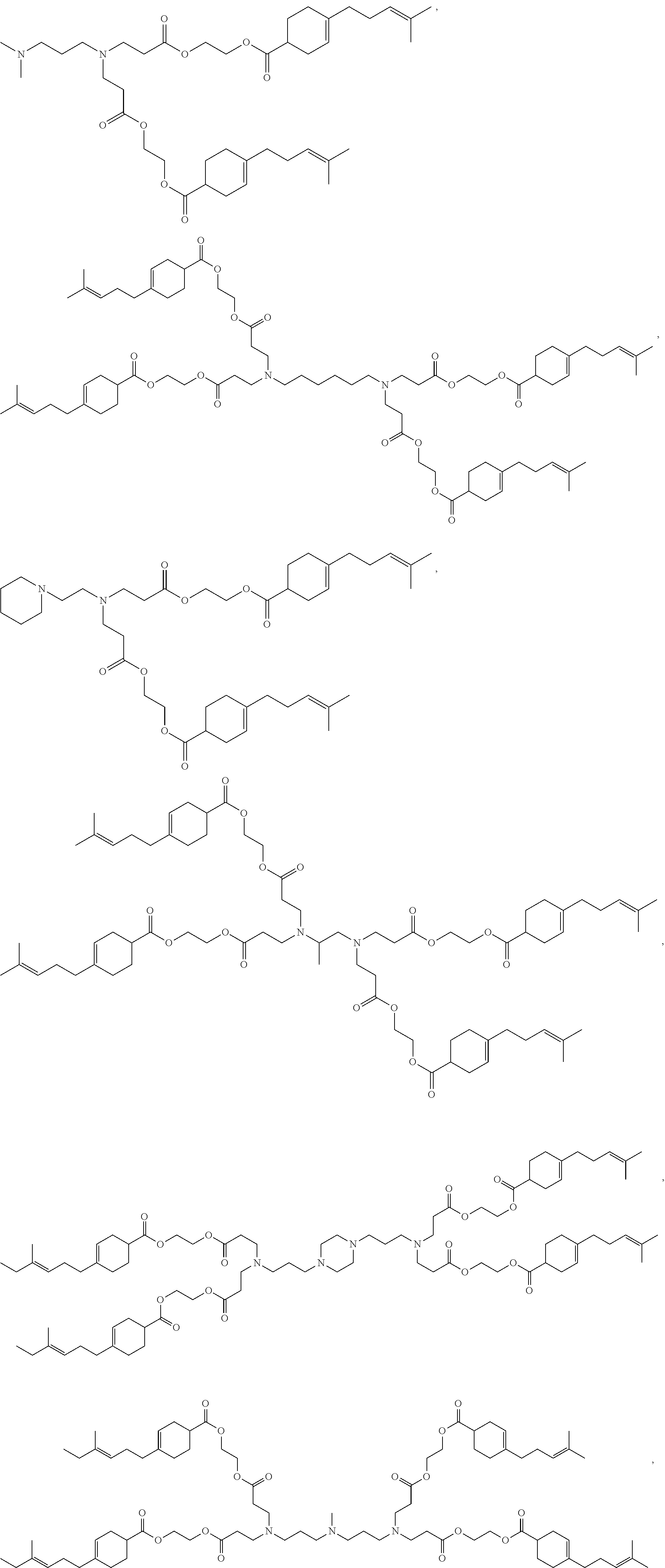

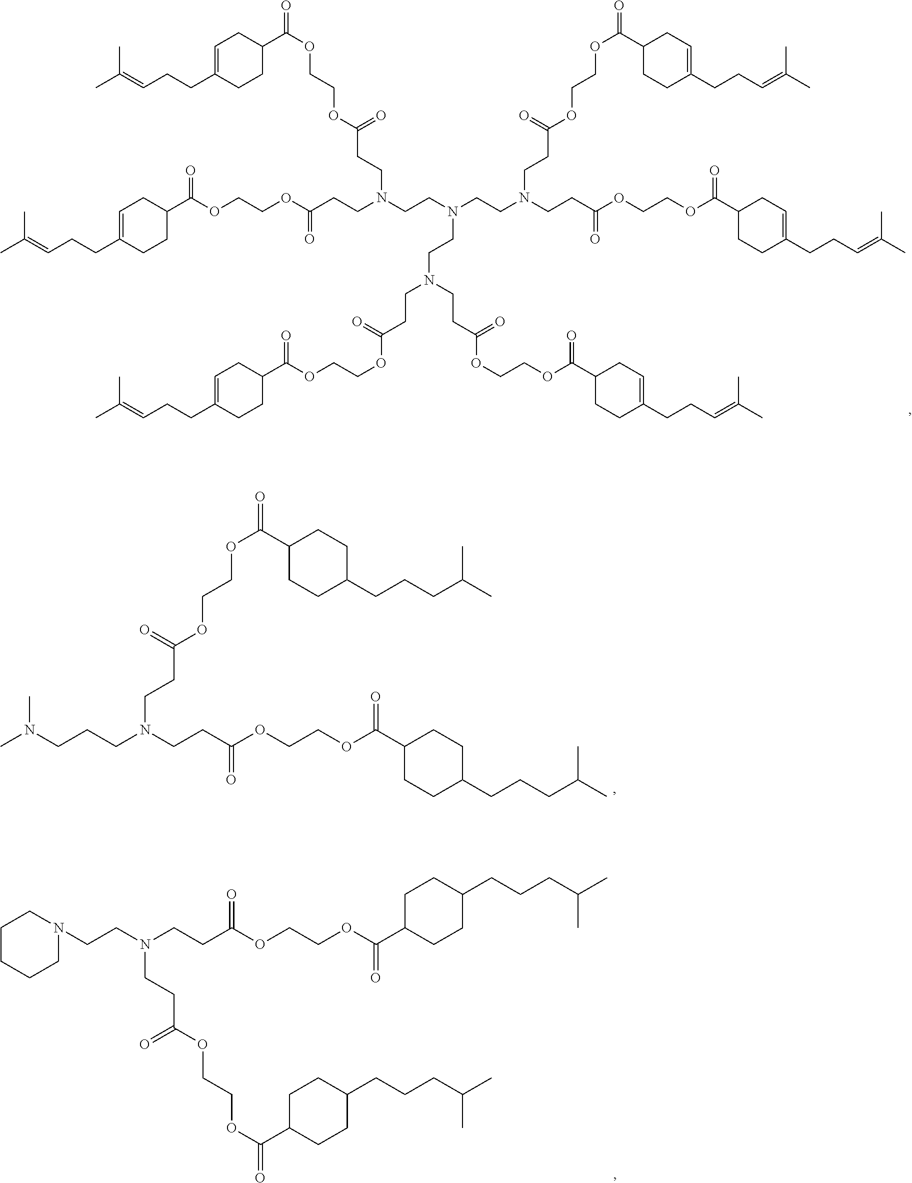

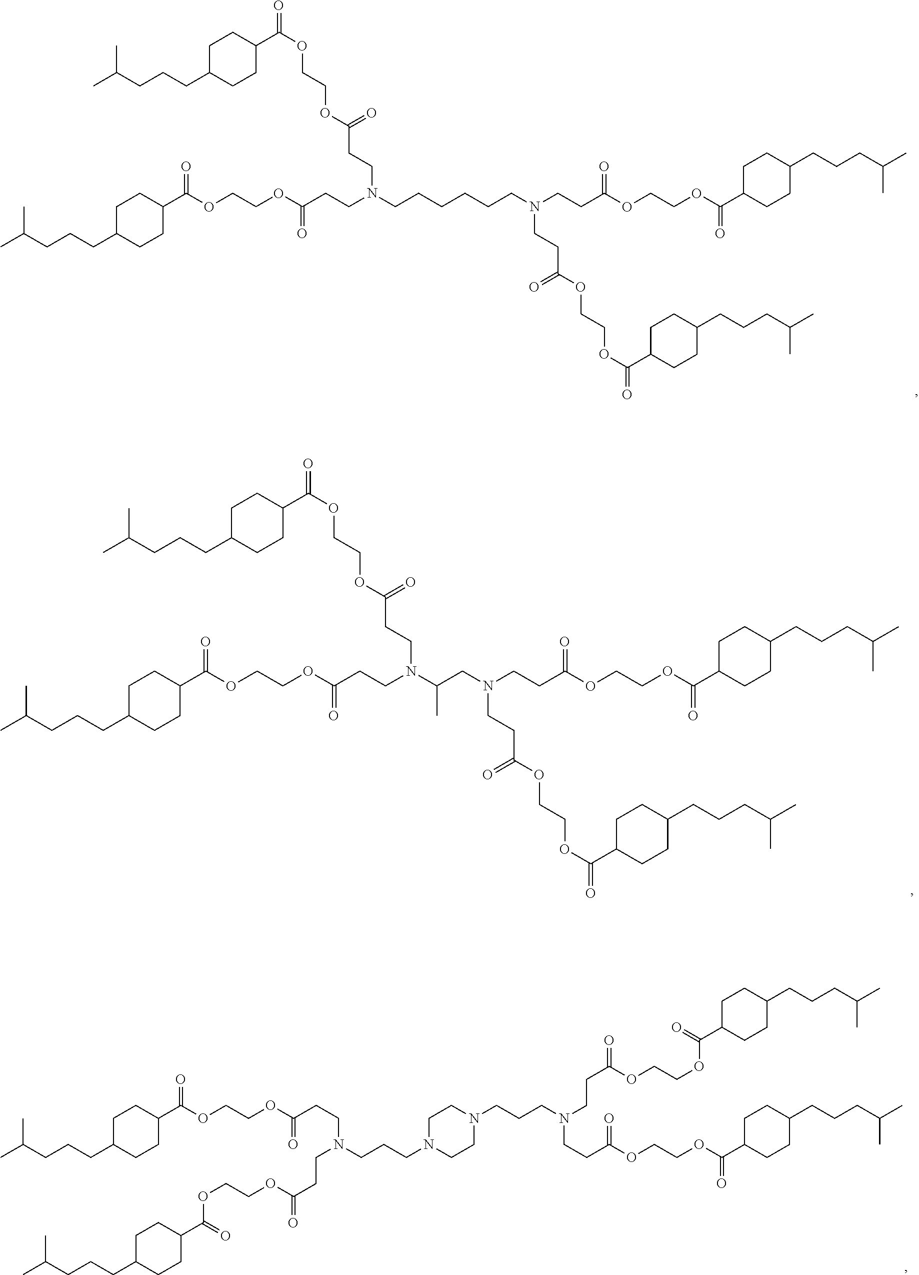

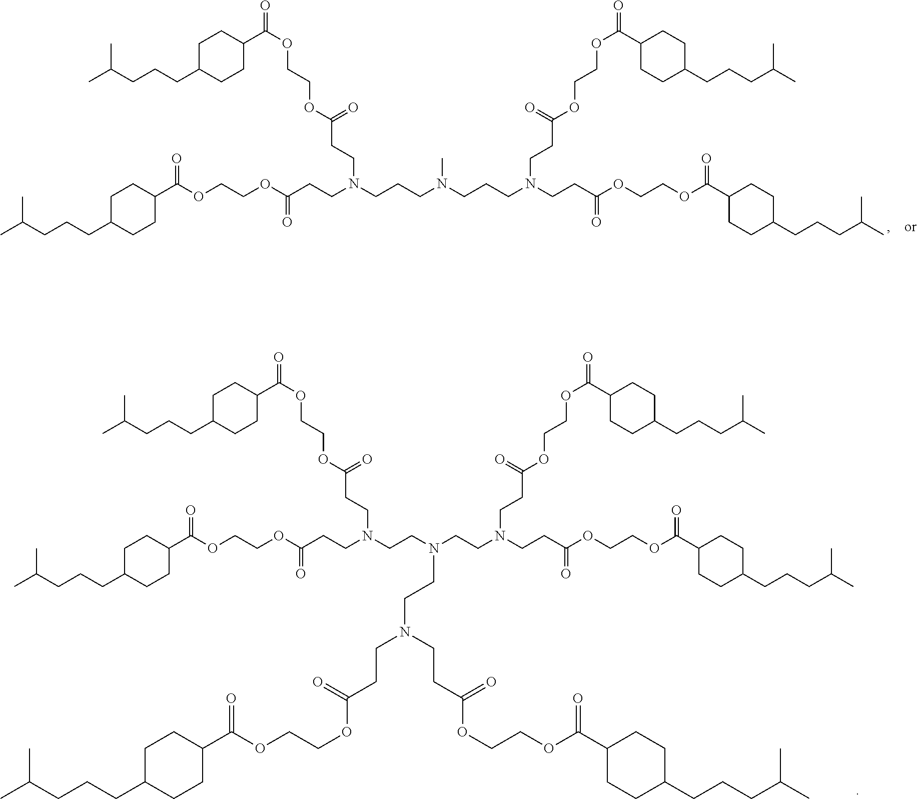

Embodiment 104. The composition of any of one of the preceding embodiments, wherein the compound of Formula (I) is:

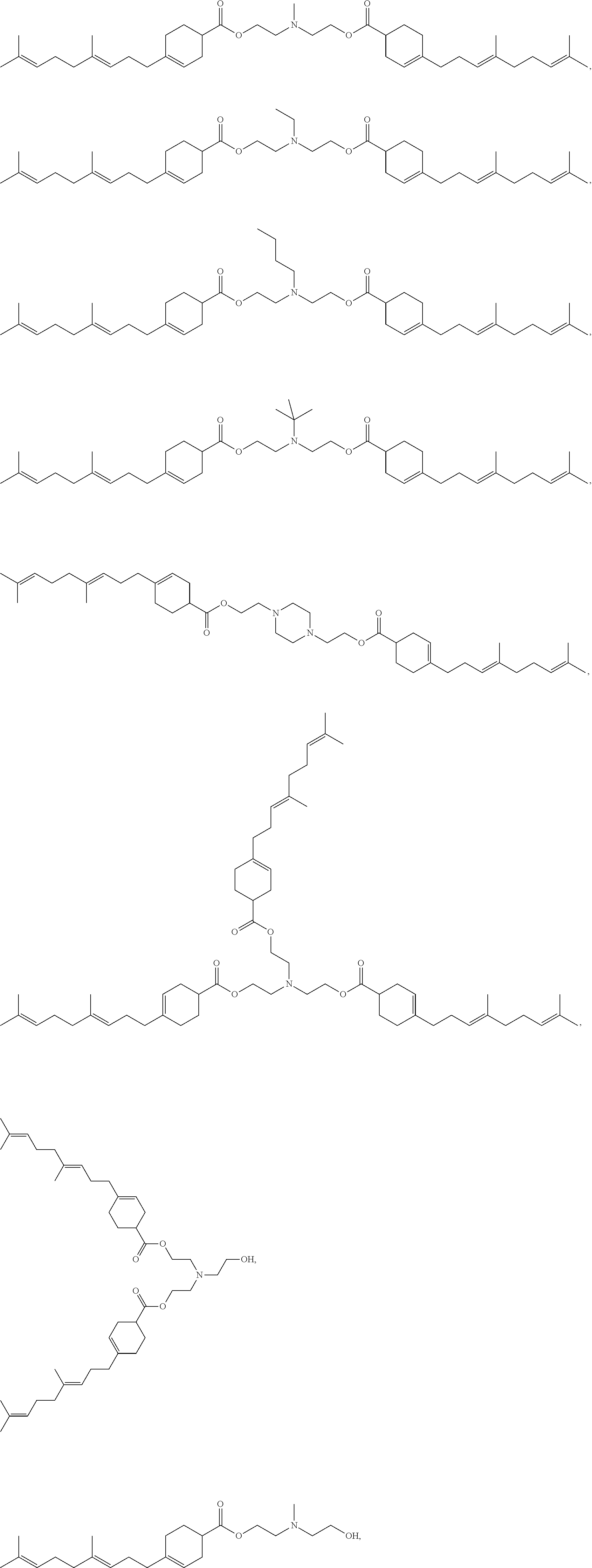

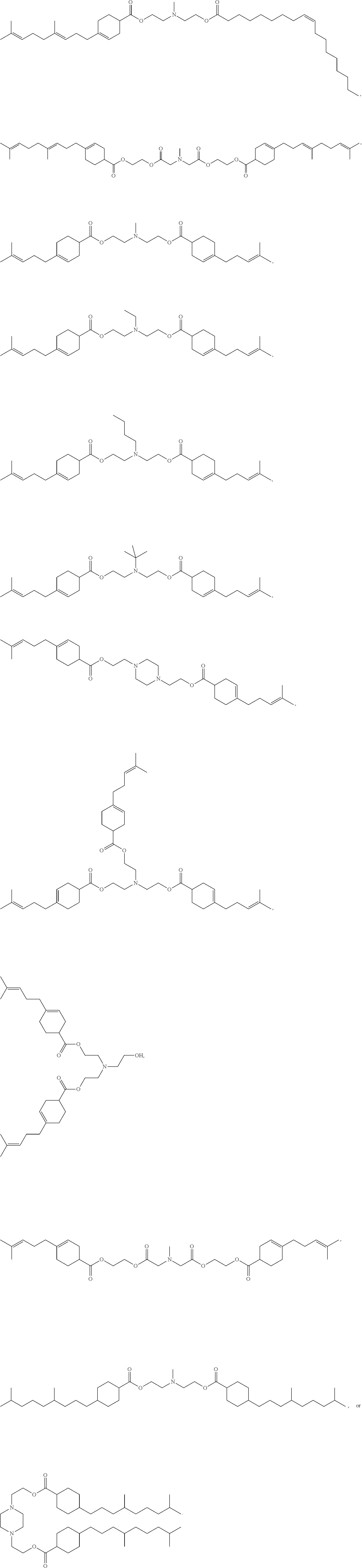

Embodiment 105. The composition according any one of the preceding embodiments, wherein the compound of Formula (II) or Formula (III) is:

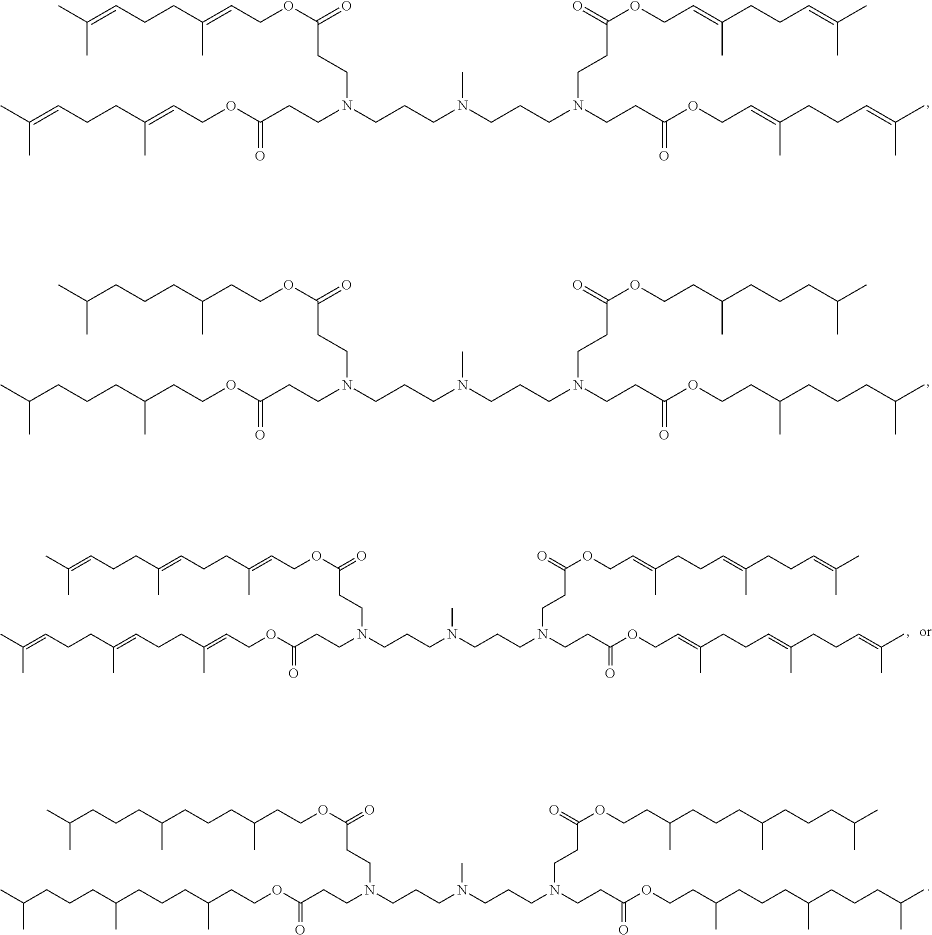

Embodiment 106. The composition of any one of the preceding embodiments, wherein the at least one compound of Formula (I) is any one of Compounds 5-49.

Embodiment 107. The composition of any one of the preceding embodiments, wherein the at least one compound of Formula (I) is:
Compound 9;
Compound 16;
Compound 17;
Compound 18;
Compound 22;
Compound 23;
Compound 29;
Compound 30;
Compound 42; or
Compound 55.

Embodiment 108. The composition of any one of the preceding embodiments, wherein the at least one compound of Formula (I) is Compound 30.

Embodiment 109. The composition of any one of the preceding embodiments, wherein the at least one nucleic acid molecule comprises at least one RNA molecule.

Embodiment 110. The composition of any one of the preceding embodiments, wherein the at least one nucleic acid molecule comprises at least one DNA molecule.

Embodiment 111. The composition of any one of the preceding embodiments, wherein the at least one nucleic acid molecule comprises at least one RNA molecule and at least one DNA molecule.

Embodiment 112. The composition of any one of the preceding embodiments, wherein the at least one RNA molecule comprises at least one mRNA molecule, at least one guide RNA molecule or any combination thereof.

Embodiment 113. The composition of any one of the preceding embodiments, wherein the at least one mRNA molecule comprises a 5′-CAP.

Embodiment 114. The composition of any one of the preceding embodiments, wherein the at least one mRNA molecule comprises at least one modified nucleic acid, preferably wherein the at least one modified nucleic acid comprise 5-methylcytidine (5-MeC)

Embodiment 115. The composition according of any one of the preceding embodiments, wherein the at least one RNA molecule comprises a nucleic acid sequence encoding at least one transposase, preferably wherein the transposase is a piggyBac™ (PB) transposase, a piggyBac-like (PBL) transposase, a Super piggyBac™ (SPB) transposase polypeptide, a Sleeping Beauty transposase, a Hyperactive Sleeping Beauty (SB100X) transposase, a helitron transposase, a Tol2 transposase, a TcBuster transposase or a mutant TcBuster transposase.

Embodiment 116. The composition of any one of the preceding embodiments, wherein the at least one DNA molecule comprises a circular DNA molecule, a DoggyBone DNA molecule, a DNA plasmid, a DNA nanoplasmid, or a linearized DNA molecule, preferably wherein the DNA molecule is a DoggyBone DNA molecule or a DNA nanoplasmid.

Embodiment 117. The composition of any one of the preceding embodiments, wherein the at least one DNA molecule comprises a nucleic acid sequence encoding at least one transposon.

Embodiment 118. The composition of any one of the preceding embodiments, wherein the at least one nucleic acid molecule comprises a nucleic acid sequence encoding at least one therapeutic protein.

Embodiment 119. The composition of any of the preceding embodiments, wherein the at least one nucleic acid molecule comprises a nucleic acid sequence encoding at least one transposon, wherein the transposon comprises a nucleic acid sequence encoding at least one therapeutic protein.

Embodiment 120. The composition of any one of the preceding embodiments, wherein the at least one therapeutic protein is:
(a) a chimeric antigen receptor (CAR);
(b) an ornithine transcarbamylase (OTC) polypeptide;
(c) a methylmalonyl-CoA mutase (MUT1) polypeptide;
(d) a Factor VIII (FVIII) polypeptide; or
(d) any combination thereof.

Embodiment 121. The composition of any one of the preceding embodiments, wherein the at least one RNA molecule comprises a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof.

Embodiment 122. A pharmaceutical composition, comprising a composition of any of the preceding embodiments and at least one pharmaceutically-acceptable excipient or diluent.

Embodiment 123. A method of delivering at least one nucleic acid to at least one cell comprising contacting the at least one cell with at least one composition of any of the preceding embodiments.

Embodiment 124. A method of genetically modifying at least one cell comprising contacting the at least one cell with at least one composition of any of the preceding embodiments.

Embodiment 125. The method of embodiment 123 or embodiment 123, wherein the at least one cell is:
(a) a liver cell, preferably wherein the liver cell is a hepatocyte, a hepatic stellate cell, Kupffer cell or liver sinusoidal endothelial cell;
(b) a T-cell, preferably wherein the T-cell is an activated T-cell, a resting T-cell or a stem memory T cell (T_{SCM} cell);
(c) is a hematopoietic stem cell (HSC).

Embodiment 126. At least one cell modified according to the method of any one of embodiments 123-125.

Embodiment 127. A method of treating at least one disease or disorder in a subject in need thereof comprising administering to the subject at least one therapeutically effective amount of the composition of any one of embodiments 1-122 or the at least one cell of embodiment 126.

Embodiment 128. The method of embodiment 127, wherein the at least one disease or disorder is a liver disease or disorder, preferably wherein the liver disease or disorder is:
(a) a metabolic liver disorder;
(b) a urea cycle disorder (UCD), preferably wherein the UCD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome) or any combination thereof.

Embodiment 129. The method of embodiment 128, wherein the at least one disease or disorder is cancer.

Embodiment 130. The method of embodiment 128, wherein the at least one disease or disorder is hemophilia A.

### EXAMPLES

### Protocol A Compounds

### Example 1—Preparation of Compound 1

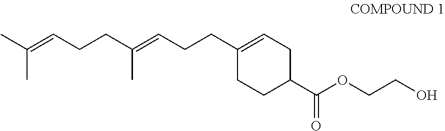 [SMILES: CC(C)=CCC/C(C)=C/CCC1=CCC(C(=O)OCCO)CC1]

### (E)-2-hydroxyethyl 4-(4,8-dimethylnona-3,7-dien-1-yl)cyclohex-3-enecarboxylate

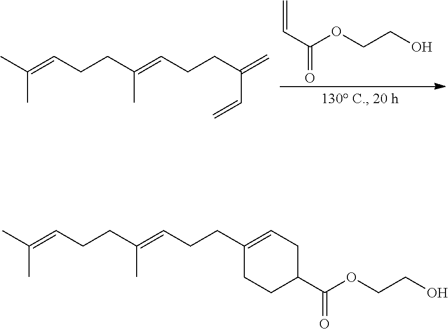

This compound was prepared as follows.

Trans-beta-farnesene (1.02 g, 4.99 mmol) and 2-hydroxyethyl acrylate (0.56 g, 4.99 mmol) were mixed in a glass tube and sealed. The reaction mixture was then stirred for 20 h at 130° C. The reaction mixture was cooled and purified by silica gel flash chromatography (eluent: 30% EtOAc/hexane).

Colorless oil; 1.31 g (86% yield). 1H NMR (500 MHZ, Chloroform-d) 8 5.42-5.31 (m, 1H), 5.12-5.00 (m, 2H), 4.23-4.16 (m, 2H), 3.83-3.76 (m, 2H), 2.63-2.32 (m, 2H), 2.26-1.90 (m, 13H), 1.82-1.49 (m, 10H).

### Example 2—Preparation of Compound 3

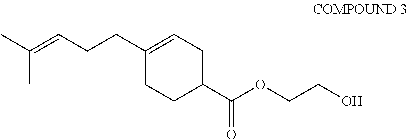 [SMILES: CC(C)=CCCC1=CCC(C(=O)OCCO)CC1]

### 2-hydroxyethyl 4-(4-methylpent-3-en-1-yl)cyclohex-3-enecarboxylate

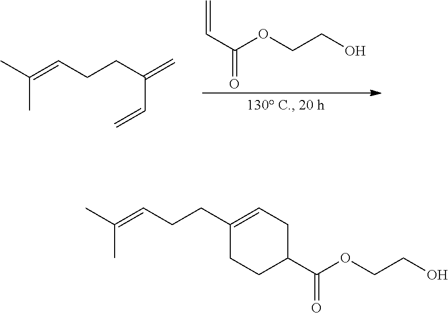

Myrcene (4.17 g, 30.6 mmol) and 2-hydroxyethyl acrylate (3.6 g, 30.61 mmol) were mixed in a glass tube and sealed. The reaction mixture was then stirred for 20 h at 130° C. The reaction mixture was cooled and purified by silica gel flash chromatography (eluent: 20% EtO Ac/hexane).

Colorless oil; 5.4 g (77%). 1H NMR (500 MHz, Chloroform-d) § 5.40-5.28 (m, 1H), 5.09-4.99 (m, 1H), 4.24-4.11 (m, 2H), 3.84-3.70 (m, 2H), 2.67-2.46 (m, 2H), 2.33-1.83 (m, 9H), 1.72-1.48 (m, 7H).

### Protocol B Compounds

### Example 3—Preparation of Compound 2

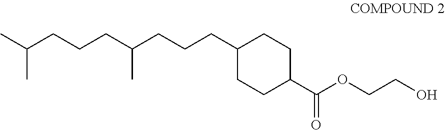 [SMILES: CC(C)CCCC(C)CCCC1CCC(C(=O)OCCO)CC1]

### 2-hydroxyethyl 4-(4,8-dimethylnonyl)cyclohexanecarboxylate

In a 100 mL round-bottom flask, compound 1 (2.1 g) was dissolved in a mixed solvent of ethanol and CH₂Cl₂ (4:1, 20 mL). To this solution, 10% Pd/C (210 mg) was added in one portion and the resulting dark suspension was stirred under a hydrogen atmosphere for 24 h at room temperature. The reaction mixture was filtered through celite and rinsed with CH₂Cl₂ (3×50 mL). The filtrate was evaporated to give compound 2 as a colorless oil.

Colorless oil; 2.06 g (97%). 5.4 g (77%). 1H NMR (500 MHz, Chloroform-d) δ 4.20-4.06 (m, 2H), 3.80-3.68 (m, 2H), 3.18-2.71 (m, 1H), 2.66-2.13 (m, 1H), 2.01-1.59 (m, 3H), 1.58-0.93 (m, 18H), 0.91-0.57 (m, 11H).

### Example 4—Preparation of Compound 4

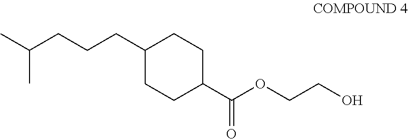 [SMILES: CC(C)CCCC1CCC(C(=O)OCCO)CC1]

### 2-hydroxyethyl 4-(4-methylpentyl)cyclohexanecarboxylate

In a 100 mL round-bottom flask, compound 3 (0.51 g) was dissolved in a mixed solvent of ethanol and CH₂Cl₂ (4:1, 20 mL). To this solution, 10% Pd/C (51 mg) was added in one portion and the resulting dark suspension was stirred under a hydrogen atmosphere for 24 h at room temperature. The reaction mixture was filtered through celite and rinsed with CH₂Cl₂ (3×50 mL). The filtrate was evaporated to give compound 4 as a colorless oil.

Colorless oil; 0.46 g (97%). 1H NMR (500 MHZ, Chloroform-d) 8 4.26-4.11 (m, 2H), 3.78 (s, 2H), 2.71-2.12 (m, 2H), 2.01-1.86 (m, 2H), 1.85-1.65 (m, 1H), 1.62-0.96 (m, 12H), 0.94-0.74 (m, 7H).

### Protocol C Compounds

### Example 5—Preparation of Compound FA

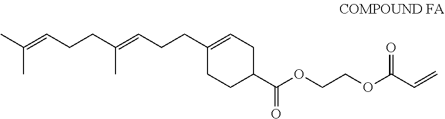 [SMILES: C=CC(=O)OCCOC(=O)C1CC=C(CC/C=C(\C)CCC=C(C)C)CC1]

### (E)-2-(acryloyloxy)ethyl 4-(4,8-dimethylnona-3,7-dien-1-yl)cyclohex-3-enecarboxylate

Compound 1 (112 mg, 0.35 mmol) was mixed with triethylamine (0.1 mL, 0.7 mmol) in anhydrous CH₂Cl₂ (5.0 mL) and the solution was cooled to 0 C with an ice/water bath. Acryloyl chloride (0.06 mL, 0.7 mmol) was added to the cooled solution dropwise over a period of 10 minutes. The resulting solution was stirred for 20 h allowing the temperature to rise to room temperature. Added saturated solution of sodium bicarbonate (20 mL) and CH₂Cl₂ (20 mL). Both layers were separated, the aqueous layer was extracted with CH₂Cl₂ (3×20 mL). Combined organic extracts were washed with brine (20 mL); dried over sodium sulfate; filtered; and evaporated on rota vapor. The crude was purified by silica gel flash chromatography (eluent: 15% EtOAc/hexane).

Colorless oil; 126 mg (97%). 1H NMR (500 MHz, Chloroform-d) δ 6.43 (dd, J=17.3, 1.6 Hz, 1H), 6.14 (ddd, J=17.3, 10.4, 1.7 Hz, 1H), 5.86 (dd, J=10.4, 1.5 Hz, 1H), 5.43-5.34 (m, 1H), 5.14-5.04 (m, 2H), 4.43-4.27 (m, 4H), 2.65-2.46 (m, 1H), 2.31-1.91 (m, 13H), 1.74-1.56 (m, 10H).

### Example 6—Preparation of Compound MA

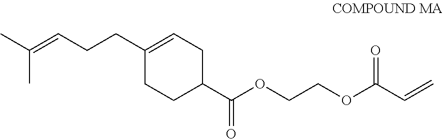 [SMILES: C=CC(=O)OCCOC(=O)C1CC=C(CCC=C(C)C)CC1]

### 2-(acryloyloxy)ethyl 4-(4-methylpent-3-en-1-yl)cyclohex-3-enecarboxylate

Compound 3 (0.46 g, 1.82 mmol) was mixed with triethylamine (0.76 mL, 3.6 mmol) in anhydrous CH₂Cl₂ (10.0 mL) and the solution was cooled to 0 C with an ice/water bath. Acryloyl chloride (0.45 mL, 3.6 mmol) was added to the cooled solution dropwise over a period of 10 minutes. The resulting solution was stirred for 20 h allowing the temperature to rise to room temperature. Added saturated solution of sodium bicarbonate (20 mL) and CH₂Cl₂ (20 mL). Both layers were separated, the aqueous layer was extracted with CH₂Cl₂ (3×20 mL). Combined organic extracts were washed with brine (20 mL); dried over sodium sulfate; filtered; and evaporated on rota vapor. The crude was purified by silica gel flash chromatography (eluent: 10% EtOAc/hexane).

Colorless oil; 417 mg (74%). 1H NMR (500 MHz, Chloroform-d) δ 6.48-6.37 (m, 1H), 6.19-6.07 (m, 1H), 5.86 (dd, J=10.4, 1.4 Hz, 1H), 5.44-5.33 (m, 1H), 5.15-5.03 (m, 1H), 4.42-4.27 (m, 4H), 2.65-2.48 (m, 1H), 2.33-1.90 (m, 9H), 1.78-1.54 (m, 7H).

### Example 7—Preparation of Compound HFA

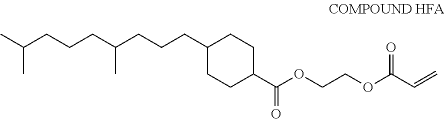 [SMILES: C=CC(=O)OCCOC(=O)C1CCC(CCCC(C)CCCC(C)C)CC1]

### 2-(acryloyloxy)ethyl 4-(4,8-dimethylnonyl)cyclohexanecarboxylate

Compound HFA was prepared in accordance with the General Procedure C. The crude was purified by silica gel flash chromatography (eluent: 15% EtOAc/hexane).

Colorless oil; ¹H NMR (500 MHz, Chloroform-d) δ 6.43 (dt, J=17.3, 1.3 Hz, 1H), 6.20-6.06 (m, 1H), 5.86 (ddd, J=10.6, 2.4, 1.5 Hz, 1H), 4.40-4.25 (m, 4H), 2.73-2.12 (m, 1H), 2.05-1.68 (m, 3H), 1.62-1.47 (m, 3H), 1.47-1.01 (m, 16H), 0.98-0.81 (m, 10H).

### Example 8—Preparation of Compound HMA

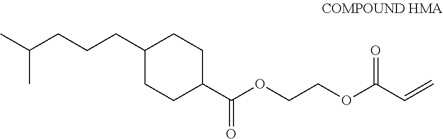 [SMILES: C=CC(=O)OCCOC(=O)C1CCC(CCCC(C)C)CC1]

### 2-(acryloyloxy)ethyl 4-(4-methylpentyl)cyclohexanecarboxylate

Compound HMA was prepared in accordance with the General Procedure C. The crude was purified by silica gel flash chromatography (eluent: 10% EtOAc/hexane).

Colorless oil; 1H NMR (500 MHz, Chloroform-d) § 6.42 (dd, J=17.3, 1.4 Hz, 1H), 6.13 (dd, J=17.3, 10.5 Hz, 1H), 5.85 (dt, J-10.4, 1.8 Hz, 1H), 4.41-4.26 (m, 4H), 2.71-2.15 (m, 1H), 2.03-1.86 (m, 2H), 1.85-1.60 (m, 2H), 1.59-0.96 (m, 11H), 0.95-0.69 (m, 7H).

### Protocol D Compounds

### Example 9—Preparation of Compound MA-404

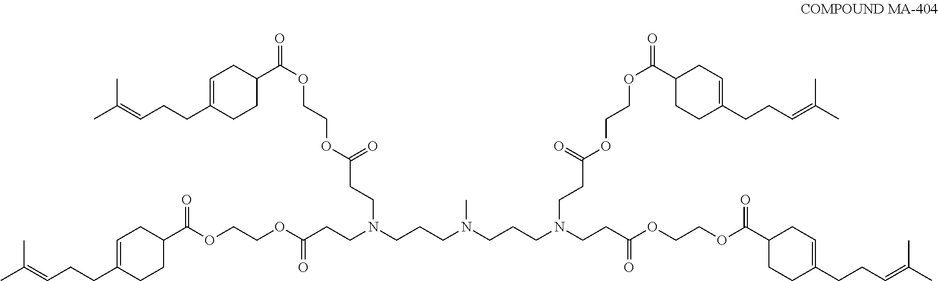 [SMILES: CC(C)=CCCC1=CCC(C(=O)OCCOC(=O)CCN(CCCN(C)CCCN(CCC(=O)OCCOC(=O)C2CC=C(CCC=C(C)C)CC2)CCC(=O)OCCOC(=O)C2CC=C(CCC=C(C)C)CC2)CCC(=O)OCCOC(=O)C2CC=C(CCC=C(C)C)CC2)CC1]

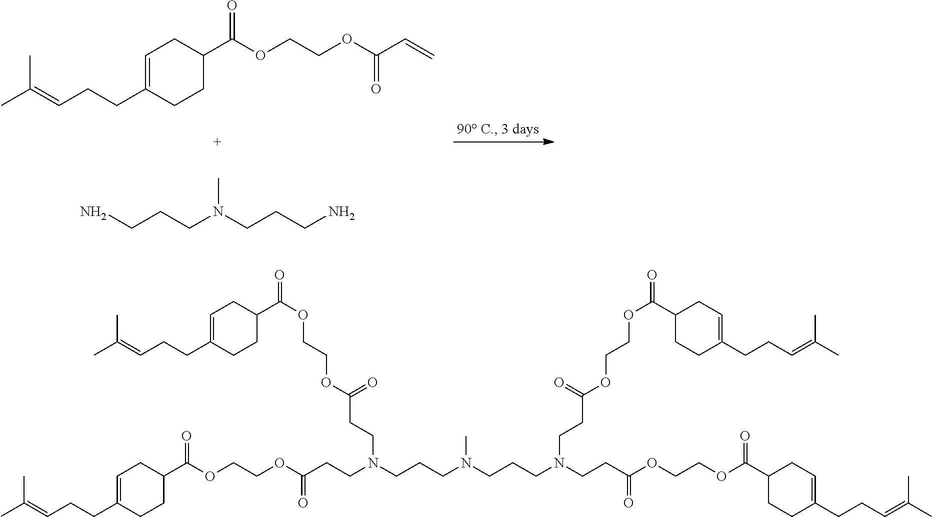

Compound MA (337 mg, 1.0 mmol) was mixed with amine 404 (28.8 mg, 0.2 mmol) in a 4-dram glass vial. The capped reaction vial was stirred at 90° C. for 3 days. The cooled reaction mixture was purified by silica gel flash chromatography (eluent: 5% MeOH/CH₂Cl₂).

Pale-yellow oil; 270 mg (99%). 1H NMR (400 MHz, Chloroform-d) δ 5.41-5.32 (m, 4H), 5.13-5.01 (m, 4H), 4.36-4.21 (m, 16H), 2.93-2.78 (m, 3H), 2.72 (t, J=6.8 Hz, 7H), 2.66-2.32 (m, 17H), 2.30-1.75 (m, 43H), 1.70-1.50 (m, 29H).

### Example 10—Preparation of Compound MA-201

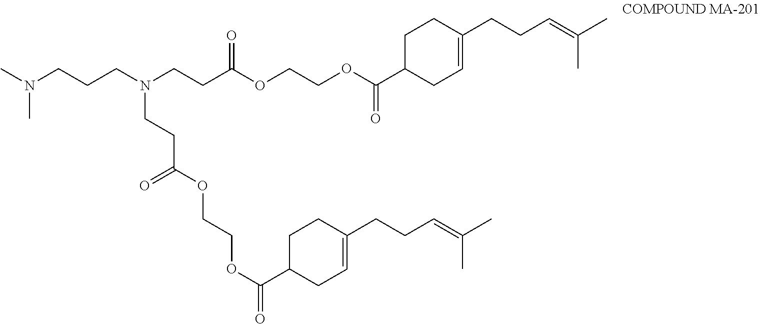 [SMILES: CC(C)=CCCC1=CCC(C(=O)OCCOC(=O)CCN(CCCN(C)C)CCC(=O)OCCOC(=O)C2CC=C(CCC=C(C)C)CC2)CC1]

### ((3,3′-((3-(dimethylamino) propyl) azanediyl)bis(propanoyl))bis(oxy))bis(ethane-2,1-diyl)bis(4-(4-methylpent-3-en-1-yl)cyclohex-3-enecarboxylate)

Compound MA-201 was prepared in accordance with the General Procedure D. The crude was purified by silica gel flash chromatography (eluent: 50% EtOAc/hexane). 1H NMR (400 MHz, Chloroform-d) δ 5.45-5.31 (m, 2H), 5.14-5.00 (m, 2H), 4.40-4.15 (m, 8H), 3.01-2.39 (m, 14H), 2.36-1.84 (m, 24H), 1.78-1.46 (m, 16H).

### Example 11—Preparation of Compound MA-202

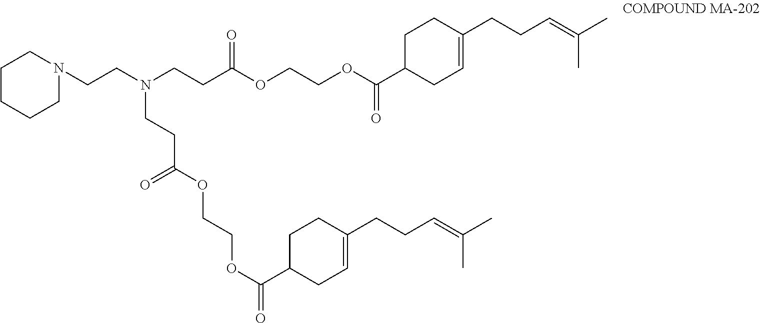 [SMILES: CC(C)=CCCC1=CCC(C(=O)OCCOC(=O)CCN(CCC(=O)OCCOC(=O)C2CC=C(CCC=C(C)C)CC2)CCN2CCCCC2)CC1]

### ((3,3′-((2-(piperidin-1-yl)ethyl) azanediyl)bis(propanoyl))bis(oxy)) bis(ethane-2,1-diyl)bis(4-(4-methylpent-3-en-1-yl)cyclohex-3-enecarboxylate)

Compound MA-202 may be prepared in accordance with the General Procedure D.

### Example 12—Preparation of Compound MA-401

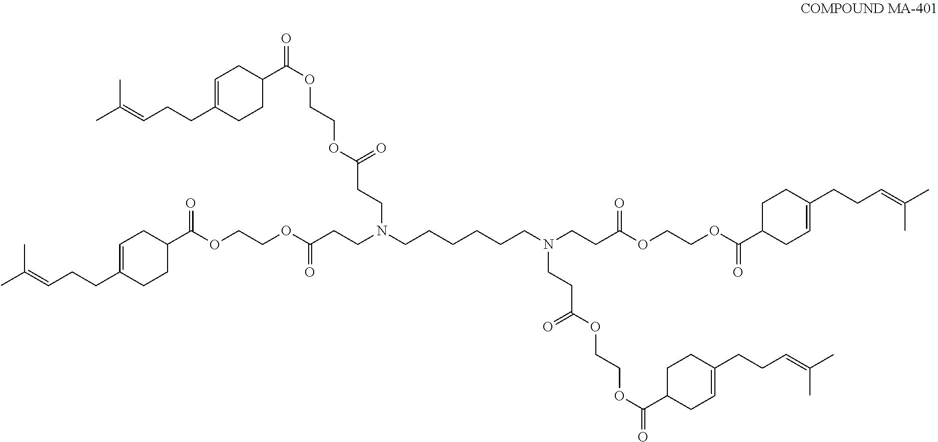 [SMILES: CC(C)=CCCC1=CCC(C(=O)OCCOC(=O)CCN(CCCCCCN(CCC(=O)OCCOC(=O)C2CC=C(CCC=C(C)C)CC2)CCC(=O)OCCOC(=O)C2CC=C(CCC=C(C)C)CC2)CCC(=O)OCCOC(=O)C2CC=C(CCC=C(C)C)CC2)CC1]

Compound MA-401 was prepared in accordance with the General Procedure D. The crude was purified by silica gel flash chromatography (eluent: 5% MeOH/DCM). 1H NMR (400 MHz, Chloroform-d) 0 5.41-5.32 (m, 4H), 5.10-5.02 (m, 4H), 4.40-4.18 (m, 16H), 3.04-2.67 (m, 6H), 2.64-2.34 (m, 14H), 2.27-1.87 (m, 40H), 1.67-1.20 (m, 36H).

### Example 13—Preparation of Compound MA-402

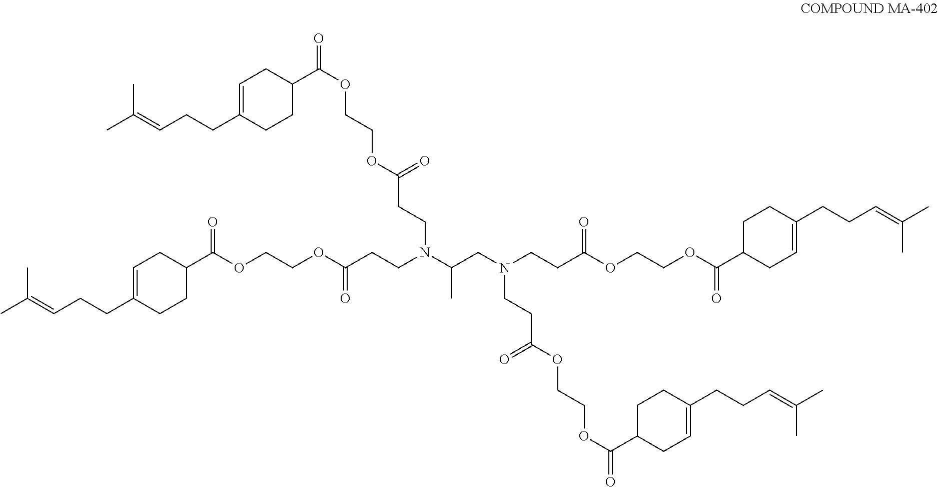 [SMILES: CC(C)=CCCC1=CCC(C(=O)OCCOC(=O)CCN(CCC(=O)OCCOC(=O)C2CC=C(CCC=C(C)C)CC2)CC(C)N(CCC(=O)OCCOC(=O)C2CC=C(CCC=C(C)C)CC2)CCC(=O)OCCOC(=O)C2CC=C(CCC=C(C)C)CC2)CC1]

Compound MA-402 was prepared in accordance with the General Procedure D. The crude was purified by silica gel flash chromatography (eluent: 5% MeOH/DCM). 1H NMR (400 MHz, Chloroform-d) § 5.47-5.28 (m, 4H), 5.14-4.99 (m, 4H), 4.33-4.20 (m, 16H), 2.87-2.35 (m, 20H), 2.26-1.88 (m, 42H), 1.69-1.54 (m, 28H).

### Example 14—Preparation of Compound MA-403

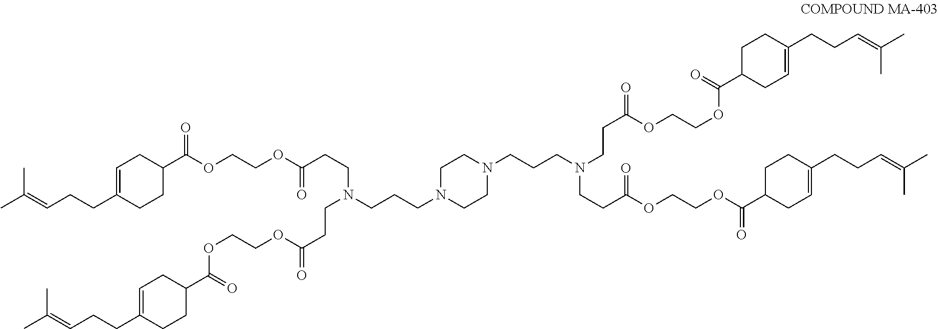 [SMILES: CC(C)=CCCC1=CCC(C(=O)OCCOC(=O)CCN(CCCN2CCN(CCCN(CCC(=O)OCCOC(=O)C3CC=C(CCC=C(C)C)CC3)CCC(=O)OCCOC(=O)C3CC=C(CCC=C(C)C)CC3)CC2)CCC(=O)OCCOC(=O)C2CC=C(CCC=C(C)C)CC2)CC1]

Compound MA-403 was prepared in accordance with the General Procedure D. The crude was purified by silica gel flash chromatography (eluent: 5% MeOH/DCM). 1H NMR (400 MHz, Chloroform-d) δ 5.41-5.28 (m, 4H), 5.11-4.95 (m, 4H), 4.36-4.17 (m, 16H), 2.71 (t, J=7.0 Hz, 8H), 2.61-2.26 (m, 24H), 2.23-1.76 (m, 40H), 1.69-1.46 (m, 32H).

### Example 15—Preparation of Compound MA-601

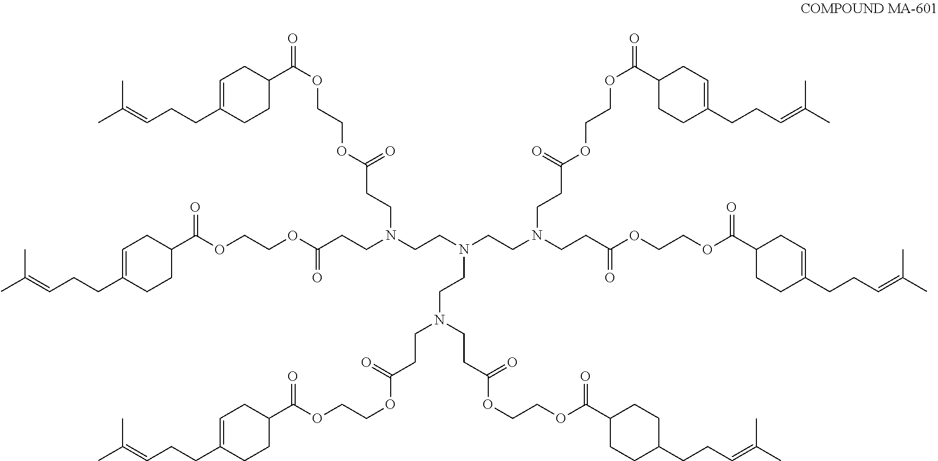 [SMILES: CC(C)=CCCC1=CCC(C(=O)OCCOC(=O)CCN(CCC(=O)OCCOC(=O)C2CC=C(CCC=C(C)C)CC2)CCN(CCN(CCC(=O)OCCOC(=O)C2CC=C(CCC=C(C)C)CC2)CCC(=O)OCCOC(=O)C2CC=C(CCC=C(C)C)CC2)CCN(CCC(=O)OCCOC(=O)C2CC=C(CCC=C(C)C)CC2)CCC(=O)OCCOC(=O)C2CCC(CCC=C(C)C)CC2)CC1]

Compound MA-601 was prepared in accordance with the General Procedure D. The crude was purified by silica gel flash chromatography (eluent: 10% MeOH/DCM). 1H NMR (400 MHZ, Chloroform-d) 8 5.43-5.29 (m, 6H), 5.14-4.97 (m, 6H), 4.37-4.12 (m, 24H), 2.75 (t, J=9.6, 4.5 Hz, 12H), 2.67-2.32 (m, 24H), 2.29-1.76 (m, 58H), 1.69-1.46 (m, 44H).

### Example 16—Preparation of Compound HMA-201

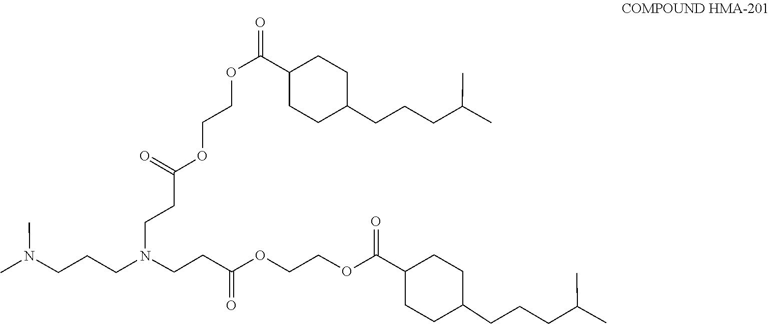 [SMILES: CC(C)CCCC1CCC(C(=O)OCCOC(=O)CCN(CCCN(C)C)CCC(=O)OCCOC(=O)C2CCC(CCCC(C)C)CC2)CC1]

Compound HMA-201 was prepared in accordance with the General Procedure D. The crude was purified by silica gel flash chromatography (eluent: 50% EtOAc/hexane). 1H NMR (400 MHZ, Chloroform-d) 8 4.37-4.19 (m, 8H), 2.83-2.75 (m, 3H), 2.75-2.62 (m, 3H), 2.50-2.40 (m, 2H), 2.37-2.15 (m, 2H), 2.04-1.67 (m, 10H), 1.60-1.33 (m, 8H), 1.29-1.07 (m, 20H), 0.87-0.77 (m, 18H).

### Example 17—Preparation of Compound HMA-202

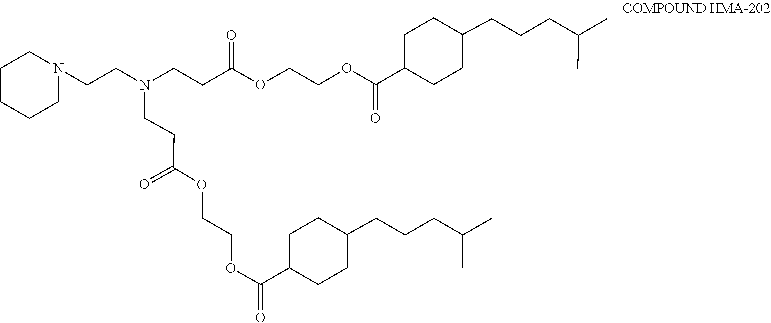 [SMILES: CC(C)CCCC1CCC(C(=O)OCCOC(=O)CCN(CCC(=O)OCCOC(=O)C2CCC(CCCC(C)C)CC2)CCN2CCCCC2)CC1]

Compound HMA-202 was prepared in accordance with the General Procedure D. The crude was purified by silica gel flash chromatography (eluent: 30% EtOAc/hexane). 1H NMR (400 MHZ, Chloroform-d) 8 4.33-4.16 (m, 8H), 3.21-2.89 (m, 6H), 2.75 (t, J=6.5 Hz, 4H), 2.54-2.43 (m, 4H), 2.37-2.14 (m, 2H), 2.09-1.73 (m, 10H), 1.69-0.96 (m, 28H), 0.83 (dd, J=6.6, 1.1 Hz, 16H).

### Example 18—Preparation of Compound HMA-401

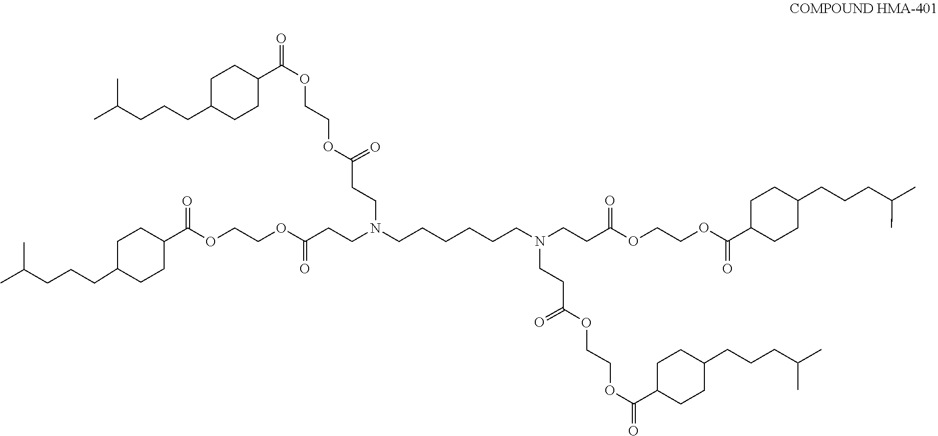 [SMILES: CC(C)CCCC1CCC(C(=O)OCCOC(=O)CCN(CCCCCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

Compound HMA-401 was prepared in accordance with the General Procedure D. The crude was purified by silica gel flash chromatography (eluent: 5% MeOH/DCM). 1H NMR (400 MHz, Chloroform-d) δ 4.36-4.14 (m, 16H), 2.75 (t, J=7.3 Hz, 6H), 2.66-2.14 (m, 16H), 1.98-1.68 (m, 14H), 1.58-1.05 (m, 54H), 0.83 (dd, J=6.7, 1.3 Hz, 30H).

### Example 19—Preparation of Compound HMA-402

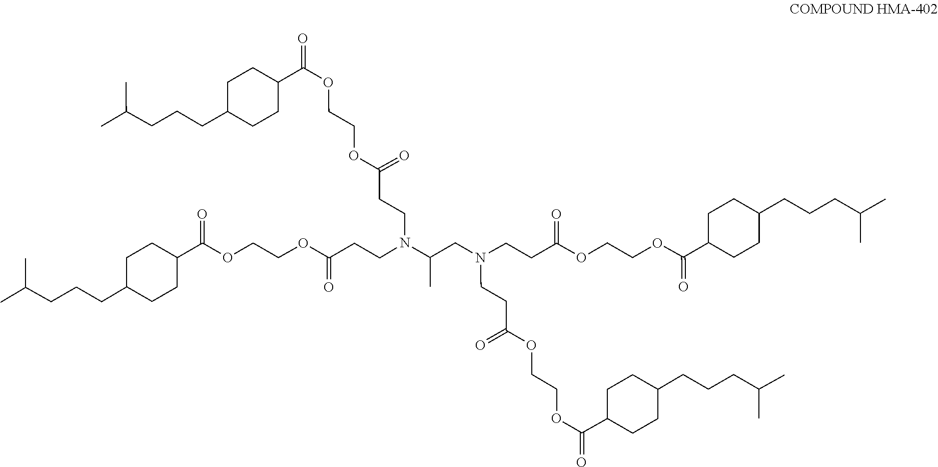 [SMILES: CC(C)CCCC1CCC(C(=O)OCCOC(=O)CCN(CCC(=O)OCCOC(=O)C2CCC(CCCC(C)C)CC2)CC(C)N(CCC(=O)OCCOC(=O)C2CCC(CCCC(C)C)CC2)CCC(=O)OCCOC(=O)C2CCC(CCCC(C)C)CC2)CC1]

Compound HMA-402 was prepared in accordance with the General Procedure D. The crude was purified by silica gel flash chromatography (eluent: 5% MeOH/DCM). 1H NMR (400 MHz, Chloroform-d) δ 4.40-4.15 (m, 16H), 2.97-2.40 (m, 12H), 2.39-2.15 (m, 5H), 2.03-1.67 (m, 15H), 1.63-0.98 (m, 51H), 0.91-0.74 (m, 31H).

### Example 20—Preparation of Compound HMA-403

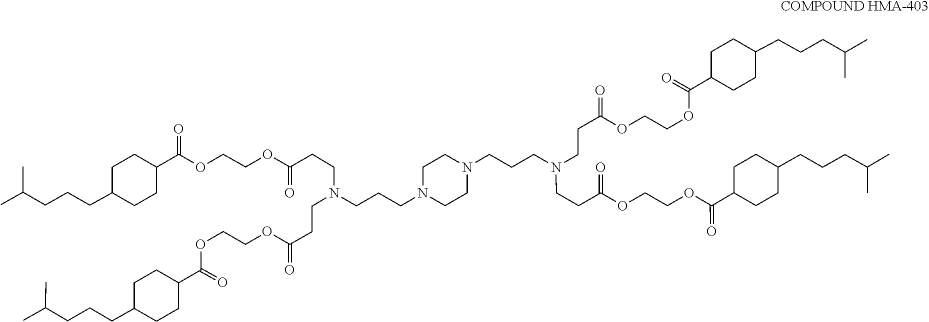

Compound HMA-403 was prepared in accordance with the General Procedure D. The crude was purified by silica gel flash chromatography (eluent: 5% MeOH/DCM). 1H NMR (400 MHz, Chloroform-d) δ 4.35-4.15 (m, 16H), 2.71 (t, J=7.1 Hz, 8H), 2.62-2.05 (m, 26H), 1.97-0.95 (m, 62H), 0.93-0.61 (m, 32H).

### Example 21—Preparation of Compound HMA-404

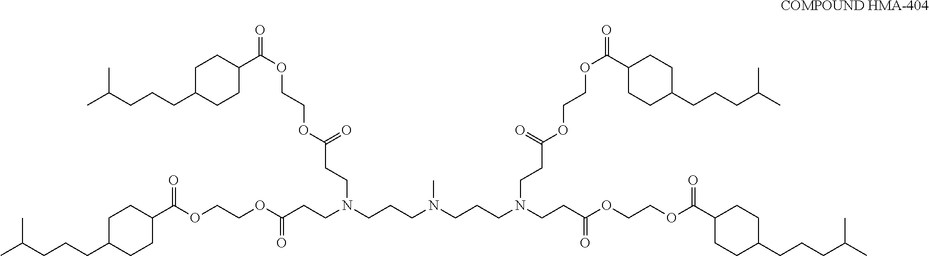

Compound HMA-404 was prepared in accordance with the General Procedure D. The crude was purified by silica gel flash chromatography (eluent: 5% MeOH/DCM). 1H NMR (400 MHz, Chloroform-d) § 4.38-4.12 (m, 16H), 2.90-2.78 (m, 4H), 2.71 (t, J=6.8 Hz, 8H), 2.61 (s, 3H), 2.57-2.36 (m, 12H), 2.35-2.04 (m, 4H), 2.01-1.62 (m, 17H), 1.60-0.96 (m, 45H), 0.94-0.67 (m, 30H).

### Example 22—Preparation of Compound HMA-601

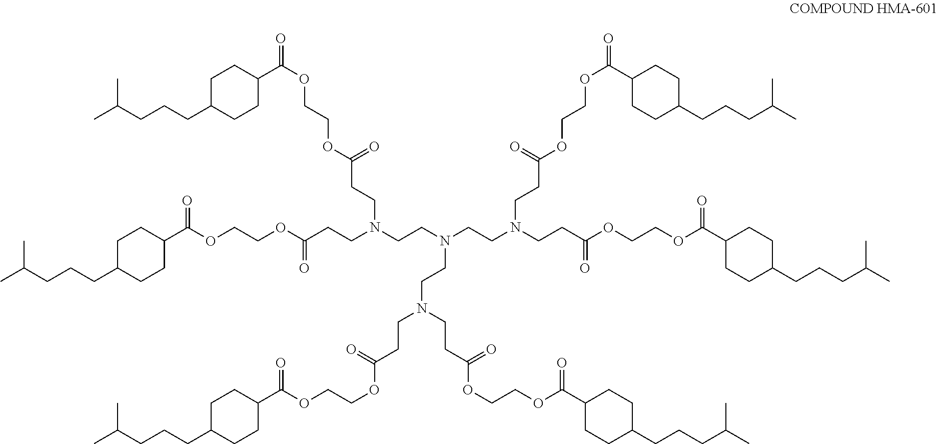 [SMILES: CC(C)CCCC1CCC(C(=O)OCCOC(=O)CCN(CCC(=O)OCCOC(=O)C2CCC(CCCC(C)C)CC2)CCN(CCN(CCC(=O)OCCOC(=O)C2CCC(CCCC(C)C)CC2)CCC(=O)OCCOC(=O)C2CCC(CCCC(C)C)CC2)CCN(CCC(=O)OCCOC(=O)C2CCC(CCCC(C)C)CC2)CCC(=O)OCCOC(=O)C2CCC(CCCC(C)C)CC2)CC1]

Compound HMA-601 was prepared in accordance with the General Procedure D. The crude was purified by silica gel flash chromatography (eluent: 10% MeOH/DCM). 1H NMR (400 MHz, Chloroform-d) ô 4.36-4.13 (m, 24H), 2.76 (t, J=7.1 Hz, 12H), 2.66-2.37 (m, 24H), 2.35-2.13 (m, 6H), 1.98-1.65 (m, 22H), 1.58-1.00 (m, 68H), 0.83 (dd, J=6.6, 1.1 Hz, 42H).

### Example 23—Preparation of Compound FA-201

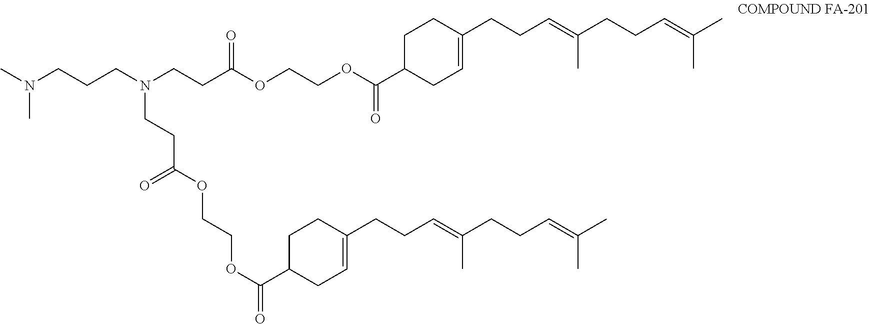 [SMILES: CC(C)=CCC/C(C)=C/CCC1=CCC(C(=O)OCCOC(=O)CCN(CCCN(C)C)CCC(=O)OCCOC(=O)C2CC=C(CC/C=C(\C)CCC=C(C)C)CC2)CC1]

### (E)-((3,3′-((3-(dimethylamino) propyl) azanediyl)bis(propanoyl))bis(oxy)) bis(ethane-2,1-diyl)bis(4-((E)-4,8-dimethylnona-3,7-dien-1-yl)cyclohex-3-enecarboxylate)

Compound FA-201 was prepared in accordance with the General Procedure D. The crude was purified by silica gel flash chromatography (eluent: 50% EtOAc/hexane). ¹H NMR (400 MHz, Chloroform-d) δ 5.43-5.30 (m, 2H), 5.17-4.95 (m, 4H), 4.38-4.15 (m, 8H), 3.93-3.75 (m, 1H), 3.64-3.03 (m, 2H), 2.97-2.79 (m, 2H), 2.76-2.62 (m, 8H), 2.59-2.31 (m, 6H), 2.29-1.76 (m, 28H), 1.75-1.35 (m, 21H).

### Example 24—Preparation of Compound FA-202

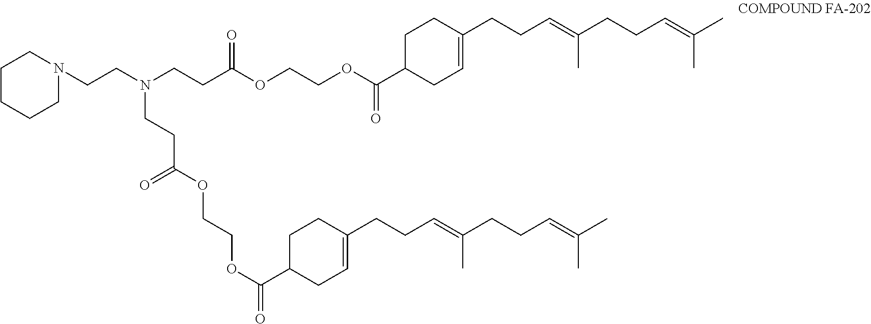 [SMILES: CC(C)=CCC/C(C)=C/CCC1=CCC(C(=O)OCCOC(=O)CCN(CCC(=O)OCCOC(=O)C2CC=C(CC/C=C(\C)CCC=C(C)C)CC2)CCN2CCCCC2)CC1]

### (E)-((3,3′-((2-(piperidin-1-yl)ethyl) azanediyl)bis(propanoyl))bis(oxy)) bis(ethane-2,1-diyl)bis(4-((E)-4,8-dimethylnona-3,7-dien-1-yl)cyclohex-3-enecarboxylate)

Compound FA-202 was prepared in accordance with the General Procedure D. The crude was purified by silica gel flash chromatography (eluent: 30% EtOAc/hexane). 1H NMR (400 MHZ, Chloroform-d) 8 5.45-5.31 (m, 2H), 5.16-5.01 (m, 4H), 4.26 (dt, J=5.1, 3.0 Hz, 8H), 3.04 (dt, J=10.5, 5.3 Hz, 6H), 2.77 (t, J=6.5 Hz, 4H), 2.50 (t, J=6.4 Hz, 6H), 2.31-1.75 (m, 32H), 1.74-1.28 (m, 24H).

### Example 25—Preparation of Compound FA-401

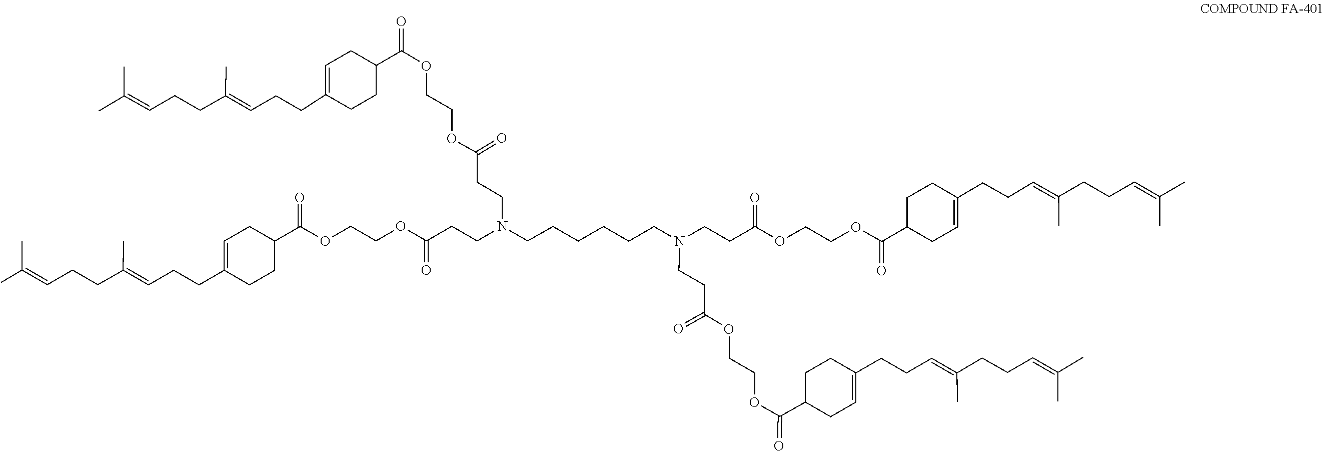 [SMILES: CC(C)=CCC/C(C)=C/CCC1=CCC(C(=O)OCCOC(=O)CCN(CCCCCCN(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CC1]

Compound FA-401 was prepared in accordance with the General Procedure D. The crude was purified by silica gel flash chromatography (eluent: 5% MeOH/DCM). 1H NMR (400 MHz, Chloroform-d) δ 5.44-5.31 (m, 4H), 5.15-5.02 (m, 8H), 4.27 (s, 16H), 2.76 (t, J=7.3 Hz, 6H), 2.66-2.35 (m, 15H), 2.31-1.86 (m, 55H), 1.69-1.18 (m, 48H).

### Example 26—Preparation of Compound FA-402

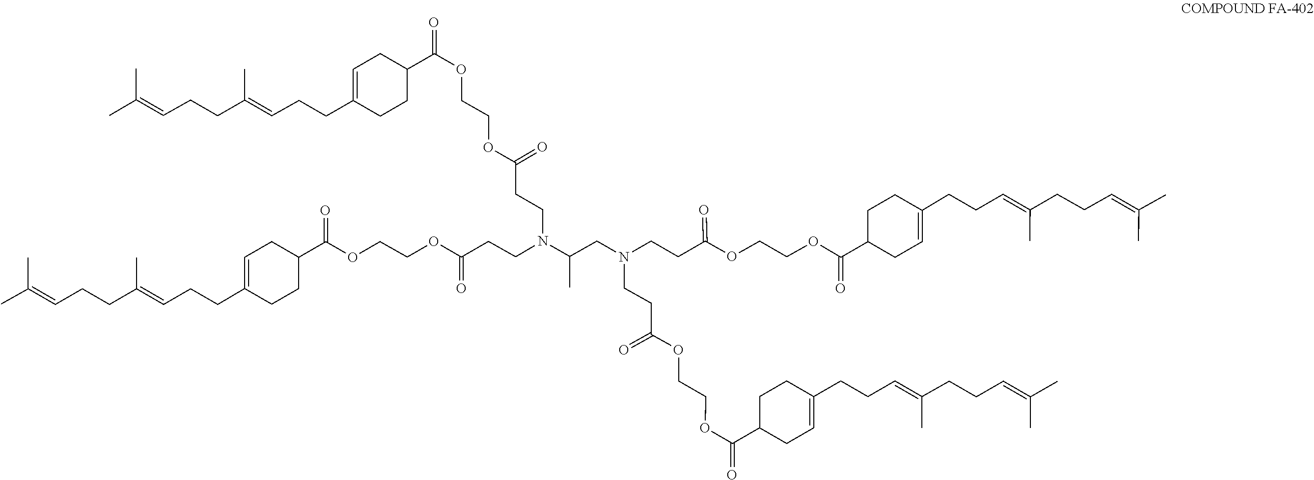 [SMILES: CC(C)=CCC/C(C)=C/CCC1=CCC(C(=O)OCCOC(=O)CCN(CCC(=O)OCCOC(=O)C2CC=C(CC/C=C(\C)CCC=C(C)C)CC2)CC(C)N(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CC1]

Compound FA-402 was prepared in accordance with the General Procedure D. The crude was purified by silica gel flash chromatography (eluent: 5% MeOH/DCM). 1H NMR (400 MHz, Chloroform-d) δ 5.45-5.31 (m, 4H), 5.16-5.01 (m, 8H), 4.43-4.15 (m, 16H), 3.03-2.31 (m, 21H), 2.30-1.78 (m, 56H), 1.77-1.46 (m, 41H).

### Example 27—Preparation of Compound FA-403

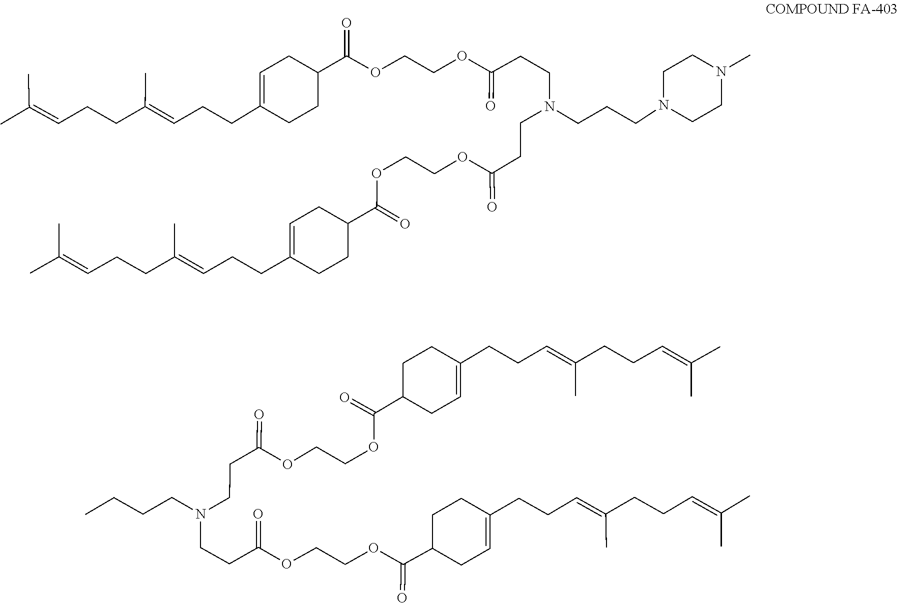

### ((3,3′,3″,3′″-((piperazine-1,4-diylbis(propane-3,1-diyl))bis(azanetriyl))tetrakis(propanoyl))tetrakis(oxy))tetrakis(ethane-2,1-diyl)tetrakis(4-((E)-4,8-dimethylnona-3,7-dien-1-yl)cyclohex-3-enecarboxylate)

Compound FA-403 was prepared in accordance with the General Procedure D. The crude was purified by silica gel flash chromatography (eluent: 5% MeOH/DCM). 1H NMR (400 MHz, Chloroform-d) δ 5.44-5.30 (m, 4H), 5.15-4.98 (m, 8H), 4.26 (s, 16H), 2.73 (t, J=7.0 Hz, 8H), 2.68-2.29 (m, 25H), 2.28-1.81 (m, 55H), 1.80-1.44 (m, 44H).

### Example 28—Preparation of Compound FA-404

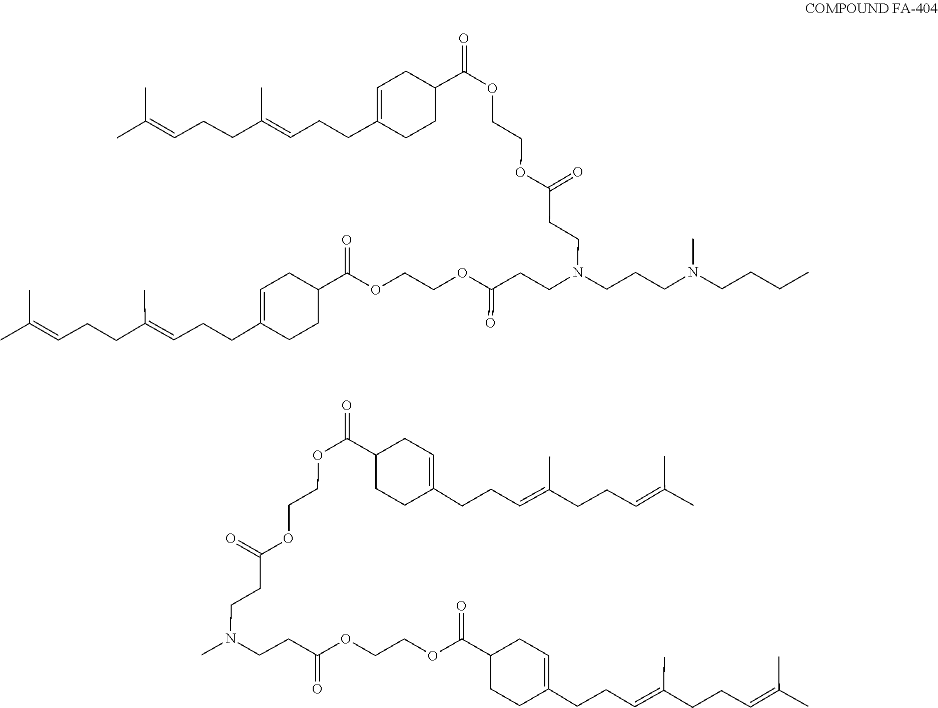

Compound FA-404 was prepared in accordance with the General Procedure D. The crude was purified by silica gel flash chromatography (eluent: 5% MeOH/DCM). 1H NMR (400 MHz, Chloroform-d) δ 5.47-5.29 (m, 4H), 5.15-5.01 (m, 8H), 4.39-4.15 (m, 16H), 2.97-2.64 (m, 8H), 2.63-2.34 (m, 14H), 2.32-1.74 (m, 61H), 1.72-1.31 (m, 44H).

### Example 29—Preparation of Compound FA-601

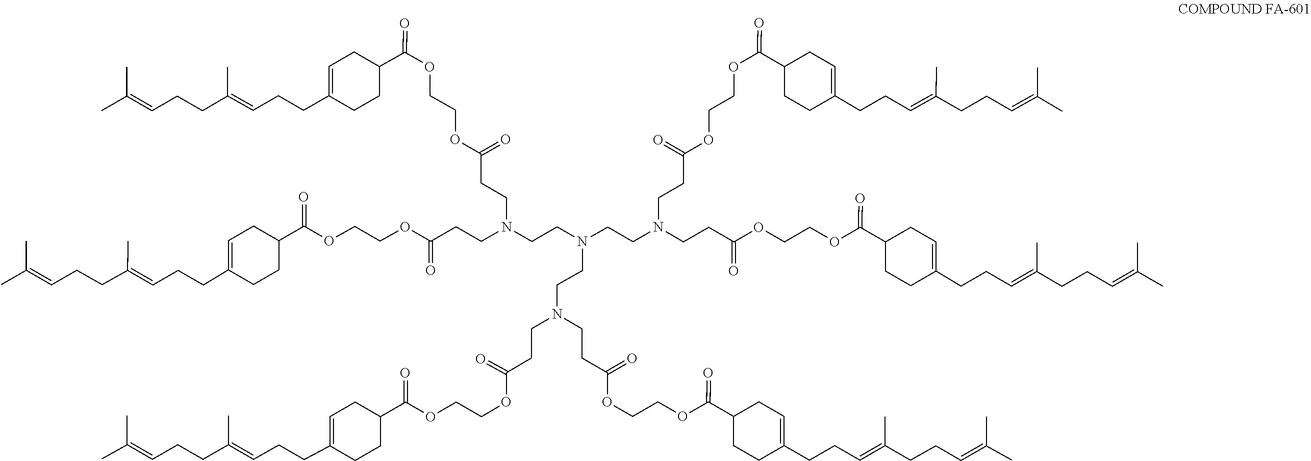 [SMILES: CC(C)=CCC/C(C)=C/CCC1=CCC(C(=O)OCCOC(=O)CCN(CCC(=O)OCCOC(=O)C2CC=C(CC/C=C(\C)CCC=C(C)C)CC2)CCN(CCN(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CCN(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CC1]

Compound FA-601 was prepared in accordance with the General Procedure D. The crude was purified by silica gel flash chromatography (eluent: 10% MeOH/DCM). 1H NMR (400 MHz, Chloroform-d) δ 5.45-5.30 (m, 6H), 5.15-4.97 (m, 12H), 4.26 (s, 24H), 2.77 (t, J=7.1 Hz, 12H), 2.69-2.30 (m, 27H), 2.28-1.77 (m, 80H), 1.74-1.28 (m, 61H).

### Example 30—Preparation of Compound HFA-201

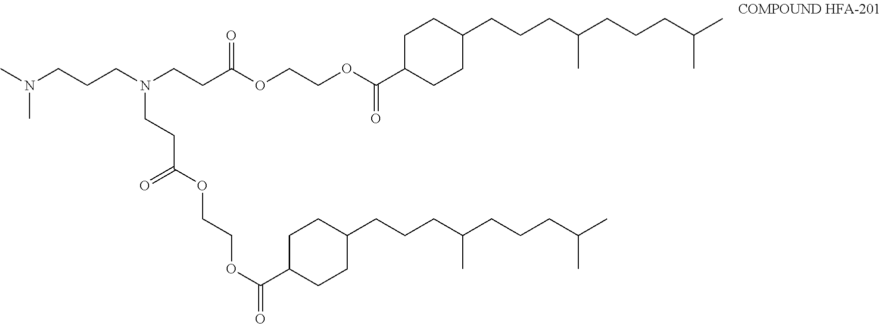 [SMILES: CC(C)CCCC(C)CCCC1CCC(C(=O)OCCOC(=O)CCN(CCCN(C)C)CCC(=O)OCCOC(=O)C2CCC(CCCC(C)CCCC(C)C)CC2)CC1]

### ((3,3′-((3-(dimethylamino) propyl) azanediyl)bis(propanoyl))bis(oxy)) bis(ethane-2,1-diyl)bis(4-(4,8-dimethylnonyl)cyclohexanecarboxylate)

Compound HFA-201 was prepared in accordance with the General Procedure D. The crude was purified by silica gel flash chromatography (eluent: 50% EtOAc/hexane). 1H NMR (400 MHz, Chloroform-d) δ 4.46-4.12 (m, 8H), 2.98-2.32 (m, 12H), 2.01-1.68 (m, 8H), 1.60-0.96 (m, 43H), 0.92-0.73 (m, 23H).

### Example 31—Preparation of Compound HFA-202

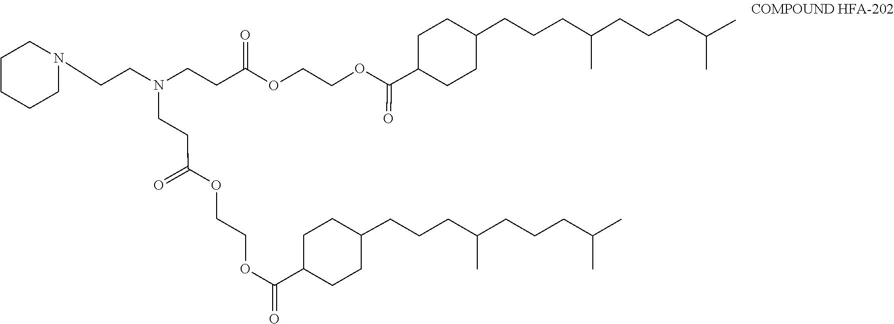 [SMILES: CC(C)CCCC(C)CCCC1CCC(C(=O)OCCOC(=O)CCN(CCC(=O)OCCOC(=O)C2CCC(CCCC(C)CCCC(C)C)CC2)CCN2CCCCC2)CC1]

### ((3,3′-((2-(piperidin-1-yl)ethyl) azanediyl)bis(propanoyl))bis(oxy)) bis(ethane-2,1-diyl)bis(4-(4,8-dimethylnonyl)cyclohexanecarboxylate)

Compound HFA-202 was prepared in accordance with the General Procedure D. The crude was purified by silica gel flash chromatography (eluent: 30% EtOAc/hexane). 1H NMR (400 MHz, Chloroform-d) δ 4.31-4.20 (m, 8H), 3.04 (dd, J=11.6, 6.1, 5.1 Hz, 8H), 2.76 (t, J=6.5 Hz, 4H), 2.49 (t, J=6.6, 2.0 Hz, 4H), 2.11-1.68 (m, 12H), 1.67-0.95 (m, 40H), 0.92-0.64 (m, 22H).

### Example 32—Preparation of Compound HFA-401

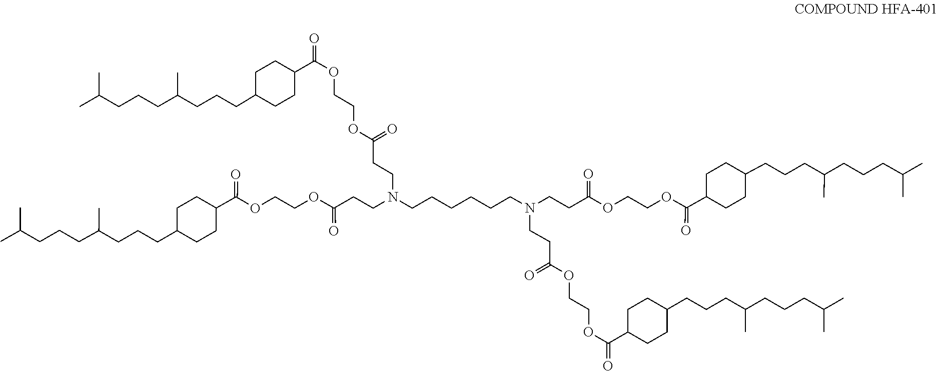 [SMILES: CC(C)CCCC(C)CCCC1CCC(C(=O)OCCOC(=O)CCN(CCCCCCN(CCC(=O)OCCOC(=O)C2CCC(CCCC(C)CCCC(C)C)CC2)CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-401 was prepared in accordance with the General Procedure D. The crude was purified by silica gel flash chromatography (eluent: 5% MeOH/DCM). 1H NMR (400 MHz, Chloroform-d) δ 4.37-4.21 (m, 16H), 2.75 (t, J=7.2 Hz, 6H), 2.51-2.33 (m, 10H), 2.00-1.67 (m, 16H), 1.59-0.97 (m, 88H), 0.88-0.76 (m, 40H).

### Example 33—Preparation of Compound HFA-402

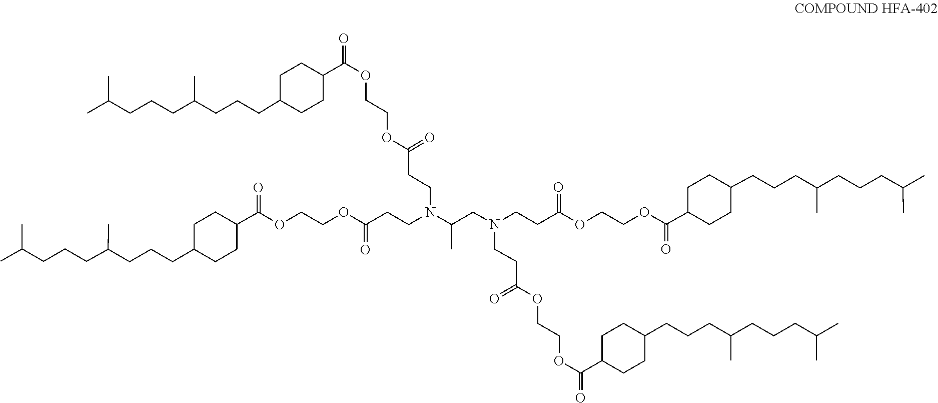 [SMILES: CC(C)CCCC(C)CCCC1CCC(C(=O)OCCOC(=O)CCN(CCC(=O)OCCOC(=O)C2CCC(CCCC(C)CCCC(C)C)CC2)CC(C)N(CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-402 was prepared in accordance with the General Procedure D. The crude was purified by silica gel flash chromatography (eluent: 5% MeOH/DCM). 1H NMR (400 MHz, Chloroform-d) δ 4.39-4.13 (m, 16H), 2.51-2.28 (m, 8H), 2.00-1.73 (m, 14H), 1.58-0.96 (m, 88H), 0.83 (dd, J=10.4, 6.6 Hz, 44H).

### Example 34—Preparation of Compound HFA-403

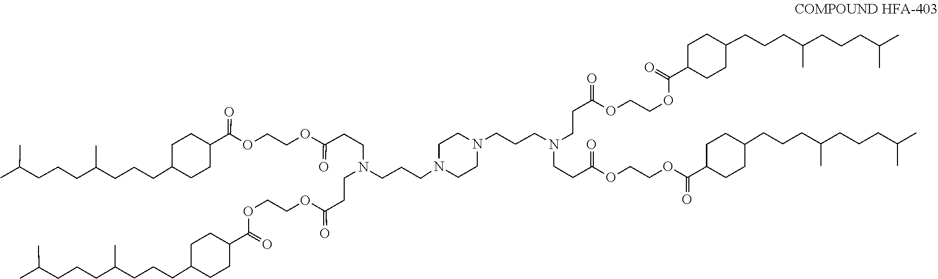 [SMILES: CC(C)CCCC(C)CCCC1CCC(C(=O)OCCOC(=O)CCN(CCCN2CCN(CCCN(CCC(=O)OCCOC(=O)C3CCC(CCCC(C)CCCC(C)C)CC3)CCC(=O)OCCOC(=O)C3CCC(CCCC(C)CCCC(C)C)CC3)CC2)CCC(=O)OCCOC(=O)C2CCC(CCCC(C)CCCC(C)C)CC2)CC1]

### ((3,3′,3″,3″-((piperazine-1,4-diylbis(propane-3,1-diyl))bis(azanetriyl))tetrakis(propanoyl))tetrakis(oxy))tetrakis(ethane-2,1-diyl)tetrakis(4-(4,8-dimethylnonyl)cyclohexanecarboxylate)

Compound HFA-403 was prepared in accordance with the General Procedure D. The crude was purified by silica gel flash chromatography (eluent: 5% MeOH/DCM). 1H NMR (400 MHz, Chloroform-d) δ 4.34-4.14 (m, 16H), 2.80-2.68 (m, 8H), 2.65-2.15 (m, 28H), 2.02-1.67 (m, 13H), 1.66-0.95 (m, 78H), 0.93-0.72 (m, 41H).

### Example 35—Preparation of Compound HFA-404

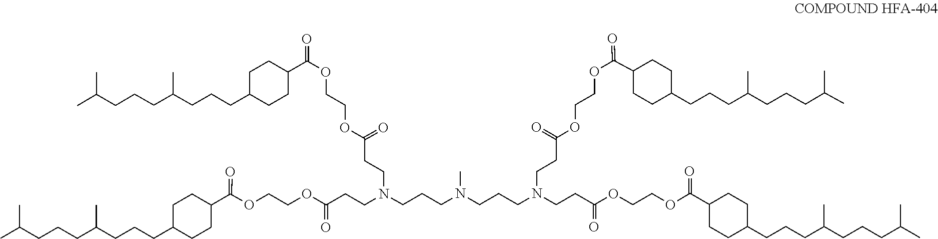 [SMILES: CC(C)CCCC(C)CCCC1CCC(C(=O)OCCOC(=O)CCN(CCCN(C)CCCN(CCC(=O)OCCOC(=O)C2CCC(CCCC(C)CCCC(C)C)CC2)CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-404 was prepared in accordance with the General Procedure D. The crude was purified by silica gel flash chromatography (eluent: 5% MeOH/DCM). 1H NMR (400 MHz, Chloroform-d) δ 4.37-4.13 (m, 16H), 2.92-2.15 (m, 26H), 2.04-1.65 (m, 15H), 1.63-0.96 (m, 78H), 0.91-0.71 (m, 44H).

### Example 36—Preparation of Compound HFA-601

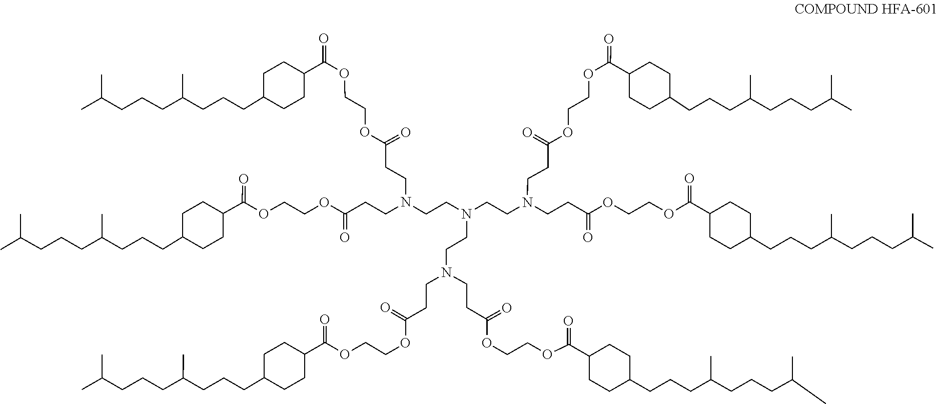 [SMILES: CC(C)CCCC(C)CCCC1CCC(C(=O)OCCOC(=O)CCN(CCC(=O)OCCOC(=O)C2CCC(CCCC(C)CCCC(C)C)CC2)CCN(CCN(CCC(=O)OCCOC(=O)C2CCC(CCCC(C)CCCC(C)C)CC2)CCC(=O)OCCOC(=O)C2CCC(CCCC(C)CCCC(C)C)CC2)CCN(CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-601 was prepared in accordance with the General Procedure D. The crude was purified by silica gel flash chromatography (eluent: 10% MeOH/DCM). 1H NMR (400 MHz, Chloroform-d) δ 4.31-4.13 (m, 24H), 2.76 (t, J-7.1 Hz, 10H), 2.66-2.37 (m, 22H), 2.00-1.66 (m, 21H), 1.61-0.94 (m, 118H), 0.91-0.72 (m, 63H).

### Example 37—Preparation of Compound FE (Diels-Alder reaction, E)

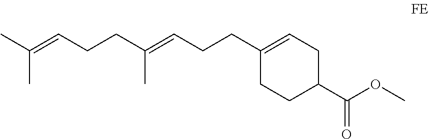 [SMILES: COC(=O)C1CC=C(CC/C=C(\C)CCC=C(C)C)CC1]

In a 4 mL sealed tube, farnesene (2.0 g, 9.8 mmol) is combined with methyl acrylate (0.93 g, 10.8 mmol). The tube was sealed and the reaction was stirred for 20 h at 130° C. The cooled reaction mixture was purified by silica gel flash column chromatography with 3% EtO Ac/hexane eluants.

Colorless oil; 2.01 g, yield 72%;

1H NMR (400 MHZ, Chloroform-d) 8 5.43-5.31 (m, 1H), 5.14-5.01 (m, 2H), 3.67-3.64 (m, 3H), 2.58-2.41 (m, 1H), 2.24-1.90 (m, 14H), 1.67-1.64 (m, 3H), 1.59-1.55 (m, 6H). 13C NMR (101 MHz, Chloroform-d) δ 176.56, 176.52, 137.39, 136.01, 135.17, 135.14, 131.30, 124.41, 124.40, 124.11, 124.08, 120.36, 118.96, 51.67, 51.64, 39.87, 39.78, 39.43, 37.75, 37.57, 30.72, 27.76, 27.75, 26.79, 26.30, 26.29, 25.76, 25.61, 25.21, 24.63, 17.75, 16.07, 16.06.

### Example 38—Preparation of Compound ME (Diels-Alder reaction, E)

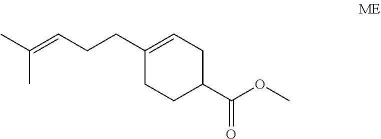 [SMILES: COC(=O)C1CC=C(CCC=C(C)C)CC1]

In a 4 mL sealed tube, myrcene (2.0 g, 14.7 mmol) is combined with methyl acrylate (1.4 g, 16.1 mmol). The tube was sealed and the reaction was stirred for 20 h at 130° C. The cooled reaction mixture was purified by silica gel flash column chromatography with 5% EtOAc/hexane eluants.

Colorless oil; 2.6 g, yield 72%;

1H NMR (400 MHZ, Chloroform-d) 8 5.40-5.32 (m, 1H), 5.10-5.01 (m, 1H), 3.67-3.62 (m, 3H), 2.57-2.40 (m, 1H), 2.24-1.86 (m, 9H), 1.70-1.58 (m, 4H), 1.56 (s, 3H). 13C NMR (101 MHz, Chloroform-d) δ 176.51, 176.47, 137.38, 136.02, 131.51, 131.47, 124.21, 124.18, 120.29, 118.90, 51.63, 51.61, 39.84, 39.40, 37.75, 37.58, 30.70, 27.73, 26.41, 26.39, 25.74, 25.58, 25.19, 24.61, 17.73, 17.72.

### Example 39—Preparation of Compound FE-211 (Transesterification reaction, G)

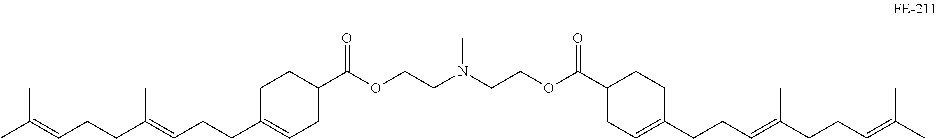 [SMILES: CC(C)=CCC/C(C)=C/CCC1=CCC(C(=O)OCCN(C)CCOC(=O)C2CC=C(CC/C=C(\C)CCC=C(C)C)CC2)CC1]

In a 4 mL glass tube, FE (304 mg, 1.0 mmol) is combined with an amine 211 (50 mg, 0.4 mmol) and triazabicyclodecene (TBD) (24 mg, 0.2 mmol). The tube was sealed and the reaction mixture was stirred for 16 h at 130° C. The cooled reaction mixture was purified by silica gel flash column chromatography with 20% EtOAc/hexane.

Colorless oil; 170 mg, yield 64%;

1H NMR (400 MHZ, Chloroform-d) 8 5.35 (s, 2H), 5.07 (s, 4H), 4.16 (t, J=5.7 Hz, 4H), 2.70 (t, J=5.7 Hz, 4H), 2.58-2.40 (m, 2H), 2.33 (s, 3H), 2.25-1.85 (m, 27H), 1.68-1.61 (m, 7H), 1.57 (s, 12H). 13C NMR (101 MHz, Chloroform-d) δ 175.91, 175.89, 137.36, 135.92, 135.11, 135.08, 131.23, 124.41, 124.37, 124.07, 124.04, 120.29, 118.88, 62.03, 61.99, 55.91, 42.87, 42.84, 39.86, 39.74, 39.43, 37.71, 37.55, 30.65, 27.71, 26.75, 26.28, 26.24, 25.74, 25.55, 25.17, 24.57, 17.73, 16.05, 16.03.

### Example 40—Preparation of Compound FE-212 (Transesterification reaction, G)

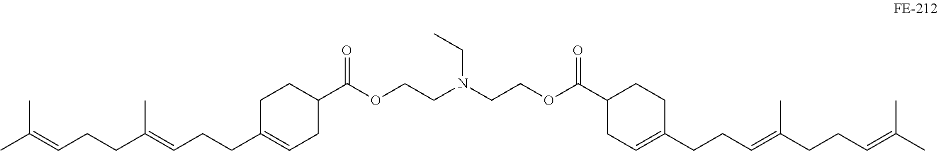 [SMILES: CCN(CCOC(=O)C1CC=C(CC/C=C(\C)CCC=C(C)C)CC1)CCOC(=O)C1CC=C(CC/C=C(\C)CCC=C(C)C)CC1]

In a 4 mL glass tube, FE (272 mg, 0.9 mmol) is combined with an amine 212 (50 mg, 0.4 mmol) and triazabicyclodecene (TBD) (11 mg, 0.1 mmol). The tube was sealed and the reaction mixture was stirred for 16 h at 130° C. The cooled reaction mixture was purified by silica gel flash column chromatography with 20% EtOAc/hexane.

Colorless oil; 144 mg, yield 59%; 1H NMR (400 MHZ, Chloroform-d) 8 5.41-5.33 (m, 2H), 5.13-5.02 (m, 4H), 4.17-4.08 (m, 4H), 2.79-2.72 (m, 4H), 2.61 (qd, J=7.1, 1.8 Hz, 2H), 2.56-2.41 (m, 2H), 2.25-2.11 (m, 4H), 2.08-1.90 (m, 23H), 1.69-1.62 (m, 7H), 1.59-1.54 (m, 12H), 1.01 (td, J=7.1, 1.5 Hz, 3H). 13C NMR (101 MHz, Chloroform-d) δ 175.99, 175.96, 137.42, 136.00, 135.17, 135.14, 131.31, 124.41, 124.12, 124.08, 120.34, 118.94, 62.59, 62.55, 52.32, 48.70, 48.68, 39.95, 39.78, 39.52, 37.76, 37.59, 30.71, 27.77, 27.75, 26.79, 26.33, 26.29, 25.79, 25.60, 25.21, 24.64, 17.77, 16.09, 16.07, 12.26.

### Example 41—Preparation of Compound FE-213 (Transesterification reaction, G)

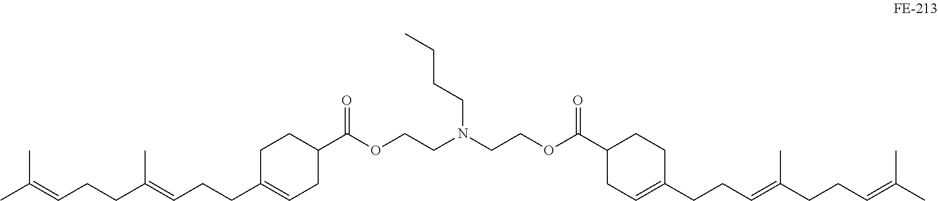 [SMILES: CCCCN(CCOC(=O)C1CC=C(CC/C=C(\C)CCC=C(C)C)CC1)CCOC(=O)C1CC=C(CC/C=C(\C)CCC=C(C)C)CC1]

In a 4 mL glass tube, FE (225 mg, 0.8 mmol) is combined with an amine 213 (50 mg, 0.3 mmol) and triazabicyclodecene (TBD) (9 mg, 0.1 mmol). The tube was sealed and the reaction mixture was stirred for 16 h at 130° C. The cooled reaction mixture was purified by silica gel flash column chromatography with 15% EtOAc/hexane.

Colorless oil; 178 mg, yield 85%; 1H NMR (400 MHZ, Chloroform-d) 8 5.36 (s, 2H), 5.13-5.02 (m, 4H), 4.17-4.06 (m, 4H), 2.74 (t, J=6.0 Hz, 4H), 2.58-2.38 (m, 4H), 2.24-1.87 (m, 27H), 1.69-1.61 (m, 7H), 1.60-1.53 (m, 12H), 1.44-1.34 (m, 2H), 1.33-1.22 (m, 2H), 0.88 (t, J=7.3 Hz, 3H). 13C NMR (101 MHZ, Chloroform-d) 8 175.97, 175.94, 137.39, 135.99, 135.14, 135.11, 131.26, 124.40, 124.10, 124.07, 120.33, 118.94, 62.61, 62.58, 54.83, 54.81, 52.90, 39.96, 39.77, 39.52, 37.75, 37.58, 30.70, 29.71, 29.70, 27.76, 27.74, 26.78, 26.32, 26.28, 25.77, 25.59, 25.20, 24.63, 20.45, 17.75, 16.07, 16.06, 14.11.

### Example 42—Preparation of Compound FE-214 (Transesterification reaction, G)

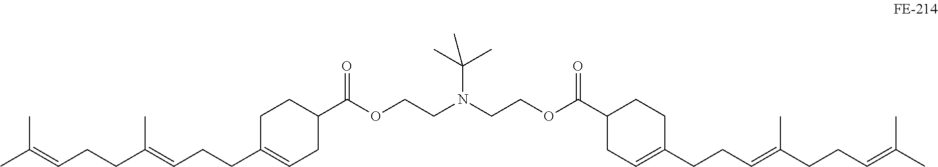 [SMILES: CC(C)=CCC/C(C)=C/CCC1=CCC(C(=O)OCCN(CCOC(=O)C2CC=C(CC/C=C(\C)CCC=C(C)C)CC2)C(C)(C)C)CC1]

In a 4 mL glass tube, FE (225 mg, 0.8 mmol) is combined with an amine 214 (50 mg, 0.3 mmol) and triazabicyclodecene (TBD) (9 mg, 0.1 mmol). The tube was sealed and the reaction mixture was stirred for 16 h at 130° C. The cooled reaction mixture was purified by silica gel flash column chromatography with 15% EtOAc/hexane.

Colorless oil; 144 mg, yield 69%; 1H NMR (400 MHZ, Chloroform-d) 8 5.41-5.34 (m, 2H), 5.12-5.04 (m, 4H), 4.05 (td, J=6.8, 2.0 Hz, 4H), 2.75 (t, J=6.9 Hz, 4H), 2.59-2.40 (m, 2H), 2.26-1.88 (m, 27H), 1.72-1.61 (m, 7H), 1.61-1.54 (m, 12H), 1.05 (s, 9H). 13C NMR (101 MHz, Chloroform-d) δ 176.05, 176.02, 137.41, 136.07, 135.17, 135.15, 131.32, 131.31, 124.44, 124.43, 124.15, 124.11, 120.35, 119.01, 64.93, 54.87, 49.56, 49.53, 40.00, 39.80, 39.56, 37.79, 37.61, 30.77, 27.82, 27.80, 27.23, 26.81, 26.35, 26.32, 25.80, 25.66, 25.27, 24.69, 17.79, 16.11, 16.09.

### Example 43—Preparation of Compound FE-215 (Transesterification reaction, G)

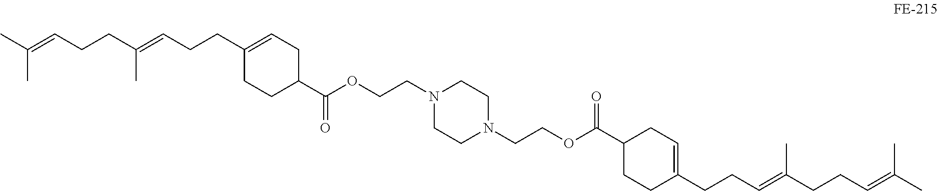 [SMILES: CC(C)=CCC/C(C)=C/CCC1=CCC(C(=O)OCCN2CCN(CCOC(=O)C3CC=C(CC/C=C(\C)CCC=C(C)C)CC3)CC2)CC1]

In a 4 mL glass tube, FE (417 mg, 1.4 mmol) is combined with an amine 215 (100 mg, 0.6 mmol) and triazabicyclodecene (TBD) (40 mg, 0.3 mmol). The tube was sealed and the reaction mixture was stirred for 16 h at 130° C. The cooled reaction mixture was purified by silica gel flash column chromatography with 50% EtOAc/hexane.

Brown oil; 200 mg, yield 50%; 1H NMR (400 MHZ, Chloroform-d) 8 5.35 (s, 2H), 5.06 (s, 4H), 4.18 (t, J=5.6 Hz, 4H), 2.66-2.36 (m, 13H), 2.27-1.76 (m, 27H), 1.71-1.37 (m, 20H). 13C NMR (101 MHz, Chloroform-d) δ 175.79, 175.76, 137.30, 135.87, 135.04, 135.02, 131.16, 124.33, 124.02, 124.00, 120.26, 118.86, 61.67, 56.63, 53.26, 39.80, 39.69, 39.38, 37.67, 37.50, 30.61, 27.67, 27.65, 26.70, 26.23, 26.20, 25.71, 25.51, 25.13, 24.50, 17.69, 16.02, 16.00.

### Example 44—Preparation of Compound FE-301 (Transesterification reaction, G)

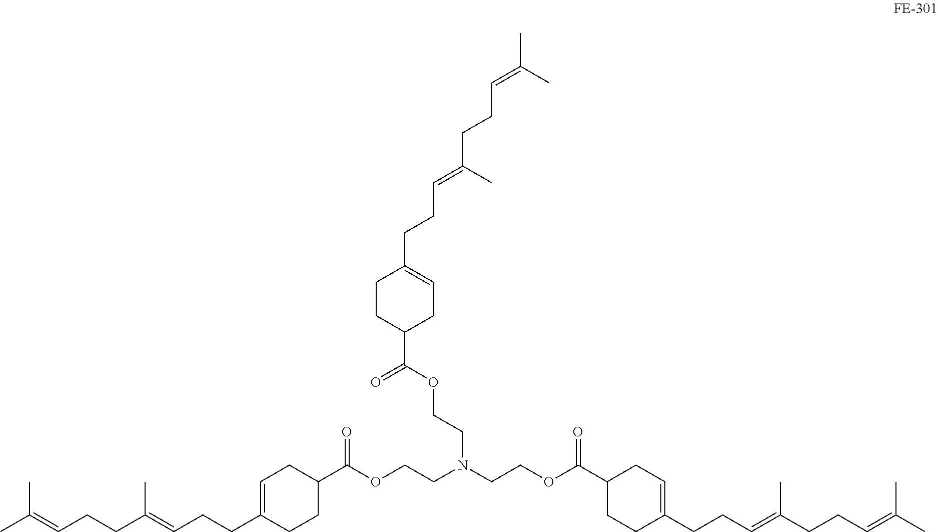 [SMILES: CC(C)=CCC/C(C)=C/CCC1=CCC(C(=O)OCCN(CCOC(=O)C2CC=C(CC/C=C(\C)CCC=C(C)C)CC2)CCOC(=O)C2CC=C(CC/C=C(\C)CCC=C(C)C)CC2)CC1]

In a 4 mL glass tube, FE (343 mg, 1.2 mmol) is combined with an amine 301 (50 mg, 0.3 mmol) and triazabicyclodecene (TBD) (28 mg, 0.2 mmol). The tube was sealed and the reaction mixture was stirred for 16 h at 130° C. The cooled reaction mixture was purified by silica gel flash column chromatography with 20% EtOAc/hexane.

Pale-brown oil; 110 mg, yield 35%; 1H NMR (400 MHZ, Chloroform-d) 8 5.36 (s, 3H), 5.14-4.98 (m, 6H), 4.12 (t, J=5.2 Hz, 6H), 2.82 (s, 6H), 2.57-2.39 (m, 3H), 2.28-1.81 (m, 40H), 1.69-1.51 (m, 29H). 13C NMR (101 MHz, Chloroform-d) δ 175.92, 175.89, 137.45, 135.96, 135.17, 135.15, 131.30, 124.41, 124.10, 124.07, 120.32, 118.88, 62.56, 53.33, 39.94, 39.78, 39.51, 37.75, 37.59, 30.70, 27.78, 27.75, 26.79, 26.33, 26.28, 25.79, 25.59, 25.22, 24.63, 17.77, 16.09, 16.07.

### Example 45—Preparation of Compound FE-301-2 (Transesterification reaction, G)

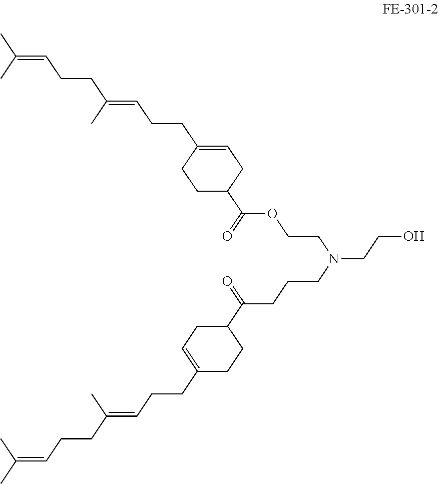 [SMILES: CC(C)=CCC/C(C)=C/CCC1=CCC(C(=O)CCCN(CCO)CCOC(=O)C2CC=C(CC/C=C(\C)CCC=C(C)C)CC2)CC1]

In the above reaction, a polar compound was isolated with 40% EtOAc/hexane eluant which turned out to be FE-301-2.

Pale-brown oil; 145 mg, yield 65%; 1H NMR (400 MHZ, Chloroform-d) 8 5.36 (s, 2H), 5.14-5.00 (m, 4H), 4.14 (s, 4H), 3.53 (t, J=4.7 Hz, 3H), 2.83 (s, 4H), 2.72 (s, 2H), 2.61-2.41 (m, 2H), 2.25-1.88 (m, 26H), 1.69-1.51 (m, 20H). 13C NMR (101 MHz, Chloroform-d) δ 176.05, 176.02, 137.46, 135.90, 135.17, 135.15, 131.30, 124.40, 124.09, 124.06, 120.31, 118.81, 62.17, 62.13, 58.83, 56.39, 56.35, 52.98, 39.94, 39.77, 39.51, 37.72, 37.57, 30.68, 27.75, 27.73, 26.78, 26.32, 26.25, 25.78, 25.54, 25.18, 24.60, 17.76, 16.08, 16.06.

### Example 46—Preparation of Compound ME-211 (Transesterification reaction, G)

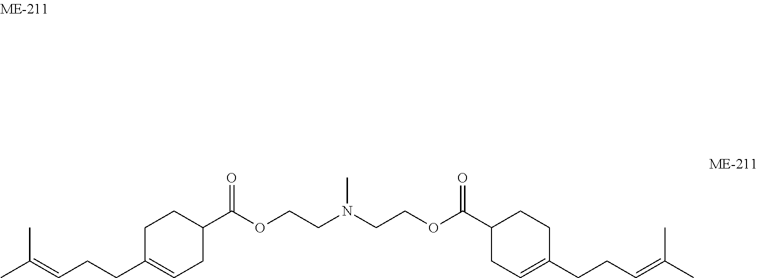 [SMILES: CC(C)=CCCC1=CCC(C(=O)OCCN(C)CCOC(=O)C2CC=C(CCC=C(C)C)CC2)CC1]

In a 4 mL glass tube, ME (233 mg, 1.1 mmol) is combined with an amine 211 (50 mg, 0.4 mmol) and triazabicyclodecene (TBD) (24 mg, 0.2 mmol). The tube was sealed and the reaction mixture was stirred for 16 h at 130° C. The cooled reaction mixture was purified by silica gel flash column chromatography with 20% EtOAc/hexane.

Colorless oil; 155 mg, yield 72%; 1H NMR (400 MHz, Chloroform-d) δ 5.33 (s, 2H), 5.08-4.98 (m, 2H), 4.21-4.05 (m, 4H), 2.69 (t, J=5.7 Hz, 4H), 2.56-2.39 (m, 2H), 2.32 (d, J=1.4 Hz, 3H), 2.22-1.86 (m, 19H), 1.66-1.60 (m, 7H), 1.55 (s, 6H). 13C NMR (101 MHZ, Chloroform-d) 8 175.86, 175.84, 137.33, 135.91, 131.43, 131.40, 124.15, 124.13, 120.20, 118.80, 61.90, 61.86, 55.79, 55.77, 42.73, 42.72, 39.81, 39.38, 37.69, 37.53, 30.62, 27.66, 26.34, 25.71, 25.49, 25.12, 24.53, 17.71, 17.69.

### Example 47—Preparation of Compound ME-212 (Transesterification reaction, G)

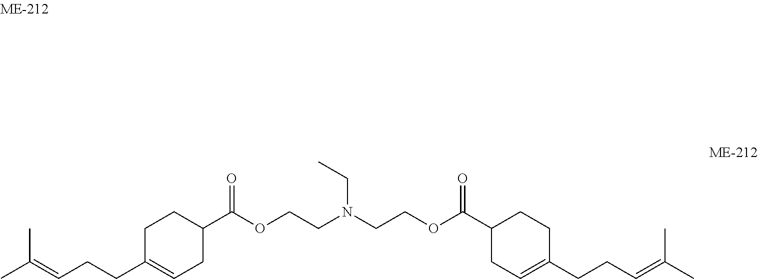 [SMILES: CCN(CCOC(=O)C1CC=C(CCC=C(C)C)CC1)CCOC(=O)C1CC=C(CCC=C(C)C)CC1]

In a 4 mL glass tube, ME (314 mg, 1.4 mmol) is combined with an amine 212 (75 mg, 0.6 mmol) and triazabicyclodecene (TBD) (40 mg, 0.1 mmol). The tube was sealed and the reaction mixture was stirred for 16 h at 130° C. The cooled reaction mixture was purified by silica gel flash column chromatography with 20% EtOAc/hexane.

Colorless oil; 174 mg, yield 60%; 1H NMR (400 MHZ, Chloroform-d) 8 5.40-5.30 (m, 2H), 5.12-4.98 (m, 2H), 4.16-4.05 (m, 4H), 2.78-2.69 (m, 4H), 2.59 (qd, J=7.1, 1.8 Hz, 2H), 2.55-2.39 (m, 2H), 2.25-1.85 (m, 18H), 1.71-1.48 (m, 14H), 0.99 (td, J=7.1, 1.4 Hz, 3H). 13C NMR (101 MHZ, Chloroform-d) 8 175.92, 175.89, 137.36, 135.98, 131.46, 131.44, 124.19, 124.17, 120.24, 118.87, 62.61, 62.57, 52.35, 48.69, 48.68, 39.90, 39.46, 37.73, 37.56, 30.68, 27.71, 26.38, 25.74, 25.54, 25.16, 24.59, 17.73, 17.72, 12.29.

### Example 48—Preparation of Compound ME-213 (Transesterification reaction, G)

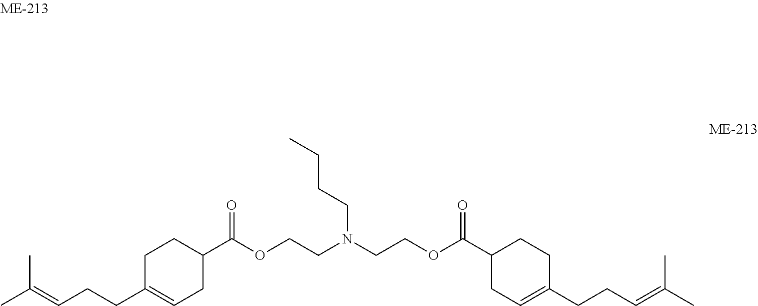 [SMILES: CCCCN(CCOC(=O)C1CC=C(CCC=C(C)C)CC1)CCOC(=O)C1CC=C(CCC=C(C)C)CC1]

In a 4 mL glass tube, ME (259 mg, 1.2 mmol) is combined with an amine 213 (75 mg, 0.5 mmol) and triazabicyclodecene (TBD) (13 mg, 0.1 mmol). The tube was sealed and the reaction mixture was stirred for 16 h at 130° C. The cooled reaction mixture was purified by silica gel flash column chromatography with 10% EtOAc/hexane.

Colorless oil; 156 mg, yield 62%; 1H NMR (400 MHZ, Chloroform-d) 8 5.40-5.29 (m, 2H), 5.12-4.97 (m, 2H), 4.16-4.05 (m, 4H), 2.74 (t, J=6.0 Hz, 4H), 2.55-2.40 (m, 4H), 2.26-1.87 (m, 19H), 1.68-1.60 (m, 7H), 1.56 (s, 6H), 1.43-1.34 (m, 2H), 1.32-1.21 (m, 2H), 0.87 (t, J=7.3 Hz, 3H). 13C NMR (101 MHz, Chloroform-d) δ 175.95, 175.93, 137.39, 136.01, 131.50, 131.47, 124.21, 124.18, 120.27, 118.89, 62.58, 62.54, 54.82, 54.80, 52.90, 39.94, 39.50, 37.75, 37.58, 30.70, 29.67, 29.66, 27.74, 27.72, 26.40, 25.76, 25.56, 25.19, 24.62, 20.44, 17.76, 17.74, 14.09.

### Example 49—Preparation of Compound ME-214 (Transesterification reaction, G)

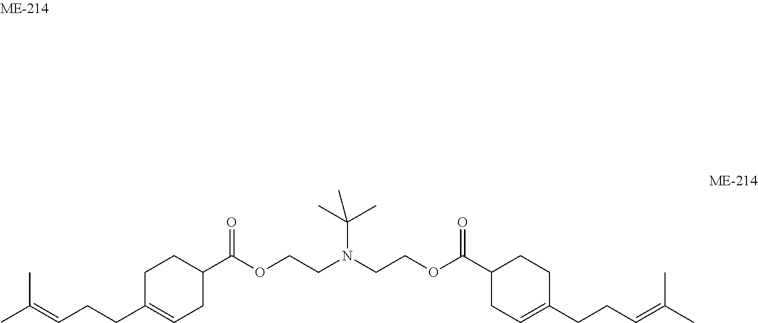 [SMILES: CC(C)=CCCC1=CCC(C(=O)OCCN(CCOC(=O)C2CC=C(CCC=C(C)C)CC2)C(C)(C)C)CC1]

In a 4 mL glass tube, ME (259 mg, 1.2 mmol) is combined with an amine 214 (75 mg, 0.5 mmol) and triazabicyclodecene (TBD) (13 mg, 0.1 mmol). The tube was sealed and the reaction mixture was stirred for 16 h at 130° C. The cooled reaction mixture was purified by silica gel flash column chromatography with 10% EtOAc/hexane.

Colorless oil; 163 mg, yield 65%; 1H NMR (400 MHZ, Chloroform-d) 8 5.42-5.30 (m, 2H), 5.13-5.00 (m, 2H), 4.04 (t, J=6.9 Hz, 4H), 2.75 (t, J=6.9 Hz, 4H), 2.61-2.36 (m, 2H), 2.24-1.88 (m, 18H), 1.75-1.48 (m, 14H), 1.05 (s, 9H). 13C NMR (101 MHz, Chloroform-d) δ 176.05, 176.02, 137.42, 136.10, 131.56, 131.53, 124.25, 124.23, 120.30, 118.96, 64.94, 54.88, 49.56, 39.99, 39.55, 37.79, 37.62, 30.77, 27.80, 27.79, 27.24, 26.44, 25.80, 25.64, 25.26, 24.68, 17.80, 17.78.

### Example 50—Preparation of Compound ME-215 (Transesterification reaction, G)

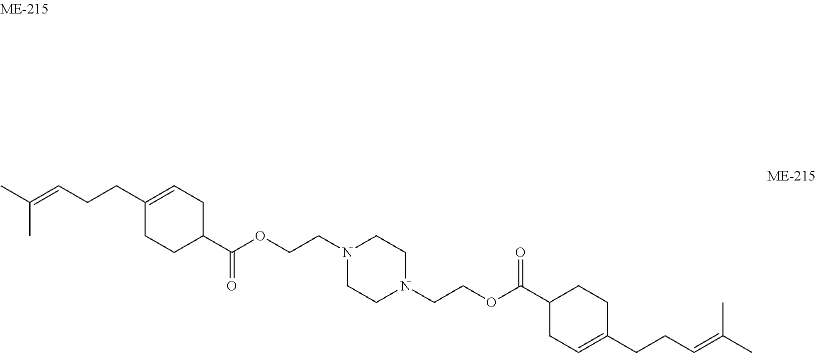 [SMILES: CC(C)=CCCC1=CCC(C(=O)OCCN2CCN(CCOC(=O)C3CC=C(CCC=C(C)C)CC3)CC2)CC1]

In a 4 mL glass tube, ME (239 mg, 1.1 mmol) is combined with an amine 215 (75 mg, 0.4 mmol) and triazabicyclodecene (TBD) (12 mg, 0.1 mmol). The tube was sealed and the reaction mixture was stirred for 16 h at 130° C. The cooled reaction mixture was purified by silica gel flash column chromatography with 40% EtOAc/hexane.

Colorless oil; 200 mg, yield 84%; 1H NMR (400 MHZ, Chloroform-d) 8 5.32 (s, 2H), 5.11-4.95 (m, 2H), 4.16 (t, J=5.7 Hz, 4H), 2.63-2.36 (m, 13H), 2.21-1.82 (m, 19H), 1.70-1.48 (m, 14H). 13C NMR (101 MHz, Chloroform-d) δ 175.81, 175.78, 137.32, 135.90, 131.42, 131.39, 124.14, 124.12, 120.21, 118.80, 61.65, 56.60, 53.20, 39.79, 39.36, 37.68, 37.52, 30.62, 27.66, 27.63, 26.33, 25.71, 25.49, 25.11, 24.49, 17.71, 17.69.

### Example 51—Preparation of Compound ME-301 (Transesterification reaction, G)

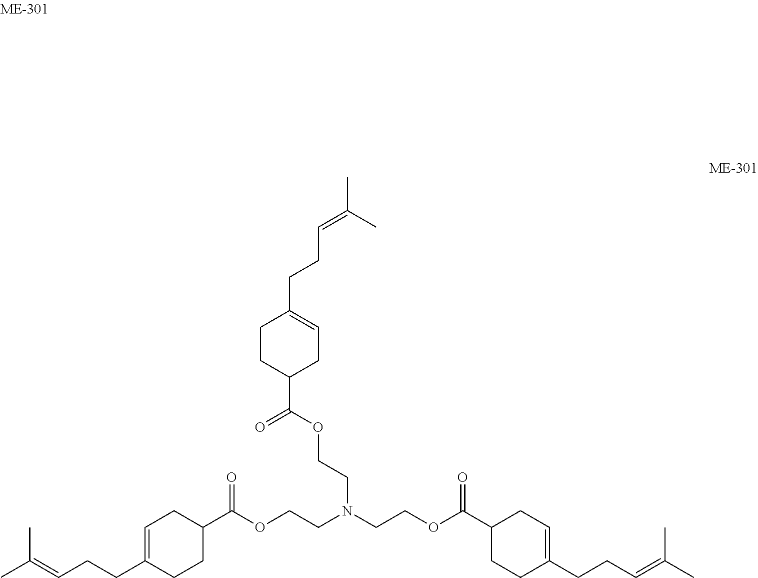 [SMILES: CC(C)=CCCC1=CCC(C(=O)OCCN(CCOC(=O)C2CC=C(CCC=C(C)C)CC2)CCOC(=O)C2CC=C(CCC=C(C)C)CC2)CC1]

In a 4 mL glass tube, ME (260 mg, 1.2 mmol) is combined with an amine 301 (50 mg, 0.3 mmol) and triazabicyclodecene (TBD) (28 mg, 0.2 mmol). The tube was sealed and the reaction mixture was stirred for 16 h at 130° C. The cooled reaction mixture was purified by silica gel flash column chromatography with 20% EtOAc/hexane.

Pale-brown oil; 118 mg, yield 49%; 1H NMR (400 MHZ, Chloroform-d) 8 5.43-5.31 (m, 3H), 5.12-5.00 (m, 3H), 4.14 (s, 6H), 2.85 (s, 6H), 2.58-2.40 (m, 3H), 2.24-1.88 (m, 29H), 1.68-1.64 (m, 10H), 1.58 (s, 9H). 13C NMR (101 MHZ, Chloroform-d) & 175.94, 175.91, 137.49, 136.00, 131.60, 131.58, 124.23, 124.20, 120.30, 118.84, 62.47, 53.31, 39.95, 39.52, 37.77, 37.61, 30.73, 27.78, 27.76, 26.44, 26.42, 25.81, 25.58, 25.22, 24.64, 17.81, 17.79.

### Example 52—Preparation of Compound ME-301-2 (Transesterification reaction, G)

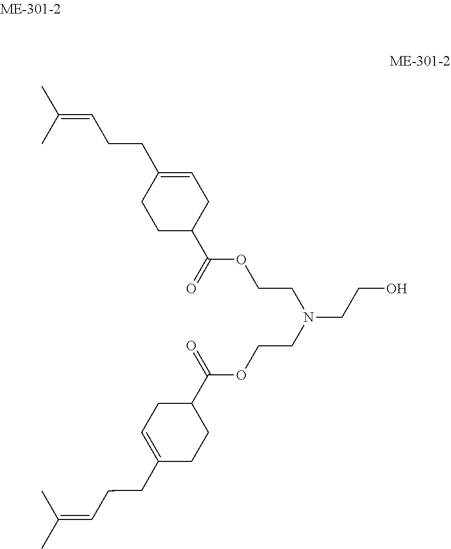 [SMILES: CC(C)=CCCC1=CCC(C(=O)OCCN(CCO)CCOC(=O)C2CC=C(CCC=C(C)C)CC2)CC1]

In the above reaction, a polar compound was isolated with 40% EtOAc/hexane eluant which turned out to be ME-301-2.

Pale-brown oil; 66 mg, yield 37%; 1H NMR (400 MHZ, Chloroform-d) 8 5.42-5.33 (m, 2H), 5.14-5.00 (m, 2H), 4.24-4.07 (m, 4H), 3.56 (t, J=5.1 Hz, 2H), 2.86 (t, J=5.5 Hz, 4H), 2.75 (t, J=4.6 Hz, 2H), 2.61-2.42 (m, 2H), 2.25-1.91 (m, 19H), 1.68-1.55 (m, 14H). 13C NMR (101 MHz, Chloroform-d) δ 176.10, 176.07, 137.53, 135.98, 131.65, 131.63, 124.25, 124.22, 120.32, 118.81, 62.12, 62.07, 58.80, 58.79, 56.48, 56.45, 53.05, 53.03, 39.98, 39.56, 37.78, 37.63, 30.74, 27.78, 26.46, 26.43, 25.83, 25.58, 25.22, 24.64, 17.83, 17.82.

### Example 53—Preparation of Compound FE-216 (Synthesis of FE-216 and ME-216 lipidoids H)

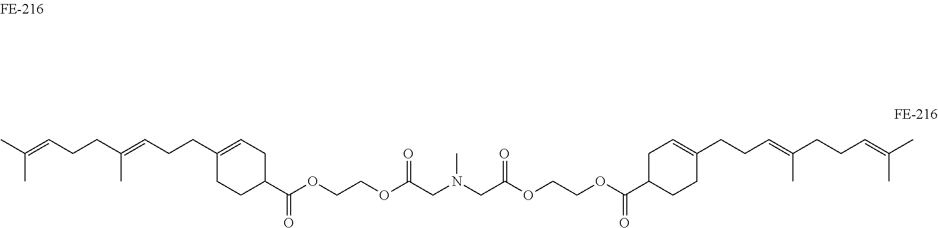 [SMILES: CC(C)=CCC/C(C)=C/CCC1=CCC(C(=O)OCCOC(=O)CN(C)CC(=O)OCCOC(=O)C2CC=C(CC/C=C(\C)CCC=C(C)C)CC2)CC1]

In a 20 mL scintillation glass vial, methyliminodiacetic acid (216) (50 mg, 0.3 mmol, 1.0 eq), 1-ethyl-3-(3-dimethylamino propyl) carbodiimide (EDC-HCl) (143 mg, 0.7 mmol, 2.2 eq), and dimethyl aminopyridine (DMAP) (91 mg, 0.7 mmol, 2.2 eq) were dissolved in dichloromethane and dimethylformamide (1:1, 5 mL). The mixture was stirred for 15 minutes at room temperature before adding Compound 1 (326 mg, 1.0 mmol, 2.2 eq) dissolved in dichloromethane (1.0 mL). The reaction was then stirred for 20 h at room temperature. The reaction mixture was transferred to a separatory funnel, added 20 mL of distilled water, and extracted with dichloromethane (3×40 mL). Combined organic extracts were dried over Na₂SO₄, filtered to a round bottom flask and the solvent was evaporated under reduced pressure. The crude residue was purified by silica gel flash column chromatography with 20% EtO Ac/hexane eluant.

Colorless oil; 137 mg, yield 53%; 1H NMR (400 MHZ, Chloroform-d) 8 5.42-5.29 (m, 2H), 5.16-4.96 (m, 4H), 4.39-4.12 (m, 8H), 3.50 (s, 4H), 2.61-2.33 (m, 5H), 2.28-1.70 (m, 27H), 1.68-1.48 (m, 19H). 13C NMR (101 MHz, Chloroform-d) δ 175.69, 175.65, 170.50, 137.38, 135.82, 135.14, 135.11, 131.24, 124.34, 124.02, 123.98, 120.28, 118.75, 62.30, 61.86, 56.89, 42.04, 39.72, 39.31, 37.67, 37.51, 30.53, 27.63, 27.61, 26.73, 26.25, 26.21, 25.73, 25.49, 25.11, 24.48, 17.71, 16.04, 16.02.

### Example 54—Preparation of Compound ME-216 (Synthesis of FE-216 and ME-216 lipidoids H)

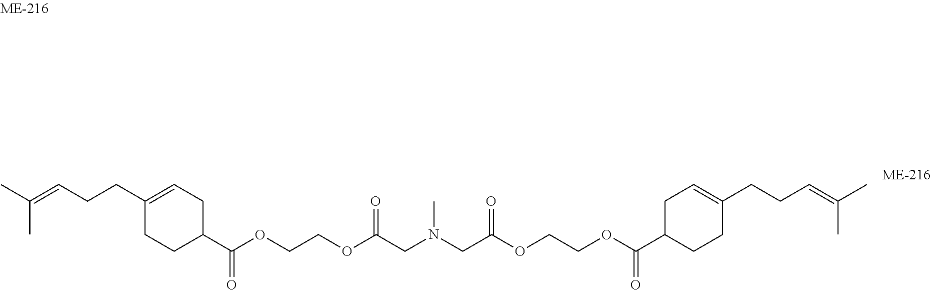 [SMILES: CC(C)=CCCC1=CCC(C(=O)OCCOC(=O)CN(C)CC(=O)OCCOC(=O)C2CC=C(CCC=C(C)C)CC2)CC1]

In a 20 mL scintillation glass vial, methyliminodiacetic acid (216) (50 mg, 0.3 mmol, 1.0 eq), 1-ethyl-3-(3-dimethylamino propyl) carbodiimide (EDC-HCl) (143 mg, 0.7 mmol, 2.2 eq), and dimethyl aminopyridine (DMAP) (91 mg, 0.7 mmol, 2.2 eq) were dissolved in dichloromethane and dimethylformamide (1:1, 5 mL). The mixture was stirred for 15 minutes at room temperature before adding Compound 3 (257 mg, 1.0 mmol, 2.2 eq) dissolved in dichloromethane (1.0 mL). The reaction was then stirred for 20 h at room temperature. The reaction mixture was transferred to a separatory funnel, added 20 mL of distilled water, and extracted with dichloromethane (3×40 mL). Combined organic extracts were dried over Na₂SO₄, filtered to a round bottom flask and the solvent was evaporated under reduced pressure. The crude residue was purified by silica gel flash column chromatography with 20% EtOAc/hexane eluant.

Colorless oil; 160 mg, yield 77%; 1H NMR (400 MHZ, Chloroform-d) 8 5.41-5.27 (m, 2H), 5.07-4.94 (m, 2H), 4.32-4.11 (m, 8H), 3.47 (s, 4H), 2.56-2.29 (m, 5H), 2.23-1.69 (m, 19H), 1.67-1.56 (m, 7H), 1.53 (s, 6H). 13C NMR (101 MHz, Chloroform-d) § 175.60, 175.55, 170.44, 137.30, 135.78, 131.42, 131.38, 124.07, 124.05, 120.17, 118.66, 62.23, 61.84, 61.81, 56.83, 41.96, 39.65, 39.23, 37.61, 37.45, 30.48, 27.54, 26.28, 25.66, 25.40, 25.02, 24.40, 17.67, 17.65.

### Example 55—Preparation of Compound FE-211-OH (Mono-transesterification reaction, I)

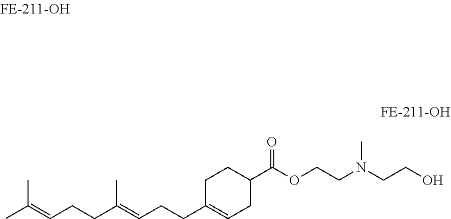 [SMILES: CC(C)=CCC/C(C)=C/CCC1=CCC(C(=O)OCCN(C)CCO)CC1]

In a 4 mL glass tube, FE (304 mg, 1.0 mmol) is combined with an amine 211 (50 mg, 0.4 mmol). The tube was sealed and the reaction mixture was stirred for 20 h at 130° C. The cooled reaction mixture was purified by silica gel flash column chromatography with 5% MeOH/DCM.

Colorless oil; 60 mg, yield 31%; 1H NMR (400 MHZ, Chloroform-d) 8 5.42-5.30 (m, 1H), 5.14-4.99 (m, 2H), 4.24-4.09 (m, 2H), 3.56 (t, J=5.3 Hz, 2H), 3.21 (s, 1H), 2.71 (t, J=11.1 Hz, 2H), 2.59 (t, J=5.3 Hz, 2H), 2.55-2.41 (m, 1H), 2.32 (s, 3H), 2.25-1.87 (m, 14H), 1.65 (s, 3H), 1.57 (s, 6H).

### Example 56—Preparation of Compound FE-211-Oleic (Synthesis of hybrid lipidoids, J)

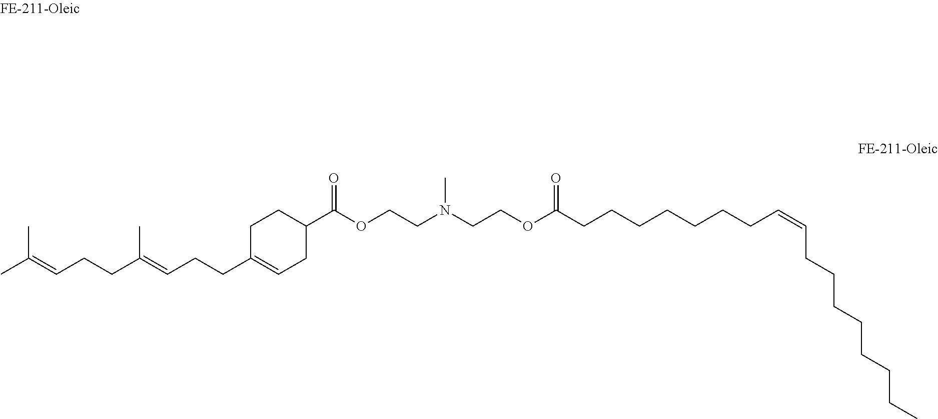 [SMILES: CCCCCCCC/C=C\CCCCCCCC(=O)OCCN(C)CCOC(=O)C1CC=C(CC/C=C(\C)CCC=C(C)C)CC1]

In a 20 mL scintillation glass vial, Oleic acid (50 mg, 0.2 mmol, 1.0 eq), 1-ethyl-3-(3-dimethyl aminopropyl) carbodiimide (EDC-HCl) (37 mg, 0.2 mmol, 1.1 eq), and dimethyl aminopyridine (DMAP) (24 mg, 0.2 mmol, 1.1 eq) were dissolved in dichloromethane (1.0 mL). The mixture was stirred for 15 minutes at room temperature before adding FE-211-OH (73 mg, 0.2 mmol, 1.1 eq) dissolved in dichloromethane (0.5 mL). The reaction was then stirred for 20 h at room temperature. The reaction mixture was transferred to a separatory funnel, added 20 mL of distilled water, and extracted with dichloromethane (3×40 mL). Combined organic extracts were dried over Na₂SO₄, filtered to a round bottom flask and the solvent was evaporated under reduced pressure. The crude residue was purified by silica gel flash column chromatography with 20% EtOAc/hexane eluant.

Colorless oil; 91 mg, yield 80%; 1H NMR (400 MHZ, Chloroform-d) 8 5.41-5.25 (m, 3H), 5.06 (s, 2H), 4.25-4.06 (m, 4H), 2.79-2.63 (m, 4H), 2.59-2.41 (m, 1H), 2.34 (s, 3H), 2.28 (t, J=7.6 Hz, 2H), 2.24-2.11 (m, 2H), 2.10-1.90 (m, 15H), 1.69-1.53 (m, 12H), 1.38-1.17 (m, 20H), 0.91-0.80 (m, 3H). 13C NMR (101 MHZ, Chloroform-d) 8 175.98, 175.95, 173.82, 137.43, 135.98, 135.19, 135.16, 131.31, 130.05, 129.79, 124.41, 124.11, 124.07, 120.33, 118.91, 61.95, 61.92, 61.80, 55.84, 42.80, 42.79, 39.91, 39.79, 39.48, 37.76, 37.59, 34.32, 32.00, 30.69, 29.85, 29.78, 29.62, 29.41, 29.27, 29.24, 29.21, 29.20, 27.76, 27.74, 27.30, 27.25, 26.80, 26.33, 26.29, 25.79, 25.59, 25.21, 24.98, 24.62, 22.78, 17.77, 16.08, 14.22.

### Example 57—Preparation of Compound HFE (Hydrogenation reaction, F)

### HFE

In a 100 mL round-bottomed flask, FE (701 mg) was dissolved in mixed solvents of ethanol and CH₂C₁₂ (4:1) (20 mL). To this solution, 10% Pd/C (72 mg, 10% w/w) was added in one portion and the resulting dark suspension was stirred under a hydrogen atmosphere for 20 h at room temperature (one hydrogen balloon). The reaction mixture was filtered through celite and rinsed with CH₂C₁₂ (3×50 mL). The filtrate was evaporated and the resulting residue proceeded to the next step without any purification.

Colorless oil; 462 mg, yield 65%; 1H NMR (400 MHZ, Chloroform-d) 8 3.66 (s, 3H), 2.70-2.15 (m, 1H), 1.99-1.66 (m, 4H), 1.56-1.46 (m, 2H), 1.39-1.04 (m, 16H), 0.93-0.78 (m, 10H).

### Example 58—Preparation of Compound HME (Hydrogenation reaction, F)

### HME

In a 100 mL round-bottomed flask, ME (1.2 g) was dissolved in mixed solvents of ethanol and CH₂C₁₂ (4:1) (20 mL). To this solution, 10% Pd/C (126 mg, 10% w/w) was added in one portion and the resulting dark suspension was stirred under a hydrogen atmosphere for 20 h at room temperature (one hydrogen balloon). The reaction mixture was filtered through celite and rinsed with CH₂Cl₂ (3×50 mL). The filtrate was evaporated and the resulting residue proceeded to the next step without any purification.

Colorless oil; 0.88 g, yield 72%; 1H NMR (400 MHZ, Chloroform-d) § 3.65 (s, 3H), 2.71-2.14 (m, 1H), 2.01-1.67 (m, 4H), 1.59-1.04 (m, 11H), 0.94-0.78 (m, 7H).

### Example 59—Preparation of Compound HFE-211 (Transesterification reaction, G)

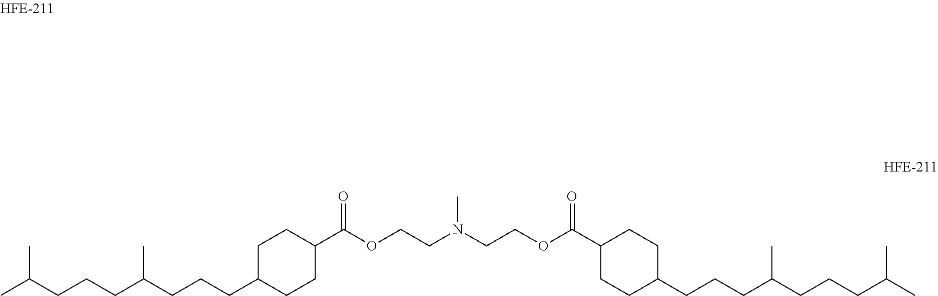 [SMILES: CC(C)CCCC(C)CCCC1CCC(C(=O)OCCN(C)CCOC(=O)C2CCC(CCCC(C)CCCC(C)C)CC2)CC1]

In a 4 mL glass tube, HFE (155 mg, 0.5 mmol) is combined with an amine 211 (25 mg, 0.2 mmol) and triazabicyclodecene (TBD) (12 mg, 0.1 mmol). The tube was sealed and the reaction mixture was stirred for 16 h at 130° C. The cooled reaction mixture was purified by silica gel flash column chromatography with 20% EtOAc/hexane.

Colorless oil; 106 mg, yield 78%; 1H NMR (400 MHZ, Chloroform-d) 8 4.16-3.98 (m, 4H), 2.70-2.57 (m, 4H), 2.25 (s, 3H), 2.21-2.02 (m, 2H), 1.91-1.55 (m, 8H), 1.46-1.34 (m, 4H), 1.31-0.89 (m, 34H), 0.76-0.67 (m, 18H). 13C NMR (101 MHZ, Chloroform-d) 8 176.21, 176.15, 61.72, 55.90, 55.87, 43.57, 43.51, 39.45, 37.74, 37.63, 37.43, 37.41, 37.38, 37.10, 37.04, 32.87, 32.85, 32.46, 32.37, 29.12, 28.07, 25.59, 24.90, 24.35, 24.26, 22.83, 22.73, 19.79.

### Example 60—Preparation of Compound HFE-215 (Transesterification reaction, G)

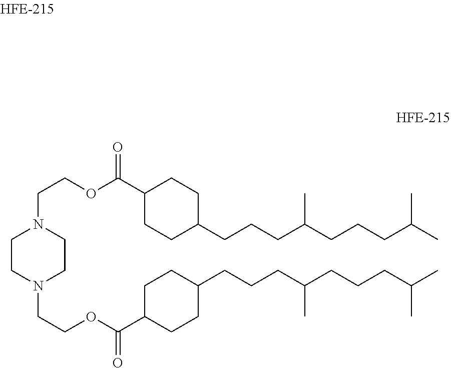 [SMILES: CC(C)CCCC(C)CCCC1CCC(C(=O)OCCN2CCN(CCOC(=O)C3CCC(CCCC(C)CCCC(C)C)CC3)CC2)CC1]

In a 4 mL glass tube, HFE (149 mg, 0.5 mmol) is combined with an amine 215 (35 mg, 0.2 mmol) and triazabicyclodecene (TBD) (12 mg, 0.1 mmol). The tube was sealed and the reaction mixture was stirred for 16 h at 130° C. The cooled reaction mixture was purified by silica gel flash column chromatography with 50% EtOAc/hexane.

Colorless oil; 92 mg, yield 65%; 1H NMR (400 MHZ, Chloroform-d) ô 4.22-4.13 (m, 4H), 2.65-2.44 (m, 12H), 2.32-2.12 (m, 2H), 1.97-1.65 (m, 8H), 1.56-1.43 (m, 4H), 1.39-0.95 (m, 34H), 0.86-0.77 (m, 18H).

For the following Examples, Compound Numbers are assigned to compounds of Formula (I) of the present disclosure according to the following:

| | | Compound | | Compound | | Compound | | Compound |
|---|---|---|---|---|---|---|---|---|
| Acrylates | FA- | No. | HFA- | No. | MA- | No. | HMA- | No. |
| Amines | 201 | 5 | 201 | 12 | 201 | 19 | 201 | 25 |
| | 202 | 6 | 202 | 13 | 202 | N.D. | 202 | 26 |
| | 401 | 7 | 401 | 14 | 401 | 20 | 401 | 27 |
| | 402 | 8 | 402 | 15 | 402 | 21 | 402 | 28 |
| | 403 | 9 | 403 | 16 | 403 | 22 | 403 | 29 |
| | 404 | 10 | 404 | 17 | 404 | 23 | 404 | 30 |
| | 601 | 11 | 601 | 18 | 601 | 24 | 601 | 31 |

| | | Compound | | Compound |
|---|---|---|---|---|
| | FE- | No. | ME- | No. |
| Amines | 211 | 32 | 211 | 42 |
| | 212 | 33 | 212 | 43 |
| | 213 | 34 | 213 | 44 |
| | 214 | 35 | 214 | 45 |
| | 215 | 36 | 215 | 46 |
| | 216 | 37 | 216 | 47 |
| | 301-1 | 38 | 301-1 | 48 |
| | 301-2 | 39 | 301-2 | 49 |
| | 211-OH | 40 | | |
| | 211-oleic | 41 | | |

### Example 61—General Method for the Preparation of LNPs of Present Disclosure Comprising RNA or DNA

The following is a nonlimiting example that provides a general method for effectively formulating a plurality of multi-component LNP compositions comprising exemplary compounds of Formula (I) and mRNA or DNA.

LNP compositions of the present disclosure comprising exemplary compounds of Formula (I) were prepared by combining various percentages of an exemplary compound of Formula (I), the phospholipid DOPE, the structural lipid cholesterol (Chol) and 1,2-dimyristoyl-sn-glycerol methoxypolyethylene glycol (DMG-PEG2000; Avanti Polar Lipids, Alabaster, Alabama, USA).

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol % as shown in Tables 4 and 5.

TABLE 4

| One of | | | | Lipid | |
|---|---|---|---|---|---|
| COMPOUNDS | | | DMG- | Blend | Lipid:RNA |
| 5-49 | DOPE | Cholesterol | PEG2000 | (mM) | (w/w) |
| 41.44 | 45.85 | 10 | 2.7 | 25 | 40:1 |

TABLE 5

| One of | | | | Lipid | |
|---|---|---|---|---|---|
| COMPOUNDS | | | DMG- | Blend | Lipid:DNA |
| 5-49 | DOPE | Cholesterol | PEG2000 | (mM) | (w/w) |
| 38 | 50 | 10.0 | 2.0 | 25 | 45:1 |

A 1 mg/ml solution of the desired mRNA or DNA to be incorporated into the LNPs was added to 150 mM sodium acetate buffer (pH 5.2) to form a stock solution and kept on ice. The aqueous mRNA or DNA was then added to the lipids as shown in Table 4 or 5 and hand-mixed vigorously with a single channel pipettor.

The resultant mRNA or DNA LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against phosphate buffered saline (PBS) (dialysate: dialysis buffer volume at least 1:200 v/v), pH 7.4 overnight at 4° C. (or alternatively room temperature for at least 4 hours), to remove the 25% ethanol and achieve a complete buffer exchange. In some experiments the LNPs were further concentrated by in an Amicon® Ultra-4 centrifugal filter unit, MWCO-30 kDa (Millipore Sigma, USA) spun at ˜4100 x g in an ultracentrifuge. The mRNA and DNA LNPs were then stored at 4° C. until further use.

### Example 62—Characterization of mRNA- and DNA-containing LNP Compositions Comprising COMPOUNDS 5-49

The following is a nonlimiting example demonstrating that a plurality of mRNA- and DNA-containing LNP compositions may be effectively prepared comprising COMPOUNDS 5-49 of Example 61.

LNP compositions of the present disclosure comprising mRNA or DNA encoding firefly luciferase (hereafter “Fluc”; TriLink) and COMPOUNDS 5-49 were prepared as described in Example 61 by combining one of COMPOUNDS 5-49, the phospholipid DOPE, the structural lipid cholesterol (Chol) and 1,2-dimyristoyl-sn-glycerol methoxypolyethylene glycol (DMG-PEG2000) in the percentages presented in Tables 6 and 7:

TABLE 6

| One of | | | | Lipid | |
|---|---|---|---|---|---|
| COMPOUNDS | | | DMG- | Blend | Lipid:RNA |
| 5-49 | DOPE | Cholesterol | PEG2000 | (mM) | (w/w) |
| 41.44 | 45.85 | 10 | 2.7 | 25 | 40:1 |

TABLE 7

| One of | | | | Lipid | |
|---|---|---|---|---|---|
| COMPOUNDS | | | DMG- | Blend | Lipid:DNA |
| 5-49 | DOPE | Cholesterol | PEG2000 | (mM) | (w/w) |
| 38 | 50 | 10.0 | 2.0 | 25 | 45:1 |

The physical characteristics of the resulting LNPs were analyzed. Table 8 shows the results of the analysis of LNPs comprising COMPOUNDS 5-49 of Example 61.

TABLE 8
LNPs comprising COMPOUNDS 5-49

| COM- | Particle | | Particle | |
|---|---|---|---|---|
| POUND | Size (nm) | PDI | Size (nm) | PDI |
| # | RNA | RNA | DNA | DNA |
| 5 | 180.3 | 0.200 | 187.9 | 0.203 |
| 6 | 202.2 | 0.260 | 167 | 0.154 |
| 7 | 441.0 | 0.099 | 299.7 | 0.221 |
| 8 | 312.8 | 0.073 | 319.6 | 0.167 |
| 9 | 314.0 | 0.129 | 330.9 | 0.123 |
| 10 | 399.8 | 0.099 | 343.5 | 0.088 |
| 11 | 355.6 | 0.145 | 396.1 | 0.152 |
| 12 | 167.6 | 0.235 | 170.6 | 0.235 |
| 13 | 120.6 | 0.272 | 174.1 | 0.232 |
| 14 | 334.8 | 0.104 | 348.8 | 0.219 |
| 15 | 362.5 | 0.115 | 297.6 | 0.183 |
| 16 | 354.0 | 0.142 | 326.3 | 0.108 |
| 17 | 293.3 | 0.145 | 287.7 | 0.129 |
| 18 | 323.3 | 0.153 | 368.6 | 0.072 |
| 19 | 220.1 | 0.133 | 213.9 | 0.131 |
| 20 | 316.9 | 0.082 | 304.3 | 0.223 |
| 21 | 296.9 | 0.102 | 268.3 | 0.206 |
| 22 | 330.9 | 0.176 | 255.5 | 0.141 |
| 23 | 434.4 | 0.184 | 332.8 | 0.189 |
| 24 | 314.9 | 0.106 | 353.7 | 0.294 |
| 25 | 177.9 | 0.143 | 188.5 | 0.227 |
| 26 | 169.4 | 0.202 | 162.1 | 0.151 |
| 27 | 293.9 | 0.149 | 253.9 | 0.177 |
| 28 | 254.2 | 0.027 | 224.7 | 0.12 |
| 29 | 422.3 | 0.197 | 317.2 | 0.081 |
| 30 | 326.7 | 0.170 | 276.9 | 0.167 |
| 31 | 339.9 | 0.145 | 267.9 | 0.063 |
| 32 | 269.6 | 0.108 | 289.2 | 0.09 |
| 33 | 332.9 | 0.079 | 302 | 0.087 |
| 34 | 386.4 | 0.127 | 290.2 | 0.128 |
| 35 | 364.7 | 0.149 | 298.2 | 0.129 |
| 36 | 260.6 | 0.09 | 286.1 | 0.091 |
| 37 | 356.2 | 0.091 | 337.2 | 0.165 |
| 38 | 359.7 | 0.128 | 398.8 | 0.208 |
| 39 | 297.1 | 0.064 | 272.5 | 0.138 |
| 40 | 255.5 | 0.179 | 251.2 | 0.146 |
| 41 | 306.1 | 0.057 | 295.1 | 0.085 |
| 42 | 304.2 | 0.121 | 289.2 | 0.039 |
| 43 | 310.1 | 0.079 | 286.7 | 0.185 |
| 44 | 329 | 0.09 | 270 | 0.102 |
| 45 | 356.1 | 0.131 | 340.4 | 0.082 |
| 46 | 334.6 | 0.078 | 309 | 0.116 |
| 47 | 363.7 | 0.122 | 357.6 | 0.223 |
| 48 | 324.9 | 0.088 | 291.3 | 0.202 |
| 49 | 245.3 | 0.045 | 308.7 | 0.104 |

### Example 63—In vitro LNP-delivery of mRNA or DNA to suspension cells

The following is a nonlimiting example demonstrating that the lipid nanoparticle compositions of the present disclosure can be used to deliver mRNA or DNA to suspension in vitro and that following delivery of the mRNA or DNA, the encoded protein is expressed by the cells.

TF1a cells were seeded in 96 well plates and grown to log confluency in RPMI+10% FBS] under a 5% CO₂ atmosphere at 37° C. A 0.125 μg amount of 5′-CleanCap-firefly luciferase mRNA (TriLink Biotech) or nanoplasmid CMV-firefly luciferase DNA (Nature Technology Corp) formulated with LNP compositions comprising one of COMPOUNDS 5-49 as prepared in Example 61 at the following percentages in Tables 9 and 10 was added to each well. Cells were exposed to four different doses of mRNA or DNA.

TABLE 9

| One of | | | | Lipid | |
|---|---|---|---|---|---|
| COMPOUNDS | | | DMG- | Blend | Lipid:RNA |
| 5-49 | DOPE | Cholesterol | PEG2000 | (mM) | (w/w) |
| 41.44 | 45.85 | 10.0 | 2.7 | 25 | 40:1 |

TABLE 10

| One of | | | | Lipid | |
|---|---|---|---|---|---|
| COMPOUNDS | | | DMG- | Blend | Lipid: DNA |
| 5-49 | DOPE | Cholesterol | PEG2000 | (mM) | (w/w) |
| 38 | 50 | 10.0 | 2.0 | 25 | 45:1 |

After incubating for 24 hr, cells were incubated with non-lytic Promega RealTime-Glo® reagent for Ihr and NanoLuc® luciferase was measured on a plate reader to assess viability. For Compounds Nos. 5-31, a 4-point dosage response curve from a concentration of .024 μg/ml to 3 μg/ml for mRNA and .078 μg/ml to Sug/ml for DNA was measured and the Area under the Curve of measured relative NanoLuc luciferase is shown in Table 11. AUC RT-glo values are a metric of viable cellular return with a higher value associated with higher tolerability of the cationic material.

TABLE 11

| LNPs comprising | AUC RTglo | AUC RTglo |
|---|---|---|
| Compound # | mRNA | DNA |
| 5 | 20977 | 14331 |
| 6 | 20850 | 30271 |
| 7 | 25001 | 45150 |
| 8 | 21610 | 30140 |
| 9 | 22763 | 28044 |
| 10 | 20096 | 31959 |
| 11 | 24157 | 38857 |
| 12 | 12583 | 17389 |
| 13 | 15735 | 22353 |
| 14 | 22784 | 32382 |
| 15 | 22444 | 30442 |
| 16 | 16552 | 20306 |
| 17 | 14914 | 19182 |
| 18 | 23613 | 36192 |
| 19 | 21182 | 24752 |
| 20 | 23424 | 33770 |
| 21 | 27036 | 42103 |
| 22 | 20492 | 28520 |
| 23 | 19123 | 22812 |
| 24 | 24824 | 33495 |
| 25 | 20353 | 15525 |
| 26 | 18062 | 22911 |
| 27 | 23395 | 33106 |
| 28 | 18907 | 23606 |
| 29 | 13929 | 15079 |
| 30 | 12094 | 12780 |
| 31 | 17901 | 24852 |

For Compound Nos. 32-49, a 4-point dosage response curve from a concentration of .04 μg/ml to 5 μg/ml for mRNA or DNA was measured and the Area under the Curve of measured relative NanoLuc luciferase is shown in Table 12.

TABLE 12

| LNPs comprising | AUC RTglo | AUC RTglo |
|---|---|---|
| Compound # | mRNA | DNA |
| 32 | 35240 | 40000 |
| 33 | 36589 | 38841 |
| 34 | 48979 | 49184 |
| 35 | 54436 | 56656 |
| 36 | 33189 | 41933 |
| 37 | 22980 | 22447 |
| 38 | 39957 | 50811 |
| 39 | 28211 | 35735 |
| 40 | 21761 | 22082 |
| 41 | 32753 | 35010 |
| 42 | 34655 | 44445 |
| 43 | 32478 | 33831 |
| 44 | 38479 | 39603 |
| 45 | 34010 | 37083 |
| 46 | 22713 | 23531 |
| 47 | 24826 | 28387 |
| 48 | 31598 | 36435 |
| 49 | 26425 | 27389 |

After reading out viability the cells were spun down, lysed, and read on the plate reader for firefly luciferase expression activity. For Compounds Nos. 5-31, a 4-point dosage response curve from a concentration of .024 μg/ml to 3 μg/ml for mRNA and .078 μg/ml to 5 μg/ml for DNA was measured and the Area under the Curve of measured relative firefly luciferase is shown in Table 13. AUC firefly luciferase values are a metric of bulk protein expression and a higher value associated with higher delivery of the cationic material.

TABLE 13

| LNPs | | |
|---|---|---|
| comprising | AUC Fluc | AUC Fluc |
| Compound # | mRNA | DNA |
| 5 | 810122 | 154283 |
| 6 | 1249552 | 221766 |
| 7 | 274161 | 30127 |
| 8 | 2603542 | 20269 |
| 9 | 1.3153076e+007 | 155667 |
| 10 | 2.8067145e+007 | 150864 |
| 11 | 4848378 | 97985 |
| 12 | 564724 | 137373 |
| 13 | 1046807 | 233460 |
| 14 | 306661 | 82843 |
| 15 | 656735 | 62763 |
| 16 | 2.5425248e+007 | 187281 |
| 17 | 1.1133395e+007 | 490806 |
| 18 | 4501321 | 339049 |
| 19 | 541317 | 35202 |
| 20 | 41869 | 20244 |
| 21 | 48346 | 35880 |
| 22 | 1.2460769e+007 | 339183 |
| 23 | 1.4815498e+007 | 630135 |
| 24 | 861492 | 56252 |
| 25 | 10857 | 14400 |
| 26 | 14055 | 16522 |
| 27 | 117733 | 16625 |
| 28 | 246598 | 7431 |
| 29 | 2.4782805e+007 | 231288 |
| 30 | 7955552 | 1778921 |
| 31 | 1.2444405e+007 | 326746 |

For Compound Nos. 32-49, a 4-point dosage response curve from a concentration of .04 μg/ml to 5 μg/ml for mRNA or DNA was measured and the Area under the Curve of measured relative firefly luciferase is shown in Table 14.

TABLE 14

| LNPs | | |
|---|---|---|
| comprising | AUC Fluc | AUC |
| Compound # | mRNA | Fluc DNA |
| 32 | 96944 | 7521 |
| 33 | 64362 | 11424 |
| 34 | 39229 | 3593 |
| 35 | 7035 | 1491 |
| 36 | 3553954 | 45706 |
| 37 | 154629 | 12176 |
| 38 | 96430 | 4337 |
| 39 | 254298 | 27038 |
| 40 | 5620 | 434.6 |
| 41 | 163623 | 16859 |
| 42 | 57886 | 2225 |
| 43 | 218345 | 3561 |
| 44 | 80630 | 3925 |
| 45 | 66511 | 12761 |
| 46 | 80454 | 3157 |
| 47 | 12752 | 4438 |
| 48 | 21245 | 2964 |
| 49 | 28103 | 60485 |

As shown in Tables 11-14, the LNP compositions comprising COMPOUNDS 5-49 were successfully able to deliver mRNA and DNA to suspension TFla cells.

### Example 64—In vitro LNP-delivery of mRNA to adherent cells (human)

The following is a nonlimiting example demonstrating that the lipid nanoparticle compositions of the present disclosure can be used to deliver mRNA to adherent cells in vitro and that following delivery of the mRNA, the encoded protein is expressed by the cells.

The details of Example 63 were followed with HepG2 (human hepatocarcinoma) cells. LNP compositions comprising one of COMPOUNDS 5-31 as prepared in Example 61 were added to HepG2 cells at the following percentages in Table 15 as described in Example 63. Cells were exposed to four different doses of mRNA.

TABLE 15

| One of | | | | Lipid | |
|---|---|---|---|---|---|
| COMPOUNDS | | | DMG- | Blend | Lipid:RNA |
| 5-31 | DOPE | Cholesterol | PEG2000 | (mM) | (w/w) |
| 41.44 | 45.85 | 10.0 | 2.7 | 25 | 40:1 |

The Area under the Curve of measured relative NanoLuc luciferase was measured in the assay as described in Example 63 and results are shown in Table 16.

TABLE 16

| LNPs comprising | AUC RTglo |
|---|---|
| Compound # | mRNA |
| 5 | 43099 |
| 6 | 41264 |
| 7 | 38470 |
| 8 | 37061 |
| 9 | 37002 |
| 10 | 41124 |
| 11 | 39628 |
| 12 | 34859 |
| 13 | 32578 |
| 14 | 35896 |
| 15 | 36738 |
| 16 | 34144 |
| 17 | 33741 |
| 18 | 36929 |
| 19 | 36745 |
| 20 | 34523 |
| 21 | 35899 |
| 22 | 34645 |
| 23 | 33337 |
| 24 | 33379 |
| 25 | 35913 |
| 26 | 38971 |
| 27 | 36325 |
| 28 | 34058 |
| 29 | 29889 |
| 30 | 29923 |
| 31 | 29645 |

The Area under the Curve of measured relative firefly luciferase was measured in the assay as described in Example 63 and results are shown in Table 17.

TABLE 17

| LNPs comprising | AUC Fluc |
|---|---|
| Compound # | mRNA |
| 5 | 172646 |
| 6 | 177028 |
| 7 | 67668 |
| 8 | 821750 |
| 9 | 6612762 |
| 10 | 11516241 |
| 11 | 6492307 |
| 12 | 140207 |
| 13 | 188513 |
| 14 | 57775 |
| 15 | 81292 |
| 16 | 12048348 |
| 17 | 6439367 |
| 18 | 3241512 |
| 19 | 2021693 |
| 20 | 28860 |
| 21 | 24231 |
| 22 | 5608617 |
| 23 | 4153426 |
| 24 | 490740 |
| 25 | 26536 |
| 26 | 50218 |
| 27 | 28091 |
| 28 | 260079 |
| 29 | 7475505 |
| 30 | 4332496 |
| 31 | 8100644 |

As shown in Tables 16 and 17, the LNP compositions comprising COMPOUNDS 5-31 were successfully able to deliver mRNA to adherent HepG2 cells.

### Example 65—In vitro LNP-delivery of RNA to adherent cells (mouse)

The following is a nonlimiting example demonstrating that the lipid nanoparticle compositions of the present disclosure can be used to deliver mRNA to adherent in vitro and that following delivery of the mRNA, the encoded protein is expressed by the cells.

The details of Example 63 were followed with AML12 (mouse hepatocyte) cells. LNP compositions comprising one of COMPOUNDS 5-31 as prepared in Example 61 were added to AML12 cells at the following percentages in Table 18 as described in Example 63. Cells were exposed to four different doses of mRNA.

TABLE 18

| One of | | | | Lipid | |
|---|---|---|---|---|---|
| COMPOUNDS | | | DMG- | Blend | Lipid:RNA |
| 5-31 | DOPE | Cholesterol | PEG2000 | (mM) | (w/w) |
| 41.44 | 45.85 | 10.0 | 2.7 | 25 | 40:1 |

The Area under the Curve of measured relative firefly luciferase was measured in the assay as described in Example 63 and results are shown in Table 19.

TABLE 19

| LNPs comprising | AUC Fluc |
|---|---|
| Compound # | mRNA |
| 5 | 15916 |
| 6 | 40326 |
| 7 | 8153 |
| 8 | 143809 |
| 9 | 494563 |
| 10 | 621180 |
| 11 | 159221 |
| 12 | 10771 |
| 13 | 58032 |
| 14 | 15379 |
| 15 | 29106 |
| 16 | 419692 |
| 17 | 940423 |
| 18 | 148693 |
| 19 | 4550 |
| 20 | 1863 |
| 21 | 5645 |
| 22 | 83044 |
| 23 | 587623 |
| 24 | 19332 |
| 25 | 1146 |
| 26 | 3308 |
| 27 | 8602 |
| 28 | 40983 |
| 29 | 943385 |
| 30 | 2050197 |
| 31 | 267136 |

As shown in Table 19, the LNP compositions comprising COMPOUNDS 5-31 were successfully able to deliver mRNA to adherent AML12 cells.

### Example 66—General Method for the Preparation of LNPs of Present Disclosure Comprising RNA or DNA

LNP compositions of the present disclosure comprising exemplary compounds of Formula (I) were prepared by combining various percentages of an exemplary compound of Formula (I), the phospholipid DOPE, the structural lipid cholesterol (Chol) and 1,2-dimyristoyl-sn-glycerol methoxypolyethylene glycol (DMG-PEG2000; Avanti Polar Lipids, Alabaster, Alabama, USA).

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol % as shown in Tables 20 and 21.

TABLE 20

| One of | | | | Lipid | | |
|---|---|---|---|---|---|---|
| COMPOUNDS | | | DMG- | Blend | Lipid:RNA | Flow |
| 5-49 | DOPE | Cholesterol | PEG2000 | (mM) | (w/w) | Rate |
| 33.5 | 32 | 33.5 | 1 | 25 | 40:1 | 20 |

TABLE 21

| One of | | | | Lipid | | |
|---|---|---|---|---|---|---|
| COMPOUNDS | | | DMG- | Blend | Lipid:DNA | Flow |
| 5-49 | DOPE | Cholesterol | PEG2000 | (mM) | (w/w) | Rate |
| 35 | 20 | 41.8 | 3.16 | 25 | 80:1 | 20 |

A 1 mg/ml solution of the desired mRNA or DNA to be incorporated into the LNPs was added to 150 mM sodium acetate buffer (pH 5.2) to form a stock solution and kept on ice. The ethanol phase was vigorously mixed with the nucleic acid in sodium acetate phase using the Precision Nanoassemblr instrument.

The resultant mRNA or DNA LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against phosphate buffered saline (PBS) (dialysate: dialysis buffer volume at least 1:200 v/v), pH 7.4 overnight at 4° C. (or alternatively room temperature for at least 4 hours), to remove the 25% ethanol and achieve a complete buffer exchange. In some experiments the LNPs were further concentrated by in an Amicon® Ultra-4 centrifugal filter unit, MWCO-30 kDa (Millipore Sigma, USA) spun at ˜4100 x g in an ultracentrifuge. The mRNA and DNA LNPs were then stored at 4° C. until further use.

### Example 67—In vivo LNP-delivery of mRNA to liver

The following is a nonlimiting example demonstrating that the lipid nanoparticle compositions of the present disclosure can be used to deliver mRNA to liver cells in vivo and expression of the encoded protein.

Adult female BALB/C mice (n=3/group) were intravenously administered 0.5 mg/kg of 5′-CleanCap-fLuciferase mRNA (TriLink Biotech) formulated with LNP compositions as described in Example 66. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results of this experiment for LNP compositions comprising selected COMPOUNDS from 5-49 are shown in Table 22.

TABLE 22

| LNPs | |
|---|---|
| comprising | Ave flux (p/s) |
| Compound # | n = 3 |
| 19 | 1.10E+06 |
| 8 | 1.10E+07 |
| 9 | 1.77E+10 |
| 16 | 3.67E+10 |
| 22 | 9.27E+09 |
| 29 | 2.44E+10 |
| 10 | 7.16E+09 |
| 17 | 2.96E+10 |
| 23 | 6.24E+09 |
| 30 | 3.26E+10 |
| 31 | 2.24E+09 |

As shown in Table 22, luciferase expression was detected in treated animals for each of the LNP compositions comprising selected COMPOUNDS from 5-49 of the present disclosure. Mice treated with each of the LNP compositions comprising selected COMPOUNDS from 5-49 exhibited higher luciferase expression, predominantly in the liver as detected and quantified by BLI, than those mice treated with the PBS vehicle control.

The mice above were also monitored for changes in body weight after LNP delivery. Mice were manually weighed on a scale before LNP dosage and then weighed 24 hrs after administration of 0.5 mg/kg of LNP article encapsulating Cleancap firefly luciferase mRNA. The average of weight change n=3 is reported below. As shown in Table 23 below, loss of weight in the mice indicated LNP delivery.

TABLE 23

| LNPs | Ave weight |
|---|---|
| comprising | change (gram) |
| Compound # | n = 3 |
| 19 | 0.70 |
| 8 | −2.53 |
| 9 | −3.03 |
| 16 | −3.60 |
| 22 | −6.40 |
| 29 | −6.07 |
| 10 | −2.03 |
| 17 | −3.10 |
| 23 | −5.97 |
| 30 | −5.67 |
| 31 | −4.10 |

The following is a nonlimiting example demonstrating that the lipid nanoparticle compositions of the present disclosure can be used to deliver DNA to liver cells in vivo as shown by expression of the encoded protein.

### Example 68—In vivo LNP-delivery of DNA to liver

Juvenile female BALB/C mice of age 14 days (n=4/group) were retroorbitally intravenously administered 0.25 mg/kg of nanoplasmid CMV-firefly luciferase (Nature Technology Corp) formulated with LNP compositions as described in Example 66. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results of this experiment for LNP compositions comprising selected COMPOUNDS from 5-49 are shown in Table 24.

TABLE 24

| LNPs | |
|---|---|
| comprising | Ave flux (p/s) |
| Compound # | n = 4 |
| 30 | 7332500000 |
| 16 | 8905000000 |
| 23 | 3012500000 |
| 29 | 9120000000 |

As shown in Table 24, luciferase expression was detected in treated animals for each of the LNP compositions comprising selected COMPOUNDS from 5-49 of the present disclosure. Mice treated with each of the LNP compositions comprising selected COMPOUNDS from 5-49 exhibited higher luciferase expression, predominantly in the liver as detected and quantified by BLI, than those mice treated with the PBS vehicle control.

### Example 69—Preparation of LNPs of Present Disclosure Comprising mRNA and In Vivo Screening

### A. Preparation

The following is a nonlimiting example that provides exemplary methods for formulating a plurality of multi-component LNP compositions comprising exemplary compounds of Formula (I) and mRNA.

To formulate the LNPs, various percentages of one of COMPOUNDS 5-49, the phospholipid DOPE, the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages. A subset of the LNP compositions is shown in Table 25.

TABLE 25

| LNPs | One of | | | | | |
|---|---|---|---|---|---|---|
| comprising | Compound | | | | Total | Lipid: |
| Compound | Nos. 5-49 | DOPE | Chol | PEG-DMG | Lipid | RNA |
| No. | (% moles) | (% moles) | (% moles) | (% moles) | (mM) | (w/w) |
| 30 | 40.68 | 10 | 47.12 | 2.2 | 25 | 100 |
| 9 | 29 | 10 | 60 | 1 | 25 | 100 |
| 16 | 60 | 10 | 29 | 1 | 20 | 100 |
| 29 | 60 | 27.8 | 10 | 2.2 | 20 | 100 |
| 17 | 29 | 10 | 60 | 1 | 25 | 40 |
| 17 | 24.29 | 28.3 | 46.41 | 1 | 25 | 100 |
| 16 | 44.5 | 44.5 | 10 | 1 | 25 | 100 |
| 22 | 46.32 | 10 | 39.48 | 4.2 | 25 | 100 |

A 1 mg/ml solution of the 5′-CleanCap--fLuciferase mRNA (TriLink Biotech) to be incorporated into the LNPs was added to 150 mM sodium acetate buffer (pH 5.2) to form a stock solution and kept on ice. The lipid phase was mixed with the aqueous mRNA phase inside a microfluidic chip using a NanoAssemblr® instrument (Precision Nanosystems, Vancouver, BC, Canada) according to the manufacturer's instructions to form LNP compositions comprising encapsulated mRNAs. Nanoassemblr process parameters for mRNA encapsulation are shown in the Table 26.

TABLE 26

| Total flow rate | Lipid phase: aqueous |
|---|---|
| (ml/min) | (RNA) phase (v/v) |
| 20 | 1:3 |

The resultant mRNA LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against phosphate buffered saline (PBS) (dialysate: dialysis buffer volume at least 1:200 v/v), pH 7.4 overnight at 4° C. (or alternatively room temperature for at least 4 hours), to remove the 25% ethanol and achieve a complete buffer exchange. In some experiments the LNPs were further concentrated in an Amicon® Ultra-4 centrifugal filter unit, MWCO-30 kDa (Millipore Sigma, USA) spun at ˜4100 x g in an ultracentrifuge. The mRNA LNPs were then stored at 4° C. until further use.

The average particle size diameter of the LNPs was approximately 75-150 nm.

### B. In Vivo Screening

Adult female BALB/C mice (n=3/group) were intravenously administered 0.5 mg/kg of 5′-CleanCap-fLuciferase mRNA (TriLink Biotech) formulated with the LNP compositions shown in Table 25. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 27.

TABLE 27

| | Mean Liver |
|---|---|
| | Luciferase |
| LNPs comprising | Flux |
| Compound No. | (p/s) |
| 30 | 1.28e11 |
| 9 | 4.62e10 |
| 16 (60% mol) | 5.92e10 |
| 29 | 3.02e10 |
| 17 (29% mol) | 2.52e10 |
| 17 (24.29% mol) | 3.43e10 |
| 16 (44.5% mol) | 3.09e10 |
| 22 | 1.84e10 |

As shown in Table 27, LNP compositions of the present disclosure successfully delivered mRNA in vivo, predominantly to cells in the liver, and the encoded protein was subsequently expressed by the cells.

In addition, the body weight of mice treated with the LNP compositions of Table 25 was assessed prior to intravenous administration and twenty-four hours post-administration and the body weights at baseline and post-treatment were compared. The average percentage of body weight change for each group of mice treated with each LNP composition of Table 25 is shown in Table 28.

TABLE 28

| LNPs comprising | % Body Weight |
|---|---|
| Compound No. | Change 24 hr |
| 30 | −2.8 |
| 9 | +1.4 |
| 16 (60% mol) | −5.2 |
| 29 | −2.4 |
| 17 (29% mol) | −5.2 |
| 17 (24.29% mol) | −10.0 |
| 16 (44.5% mol) | −7.4 |
| 22 | +1.2 |

As shown in Table 28, the LNP compositions of the present disclosure were well tolerated with most treated mice retaining original body weights or even slightly gaining weight.

### Example 70—Preparation of LNPs of Present Disclosure Comprising mRNA and In Vivo Screening

The following is a nonlimiting example that provides exemplary methods for formulating a plurality of multi-component LNP compositions comprising exemplary compounds of Formula (I) and mRNA.

LNP compositions comprising various percentages of one of COMPOUNDS 5-49, the phospholipid (DOPE or DOPC), the structural lipid cholesterol (Chol) and 1,2-dimyristoyl-sn-glycerol methoxypolyethylene glycol (DMG-PEG2000; Avanti Polar Lipids, Alabaster, Alabama, USA) as shown in Table 29 were prepared as described in Example 69.

TABLE 29

| LNPs | One of | | | | | |
|---|---|---|---|---|---|---|
| comprising | Compound | | | PEG- | Total | |
| Compound | Nos. 5-49 | Phospholipid | | DMG | Lipid | |
| No. | (% moles) | (% moles) | Chol (% moles) | (% moles) | (mM) | Lipid:RNA (w/w) |
| 30 | 40.68 | 10% DOPE | 47.12 | 2.2 | 25 | 100 |
| 30 | 40.68 | 10% DOPC | 47.12 | 2.2 | 25 | 100 |
| 16 | 60 | 10% DOPE | 29 | 1 | 20 | 100 |
| 16 | 60 | 10% DOPC | 29 | 1 | 20 | 100 |
| 17 | 24.29 | 28.3% DOPE | 46.41 | 1 | 25 | 100 |
| 17 | 24.29 | 28.3% DOPC | 46.41 | 1 | 25 | 100 |

Adult female BALB/C mice (n=2/group) were intravenously administered 0.5 mg/kg of 5′-CleanCap--fLuciferase mRNA (TriLink Biotech) formulated with the LNP compositions shown in Table 29. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 30.

TABLE 30

| | Mean Liver |
|---|---|
| | Luciferase |
| LNPs comprising | Flux |
| Compound No. | (p/s) |
| 30 (10% DOPE) | 8.58e10 |
| 30 (10% DOPC) | 9.05e10 |
| 16 (10% DOPE) | 1.61e11 |
| 16 (10% DOPC) | 1.26e11 |
| 17 (28.3% DOPE) | 8.0e10 |
| 17 (28.3% DOPC) | 2.46e9 |

As shown in Table 30, LNP compositions of the present disclosure successfully delivered mRNA in vivo, predominantly to cells in the liver, and the encoded protein was subsequently expressed by the cells.

### Example 71—Preparation of LNPs of Present Disclosure Comprising DNA and In Vivo Screening

The following is a nonlimiting example that provides exemplary methods for formulating a plurality of multi-component LNP compositions comprising exemplary compounds of Formula (I) and DNA.

LNP compositions of the present disclosure comprising DNA encoding firefly luciferase (Nature Technology Corporation) were prepared as described in Example 69. The LNP compositions are shown in Table 31.

TABLE 31

| LNPs | One of | | | | | |
|---|---|---|---|---|---|---|
| comprising | Compound | | | PEG- | Total | |
| Compound | Nos. 5-49 | Phospholipid | | DMG | Lipid | |
| No. | (% moles) | (% moles) | Chol (% moles) | (% moles) | (mM) | Lipid:RNA (w/w) |
| 30 | 40.68 | 10% DOPE | 47.12 | 2.2 | 25 | 100 |
| 30 | 40.68 | 10% DOPC | 47.12 | 2.2 | 25 | 100 |
| 16 | 60 | 10% DOPE | 29 | 1 | 20 | 100 |
| 16 | 60 | 10% DOPC | 29 | 1 | 20 | 100 |
| 17 | 24.29 | 28.3% DOPE | 46.41 | 1 | 25 | 100 |
| 17 | 24.29 | 28.3% DOPC | 46.41 | 1 | 25 | 100 |

Adult BALB/C mice were administered 0.5 mg/kg of total DNA (n=4) of the LNP composition listed in Table 31. The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 32.

TABLE 32

| | Mean Liver |
|---|---|
| | Luciferase |
| LNPs comprising | Flux |
| Compound No. | (p/s) |
| 30 (10% DOPE) | 1.95e7 |
| 30 (10% DOPC) | 1.28e7 |
| 16 (10% DOPE) | 4.12e7 |
| 16 (10% DOPC) | 1.67e7 |
| 17 (28.3% DOPE) | 1.45e7 |
| 17 (28.3% DOPC) | 9.21e5 |

As shown in Table 32, LNP compositions of the present disclosure successfully delivered DNA to liver cells and the encoded transgene was subsequently expressed in the liver cells.

### Example 72—LNP Compositions of the Present Disclosure Reduce Immune Response

The following is a non-limiting example demonstrating that the in vitro administration of certain LNP compositions of the present disclosure resulted in a decrease in complement activation as measured by serum levels of C₃a in human serum.

LNP compositions were prepared as described in Example 69. The LNP compositions encapsulated RNA molecules comprising a sequence encoding HA-tagged SPB.

Normal human serum (NHS) was thawed at 37° C. and 100 μL was aliquoted into a 1.5 mL centrifuge tube. NHS was then treated with 16 μL of an LNP composition at 0.1 mg/mL and incubated for 30 mins at 37° C. The reaction mixtures were then diluted 1:5000 and analyzed using a C₃a ELISA kit (Quidel).

The levels of C₃a in the samples treated with certain LNP compositions of the present disclosure comprising DOPC were compared to the levels of C₃a in the samples treated with certain LNP compositions of the present disclosure comprising DOPE and the values are reported in Table 33.

TABLE 33

| LNPs comprising | C3a Concentration |
|---|---|
| Compound No. | (ng/mL) |
| 30 (10% DOPE) | 11203 |
| 30 (10% DOPC) | 6249 |
| 16 (10% DOPE) | 7244 |
| 16 (10% DOPC) | 5277 |

As shown in Table 33, certain LNP compositions of the present disclosure exhibited a significantly reduced immune response profile as compared to other LNP compositions as shown by the reduced C₃a serum levels.

### Example 73—LNP Compositions of Present Disclosure Deliver RNA with High Specificity to the Liver In Vivo

This experiment shows the ability of LNP compositions of the present disclosure to deliver Cas-CLOVER mRNA to the liver, targeted by a pair of gRNAs to the psk9 gene, resulting in subsequent in vivo gene editing of the psk9 gene. Pcsk9 protein is secreted by hepatocytes and binds to the LDL receptor, inducing its internalization and lysosomal degradation, resulting in increased circulating levels of LDL-cholesterol.

In this experiment, each group of adult female BALB/C mice (n=2/group) was intravenously co-administered mRNA encoding 5′-CleanCap-5MeC-Cas-CLOVER (SEQ ID NO: 5) and a pair of gRNAs (SEQ ID NOs: 1 and 2) targeted to the first exon of the mouse pcsk9 gene. The mRNA and gRNA molecules were formulated within LNP compositions of the present disclosure comprising one of COMPOUNDS 5-49, a phospholipid (DOPE or DOPC), Cholesterol and DMG-PEG2000 at the molar ratios shown in Table 34. All cytidine residues in the mRNA were 5-methylcytidine (5-MeC).

TABLE 34

| LNPs | One of | | | | | |
|---|---|---|---|---|---|---|
| comprising | Compound | | | PEG- | Total | |
| Compound | Nos. 5-49 | Phospholipid | | DMG | Lipid | |
| No. | (% moles) | (% moles) | Chol (% moles) | (% moles) | (mM) | Lipid:RNA (w/w) |
| 30 | 40.68 | 10% DOPE | 47.12 | 2.2 | 25 | 100 |
| 30 | 40.68 | 10% DOPC | 47.12 | 2.2 | 25 | 100 |
| 16 | 60 | 10% DOPE | 29 | 1 | 20 | 100 |
| 16 | 60 | 10% DOPC | 29 | 1 | 20 | 100 |

LNP compositions of the present disclosure (1.5 mg/kg) were administered to the mice from each group. One group of mice was administered a dose of Cas-CLOVER mRNA and a pair of pcsk9 gRNA, both co-encapsulated in an LNP composition of the present disclosure. One group of mice was treated with vehicle (PBS, Thermo Fisher Scientific, USA) as a negative control.

After seven days post-administration, DNA was isolated from four tissue types from the mice in each group: liver, spleen, lung, and kidney. Briefly, tissues were resected after euthanasia, flash frozen in liquid nitrogen, mixed with lysis buffer (15 mg of tissue in 200 μL of lysis buffer+10 uL Proteinase K) and pulverized in a TissueLyser II (Qiagen) using Triple-Pure zirconium beads (Fisher Scientific). Homogenized tissue was then incubated at 56C for 30 minutes, and column-purified using a Monarch Genomic DNA Purification kit from New England Biolabs under manufacturer's instructions. Final DNA elution was done in 50 μL of elution buffer (10 mM Tris-C₁, pH 8.5). Concentration and purity of DNA samples was assessed by measuring absorbance at 260 and 280 nm using a Nanodrop. Also, blood samples were drawn for LDL-C quantification. Briefly, 500 μL of blood was collected after euthanasia via cardiac puncture using 2 ml syringes and 25G needles, transferred to microcentrifuge tubes, incubated at room temperature for 1 hour, and centrifuged at 1500 g for 15 minutes to separate the cellular fraction from serum. Serum fraction (200 uL) was transferred to a new tube and stored at −80C until further analysis.

Serum levels of the pcsk9 protein in the mice were measured 7 days after administration and the results are shown in Table 35. Briefly, a mouse Pcsk9 ELISA kit (Biolegend) was used to determine Pcsk9 in each serum sample following manufacturer's instructions. All serum samples were assayed in triplicate and results were expressed as percentage reduction in Pcsk9 levels compared with Pcsk9 levels of PBS-treated mice.

TABLE 35

| LNPs comprising | Pcsk9 |
|---|---|
| Compound No. | (pg/mL) |
| PBS control | 184 ± 25 |
| 30 (10% DOPE) | 71 ± 19 |
| 30 (10% DOPC) | 153 ± 22 |
| 16 (10% DOPE) | 45 ± 3 |
| 16 (10% DOPC) | 60 ± 17 |

The results of Table 35 show that Cas-CLOVER mRNA delivered by LNP compositions of the present disclosure is effective at editing the pcsk9 gene in the liver in vivo.

### Example 74—LNPs of the Present Disclosure for the Treatment of Hemophilia

The following is a non-limiting example demonstrating the compositions and methods of the present disclosure can be used to in the treatment of hemophilia, and more specifically hemophilia A.

On Day 13-16 of life, wild-type BALB/c juvenile mice (N=4-6) were administered either (1) LNPs encapsulating FVIII transposon co-delivered with LNPs encapsulating SPB (delivery method denoted as “Dual-LNP”) or (2) a single co-encapsulated LNP encapsulating both FVIII transposon and SPB (delivery method denoted as “Co-encapsulated LNP”). LNPs were administered at a 1:1, 2:1 or 4:1 mRNA: DNA ratio for both Dual-LNP and Co-encapsulated LNP. Mice receiving Dual-LNP treatment at 1:1 received 0.25 mg/kg of mRNA-LNP co-dosed with 0.25 mg/kg of DNA-LNP; at 2:1 received 0.5 mg/kg of mRNA-LNP co-dosed with 0.25 mg/kg of DNA-LNP; and at 4:1 received 1.0 mg/kg of mRNA-LNP co-dosed with 0.25 mg/kg of DNA-LNP. Mice receiving Co-encapsulated LNP treatment at 1:1 received 0.5 mg/kg of co-encapsulated LNP encapsulating mRNA and DNA at 1:1 mRNA: DNA ratio; at 2:1 received 0.75 mg/kg of co-encapsulated LNP encapsulating mRNA and DNA at 2:1 mRNA: DNA ratio, and at 4:1 received 1.25 mg/kg of co-encapsulated LNP encapsulating mRNA and DNA at 4:1 mRNA: DNA ratio. A separate group of mice were administered a benchmark C₁₂-200 Dual-LNP composition at a 2:1 mRNA: DNA ratio (0.5 mg/kg of mRNA-LNP co-dosed with 0.25 mg/kg of DNA-LNP).

The LNPS encapsulating FVIII transposon were LNPs of the present disclosure comprising nanoplasmid DNA (SEQ ID NO: 6) comprising a transposon, wherein the transposon comprised an expression cassette comprising, a first piggyBac inverted terminal repeat (right ITR), followed by a first insulator sequence, followed by three tandem copies of the SERPINA1 enhancer, followed by a Transthyretin (TTR) enhance/promoter and minute virus of mice (MVM) intron sequence, followed by a codon optimized nucleic acid sequence encoding for modified human Factor VIII (FVIII), followed by the AES untranslated region (UTR), followed by the mTRNR1 UTR, followed by an SV40-late polyadenylation and cleavage signal sequence, followed by a second insulator sequence, followed by a second piggyBac inverted terminal repeat (left ITR). The LNPs encapsulating FVIII transposon comprised one of COMPOUNDS 5-49, a phospholipid (DOPE or DOPC), Cholesterol and DMG-PEG2000 at the molar ratios shown in Table 36. The benchmark FVIII transposon LNPs comprised C₁₂-200, DOPE, Cholesterol and DMG-PEG2000 at a molar ratio of 0.35:0.2:0.4184:0.0316 and had a lipid: DNA ratio of 80:1 (w/w).

The LNPs encapsulating SPB LNPs were INPs of the present disclosure comprising mRNA encoding active SPB, and one of COMPOUNDS 5-49, a phospholipid (DOPE or DOPC), Cholesterol and DMG-PEG2000 at the molar ratios shown in Table 36. All cytidine residues in the mRNA were 5-methylcytidine (5-MeC). The benchmark SPB LNPs comprised C₁₂-200, DOPE, Cholesterol and DMG-PEG2000 at a molar ratio of 0.335:0.32:0.335:0.1 and had a lipid: RNA ratio of 40:1 (w/w).

TABLE 36

| | | | | | | Lipid: Nucleic Acid |
|---|---|---|---|---|---|---|
| LNPs | One of | | | | | (Lipid:DNA, |
| comprising | Compound Nos. | | | | Total | Lipid: RNA, or |
| Compound | 5-49 | Phospholipid | Chol | PEG-DMG | Lipid | Lipid:RNA + DNA) |
| No. | (% moles) | (% moles) | (% moles) | (% moles) | (mM) | (w/w) |
| 30 | 40.68 | 10% DOPE | 47.12 | 2.2 | 25 | 100 |
| 30 | 40.68 | 10% DOPC | 47.12 | 2.2 | 25 | 100 |
| 16 | 60 | 10% DOPE | 29 | 1 | 20 | 100 |
| 16 | 60 | 10% DOPC | 29 | 1 | 20 | 100 |

The single co-encapsulated LNP encapsulating both FVIII transposon and SPB was an LNP of the present disclosure comprising the nanoplasmid DNA of SEQ ID NO: 6 and mRNA encoding active SPB as put forth in Table 36.

Prior to administration, juvenile mice were placed under anesthesia induced by isoflurane. For Dual-LNP administration, both DNA-LNP and mRNA-LNPs were mixed together in a tube, 50-80 uL drawn into a single 29 gauge insulin syringe, and delivered via intravenous (IV) through the retro-orbital sinus. For co-encapsulated LNP delivery, 50-80 uL of co-encapsulated LNP was drawn into a single 29 gauge insulin syringe, and delivered via intravenous (IV) through the retro-orbital sinus. Juvenile mice were brought back to normal body temperature on a 37° C. heat pad before being placed back with their mother. On Day 7 post-treatment, plasma was collected from treated mice. For plasma collection, treated mice were put under anesthesia with isoflurane, approximately 150 uL whole blood was retro-orbitally collected, whole blood was mixed with 10% volume of 3.2% sodium citrate, centrifuged at 15,000 g for 15 minutes at 20° C., and plasma supernatant was collected. hFVIII antigen levels were measured using the Visualize™ Factor VIII Antigen Plus Kit (Affinity Biologicals™ Inc.).

The results of this analysis for the 2:1 mRNA: DNA treatment groups are shown in Table 37.

TABLE 37

| | % of Normal hFVIII Plasma Ag | |
|---|---|---|
| LNPs comprising | | Co-encapsulated |
| Compound No. | Dual LNP (2:1) | LNP (2:1) |
| C12 control | 197.5 | ND |
| 30 (10% DOPC) | 10.9 | 139.3 |
| 16 (10% DOPC) | 19.3 | 18.0 |

Table 37 shows that LNPs of the present disclosure can be used to drive FVIII expression in vivo, with DNA/mRNA either co-delivered in separate LNP compositions or co-encapsulated in a single LNP composition, thereby demonstrating that the compositions and methods of the present disclosure can be used to treat hemophilia A. Further, the results presented in this example show that certain LNPs of the present disclosure co-encapsulating both mRNA and DNA demonstrated equivalent or superior performance compared to co-delivery of mRNA with DNA.

### Example 75—LNPs of the Present Disclosure for the Treatment of Hemophilia

The following is a non-limiting example demonstrating the compositions and methods of the present disclosure can be used in the treatment of hemophilia, and more specifically hemophilia A.

On Day 13-16 of life, wild-type BALB/c juvenile mice (N=4-5) or hemophilia A (B6;129S-F8tmlKaz/J) juvenile mice (N=4-6) were administered either (1) LNPs encapsulating FVIII transposon co-delivered with LNPs encapsulating SPB (delivery method denoted as “Dual-LNP”) or (2) a single co-encapsulated LNP encapsulating both FVIII transposon and SPB (delivery method denoted as “Co-encapsulated LNP”). Mice receiving Dual-LNP treatment received 0.6 mg/kg of mRNA-LNP co-dosed with 0.15 mg/kg of DNA-LNP. Mice receiving Co-encapsulated LNP treatment received 0.75 mg/kg of co-encapsulated LNP encapsulating mRNA and DNA at 4:1 mRNA DNA ratio. A separate group of mice were administered a benchmark C₁₂-200 Dual-LNP composition at a 2:1 mRNA: DNA ratio (0.5 mg/kg of mRNA-LNP co-dosed with 0.25 mg/kg of DNA-LNP).

The LNPS encapsulating FVIII transposon were LNPs of the present disclosure comprising nanoplasmid DNA (SEQ ID NO: 6) comprising a transposon, wherein the transposon comprised an expression cassette comprising, a first piggyBac inverted terminal repeat (right ITR), followed by a first insulator sequence, followed by three tandem copies of the SERPINA1 enhancer, followed by a Transthyretin (TTR) enhance/promoter and minute virus of mice (MVM) intron sequence, followed by a codon optimized nucleic acid sequence encoding for modified human Factor VIII (FVIII), followed by the AES untranslated region (UTR), followed by the mTRNR1 UTR, followed by an SV40-late polyadenylation and cleavage signal sequence, followed by a second insulator sequence, followed by a second piggyBac inverted terminal repeat (left ITR). The LNPs encapsulating FVIII transposon comprised COMPOUND NO. 30, DOPC, Cholesterol and DMG-PEG2000 at the molar ratios shown in Table 38. The benchmark FVIII transposon LNPs comprised C₁₂-200, DOPE, Cholesterol and DMG-PEG2000 at a molar ratio of 0.35:0.2:0.4184:0.0316 and had a lipid: DNA ratio of 80:1 (w/w).

The LNPs encapsulating SPB LNPs were LNPs of the present disclosure comprising mRNA encoding active SPB, COMPOUND NO. 30, DOPC, Cholesterol and DMG-PEG2000 at the molar ratios shown in Table 38. All cytidine residues in the mRNA were 5-methylcytidine (5-MeC). The benchmark SPB LNPs comprised C₁₂-200, DOPE, Cholesterol and DMG-PEG2000 at a molar ratio of 0.335:0.32:0.335:0.1 and had a lipid: RNA ratio of 40:1 (w/w).

TABLE 38

| | | | | | | Lipid:Nucleic |
|---|---|---|---|---|---|---|
| | | | | | | Acid |
| | | | | | | (Lipid:DNA, |
| | Compound No. | | | | Total | Lipid:RNA, or |
| | 30 | DOPC | Chol | PEG-DMG | Lipid | Lipid: RNA + DNA) |
| LNP ID | (% moles) | (% moles) | (% moles) | (% moles) | (mM) | (w/w) |
| 18 | 32.5 | 5 | 60 | 2.5 | 25 | 120 |
| 42 | 40.8(40.75) | 5.0 | 51.8(51.75) | 2.5 | 25 | 120 |
| 55 | 40.7(40.68) | 10.0 | 47.1(47.12) | 2.2 | 25 | 100 |

The single co-encapsulated LNP encapsulating both FVIII transposon and SPB was an LNP of the present disclosure comprising the nanoplasmid DNA of SEQ ID NO: 6 and mRNA encoding active SPB and COMPOUND NO. 30, DOPC, Cholesterol and DMG-PEG2000 at the molar ratios shown in Table 38.

Prior to administration, juvenile mice were placed under anesthesia induced by isoflurane. For Dual-LNP administration, both DNA-LNP and mRNA-LNPs were mixed together in a tube, 50-80 uL drawn into a single 29 gauge insulin syringe, and delivered via intravenous (IV) through the retro-orbital sinus. For co-encapsulated LNP delivery, 50-80 uL of co-encapsulated LNP was drawn into a single 29 gauge insulin syringe, and delivered via intravenous (IV) through the retro-orbital sinus. Juvenile mice were brought back to normal body temperature on a 37° C. heat pad before being placed back with their mother. On Day 6 post-treatment, plasma was collected from treated mice. For plasma collection, treated mice were put under anesthesia with isoflurane, approximately 150 uL whole blood was retro-orbitally collected, whole blood was mixed with 10% volume of 3.2% sodium citrate, centrifuged at 15,000 g for 15 minutes at 20° C., and plasma supernatant was collected. hFVIII antigen levels were measured using the Visualize™ Factor VIII Antigen Plus Kit (Affinity Biologicals™ Inc.). FVIII activity levels were measured using the Chromogenix Coatest® SP4 Factor VIII kit (Diapharma®).

The results of the analysis for hFVIII antigen levels are shown in Table 39 and FVIII activity levels are shown in Table 40.

TABLE 39

| | BALB/c mice | | Hemophilia A diseased | |
|---|---|---|---|---|
| | % of Normal hFVIII | | mice % of Normal | |
| | Plasma Ag | | hFVIII Plasma Ag | |
| | | Co- | | Co- |
| | | encapsulated | | encapsulated |
| LNP ID | Dual LNP | LNP | Dual LNP | LNP |
| C12 control | 329.7 | ND | 62.6 | ND |
| 18 | 74.1 | 208.2 | 64.0 | 87.3 |
| 42 | 180.9 | 302.5 | 104.6 | 155.6 |
| 55 | 64.3 | 73.2 | 40.4 | 71.3 |

TABLE 40

| | Hemophilia A diseased mice | |
|---|---|---|
| | % of Normal hFVIII Activity | |
| | | Co- |
| | | encapsulated |
| LNP ID | Dual LNP | LNP |
| C12 control | 20.5 | ND |
| 18 | 15.4 | 14.1 |
| 42 | 23.1 | 19.3 |
| 55 | 5.1 | 1.1 |

Tables 39 and 40 show that LNPs of the present disclosure can be used to drive FVIII expression in vivo, including in Hemophilia A diseased mice, with DNA/mRNA either co-delivered in separate LNP compositions or co-encapsulated in a single LNP composition, thereby demonstrating that the compositions and methods of the present disclosure can be used to treat hemophilia A. Further, the results presented in this example show that certain LNPs of the present disclosure co-encapsulating both mRNA and DNA demonstrated equivalent or superior performance compared to co-delivery of mRNA with DNA.

### Example 76—Preparation of LNPs of Present Disclosure Comprising DNA and In Vivo Screening

The following is a nonlimiting example that provides exemplary methods for formulating a plurality of multi-component LNP compositions comprising exemplary compounds of Formula (I) and DNA.

LNP compositions of the present disclosure comprising Compound No. 30 and DNA encoding firefly luciferase (Nature Technology Corporation) were prepared as described in Example 69. The LNP compositions are shown in Table 41.

TABLE 41

| | | | | PEG- | | |
|---|---|---|---|---|---|---|
| | Compound No. | | | DMG | Total | |
| | 30 | DOPC | Chol | (% | Lipid | Lipid: DNA |
| LNP ID | (% moles) | (% moles) | (% moles) | moles) | (mM) | (w/w) |
| A1 | 35.3 (35.28) | 8.3 (8.35) | 53.7 (53.68) | 2.7 (2.69) | 25 | 120 |
| A4 | 37.8 (37.77) | 5.0 (5.00) | 55.1 (55.07) | 2.2 (2.15) | 25 | 120 |
| A6 | 44.4 (44.38) | 5.4 (5.43) | 48.5 (48.46) | 1.7 (1.73) | 25 | 120 |
| A7 | 40.0 (39.98) | (7.17) 7.2 | (50.47) 50.5 | (2.39) 2.4 | 25 | 120 |
| A8 | 37.0 (36.97) | 14.3 (14.34) | 46.7 (46.69) | 2.0 (2.00) | 25 | 120 |
| A9 | 34.0 (33.96) | 12.9 (12.89) | 51.4 (51.44) | 1.7 (1.70) | 25 | 120 |
| A10 | 45.0 (44.95) | 12.4 (12.40) | 40.8 (40.77) | 1.9 (1.88) | 25 | 120 |
| A11 | 43.1 (43.11) | 9.6 (9.59) | 45.1 (45.09) | 2.2 (2.21) | 25 | 120 |
| A12 | 41.0 (41.02) | 13.5 (13.55) | 42.8 (42.83) | 2.6 (2.61) | 25 | 120 |
| A14 | 36.7 (36.71) | 12.5 (12.46) | 48.4 (48.44) | 2.4 (2.39) | 25 | 120 |
| 34 | 45.1 (45.12) | 12.6 (12.55) | 40.6 (40.58) | 1.8 (1.75) | 25 | 100 |
| 42 | 40.8 (40.75) | 5.0 (5.00) | 51.8 (51.75) | 2.5 (2.50) | 25 | 120 |
| 55 | 40.7 (40.68) | 10.0 (10.00) | 47.1 (47.12) | 2.2 (2.20) | 25 | 100 |

Adult BALB/C mice (n=3) were administered 0.5 mg/kg of total DNA of an LNP composition listed in Table 41. The location and extent of luciferase expression in treated and control mice were determined at 4 hr and 48 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results for 4 hr are shown in Table 42 and the results for 48 hr are shown in Table 43.

TABLE 42

| | Mean Liver |
|---|---|
| | Luciferase Flux |
| LNP ID. | (p/s) |
| A4 | 2.86E+07 |
| A6 | 3.85E+07 |
| A8 | 3.94E+07 |
| A9 | 5.98E+07 |
| A10 | 4.73E+07 |
| A12 | 3.32E+07 |
| 34 | 8.37E+07 |
| 42 | 4.22E+07 |
| 55 | 5.39E+07 |

TABLE 43

| | Mean Liver |
|---|---|
| | Luciferase Flux |
| LNP ID. | (p/s) |
| A1 | 1.32E+08 |
| A4 | 1.87E+08 |
| A6 | 2.83E+08 |
| A7 | 1.10E+08 |
| A8 | 1.48E+08 |
| A9 | 1.11E+08 |
| A11 | 1.76E+08 |
| A12 | 1.38E+08 |
| A14 | 1.02E+08 |
| 34 | 1.09E+08 |
| 42 | 2.13E+08 |
| 55 | 1.16E+08 |

As shown in Tables 42 and 43, LNP compositions of the present disclosure successfully delivered DNA to liver cells and the encoded transgene was subsequently expressed in the liver cells.

In addition, administration of LNP compositions of Table 41 were well tolerated in the mice. Most of the mice lost some weight 24 hours after administration but then recovered.

### Example 77—Preparation of LNPs of Present Disclosure Comprising mRNA and In Vivo Screening

The following is a nonlimiting example that provides exemplary methods for formulating a plurality of multi-component LNP compositions comprising exemplary compounds of Formula (I) and mRNA.

LNP compositions of the present disclosure comprising Compound No. 30 and RNA encoding firefly luciferase (TriLink BioTechnologies) were prepared as described in Example 69. The LNP compositions are shown in Table 44.

TABLE 44

| | | | | PEG- | | |
|---|---|---|---|---|---|---|
| | Compound No. | | | DMG | Total | |
| | 30 | DOPC | Chol | (% | Lipid | Lipid: DNA |
| LNP ID | (% moles) | (% moles) | (% moles) | moles) | (mM) | (w/w) |
| A6 | 44.4 (44.38) | 5.43 (5.43) | 48.5 (48.46) | 1.7 (1.73) | 25 | 120 |
| 42 | 40.8 (40.75) | 5.0 | 51.8 (51.75) | 2.5 | 25 | 120 |
| A4 | 37.8 (37.77) | 5.0 | 55.1 (55.07) | 2.2 (2.15) | 25 | 120 |
| A11 | 43.1 (43.11) | 9.6 (9.59) | 45.1 (45.09) | 2.2 (2.21) | 25 | 120 |
| A8 | 37.0 (36.97) | 14.3 (14.34) | 46.7 (46.69) | 2.0 | 25 | 120 |

Adult female BALB/C mice (n=3/group) were intravenously administered 0.5 mg/kg of 5′-CleanCap--fLuciferase mRNA (TriLink Biotech) formulated with the LNP compositions shown in Table 44. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 45.

TABLE 45

| | Mean Liver |
|---|---|
| | Luciferase Flux |
| LNP ID. | (p/s) |
| A6 | 3.97e11 |
| 42 | 3.00e11 |
| A4 | 2.93e11 |
| A11 | 3.32e11 |
| A8 | 2.70e11 |

As shown in Table 45, LNP compositions of the present disclosure successfully delivered mRNA in vivo, predominantly to cells in the liver, and the encoded protein was subsequently expressed by the cells.

### Example 78—pKa Values for LNPs of Present Disclosure

pKa values for LNP compositions of the present disclosure were determined using the 2-(p-toluidino)-6-napthalene sulfonic acid (TNS) assay. TNS is an anionic molecule that is non-fluorescent in aqueous solutions but upon binding to cationic lipids exhibits strong fluorescence. LNPs were standardized by diluting to approximately 1.7nmol of ionizable lipid per well and were titrated using pH buffered solutions from pH 2 to 12 in a 96-well plate. The TNS reagent (Sigma-Aldrich #T9792) was prepared at 0.05 mg/mL in water, 5 μL of reagent was dispensed to each well, and fluorescence was measured on a TECAN Infinite M200PRO microplate reader (Excitation: 322 nm and Emission: 431 nm, gain 75). Fluorescence values for each LNP were normalized to the maximum TNS signal. GraphPad Prism was used to plot the pH and normalized fluorescence values using the XY graphing function. This data was analyzed using nonlinear regression and log (inhibitor) vs normalized response functions to yield the pKa values for each sample, shown in Table 46.

TABLE 46

| LNP ID. | pKa |
|---|---|
| A1 | 7.10 |
| A4 | 7.13 |
| A6 | 7.12 |
| A7 | 6.62 |
| A8 | 7.07 |
| A9 | 7.05 |
| A10 | 6.98 |
| A11 | 7.13 |
| A12 | 6.70 |
| A14 | ND |
| 34 | ND |
| 42 | 7.11 |
| 55 | 7.14 |

### Example 79—Preparation of LNPs of Present Disclosure Comprising DNA and In Vivo Screening

The following is a nonlimiting example that provides exemplary methods for formulating a plurality of multi-component LNP compositions comprising exemplary compounds of Formula (I) and DNA.

LNP compositions of the present disclosure comprising nanoplasmid DNA encoding firefly luciferase (Nature Technology Corporation) under the control of a liver specific promoter-AM-TTR were prepared as described in Example 69. The LNP compositions are shown in Table 47.

TABLE 47

| LNPs | | | | | | |
|---|---|---|---|---|---|---|
| comprising | | | | | | |
| Compound | | | | | | |
| No. 30 | | | | | | |
| Prepared | Compound No. | | | | Total | |
| According | 30 | | | PEG-DMG | Lipid | Lipid: DNA |
| to: | (% moles) | DOPC (% moles) | Chol (% moles) | (% moles) | (mM) | (w/w) |
| Example | 40.75 | 5 | 51.75 | 2.5 | 25 | 120 |
| 21 | | | | | | |
| Alternative | 40.75 | 5 | 51.75 | 2.5 | 25 | 120 |
| General | | | | | | |
| Procedure | | | | | | |
| K | | | | | | |
| Alternative | 40.75 | 5 | 51.75 | 2.5 | 25 | 120 |
| General | | | | | | |
| Procedure | | | | | | |
| L | | | | | | |

Adult BALB/C mice were administered 0.5 mg/kg of total DNA (n=3) of the LNP composition listed in Table 47. The location and extent of luciferase expression in treated and control mice were determined at 24 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 48.

TABLE 48

| LNPs | |
|---|---|
| comprising | |
| Compound No. | Mean Liver |
| 30 Prepared | Luciferase Flux |
| According to: | (p/s) |
| Example 21 | 21266666.67 |
| Alternative | 31400000 |
| General | |
| Procedure K | |
| Alternative | 29600000 |
| General | |
| Procedure L | |

As shown in Table 48, LNP compositions of the present disclosure comprising Compound No. 30 prepared according to three different synthetic routes successfully delivered DNA to liver cells and the encoded transgene was subsequently expressed in the liver cells.

### Example 80—In vivo LNP delivery of mRNA to liver

The following is a non-limiting example demonstrating that the lipid nanoparticle compositions of the present disclosure can be used to deliver mRNA to liver cells in vivo and expression of encoded proteins over a wide range of lipid: RNA ratio.

LNP compositions of the present disclosure comprising Compound No. 30 and RNA encoding firefly luciferase (TriLink BioTechnologies) were prepared as described in Example 69. The LNP compositions are shown in Table 49.

TABLE 49

| | | | | PEG- | | |
|---|---|---|---|---|---|---|
| | Compound No. | | | DMG | Total | |
| | 30 | | | (% | Lipid | Lipid: DNA |
| LNP ID | (% moles) | DOPC (% moles) | Chol (% moles) | moles) | (mM) | (w/w) |
| 42 | 40.8 (40.75) | 5.0 | 51.8 (51.75) | 2.5 | 25 | 120 |
| 42 | 40.8 (40.75) | 5.0 | 51.8 (51.75) | 2.5 | 25 | 80 |
| 42 | 40.8 (40.75) | 5.0 | 51.8 (51.75) | 2.5 | 25 | 60 |
| 42 | 40.8 (40.75) | 5.0 | 51.8 (51.75) | 2.5 | 25 | 40 |

Adult female BALB/C mice (n=3/group) were intravenously administered 0.25 mg/kg or 0.5 mg/kg of 5′-CleanCap--fLuciferase mRNA (TriLink Biotech) formulated with the LNP compositions shown in Table 49. One group of mice was treated with vehicle (PBS, Thermo Fisher Scientific, USA) as a negative control.

The location and extent of luciferase expression in treated and control mice were determined at 4 hr by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results are shown in Table 50.

TABLE 50

| | Mean Liver | Mean Liver |
|---|---|---|
| LNP ID. 42, | Luciferase Flux | Luciferase Flux |
| Lipid:RNA | (p/s) | (p/s) |
| (w/w) | 0.25 mg/kg dose | 0.5 mg/kg dose |
| 120 | 1.27e11 | 3.18e11 |
| 80 | 1.96e11 | 3.50e11 |
| 60 | 1.46e11 | 3.41e11 |
| 40 | 1.68e11 | 3.28e11 |

As shown in Table 50, LNP compositions of the present disclosure successfully delivered RNA in vivo, predominantly to cells in the liver, and the encoded protein was subsequently expressed by the cells. Delivery efficacy was similar across varying ratios of lipid: RNA payload.

### Example 81—In vivo LNP delivery of mRNA to liver

The following is a non-limiting example demonstrating that the lipid nanoparticle compositions of the present disclosure can be used to deliver mRNA to liver cells in vivo and expression of encoded proteins over a wide range of lipid: RNA ratio with good tolerability.

A. Delivery of SPB mRNA to Liver Cells

LNP compositions of the present disclosure comprising Compound No. 30 and RNA encoding SPB protein were prepared as described in Example 69. The LNP compositions are shown in Table 51.

TABLE 51

| | | | | PEG- | | |
|---|---|---|---|---|---|---|
| | Compound No. | | | DMG | Total | |
| | 30 | | | (% | Lipid | Lipid: DNA |
| LNP ID | (% moles) | DOPC (% moles) | Chol (% moles) | moles) | (mM) | (w/w) |
| 42 | 40.8 (40.75) | 5.0 | 51.8 | 2.5 | 25 | 120 |
| | | | (51.75) | | | |
| 42 | 40.8 (40.75) | 5.0 | 51.8 | 2.5 | 25 | 80 |
| | | | (51.75) | | | |
| 42 | 40.8 (40.75) | 5.0 | 51.8 | 2.5 | 25 | 60 |
| | | | (51.75) | | | |
| 42 | 40.8 (40.75) | 5.0 | 51.8 | 2.5 | 25 | 40 |
| | | | (51.75) | | | |

Adult female BALB/C mice (n=3/group) were intravenously administered 1 mg/kg or 2 mg/kg of 5MeC-mRNA encoding SPB formulated with the LNP compositions shown in Table 51. One group of mice was treated with vehicle (PBS, Thermo Fisher Scientific, USA) as a negative control.

After four hours post-administration, liver tissue was collected from the mice in each group. Briefly, tissues were resected after euthanasia, flash frozen in liquid nitrogen, mixed with lysis buffer (15 mg of tissue in 200 μL of lysis buffer+10 uL Proteinase K) and pulverized in a TissueLyser II (Qiagen) using Triple-Pure zirconium beads (Fisher Scientific). SPB protein was measured in homogenized tissue using an SPB immunoassay (Meso Scale Diagnostics LLC). The results are shown in Table 52.

TABLE 52

| LNP ID. 42, | ng SPB/mg | ng SPB/mg |
|---|---|---|
| Lipid:RNA | protein | protein |
| (w/w) | 1 mg/kg dose | 2 mg/kg dose |
| 120 | 3.81 | 6.93 |
| 80 | 4.17 | 6.38 |
| 60 | 4.91 | 8.58 |
| 40 | 3.10 | 4.59 |

As shown in Table 52, LNP compositions of the present disclosure successfully delivered mRNA in vivo, predominantly to cells in the liver, and the encoded protein was subsequently expressed by the cells. Delivery efficacy was similar across varying ratios of lipid: RNA payload.

### B. Tolerability Studies

Adult female BALB/C mice (n=3/group) were intravenously administered 0.5 mg/kg, 1 mg/kg or 1.5 mg/kg of 5MeC-mRNA encoding SPB formulated with the LNP compositions shown in Table 51. One group of mice was treated with vehicle (PBS, Thermo Fisher Scientific, USA) as a negative control.

The levels of four liver enzymes present in serum were evaluated at 24 hours after LNP administration for each of the tested concentrations as a measure of potential hepatoxicity. Briefly, blood was drawn at 24 hours and each sample was allowed to clot for 20 minutes and subject to centrifugation at 13K rpms for 3 minutes to remove undesired cells and debris. The samples were placed on wet ice for transport, and stored at −80° C. until analyzed. Enzyme levels were determined using standardized assays (Idexx). The levels of the liver enzymes alkaline phosphatase (ALP), aspartate transaminase (AST), alanine transaminase (ALT) and creatine kinase (CK) are shown in Tables 53 a-d, respectively.

TABLE 53a
ALP levels (Units/L)

| LNP dose | Lipid:RNA | Lipid:RNA | Lipid:RNA | Lipid:RNA |
|---|---|---|---|---|
| (mg/kg) | 120:1 | 80:1 | 60:1 | 40:1 |
| 0.5 | 109.3 | 116.3 | 112.3 | 99.0 |
| 1 | 135.0 | 108.0 | 96.3 | 112.7 |
| 1.5 | 157.0 | 136.0 | 110.3 | 105.7 |

TABLE 53b
AST levels (Units/L)

| LNP dose | Lipid:RNA | Lipid:RNA | Lipid:RNA | Lipid:RNA |
|---|---|---|---|---|
| (mg/kg) | 120:1 | 80:1 | 60:1 | 40:1 |
| 0.5 | 53.7 | 61.3 | 58.3 | 56.7 |
| 1 | 240.7 | 121.7 | 105.7 | 81.0 |
| 1.5 | 409.0 | 179.0 | 150.0 | 54.7 |

TABLE 53c
ALT levels (Units/L)

| LNP dose | Lipid:RNA | Lipid:RNA | Lipid:RNA | Lipid:RNA |
|---|---|---|---|---|
| (mg/kg) | 120:1 | 80:1 | 60:1 | 40:1 |
| 0.5 | 72.0 | 28.3 | 83.0 | 91.3 |
| 1 | 60.7 | 105.7 | 133.3 | 44.7 |
| 1.5 | 446.5 | 87.0 | 73.0 | 40.3 |

TABLE 53d
CK levels (Units/L)

| LNP dose | Lipid:RNA | Lipid:RNA | Lipid:RNA | Lipid:RNA |
|---|---|---|---|---|
| (mg/kg) | 120:1 | 80:1 | 60:1 | 40:1 |
| 0.5 | 121.3 | 168.3 | 90.7 | 159.3 |
| 1 | 784.3 | 363.3 | 335.0 | 275.3 |
| 1.5 | 327.5 | 167.0 | 154.0 | 104.3 |

In addition to serum liver enzymes, the levels of four proinflammatory cytokines present in serum were evaluated at 4 hours after LNP administration for each of the tested concentrations. Briefly, serum samples were prepared as described for liver enzyme analysis and the serum concentration of each cytokine was determined using commercially available ELISA kits (e.g., R&D Systems Quantikine ELISA kits). The levels of the proinflammatory cytokines interleukin-6 (IL-6), interferon gamma (INF-G), tumor necrosis factor alpha (TNF-α) and macrophage inflammatory protein-1 alpha (MIP-1a) at 4 hours are shown in Tables 54 a-d, respectively.

TABLE 54a
IL-6 levels (pg/mL)

| LNP dose | Lipid:RNA | Lipid:RNA | Lipid:RNA | Lipid:RNA |
|---|---|---|---|---|
| (mg/kg) | 120:1 | 80:1 | 60:1 | 40:1 |
| 0.5 | 1583.9 | 1576.5 | 1399.8 | 757.0 |
| 1 | 22150.6 | 10587.8 | 4179.2 | 1105.6 |
| 1.5 | 28219.1 | 46882.7 | 72789.3 | 1685.3 |

TABLE 54b
INF-G levels (pg/mL)

| LNP dose | Lipid:RNA | Lipid:RNA | Lipid:RNA | Lipid:RNA |
|---|---|---|---|---|
| (mg/kg) | 120:1 | 80:1 | 60:1 | 40:1 |
| 0.5 | 53.5 | 52.7 | 54.0 | 51.1 |
| 1 | 100.3 | 69.1 | 57.3 | 52.2 |
| 1.5 | 79.8 | 106.6 | 107.0 | 54.9 |

TABLE 54c
TNF-a levels (pg/mL)

| LNP dose | Lipid:RNA | Lipid: RNA | Lipid: RNA | Lipid:RNA |
|---|---|---|---|---|
| (mg/kg) | 120:1 | 80:1 | 60:1 | 40:1 |
| 0.5 | 86.5 | 68.1 | 10.1 | 24.8 |
| 1 | 339.5 | 200.6 | 119.9 | 55.7 |
| 1.5 | 541.4 | 326.4 | 374.1 | 147.9 |

TABLE 54d
MIP-1a levels (pg/mL)

| LNP dose | Lipid:RNA | Lipid: RNA | Lipid: RNA | Lipid:RNA |
|---|---|---|---|---|
| (mg/kg) | 120:1 | 80:1 | 60:1 | 40:1 |
| 0.5 | 122.8 | 99.8 | 81.6 | 58.4 |
| 1 | 380.3 | 289.9 | 206.1 | 90.9 |
| 1.5 | 328.0 | 489.5 | 477.6 | 198.3 |

The body weight of mice treated with the LNP compositions of Table 51 was assessed prior to intravenous administration and 24 hours post-administration and the body weights at baseline and post-treatment were compared. The average percentage of body weight change for each group of mice treated with each LNP composition of Table 51 is shown in Table 55.

TABLE 55
% Body weight change 24 hr

| LNP dose | Lipid:RNA | Lipid:RNA | Lipid: RNA | Lipid:RNA |
|---|---|---|---|---|
| (mg/kg) | 120:1 | 80:1 | 60:1 | 40:1 |
| 0.5 | −3.4 | −2.4 | −1.1 | −1.1 |
| 1 | −8.8 | −4.4 | −4.6 | −2.2 |
| 1.5 | −8.5 | −7.2 | −5.8 | −1.9 |

The results of this example show that LNP compositions of the present disclosure successfully delivered mRNA in vivo, predominantly to cells in the liver, and the encoded protein was subsequently expressed by the cells. Delivery efficacy was similar across varying ratios of lipid: RNA payload. The LNP compositions exhibited significantly reduced toxicity profiles with decreasing lipid: payload ratio, as shown by reduced liver enzyme responses, reduced cytokine responses, and reduced body weight loss at all doses tested.

### Example 82—LNPs of the present disclosure for the treatment of hemophilia

The following is a non-limiting example demonstrating that single lipid nanoparticle compositions of the present disclosure can be used to deliver both mRNA and DNA in vivo, across a varying range of mRNA: DNA ratios and a varying range of lipid: RNA+DNA payload.

Wild-type BALB/c juvenile mice (N=4-6) were dosed with a single co-encapsulated LNP encapsulating both FVIII transposon and SPB (delivery method denoted as “Co-encapsulated LNP”) and bled on Day 6 for hFVIII Ag ELISA as described in Example 74. Mice received 0.5 mg/kg of co-encapsulated LNP encapsulating mRNA and DNA at 1:2, 1:1, 2:1 and 4:1 mRNA: DNA ratios; or 0.75 mg/kg of co-encapsulated LNP encapsulating mRNA and DNA at 2:1 and 4:1 mRNA: DNA ratios. A separate group of mice were administered a benchmark C₁₂-200 Dual-LNP composition (as described in Example 74) at a 1:1 mRNA: DNA ratio for the 0.5 mg/kg dose and at 2:1 and 4:1 mRNA: DNA ratio for the 0.75 mg/kg dose.

The single co-encapsulated LNPs encapsulating both FVIII transposon and SPB were LNPs of the present disclosure comprising the nanoplasmid DNA of SEQ ID NO: 6 and mRNA encoding active SPB. The single co-encapsulated LNPs also comprised COMPOUND No. 30, DOPC, Cholesterol and DMG-PEG2000 at the molar ratios shown in Table 56.

TABLE 56

| | | | | DMG | | |
|---|---|---|---|---|---|---|
| | Compound No. | | | PEG- | Lipid | Lipid: |
| | 30 | | | (% | Total | RNA + DNA |
| LNP ID | (% moles) | DOPC (% moles) | Chol (% moles) | moles) | (mM) | (w/w) |
| 42 | 40.8 (40.75) | 5.0 | 51.75 | 2.5 | 25 | 100 |
| | | | (51.75) | | | |
| A4 | 37.8 (37.77) | 5.0 | 55.1 (55.07) | 2.2 | 25 | 100 |
| | | | | (2.15) | | |

The results of this analysis are shown in Table 57a for the 0.5 mg/kg mRNA: DNA treatment group and Table 57b for the 0.75 mg/kg mRNA: DNA treatment group, respectively.

TABLE 57a
% of Normal hFVIII Plasma Ag

| | | Co- | Co- | Co- | Co- |
|---|---|---|---|---|---|
| | Dual | encapsulated | encapsulated | encapsulated | encapsulated |
| LNP ID | LNP | LNP (1:2) | LNP (1:1) | LNP (2:1) | LNP (4:1) |
| C12 | 156.3 | ND | ND | ND | ND |
| control | | | | | |
| 42 | ND | 268.5 | 371.3 | 130.5 | 97.2 |
| A4 | ND | 635.3 | 311.9 | 120.3 | 83.9 |

TABLE 57b
% of Normal hFVIII Plasma Ag

| | | | Co- | Co- |
|---|---|---|---|---|
| | Dual LNP | Dual LNP | encapsulated | encapsulated |
| LNP ID | (2:1) | (4:1) | LNP (2:1) | LNP (4:1) |
| C12 control | 202.5 | 105.4 | ND | ND |
| 42 | ND | ND | 228.3 | 50.5 |
| A4 | ND | ND | 322.3 | 84.3 |

In another experiment, wild-type BALB/c juvenile mice (N=4-6) and wild-type BALB/c adult mice (N=4-6) were dosed with a single co-encapsulated LNP encapsulating both FVIII transposon and SPB and bled on Day 6 for hFVIII Ag ELISA as described in Example 74. Mice received 0.5 mg/kg of co-encapsulated LNP encapsulating mRNA and DNA at 1:1, 1:2, 1:3 or 1:4 mRNA: DNA ratios. A separate group of mice were left untreated as a control.

The single co-encapsulated LNPs encapsulating both FVIII transposon and SPB were LNPs of the present disclosure comprising the nanoplasmid DNA of SEQ ID NO: 6 and mRNA encoding active SPB. The single co-encapsulated LNPs also comprised COMPOUND No. 30, DOPC, Cholesterol and DMG-PEG2000 at the molar ratios shown in Table 56.

The results of this analysis are shown in Table 58a for the adult mice and Table 58b for the juvenile mice.

TABLE 58a
% of Normal hFVIII Plasma Ag

| | Co- | Co- | Co- | Co- |
|---|---|---|---|---|
| | encapsulated | encapsulated | encapsulated | encapsulated |
| LNP ID | LNP (1:1) | LNP (1:2) | LNP (1:3) | LNP (1:4) |
| 42 | 81.7 | 108.7 | 93.2 | 108.0 |
| A4 | 92.1 | 115.0 | 107.5 | 84.8 |

TABLE 58b
% of Normal hFVIII Plasma Ag

| | Co- | Co- | Co- | Co- |
|---|---|---|---|---|
| | encapsulated | encapsulated | encapsulated | encapsulated |
| LNP ID | LNP (1:1) | LNP (1:2) | LNP (1:3) | LNP (1:4) |
| A4 | 433.4 | 586.8 | ND | 907.4 |

Tables 57a-b and 58a-b show that LNPs of the present disclosure with mRNA/DNA at varying ratios co-encapsulated in a single LNP composition can be used to drive FVIII expression in vivo, thereby demonstrating that the compositions and methods of the present disclosure can be used to treat hemophilia A.

In an additional experiment, wild-type C₅₇BL/6 adult mice (N=4-6) were dosed with a single co-encapsulated LNP encapsulating both FVIII transposon and SPB and bled on Day 6 for hFVIII Ag ELISA as described in Example 74. Mice received 0.5 mg/kg of co-encapsulated LNP encapsulating mRNA and DNA at a 1:2 mRNA: DNA ratio. A separate group of mice were left untreated as a control.

The single co-encapsulated LNPs encapsulating both FVIII transposon and SPB were LNPs of the present disclosure comprising the nanoplasmid DNA of SEQ ID NO: 6 and mRNA encoding active SPB. The single co-encapsulated LNPs also comprised COMPOUND No. 30, DOPC, Cholesterol and DMG-PEG2000 at the molar ratios shown in Table 59.

TABLE 59

| | Compound | | | | Total | Lipid: |
|---|---|---|---|---|---|---|
| | No. 30 | | | PEG-DMG | Lipid | RNA + DNA |
| LNP ID | (% moles) | DOPC (% moles) | Chol (% moles) | (% moles) | (mM) | (w/w) |
| 42 | 40.8 (40.75) | 5.0 | 51.8 (51.75) | 2.5 | 25 | 120 |
| 42 | 40.8 (40.75) | 5.0 | 51.8 (51.75) | 2.5 | 25 | 100 |
| 42 | 40.8 (40.75) | 5.0 | 51.8 (51.75) | 2.5 | 25 | 80 |

The results of this analysis are shown in Table 60. Delivery efficacy was similar across varying ratios of lipid: RNA+DNA payload.

TABLE 60

| LNP ID. 42, | % of Normal |
|---|---|
| Lipid: | hFVIII |
| RNA + DNA | Plasma |
| (w/w) | Ag |
| 120 | 17.6 |
| 100 | 40.3 |
| 80 | 61.7 |

This example shows that LNPs of the present disclosure can be used to drive FVIII expression in vivo, with DNA/mRNA co-encapsulated in a single LNP composition, thereby demonstrating that the compositions and methods of the present disclosure can be used to treat hemophilia A. Further, the results presented in this example show that certain LNPs of the present disclosure co-encapsulating both mRNA and DNA demonstrated efficacy across a varying range of mRNA: DNA ratios and a varying range of lipid: nucleic acid payload.

### Example 83—Preparation of LNPs of Present Disclosure Comprising mRNA or DNA and In Vivo Screening

The following is a nonlimiting example that provides exemplary methods for formulating a plurality of multi-component LNP compositions comprising exemplary compounds of Formula (I) and mRNA or DNA.

LNP compositions of the present disclosure comprising Compound No. 23 or 30 and RNA encoding firefly luciferase (TriLink BioTechnologies) or DNA encoding firefly luciferase (Nature Technology Corporation) were prepared as described in Example 69. The LNP compositions are shown in Table 61.

TABLE 61

| | | | | | | Lipid:DNA |
|---|---|---|---|---|---|---|
| | Compound No. | | | | Total | or |
| | 23 or 30 | | | PEG-DMG | Lipid | Lipid: RNA |
| LNP ID | (% moles) | DOPC (% moles) | Chol (% moles) | (% moles) | (mM) | (w/w) |
| A4 | 37.8% (37.77%) | 5.0 | 55.1 (55.07) | 2.2 (2.15) | 25 | 120 |
| | moles of | | | | | |
| | Compound No. | | | | | |
| | 23 | | | | | |
| A6 | 44.4% (44.38%) | 5.43 | 48.5 (48.46) | 1.7 (1.73) | 25 | 120 |
| | moles of | | | | | |
| | Compound No. | | | | | |
| | 23 | | | | | |
| 42 | 40.8% (40.75%) | 5.0 | 51.8 (51.75) | 2.5 | 25 | 120 |
| | moles of | | | | | |
| | Compound No. | | | | | |
| | 23 | | | | | |
| 42 | 40.8% (40.75%) | 5.0 | 51.8 (51.75) | 2.5 | 25 | 120 |
| | moles of | | | | | |
| | Compound No. | | | | | |
| | 30 | | | | | |

Adult BALB/C mice (n=3) were administered 0.5 mg/kg of total mRNA or DNA formulated in an LNP composition listed in Table 61. One group of mice was treated with an LNP composition of the present disclosure comprising Compound No. 30 as a positive control.

The location and extent of luciferase expression in treated and control mice were determined at 4 hr for RNA delivery, or 48 hr for DNA delivery, by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed. The results for 4 hr are shown in Table 62 and the results for 48 hr are shown in Table 63.

TABLE 62

| | Mean Liver |
|---|---|
| | Luciferase Flux |
| LNP ID. | (p/s) |
| A4 | 2.78e11 |
| (Compound No. 23) | |
| A6 | 2.82e11 |
| (Compound No. 23) | |
| 42 | 2.57e11 |
| (Compound No. 23) | |
| 42 | 3.79e11 |
| (Compound No. 30) | |

TABLE 63

| | Mean Liver |
|---|---|
| | Luciferase Flux |
| LNP ID. | (p/s) |
| A4 | 1.08e8 |
| (Compound No. 23) | |
| A6 | 1.42e8 |
| (Compound No. 23) | |
| 42 | 8.54e7 |
| (Compound No. 23) | |
| 42 | 7.95e7 |
| (Compound No. 30) | |

As shown in Tables 62 and 63, LNP compositions of the present disclosure successfully delivered RNA or DNA to liver cells and the encoded transgene was subsequently expressed in the liver cells.

### Example 84—Preparation of LNPs of Present Disclosure Comprising mRNA and In Vivo Expression

The following is a nonlimiting example that provides exemplary methods for formulating a plurality of multi-component LNP compositions comprising exemplary compounds of Formula (I) and mRNA.

LNP compositions of the present disclosure comprising Compound No. 30 were prepared as described in Example 69. The LNP compositions are shown in Table 64.

TABLE 64

| | Compound No. | | | | Total | |
|---|---|---|---|---|---|---|
| | 30 | | | PEG-DMG | Lipid | Lipid:RNA |
| LNP ID | (% moles) | DOPC (% moles) | Chol (% moles) | (% moles) | (mM) | (w/w) |
| 1.1 | 34.40 | 7.86 | 56.18 | 1.54 | 11.18 | 100 |
| 1.2 | 47.03 | 3.76 | 46.28 | 2.93 | 11.68 | 100 |
| 1.3 | 44.48 | 4.68 | 48.46 | 2.34 | ND | 100 |
| 1.4 | 30.30 | 17.27 | 50.45 | 1.98 | 11.26 | 100 |
| 1.5 | 66.98 | 0.35 | 30.96 | 1.71 | 13.68 | 100 |
| 1.6 | 30 | 12 | 55 | 3 | 9.98 | 100 |
| 1.7 | 51.71 | 5.99 | 40.46 | 1.85 | 9.67 | 100 |
| 1.8 | 52.5 | 16 | 30 | 1.5 | 12.59 | 100 |
| 1.9 | 47 | 20 | 30 | 3 | 12.5 | 100 |
| 1.10 | 54.11 | 9.49 | 33.40 | 3.00 | 12.87 | 100 |

Ai14 mice (n=2/group) were intravenously administered 1.5 mg/kg of CleanCap NLS-Cre Recombinase mRNA (5moUJ) (TriLink BioTechnologies) formulated with the LNP compositions shown in Table 49. Successful delivery of Cre recombinase RNA to cells in Ai14 mice leads to tdTomato fluorescence triggered by Cre-mediated recombination. One group of mice was treated with vehicle (PBS, Thermo Fisher Scientific, USA) as a negative control.

The extent of tdTomato expression in hematopoietic stem cells (CD45+, Live+, Lin-, c-kit (117)+, Sca-1+) (more commonly known as LSK cells) of treated and control mice were determined at 4 hr by flow cytometry (BD Fortessa). Mice were sacrificed 4 hours post injection and bone marrow was removed from each mouse and placed in cold PBS. The bone marrow was then crushed using a mortar and pestle with added FACS buffer (PBS, fetal bovine serum, EDTA). After crushing, the bone marrow was filtered with ACK (ammonium-chloride-potassium) lysis buffer to remove red blood cells. The samples were then washed with FACS buffer to remove the ACK buffer and stained to look for LSK cells. The samples were run on a flow cytometer and approximately 100,000 live Lin-cells were collected for analysis of % positive tdTomato expression. The results are shown in Table 65.

TABLE 65

| | % positive |
|---|---|
| | tdTomato |
| LNP ID. | expression |
| 1.1 | 28.3 |
| 1.2 | 31.55 |
| 1.3 | 48.85 |
| 1.4 | 22.45 |
| 1.5 | 6.7 |
| 1.6 | 8.255 |
| 1.7 | 37.15 |
| 1.8 | 13.4 |
| 1.9 | 29.35 |
| 1.10 | 8.57 |
| 1.11 | 27.55 |
| 1.12 | 24.05 |

As shown in Table 65, LNP compositions of the present disclosure successfully delivered mRNA in vivo to hematopoietic stem cells, and the encoded Cre recombinase was subsequently expressed in the cells, leading to reporter molecule expression. In this experiment, certain LNP compositions of the present disclosure delivered RNA to LSK cells at levels of around 40% or higher.

### Example 85—LNP compositions of present disclosure deliver RNA and DNA with high specificity to the liver in vivo

This experiment shows the ability of LNP compositions of the present disclosure to deliver Cas-CLOVER mRNA and transgene DNA to the liver, targeted by a pair of gRNAs to the mouse Albumin (Alb) gene, resulting in subsequent in vivo transgene insertion of a luciferase reporter into the Alb gene by homology independent transgene insertion. Successful insertion of the reporter into the target gene is dependent on Cas-CLOVER activity and was measured by luciferase expression in the liver in vivo and assessment of target site editing.

In this experiment, two groups of wild-type BALB/C mice (n=4/group, 3- or 10-week-old) were intravenously administered: (1) a single co-encapsulated LNP encapsulating mRNA encoding 5′-CleanCap-5MeC-Cas-CLOVER (SEQ ID NO: 5), a pair of gRNAs (SEQ ID NOs: 11 and 12) targeted to mouse albumin intron 3, and plasmid DNA encoding AKAluc (SEQ ID NO: 15) (delivery method denoted as “Co-encapsulated LNP”), (2) LNPs encapsulating mRNA encoding 5′-CleanCap-5MeC-Cas-CLOVER (SEQ ID NO: 5) and a pair of gRNAs (SEQ ID NOs: 11 and 12) targeted to mouse albumin intron 3 co-delivered with LNPs encapsulating plasmid DNA encoding AKAluc (SEQ ID NO: TBD) (delivery method denoted as “Dual-LNP”), or (3) LNPs encapsulating mRNA encoding 5′-CleanCap-5MeC-Cas-CLOVER (SEQ ID NO: 5) and a pair of non-targeting gRNAs (SEQ ID NOs: 13 and 14) co-delivered with LNPs encapsulating plasmid DNA encoding AKAluc (SEQ ID NO: 15). Mice receiving Co-encapsulated LNP treatment received 1.5 mg/kg of co-encapsulated LNP encapsulating mRNA, gRNAs and DNA at 3:1:1 mRNA: gRNA: DNA ratio. Mice receiving Dual-LNP treatment received 0.75 mg/kg of mRNA-LNP co-dosed with 0.25 mg/kg of DNA-LNP. One group of mice was treated with vehicle (PBS, Thermo Fisher Scientific, USA) as a negative control.

For the Co-encapsulated LNP method, the mRNA, gRNA and DNA molecules were formulated within LNP compositions of the present disclosure comprising COMPOUND 30, DOPC, Cholesterol and DMG-PEG2000 as shown in Table 66. All cytidine residues in the mRNA were 5-methylcytidine (5-MeC).

TABLE 66

| | | | | PEG- | | Lipid: RNA |
|---|---|---|---|---|---|---|
| | Compound | | | DMG | Total | (mRNA + gRNAs) |
| | No. 30 | | | (% | Lipid | +DNA |
| LNP ID | (% moles) | DOPC (% moles) | Chol (% moles) | moles) | (mM) | (w/w) |
| A4 | 37.8 (37.77) | 5.0 | 55.1 (55.07) | 2.2 (2.15) | 25 | 120 |
| 42 | 40.8 (40.75) | 5.0 | 51.8 (51.75) | 2.5 | 25 | 120 |

For the Dual-LNP method, RNA (mRNA and gRNAs) was formulated within LNP compositions comprising C₁₂-200, DOPE, Cholesterol and DMG-PEG2000 at a molar ratio of 0.335:0.32:0.335:0.1 and a lipid: RNA ratio of 40:1 (w/w); DNA was formulated within LNP compositions comprising C12-200, DOPE, Cholesterol and DMG-PEG2000 at a molar ratio of 0.35:0.2:0.4184:0.0316 and a lipid: DNA ratio of 80:1 (w/w).

The location and extent of luciferase expression in treated and control mice were determined at 7 days, 15 days, and 21 days by bioluminescent imaging (BLI) of anesthetized mice using an IVIS Lumina in vivo imaging system (Perkin Elmer) according to the manufacturer's instructions. Mice were anesthetized using isoflurane in oxygen, and placed supine on a heated stage. Mice were then administered D-luciferin (Perkin-Elmer #122799) IP, and BLI was performed. The results are shown in Table 67a for the 3-week-old mice and Table 67b for the 10-week-old mice.

TABLE 67a

| | Mean Liver Luciferase Flux (p/s) | | | | |
|---|---|---|---|---|---|
| | | | Dual LNP | LNP ID A4, | LNP ID 42, |
| | | Dual LNP | (non- | Co- | Co- |
| Time | | (targeting | targeting | encapsulated | encapsulated |
| point | Vehicle | gRNAs) | gRNAs) | LNP | LNP |
| 7 days | 1.17E6 | 1.47E8 | 1.04E6 | 2.09E8 | 1.45E8 |
| 15 days | 0.7E6 | 8.9E7 | 0.5E6 | 1.2E8 | 1.2E8 |
| 21 days | 0.13E6 | 1.11E8 | 0.45E6 | 1E8 | 9.57E7 |

TABLE 67b

| | Mean Liver Luciferase Flux (p/s) | | | | |
|---|---|---|---|---|---|
| | | | Dual LNP | LNP ID A4, | LNP ID 42, |
| | | Dual LNP | (non- | Co- | Co- |
| Time | | (targeting | targeting | encapsulated | encapsulated |
| point | Vehicle | gRNAs) | gRNAs) | LNP | LNP |
| 7 days | 3.9E7 | 2.5E7 | 1.18E6 | 7.11E7 | 4.77E7 |
| 15 days | 8.6E5 | 2.95E7 | 1.06E6 | 6.11E7 | 6.82E7 |
| 21 days | 7.8E5 | 1.83E7 | 5.2E5 | 1.81E7 | 2.23E7 |

Tables 67a and 67b show that robust luciferase expression persisted for 21 days in both juvenile and adult mice. LNPs of the present disclosure comprising co-encapsulated mRNA, gRNAs, and DNA performed similarly or better than dual LNPs comprising benchmark C12-200 LNP compositions.

In another assay, target site gene editing by the Cas-CLOVER mRNA delivered to the mice was measured by Droplet Digital PCR (ddPCR). Briefly, 50 ng of genomic DNA, primers and labeled probes were fractionated into 20000 droplets and PCR amplified with the following cycling parameters: 95C for 10 min, 40 cycles of 94C (30 sec), 55C (1 min), and a final step of 98C for 10 min. Then, samples were read with a QX200 droplet reader (Biorad) following manufacturer instructions. Percentage of indels was calculated as (# of NEHJ droplets×100)/(# of NHEJ+ # of reference droplets). Results of the ddPCR are provided in Tables 68a and 68b as indel percentages found in at the albumin intron 3 insertion site.

TABLE 68a
juvenile mice

| | Alb intron 3 |
|---|---|
| | indel % |
| Vehicle | 0 |
| Dual LNP (targeting | 54.8 |
| gRNAS | |
| Dual LNP (non-targeting | 0 |
| gRNAs) | |
| LNP ID A4, Co- | 63.3 |
| encapsulated LNP | |
| LNP ID 42, | 63.2 |
| Co-encapsulated LNP | |

TABLE 68b
adult mice

| | Alb intron 3 |
|---|---|
| | indel % |
| Vehicle | 0 |
| Dual LNP (targeting | 48.8 |
| gRNAs) | |
| Dual LNP (non-targeting | 0 |
| gRNAs) | |
| LNP ID A4, Co- | 59.7 |
| encapsulated LNP | |
| LNP ID 42, | 65.9 |
| Co-encapsulated LNP | |

As shown in Tables 68a and 68b, LNP compositions of the present disclosure successfully delivered Cas-CLOVER mRNA and transgene DNA to the liver as shown by subsequent gene editing of the Alb gene, with indel rates better than benchmark C₁₂-200 based compositions. A single dose of an LNP of the present disclosure achieved 50-65% editing at mouse albumin intron 3.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A composition comprising at least one lipid nanoparticle comprising at least one compound of Formula (1), Formula (II), or Formula (III): 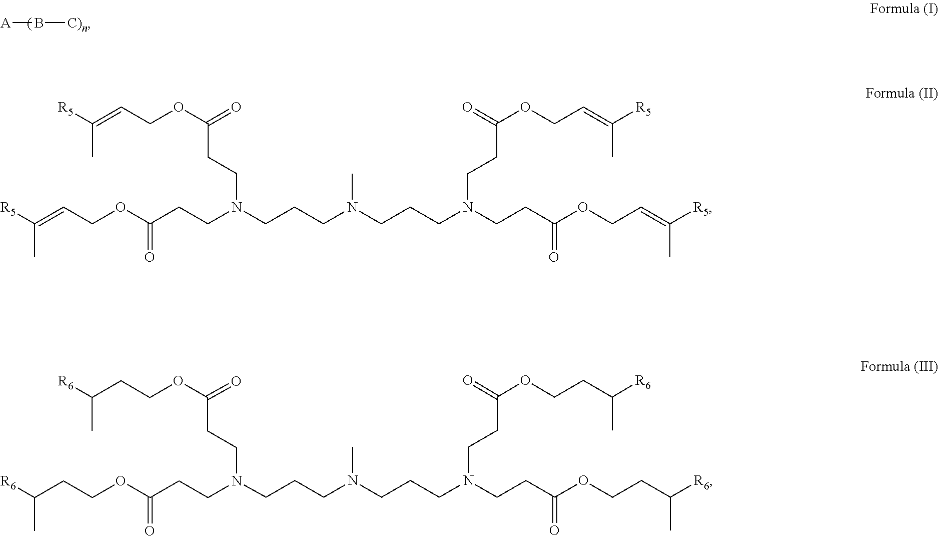
or a salt thereof,
wherein:
A is A′ or A″,
A′ is: 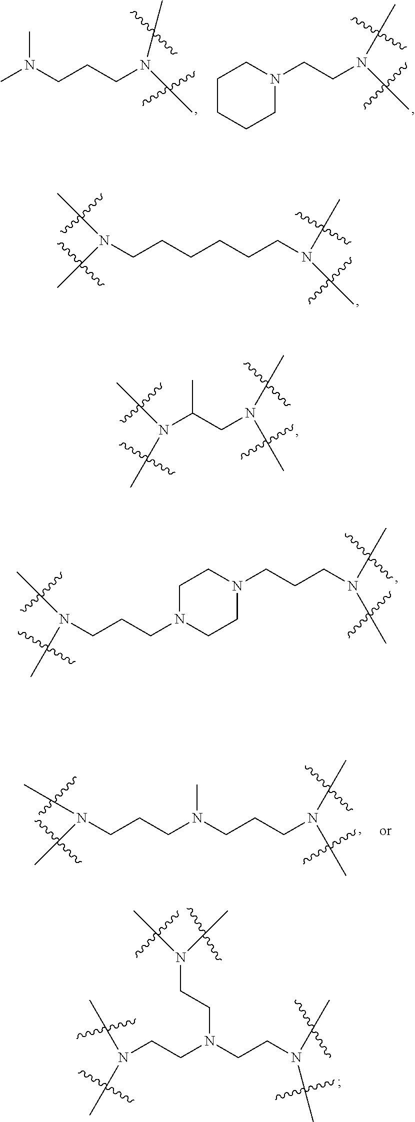
and 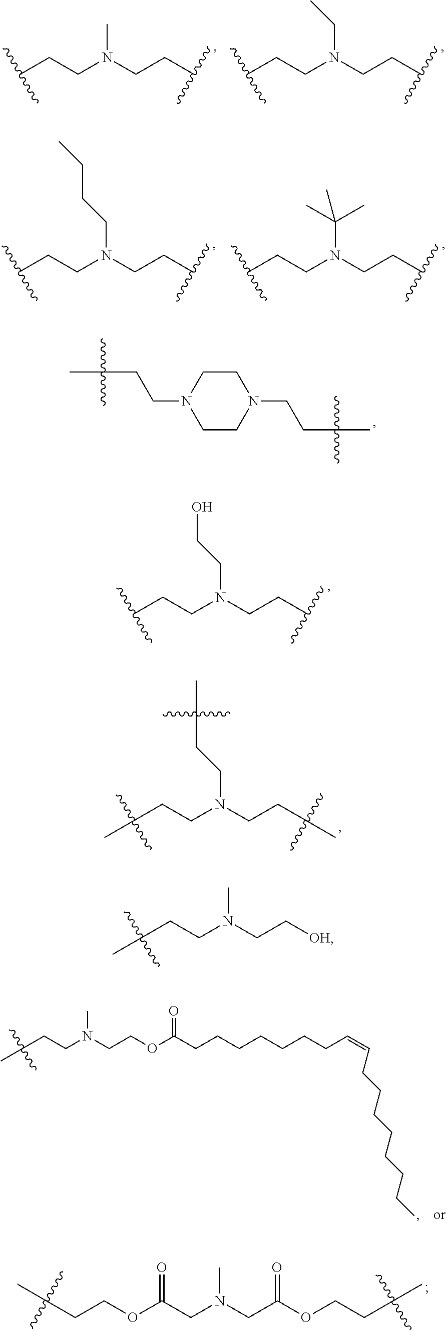
A″ is:
B is B′ or B″,
B′ is: 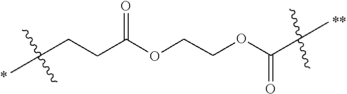 [SMILES: *CCC(=O)OCCOC(*)=O]
in which * indicates attachment to A, and ** indicates attachment to C:
B″ is: 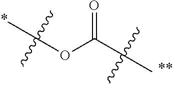 [SMILES: *OC(*)=O]
in which * indicates attachment to A, and ** indicates attachment to C;
wherein when A is A′, then Bis B′, and wherein when A is A″, then B is B″;
C is: 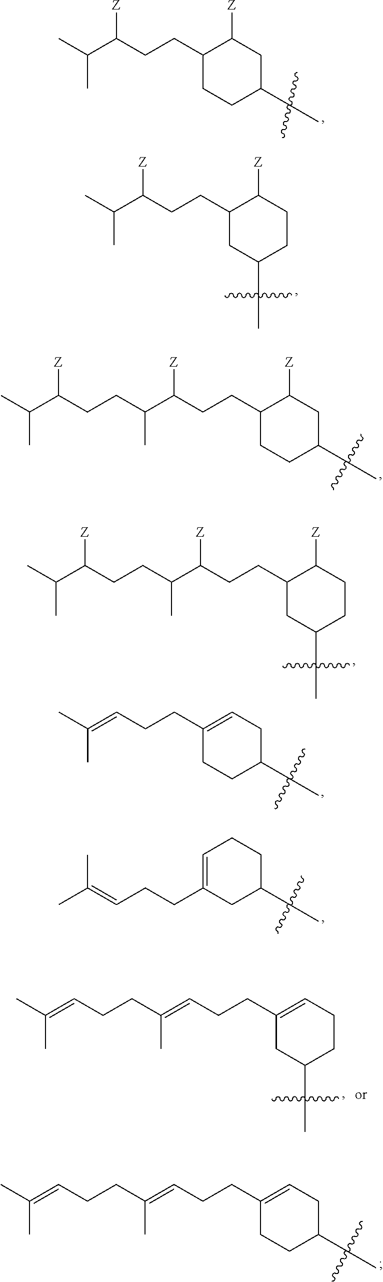
n is an integer between 1 to 6;
each Z is independently H or —S—R₁;
each R₁ is independently alkyl, 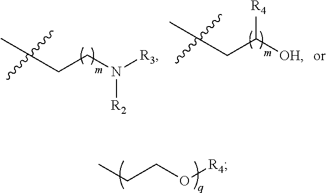
each R₂ is independently hydrogen, C₁-C₆ alkyl, C₁-C₆ alkenyl, aralkyl, or hydroxyalkyl;
each R₃ is independently hydrogen, C₁-C₆ alkyl, C₁-C₆ alkenyl, aralkyl, or hydroxyalkyl;
each R₄ is independently hydrogen or C₁-C₃ alkyl;
each R₅ is independently 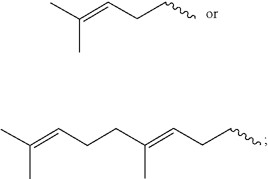
each R₆ is independently 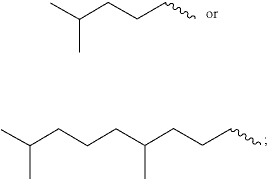
each m is an integer independently selected from 1-10; and
each q is an integer independently selected from 1-200,
wherein the at least one lipid nanoparticle further comprises at least one nucleic acid molecule.

2. The composition of claim 1, wherein the at least one lipid nanoparticle comprises about 10% to about 70% of the at least one compound of Formula (I), Formula (II) or Formula (III) by moles.

3. The composition of claim 2, wherein the at least one lipid nanoparticle comprises
i) about 20% to about 60%; or
ii) about 30% to about 50%
of the at least one compound of Formula (I), Formula (II) or Formula (III) by moles, preferably wherein the at least one lipid nanoparticle comprises about 20% to about 60% of the at least one compound of Formula (I), Formula (II) or Formula (III) by moles.

4. The composition of any one of the preceding claims, wherein the at least one lipid nanoparticle further comprises at least one structural lipid, at least one phospholipid, at least one PEGylated lipid, or any combination thereof.

5. The composition of claim 4, wherein the at least one structural lipid is cholesterol.

6. The composition of claim 4 or claim 5, wherein the at least one phospholipid is DOPE or DOPC.

7. The composition of any one of claims 4-6, wherein the PEGylated lipid is DMG-PEG2000.

8. The composition of any one of the preceding claims, wherein the at least one lipid nanoparticle has a combination of at least one compound of Formula (I), Formula (II) or Formula (III), at least one structural lipid, at least one phospholipid and at least one PEGylated lipid at molar percentages put forth in Table A.

9. The composition of any one of the preceding claims, wherein the at least one nucleic acid molecule comprises at least one RNA molecule and/or at least one DNA molecule, preferably wherein:
i) the at least one DNA molecule comprises a nucleic acid sequence encoding at least one transposon, preferably wherein the at least one transposon comprises a nucleic acid sequence encoding at least one therapeutic protein; and/or
ii) the at least one RNA molecule comprises:
a) a nucleic acid sequence encoding at least one transposase; or
b) a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof.

10. The composition of any one of the preceding claims, wherein the ratio of lipid to nucleic acid in the at least one nanoparticle is:
i) about 120:1 (w/w);
ii) about 100:1 (w/w);
iii) about 80:1 (w/w);
iv) about 60:1 (w/w); or
v) about 40:1 (w/w).

11. The composition of any of one of the preceding claims, wherein each C in the compound of Formula (I) is:

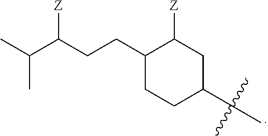

12. The composition of any of one of claims 1-10, wherein each C in the compound of Formula (I) is:

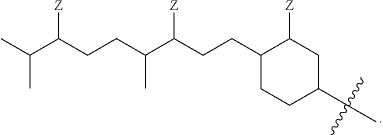

13. The composition of any of one of claims 1-10, wherein each C in the compound of Formula (I) is:

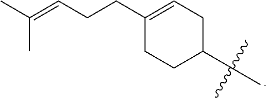 [SMILES: *C1CC=C(CCC=C(C)C)CC1]

14. The composition of any of one of claims 1-10, wherein each C in the compound of Formula (I) is:

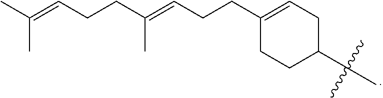 [SMILES: *C1CC=C(CC/C=C(\C)CCC=C(C)C)CC1]

15. The composition according to claim 11 or 12, wherein Z is H.

16. The composition according to claim 11 or 12, wherein Z is —S—R₁, and wherein at least one R₁ is alkyl.

17. The composition according to claim 16, wherein the at least one R₁ is C₁₀H₂₁.

18. The composition according to claim 16, wherein the least one R₁ is C₂H₄ (CH) (CH₃) 2.

19. The composition according to any of claims 1-18, wherein the A in the compound of Formula (I) is A′ and the B in the compound of Formula (I) is B′.

20. The composition according to any of claims 1-18, wherein the A in the compound of Formula (I) is A′ and A′ is

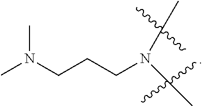 [SMILES: *N(*)CCCN(C)C]

21. The composition according to any of claims 1-18, wherein the A in the compound of Formula (I) is A′ and A′ is

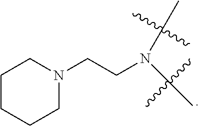 [SMILES: *N(*)CCN1CCCCC1]

22. The composition according to any of claims 1-18, wherein the A in the compound of Formula (I) is A′ and A′ is

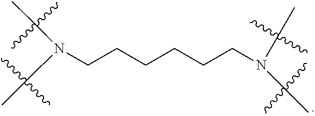 [SMILES: *N(*)CCCCCCN(*)*]

23. The composition according to any of claims 1-18, wherein the A in the compound of Formula (I) is A′ and A′ is

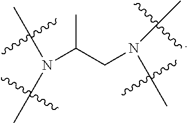 [SMILES: *N(*)CC(C)N(*)*]

24. The composition according to any of claims 1-18, wherein the A in the compound of Formula (I) is A′ and A′ is

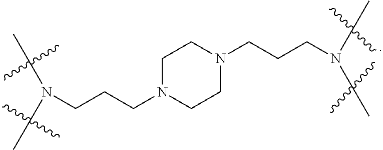 [SMILES: *N(*)CCCN1CCN(CCCN(*)*)CC1]

25. The composition according to any of claims 1-18, wherein the A in the compound of Formula (I) is A′ and A′ is

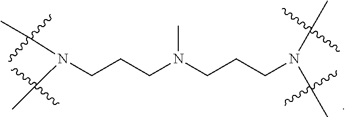 [SMILES: *N(*)CCCN(C)CCCN(*)*]

26. The composition according to any of claims 1-10, wherein the A in the compound of Formula (I) is A′, A′ is 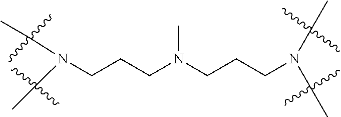 [SMILES: *N(*)CCCN(C)CCCN(*)*]
and B is B′.

27. The composition of claim 26, wherein C is 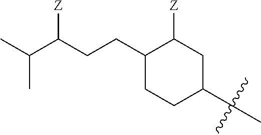
and each Z is independently H.

28. The composition of claim 27, wherein n is 4.

29. The composition according to any of claims 1-18, wherein the A in the compound of Formula (I) is A′ and A′ is

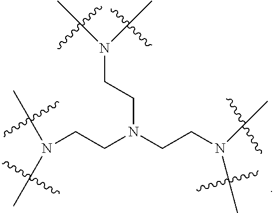 [SMILES: *N(*)CCN(CCN(*)*)CCN(*)*]

30. The composition according to any of claims 1-18, wherein the A in the compound of Formula (I) is A″ and B is B″.

31. The composition according to any of claims 1-18, wherein the A in the compound of Formula (I) is A″ and A″ is

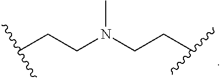 [SMILES: *CCN(C)CC*]

32. The composition according to any of claims 1-18, wherein the A in the compound of Formula (I) is A″ and A″ is

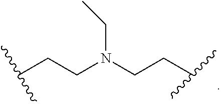 [SMILES: *CCN(CC)CC*]

33. The composition according to any of claims 1-18, wherein the A in the compound of Formula (I) is A″ and A″ is

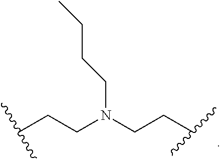 [SMILES: *CCN(CC*)CCCC]

34. The composition according to any of claims 1-18, wherein the A in the compound of Formula (I) is A″ and A″ is

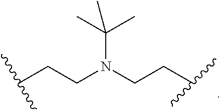 [SMILES: *CCN(CC*)C(C)(C)C]

35. The composition according to any of claims 1-18, wherein the A in the compound of Formula (I) is A″ and A″ is

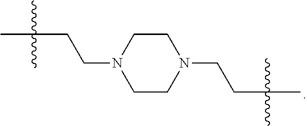 [SMILES: *CCN1CCN(CC*)CC1]

36. The composition according to any of claims 1-18, wherein the A in the compound of Formula (I) is A″ and A″ is

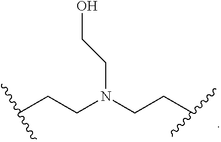 [SMILES: *CCN(CC*)CCO]

37. The composition according to any of claims 1-18, wherein the A in the compound of Formula (I) is A″ and A″ is

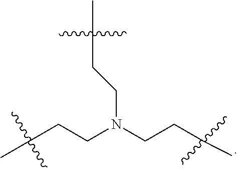 [SMILES: *CCN(CC*)CC*]

38. The composition according to any of claims 1-18, wherein the A in the compound of Formula (I) is A″ and A″ is

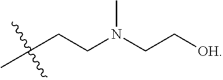 [SMILES: *CCN(C)CCO]

39. The composition according to any of claims 1-18, wherein the A in the compound of Formula (I) is A″ and A″ is

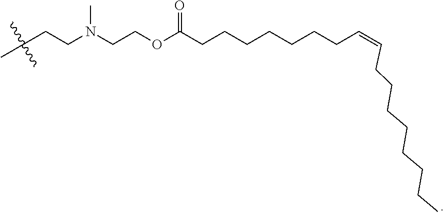 [SMILES: *CCN(C)CCOC(=O)CCCCCCC/C=C\CCCCCCCC]

40. The composition according to any of claims 1-18, wherein the A in the compound of Formula (I) is A″ and A″ is

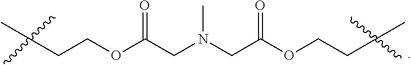 [SMILES: *CCOC(=O)CN(C)CC(=O)OCC*]

41. The composition of any of one of claims 1-10, wherein the compound of Formula (I) is:

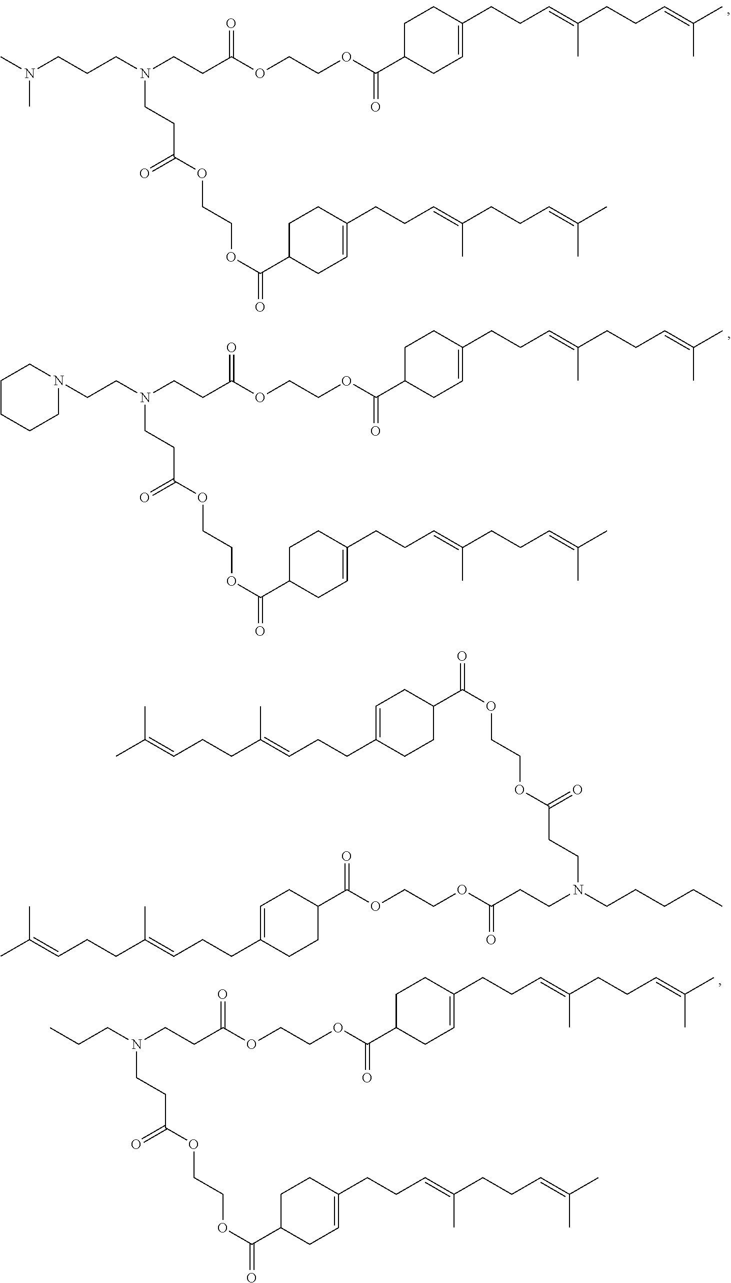

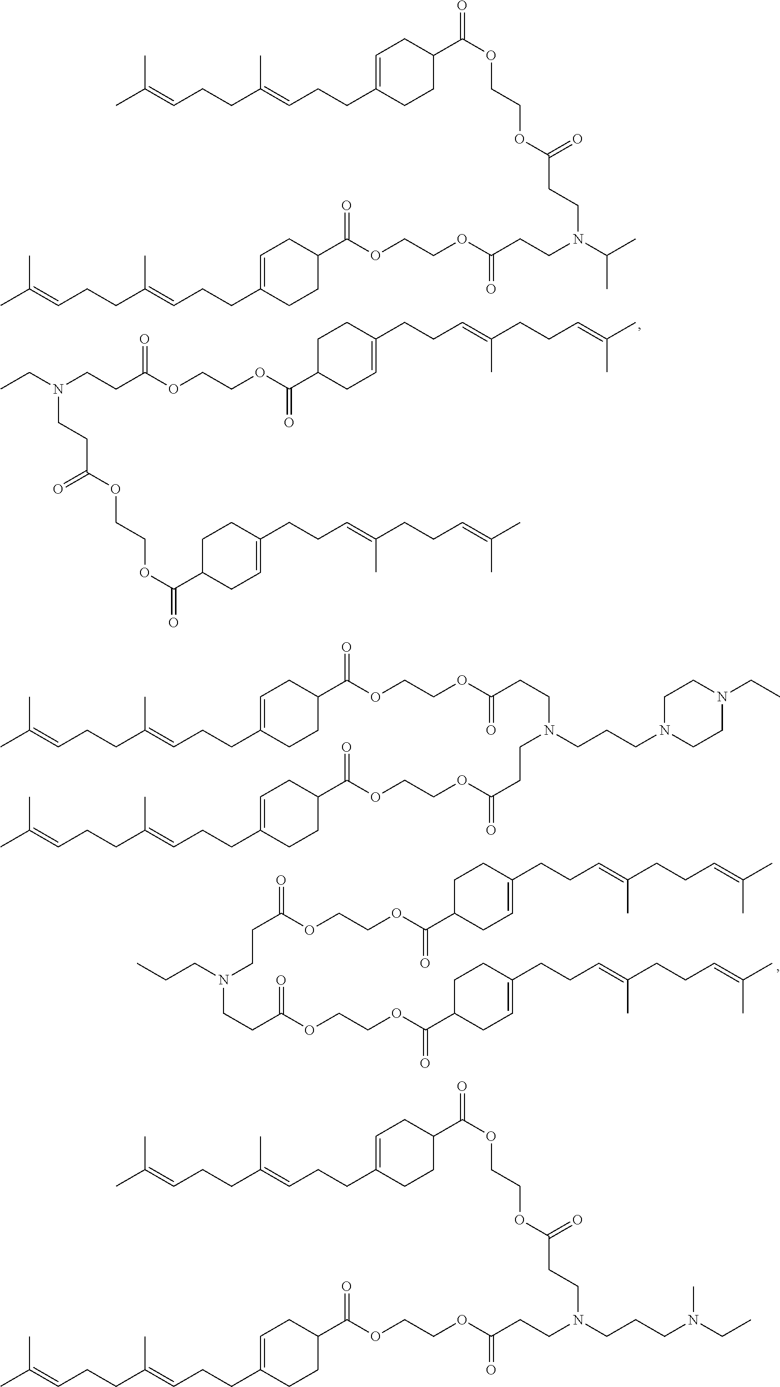

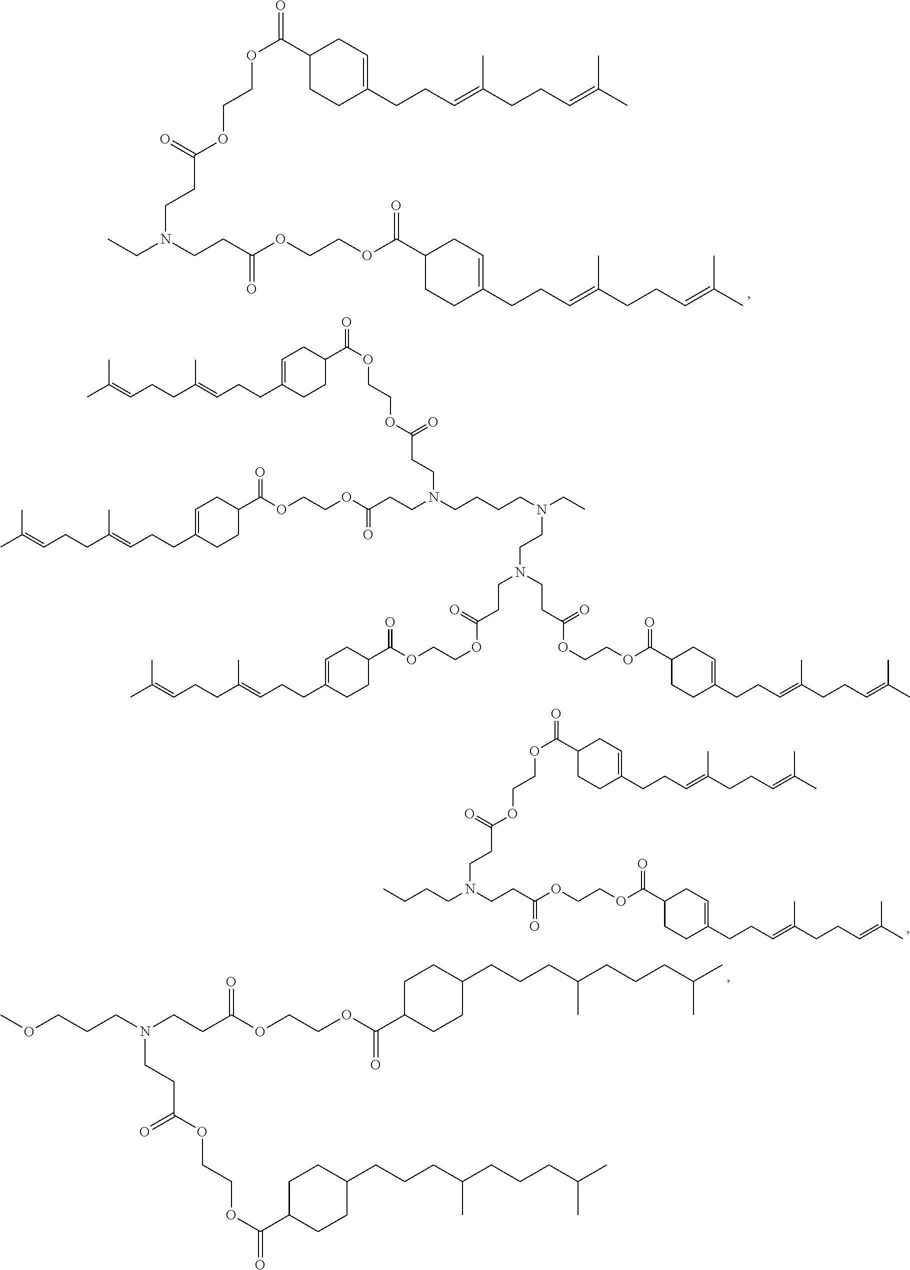

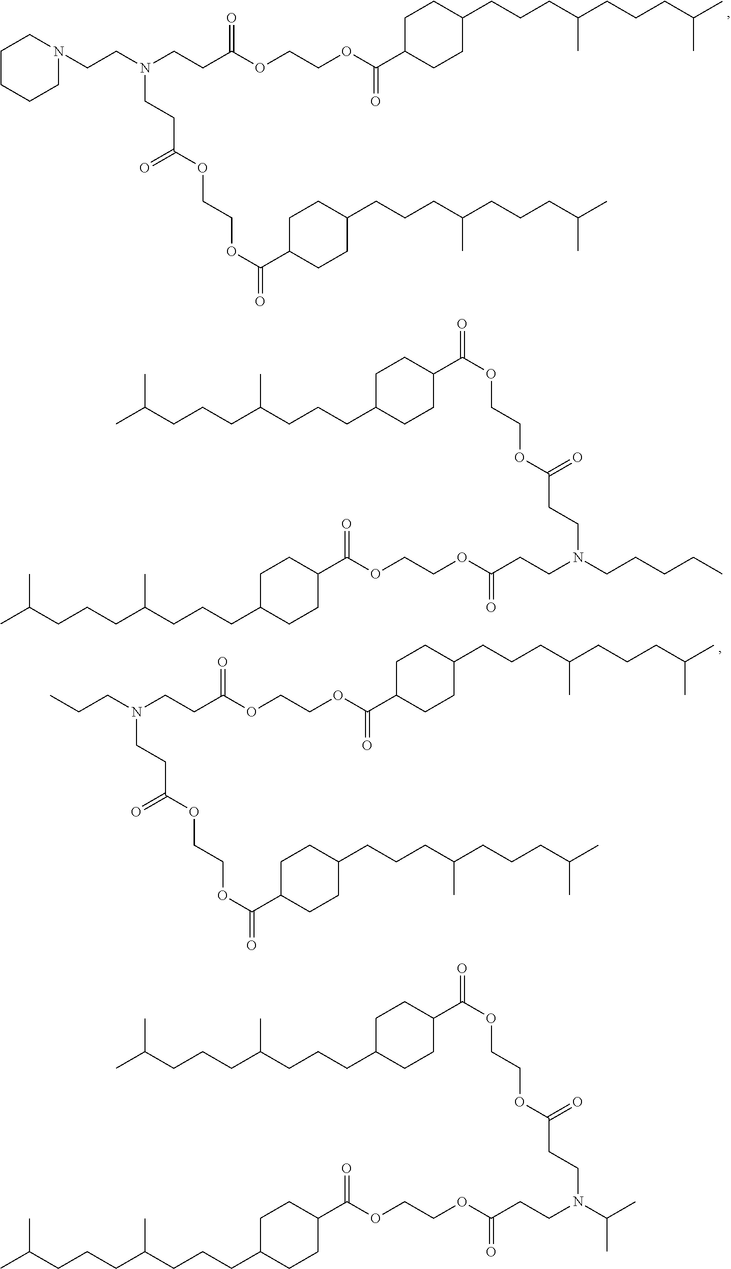

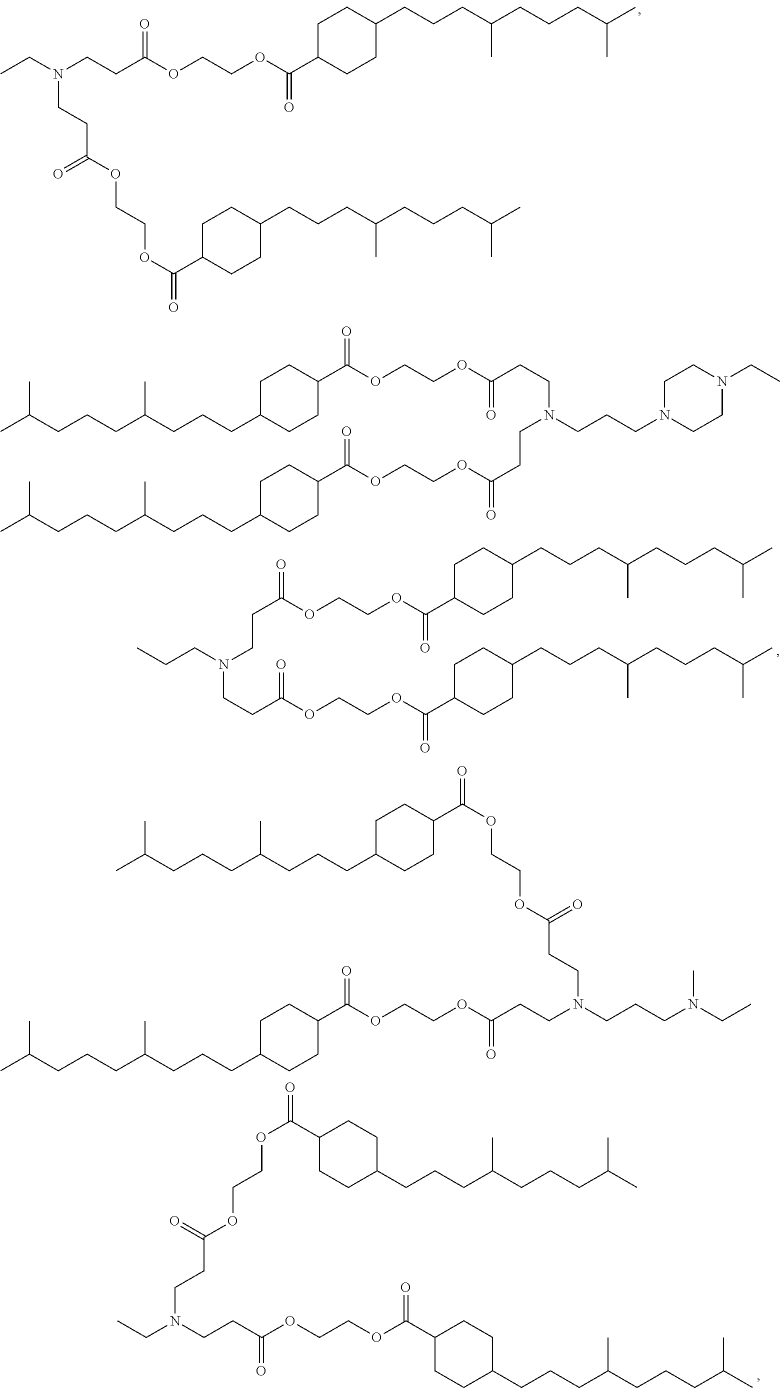

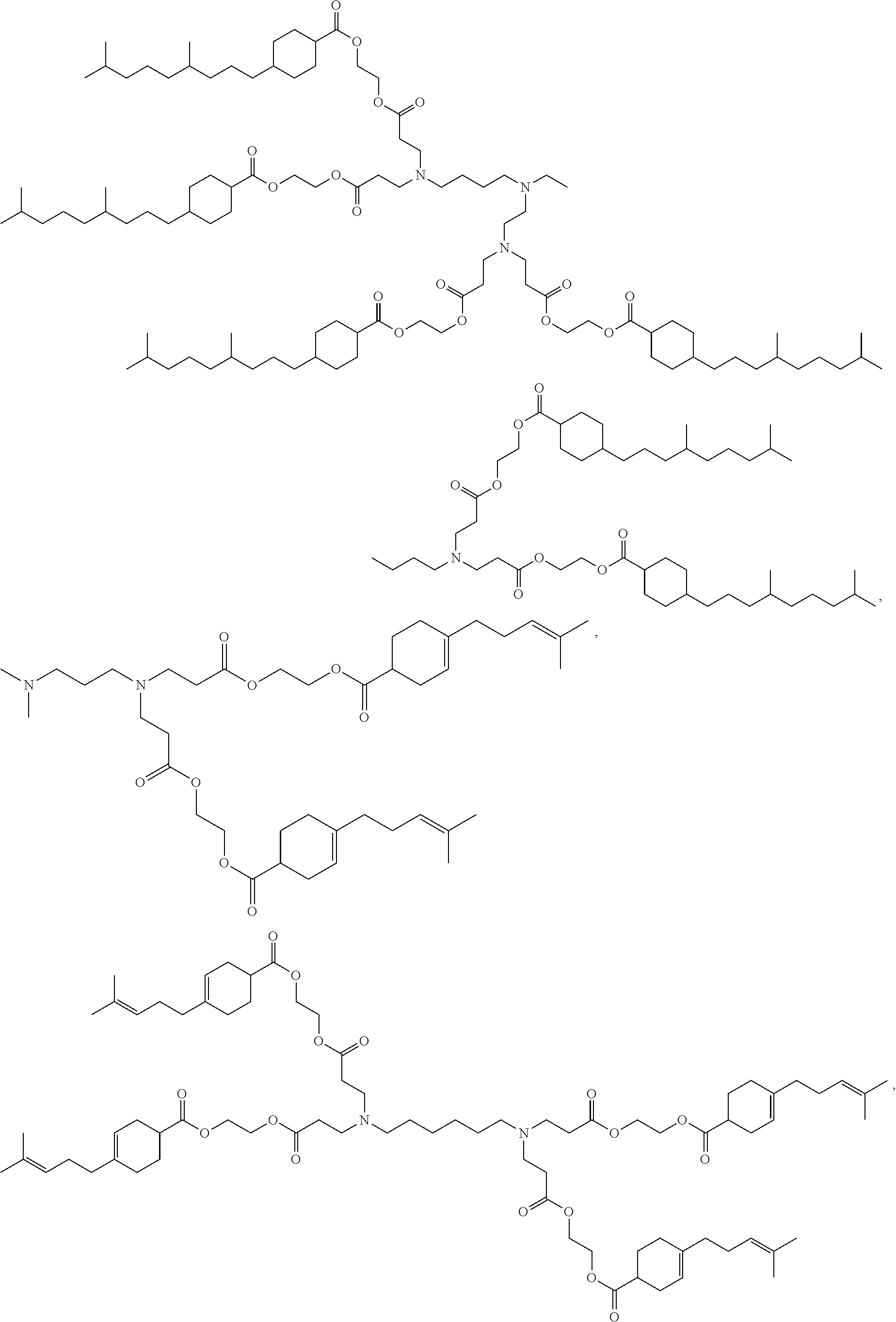

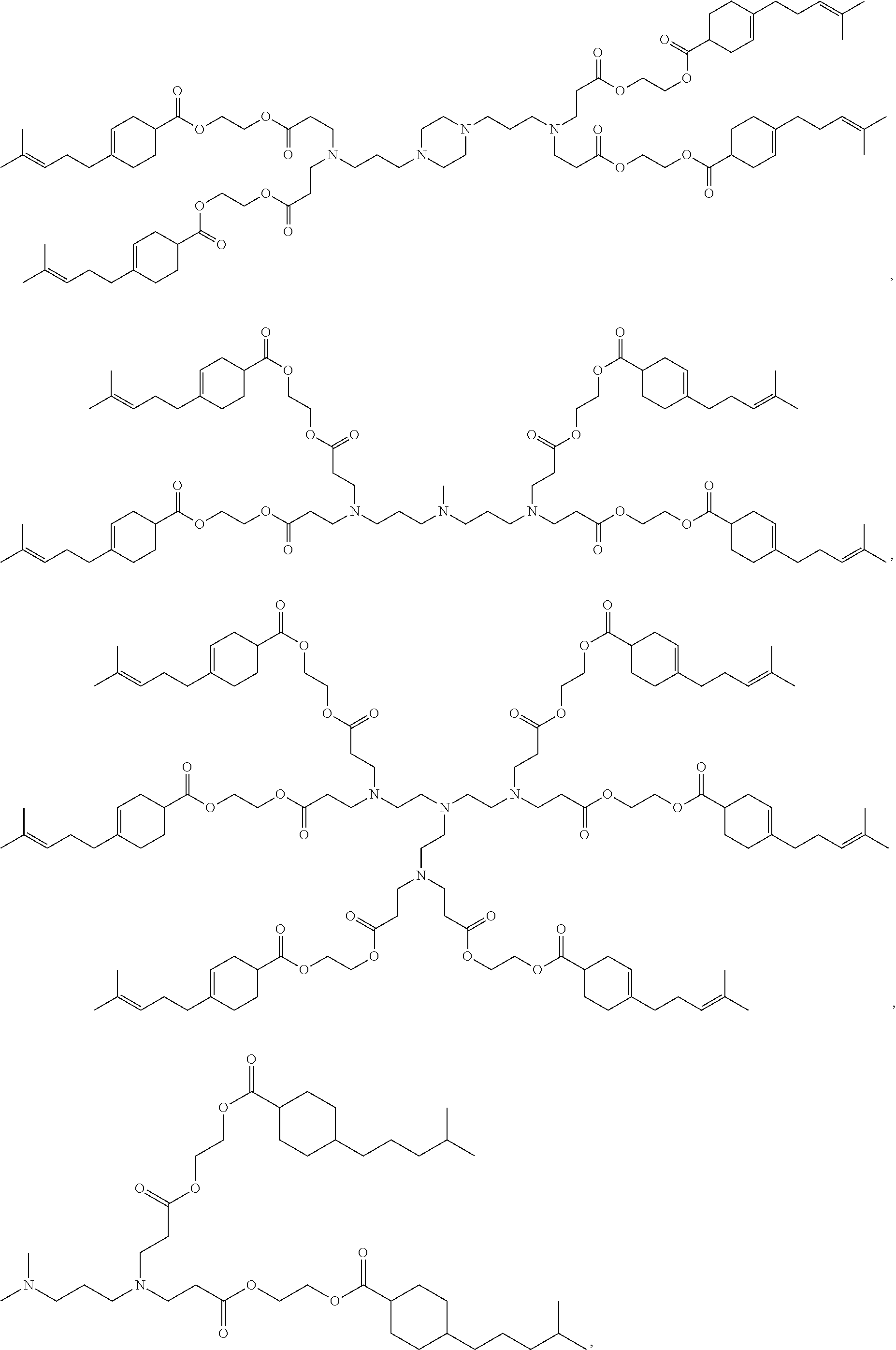

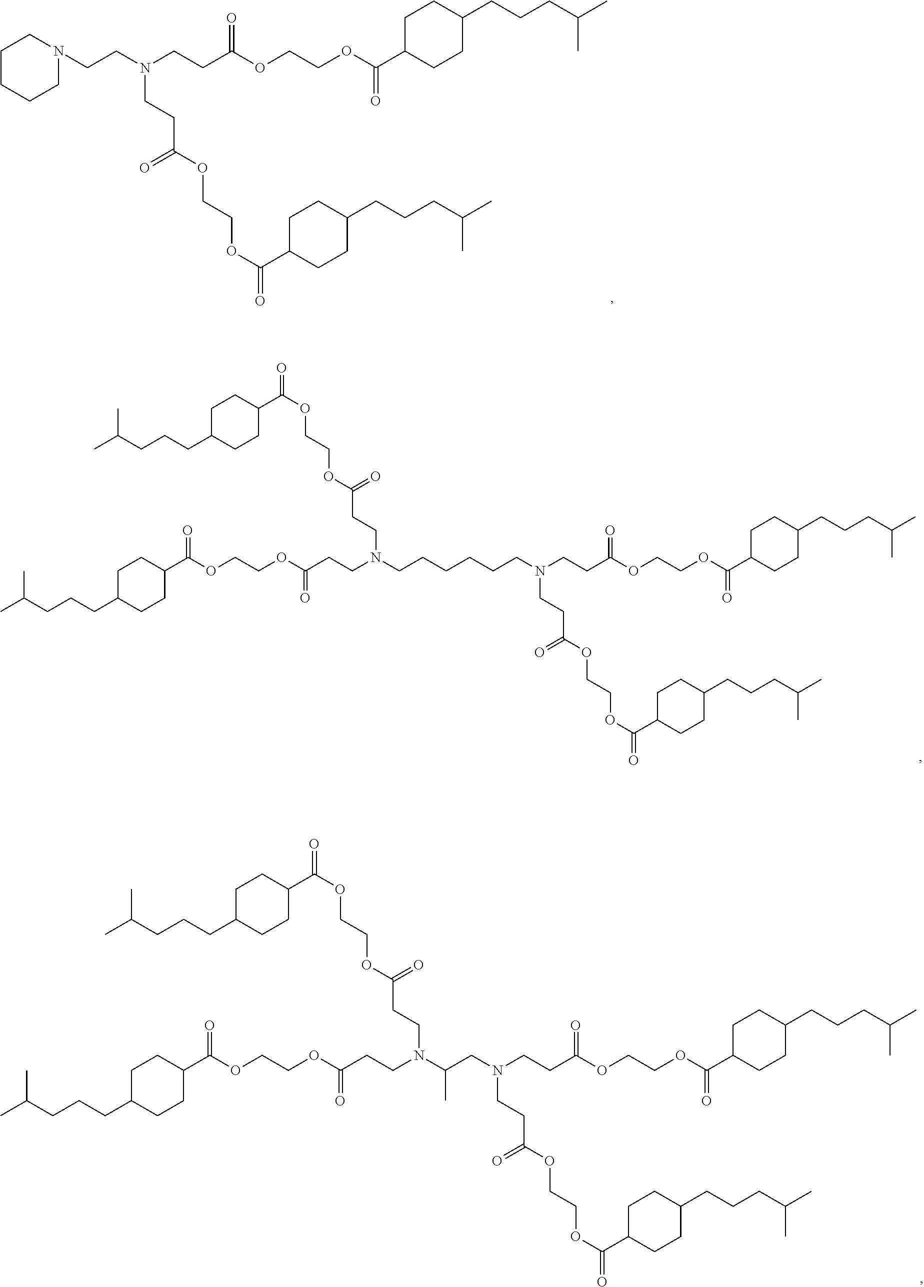

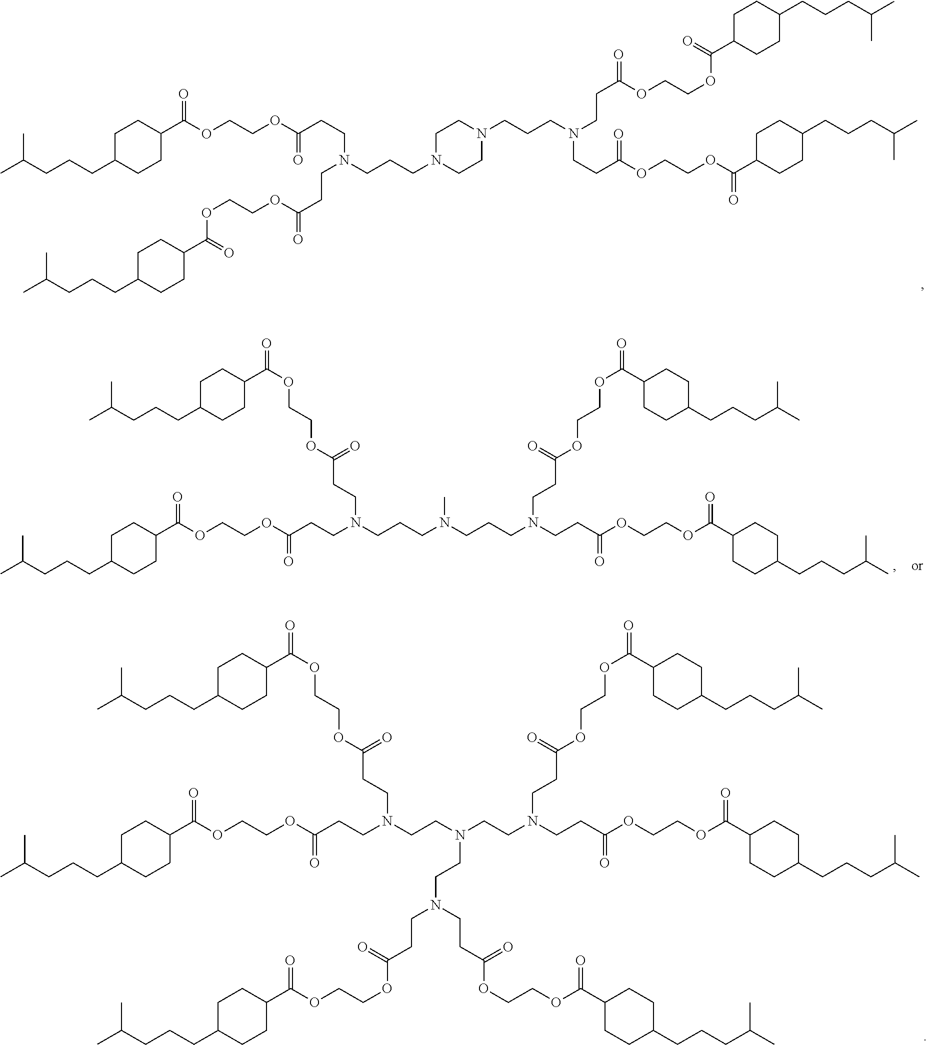

42. The composition of any of claims 1-10, wherein the compound of Formula (I) is:

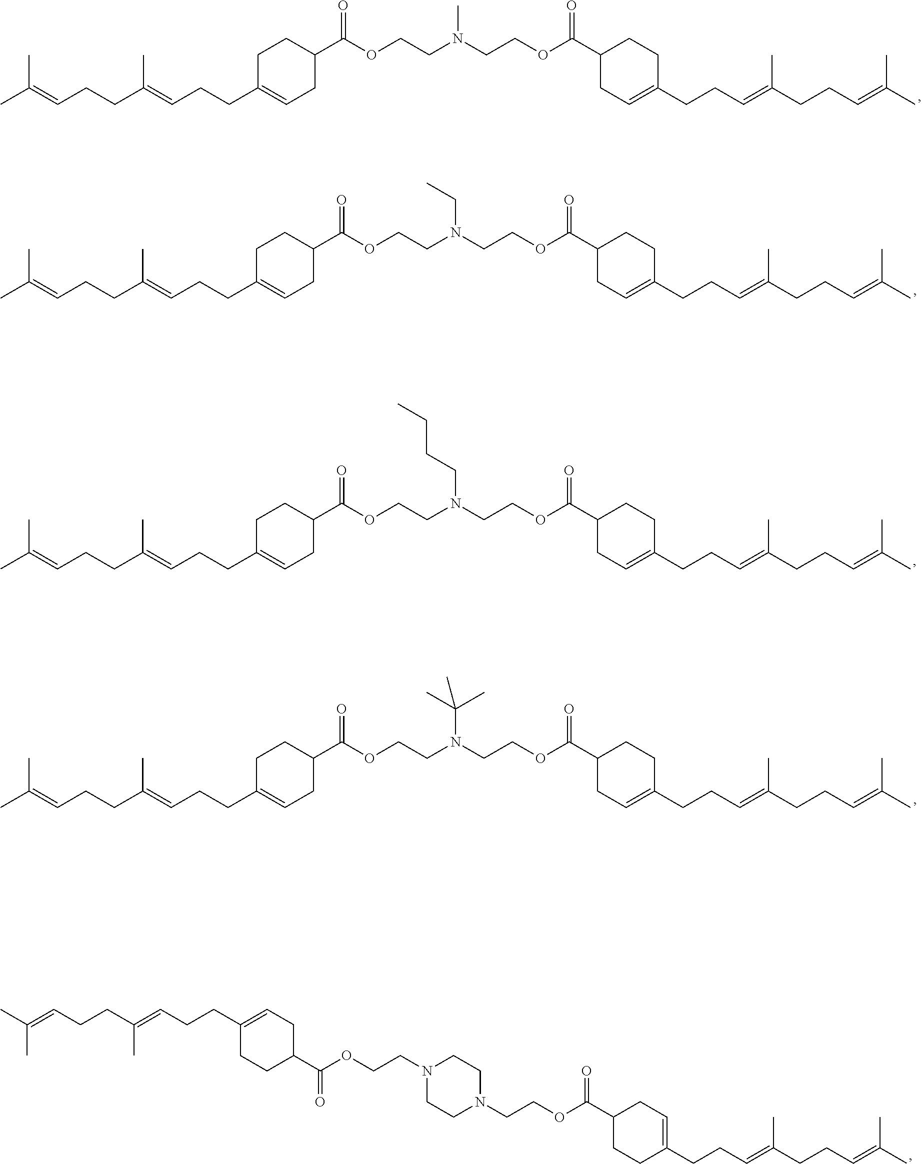

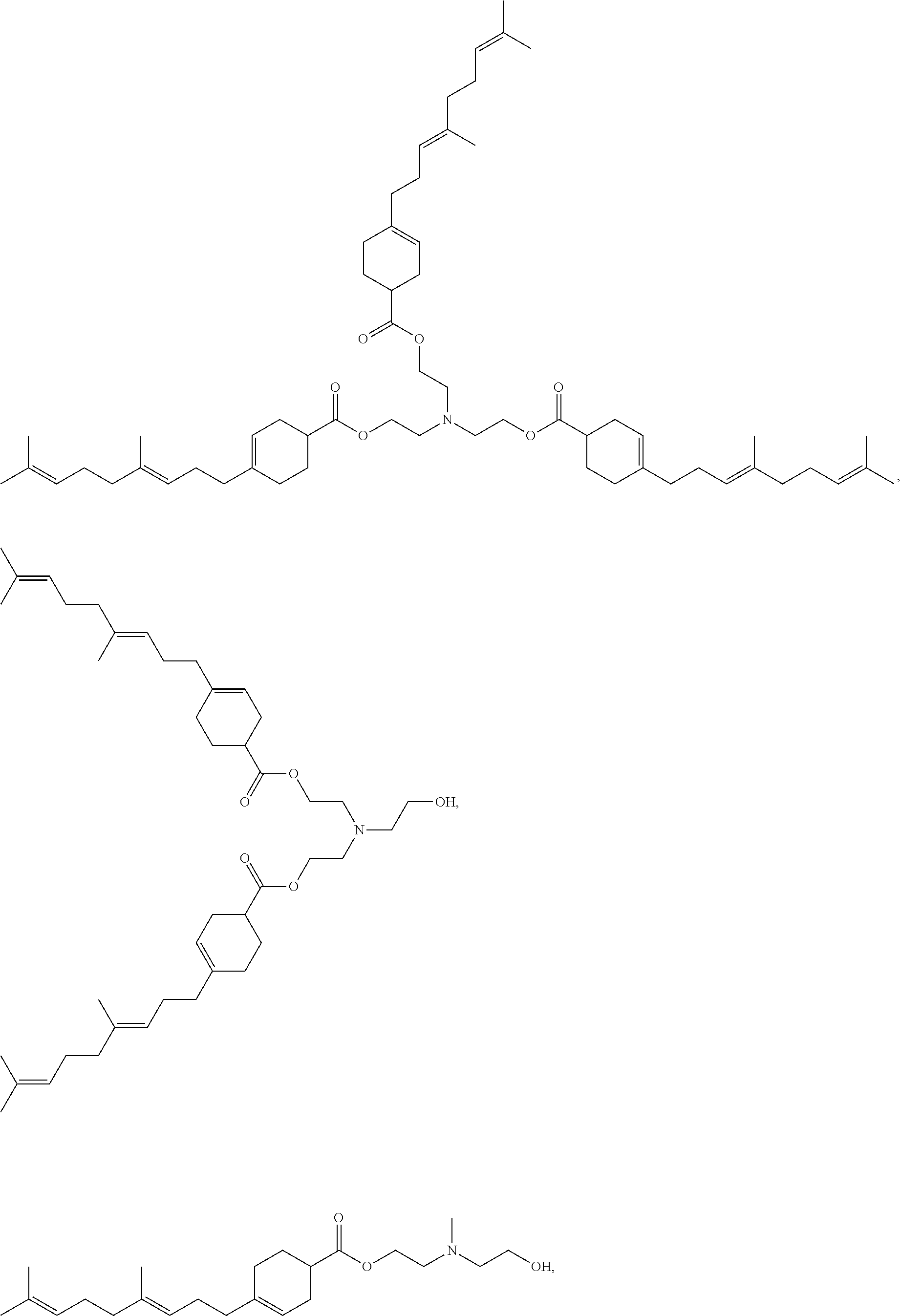

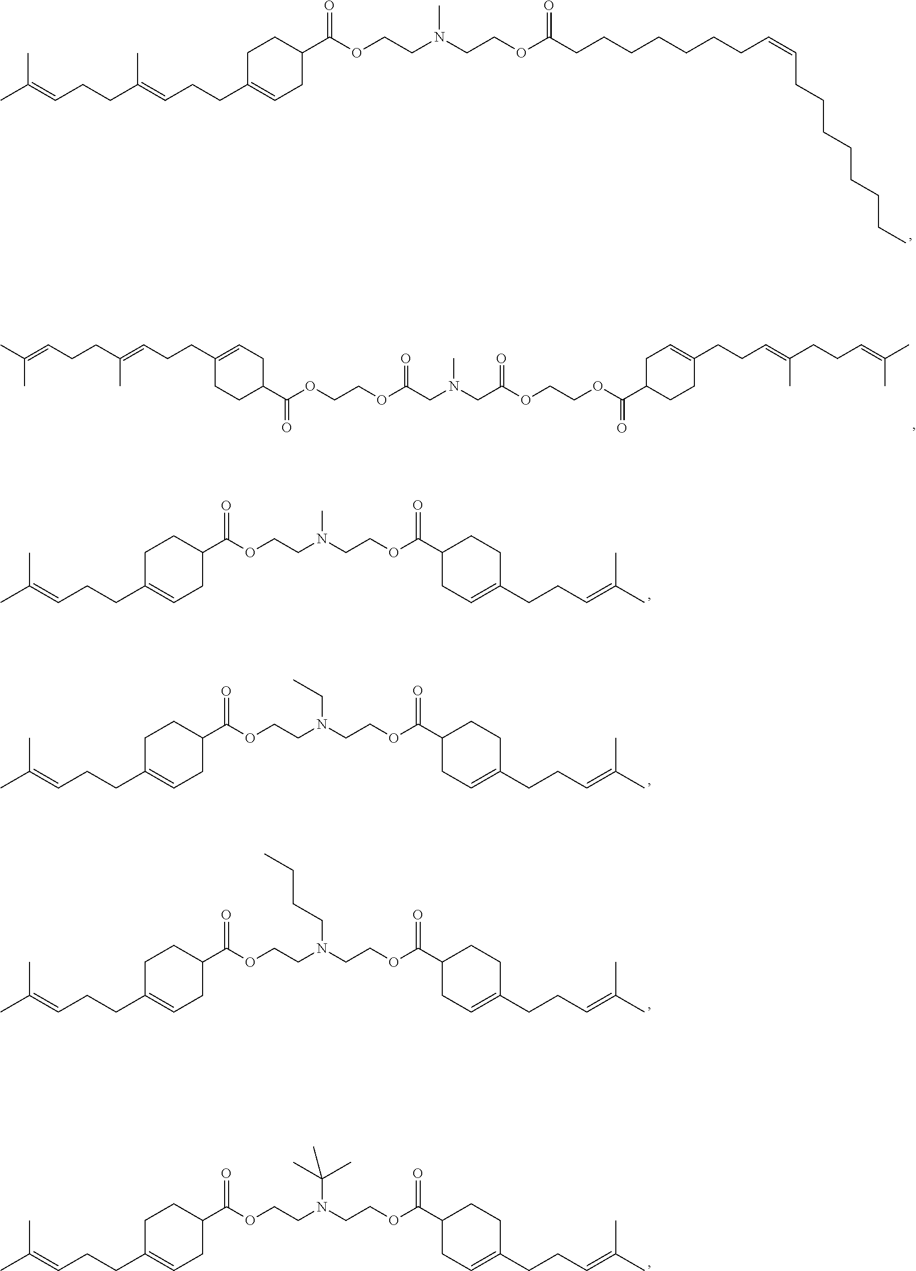

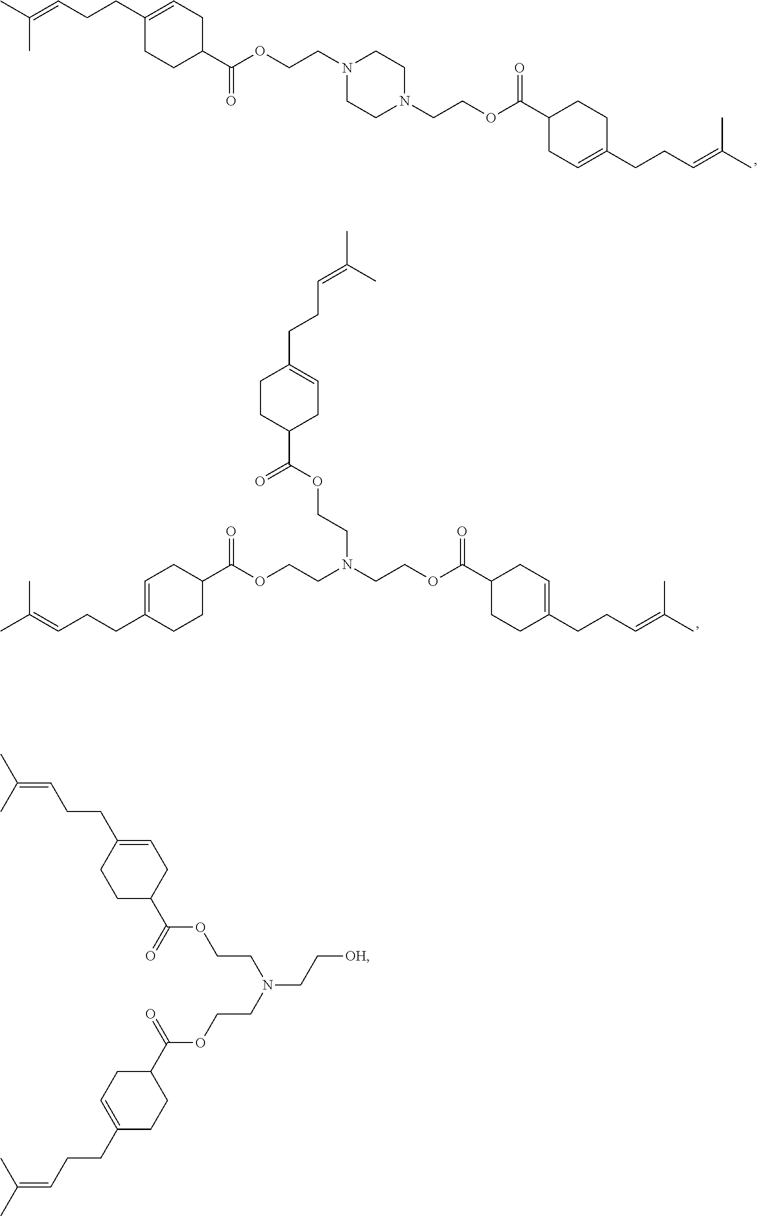

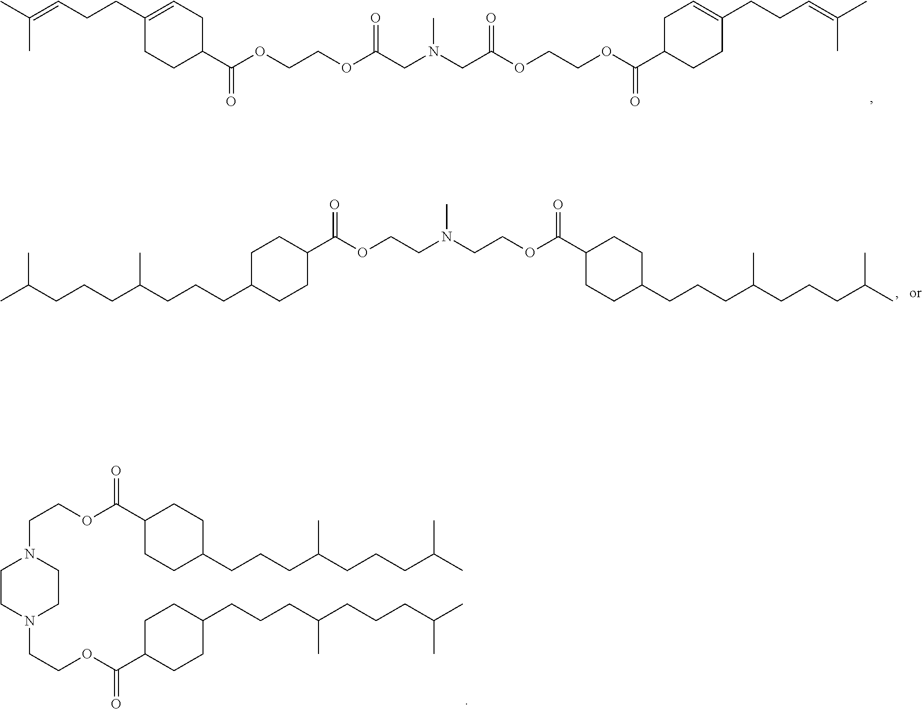

43. The composition of any one of claims 1-10, wherein the compound of Formula (II) or Formula (III) is:

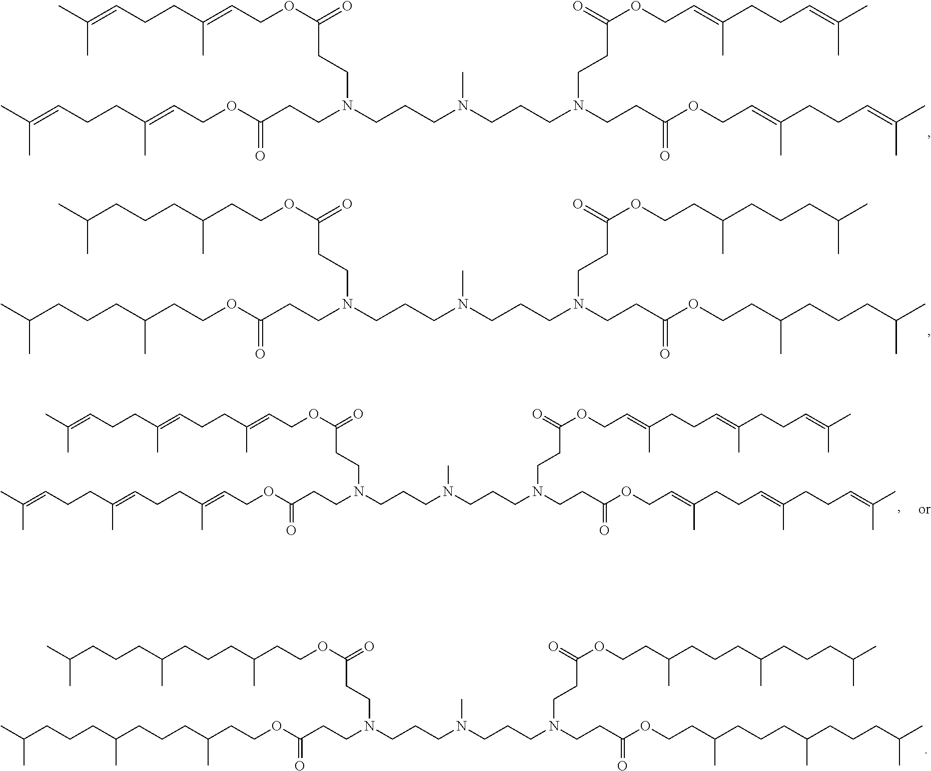

44. The composition of any one of claims 1-10, wherein the at least one compound of Formula (I) is Compound 30.

45. A pharmaceutical composition, comprising a composition of any of the preceding claims and at least one pharmaceutically-acceptable excipient or diluent.

46. A method of delivering at least one nucleic acid to at least one cell comprising contacting the at least one cell with at least one composition of any of the preceding claims.

47. A method of genetically modifying at least one cell comprising contacting the at least one cell with at least one composition of any of the preceding claims, preferably wherein the at least one cell is
(a) a liver cell, preferably wherein the liver cell is a hepatocyte, a hepatic stellate cell, Kupffer cell or liver sinusoidal endothelial cell;
(b) a T-cell, preferably wherein the T-cell is an activated T-cell, a resting T-cell or a stem memory T cell (T_{SCM} cell);
(c) is a hematopoietic stem cell (HSC).

48. At least one cell modified according to the method claim 46 or claim 47.

49. A method of treating at least one disease or disorder in a subject in need thereof comprising administering to the subject at least one therapeutically effective amount of the composition of any one of claims 1-45 or the at least one cell of claim 48, preferably wherein:
i) the at least one disease or disorder is a liver disease or disorder, preferably wherein the liver disease or disorder is:
(a) a metabolic liver disorder;
(b) a urea cycle disorder (UCD), preferably wherein the UCD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome) or any combination thereof;
ii) the at least one disease or disorder is cancer; or
iii) the at least one disease or disorder is hemophilia A.


SEQUENCE LISTING (15 sequences)

SEQ ID NO: 1 - RNA, 100 nt, synthetic construct
  source 1..100: mol_type = other RNA
cctgggcaccagcgccggtggttttagagctagaaatagcaagttaaaataaggctagtccgttatcaacttgaaaaagtggcaccgagtcggtgctttt

SEQ ID NO: 2 - RNA, 100 nt, synthetic construct
  source 1..100: mol_type = other RNA
gatgctcgccctcccgtcccgttttagagctagaaatagcaagttaaaataaggctagtccgttatcaacttgaaaaagtggcaccgagtcggtgctttt

SEQ ID NO: 3 - protein, 1369 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 4 - protein, 199 aa, unidentified
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 5 - protein, 1591 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 6 - DNA, 7975 bp, synthetic construct
  source 1..7975: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgagctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctggtctcatgacgcgtagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 7 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 8 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 9 - protein, 593 aa, synthetic construct
GSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 10 - protein, 593 aa, synthetic construct
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 11 - RNA, 20 nt, synthetic construct
  source 1..20: mol_type = other RNA
gatgaccattggtattggag

SEQ ID NO: 12 - RNA, 20 nt, synthetic construct
  source 1..20: mol_type = other RNA
gaataacatgcagactaagc

SEQ ID NO: 13 - RNA, 20 nt, synthetic construct
  source 1..20: mol_type = other RNA
ggccacggagcgagacatct

SEQ ID NO: 14 - RNA, 20 nt, synthetic construct
  source 1..20: mol_type = other RNA
actctctctttctggcctgg

SEQ ID NO: 15 - DNA, 4731 bp, synthetic construct
  source 1..4731: mol_type = other DNA
tgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtatgtacagcggccgccctctccaataccaatggtcatccctaaaacatacgtatgaataacatgcagactaagcaggagagtagtccagggtttccgagggtttccgtcgactcagctcgtctcgagggcgtactaactgggccctttcccttttttttcctcagatgggttctggagctacaaacttcagcctcctcaagcaagctggagacgtggaagagaacccaggacctgaagacgctaagaacatcaagaagggacccgctcccttttaccctctggaggatgggaccgccggagaacagctccataaggctatgaaacggtatgcattggttcccggggctatagccttcaccgatgctcatattcaagtagacgtaacctatgcagagtacttcgagatgtcagtccggctcgcagaagcaatgcggcgatacggtttgaacacaaatcaccgcatagtcgtttgttcagaaaactcctctcaattttttatgcccgtactcggagcactctttattggtgttgcagtggcccccgctaacgacatatacaacgagagggagttgctgaactcaatgggaattagccaaccaacagtggtattcgtttcaaagaagggactgcgcaaagttcttaacgtacagaagaaattgcccataatccgaaaaatcattattatggactccaaaaccgactatcaggggtttcagagcatgtataccttcgtcacctctcaccttcctccctcattcaacgagtatgattttgttcccgaaagttttgaccgcgacaaaaccattgcactgatcatgaatagtagtgggagcacagggcttccaaaaggagttgctttgcctcaccgaacagcttgcgtgcggttcagccatgctagagaccctatctttggctaccagaacattcccgatacagccatcctcagtgtagtcccattccaccatggtttcggaatgtttactacactcggatatttgatttgcgggtttcgcgtcgtgctcatgtatcgcttcgaagaggagttgtttctccgctccctgcaagattacaaaattcaatctgccttgctggtgcctactcttttcagttgtctggccaagtcaacccttatcgacaagtacgatctctcatcattgcgggaaatagcctctggcggcgcaccactgtcaaaagaagtaggtgaggcagtagcaaagcgctttagattgccaggcatcaggcagggttatggtctgacagaaacaaccaatgctgttatgattactccagagggcgaccggaagccaggttctgtgggtaaagtagtccctttcttcgaggcaaaggtagtcgacttggtgacagggaagaccctcggcgtgaaccagcggggggaattgtgtgtacggggtcctatgatcatgagtggctatgtcaacaatcctgaagcaaccaatgctctcatcgataaggatggctggttgcatagtggtgatattgcttactgggatgaggacgagcacttttttattgttgataggctcaaatccctgatcaaatacaagggataccaggtggccccagcagaacttgagggaatactcctccagcacccatacattttcgatgctggagttgccggtttgccagacgatgatgcaggagagcttccagctgcagtggttgttcttgaacacggaaaaactatgacagagaaggagatcgtagattatgtagctagccaggtgactacagccaagaagctcagaggaggagtcgtattcgtagatgaagtaccacggggatcaacaggcaaactggatgcacggaaaatcagagagattttgaccaaagccaagaaggatggaaaaattgccgtgtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggccgaaccggccgatatcaagcttcgaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaattctggcccgcattaccggtcatcctgcagcctacacggcgatctttccgtccttcttggccttagtgagaatctcgcggatcttgcgggcgtctaacttgccggtcgatcctctaggtacctcatccacaaacacaacaccaccgcgcagcttcttggcggttgtaacctggctggccacatagtccacgatctctttctcggtcatggttttaccgtgttccaacacgacgactgcggcgggcagctcgccggcatcgtcgtcgggcaggccggcgactccggcgtcgaagatgtaggggtgttgcagcaggatgccctccagttcggctggggctacctggtagcccttgtatttgatcagggacttcagccggtccacgatgaagaagtgctcgtcctcgtcccagtaggcgatgtcgccgctgtgcagccagccgtccttgtcgatgagagcgttcgtagcctcggggttgttaacgtagccgctcatgatcatggggccacggacgcacagctcaccgcgctggttcacacccagtgtcttacccgtgaccaagtctaccaccttagcctcgaagaagggcaccaccttgcctactgagccaggcttacggtccccctcgggggtgatcatgacggcgttggttgtttctgtcaggccatagccctggcggatgcctggtaggcggaagcgtttggccacggcctcacctacctccttgctgagcggcgccccgccgctggcgatctcgcgcaagctgcttaggtcgtacttgtcgatgagagtgctcttagcgaggcagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgttctggtagccgaagatggggtcgcgggcatgactgaatcggacacaagcggtgcggtgcggtagggctacgcccttgggtaatcctgtactaccactactgttcatgatcagggcgatggttttgtcccggtcgaagctctcgggcacgaagtcgtactcgttgaagctgggtggcaaatgggaagtcacgaaggtgtacatgctttggaagccctggtagtcggtcttgctatccatgatgatgatctttcgtatgatcggtagcttcttttgcacgttgaggacctttcgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcgagctattctcgctgcacaccacgatccgatggtttgtattcagcccatagcgcctcatagcttctgccagccgaacgctcatctcgaagtactcggcgtaggtaacgtccacctgaatatgtgcgtcggtaaaggcgatggcgccgggcaccagggcgtagcgcttcatggctttgtgcagctgctcgccggcggtcccgtcttcgagtgggtagaacggcgctgggcccttcttaatgtttttggcgtcttcgggtccgggattctcctcaacatccccgcatgtaagaagactccccctgccctcgccggagcccatgtcaaaggcatcctgcagttgggggagaggagggaagaaaaagaaagagtattagagagagagcagagaatgccaccctgaagagccctctccaataccaatggtcatccctaaaacatacgtatgaataacatgcagactaagcagggcggccgcagctagccttaagggcgcagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgt